(19) United States
(12) Patent Application Publication
(10) Pub. No.: US 2026/0250688 A1
(43) Pub. Date: Aug. 27, 2026

(54) TRANSLATION ENHANCING NUCLEIC ACID COMPOUNDS FOR PKD1 AND USES THEREOF

(71) Applicant: Arnatar Therapeutics, Inc., San Diego, CA (US)

(72) Inventors: Xuehai Liang, San Siego, CA (US); Lingdi Zhang, San Diego, CA (US); Derek Steiner, San Diego, CA (US); Hengbo Zhou, San Diego, CA (US)

(21) Appl. No.: 19/406,818

(22) Filed: Dec. 2, 2025

Related U.S. Application Data
(63) Continuation of application No. PCT/US25/57339, filed on Nov. 26, 2025.
(60) Provisional application No. 63/725,440, filed on Nov. 26, 2024.
(60) Provisional application No. 63/727,491, filed on Dec. 3, 2024.
(60) Provisional application No. 63/817,917, filed on Jun. 4, 2025.
(60) Provisional application No. 63/901,403, filed on Oct. 17, 2025.

(51) Int. Cl. C12N 15/113; A61K 31/7115; A61K 31/712; A61K 31/7125; A61P 13/12

(52) U.S. Cl. CPC C12N 15/1138; A61K 31/7115; A61K 31/712; A61K 31/7125; A61P 13/12; C12N 2310/113; C12N 2310/315; C12N 2310/321; C12N 2310/3231; C12N 2310/3341; C12N 2310/3515; C12N 2310/3519; C12N 2320/30

(57) ABSTRACT

Disclosed herein are methods and compounds for enhancing PKD1 protein expression by compounds targeting PKD1 mRNA. Such methods and compounds are useful for increasing expression of in PKD1 mutation patients for the treatment of a haploinsufficiency and/or loss of function disease, disorder and/or condition such as Autosomal Dominant Polycystic Kidney Disease (ADPKD) and/or Polycystic Liver Disease (PLD).

### CROSS-REFERENCE TO RELATED APPLICATIONS

This subject application claims priority under 35 U.S.C. § 111(A) to PCT Application No. PCT/US25/57339 filed Nov. 26, 2025, which application claims the benefit of U.S. Provisional Application Nos. 63/725,440, filed Nov. 26, 2024; 63/727,491, filed December 3,2024; 63/817,917, filed June 4,2025; and 63/901,403, filed Oct. 17, 2025, the entire contents of which are incorporated herein in their entireties by this reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: the text file named “2025_11_26_Seq_List_PKD1”(617,224 Bytes), which was created on Nov. 25, 2025.

Throughout this application various publications are referenced. All publications, gene transcript identifiers, patents, and patent applications mentioned in this specification are herein incorporated by reference to the same extent as if each individual publication, gene transcript identifiers, patent, or patent application was specifically and individually indicated to be incorporated by reference.

### FIELD

Certain embodiments are directed to nucleic acid compounds for enhancing PKD1 gene expression and methods of using the compounds. Such methods and compounds are useful for increasing expression of PKD1 (also known as Polycistin-1 (PC1)) protein which is associated with Autosomal Dominant Polycystic Kidney Disease (ADPKD).

### BACKGROUND

Autosomal Dominant Polycystic Kidney Disease (ADPKD) is a rare disease with a prevalence of around 1:1000, affecting approximately 12 million people worldwide (P. Igarashi P. et al., 2002, J Am Soc Nephrol, 13 (9): 2384-2398). ADPKD is the most commonly inherited cause of end-stage kidney disease (ESKD). It's a systemic disorder that causes fluid-filled cysts to form in the kidneys and can also lead to cyst formation in other organs like the liver and pancreas. About 7 in 10 patients with ADPKD progress to kidney failure, with patients generally starting dialysis in the latter half of the fifth decade of life (median age of 58 years) (Spithoven E M., et al., 2014, Kidney Int, 86 (6): 1244-1252). ADPKD is the fourth leading cause of kidney failure in the United States and worldwide (Collins A. J., et al., Am J Kidney Dis, 59 (1): A7; Chebib F T., et al., Am J Kidney Dis, 67 (5):792-810).

Current treatments for ADPKD mainly focus on controlling the symptoms (e.g., pain management) and trying to slow the progression of the disease. At the early stages of ADPKD, treatments may consist of medications like tolvaptan, the only approved drug for ADPKD, to slow cyst growth, and lifestyle changes like eating a low-sodium diet and exercising more to help control blood pressure. Treatment at later stages includes dialysis or a kidney transplant if the disease progresses to end-stage kidney disease (ESKD) (Liebau M. C., et al., 2023, Kiney Med. 5(3): 100596). However, there is no cure for ADPKD. Current treatment medication may have side effects such as severe allergic reactions, central pontine myelinolysis, liver damage, and blurred vision, therefore, there is still a high unmet medical need for therapeutic agents to treat ADPKD.

Although antisense oligonucleotides (ASOs) that directly bind to miRNAs can inhibit miRNA function (Lima J. F., et al., RNA Biol. 2018; 15(3):338-352), this approach may have off-target concerns since: 1) one miRNA can modulate protein expression of hundreds of different mRNAs; and, 2) currently used anti-miRNA ASOs are designed as short oligomers (e.g., 9 nt), dramatically increasing the possibility of base-pairing with non-target mRNAs and leading to off-target effects. Thus, more specific and effective approaches are desired. Herein, we describe approaches to increase PKD1 protein levels in a controllable way in cells and animals.

The antisense oligomeric compounds of the invention specifically increase the translation of PKD1 mRNA, thus increasing PKD1 protein levels to treat a haploinsufficiency and/or loss of function disease, disorder and/or condition such as ADPKD and/or Polycystic Liver Disease (PLD), Antisense compounds using Arnatar's proprietary Antisense Oligonuclcotidc (ASO) Coupled Translation—Upregulation 1 (ACT-UP1) technology are among the compounds disclosed herein for increasing the expression of PKD1 protein.

### SUMMARY OF THE INVENTION

Several embodiments provided herein relate to the discovery of certain ARNATAR design modified antisense compounds targeting PKD1 that can enhance their effectiveness in modulating PKD1 gene expression. In one embodiment, the antisense compound, or salt thereof, is an antisense oligonucleotide targeting the mRNA transcript of PKD1, wherein the compound enhances translation of PKD1 protein. In one embodiment, the antisense compound, or salt thereof, is an ACT-UP1 compound (e.g., as described in WO2025165891, incorporated-by-reference herein) targeting the mRNA transcript of PKD1, wherein the compound enhances translation of PKD1 protein.

In certain embodiments, a pharmaceutical composition for enhancing PKD1 gene expression comprises an antisense compound targeting PKD1, or salt thereof, alone or in combination with a pharmaceutically acceptable carrier or excipient.

Certain embodiments provide a method for increasing translation of PKD1 mRNA in a cell comprising administering an antisense compound targeting PKD1, or salt thereof, to a cell, in an amount sufficient to increase translation of the PKD1 mRNA.

Certain embodiments provide a method for increasing the amount of PKD1 mRNA and/or the expression of PKD1 protein in a subject comprising administering an antisense compound targeting PKD1, or salt thereof, to a cell, in an amount sufficient to increase the amount of PKD1 mRNA and/or the expression of PKD1 protein.

Certain embodiments provide a method for treating a haploinsufficiency and/or loss-of-function disorder in a subject comprising administering an antisense compound targeting PKD1, or salt thereof, to the subject, in an amount sufficient to treat the haploinsufficiency and/or loss-of-function disorder in the subject.

Certain embodiments provide a method for treating ADPKD and/or Polycystic Liver Disease (PLD) in a subject comprising administering an antisense compound targeting PKD1, or salt thereof, to the subject, in an amount sufficient to treat the subject.

Certain embodiments provide a process or method for making a compound targeting PKD1, or salt thereof, of the invention comprising synthesizing an oligonucleotide on a solid support using phosphoramidite chemistry thereby making the PKD1 targeting antisense compound.

### BRIEF DESCRIPTION OF THE DRAWINGS

FIG. 1A-B: Shows that different compounds can increase PKD1 protein levels in human HeLa or HEK293 cells: A) Western Blot; and B) quantification of PKD1 protein levels from the Western Blot. ATXL157 was used as a non-PKD1 targeting control. Tubulin protein was used as a loading standard for quantification.

FIG. 2A-B: Shows that different compounds transfected at 10 nM and/or 20 nM can increase PKD1 protein levels in human HK-2 cells: A) Western Blot; and B) quantification of PKD1 protein levels from the Western Blot. “**” is a non-specific (i.e., non-PKD1) protein band used as a loading standard for quantification.

FIG. 3A-B: Shows that different compounds can increase Pkd1 protein levels in mouse NIH3T3 cells 26 hours after transfection: A) Western Blot; and B) quantification of Pkd1 protein levels from the Western Blot. Gapdh protein was used as a loading standard for quantification.

FIG. 4A-C: Shows that different compounds can increase kidney Pkd1 protein levels at Day 15 in treated mice: A) schematic of the dosing strategy, B) Western Blot; and C) quantification of PKD1 protein levels from the Western Blot. Hsp90 protein was used as a loading standard for quantification.

FIG. 5A-C: Shows that different compounds can increase kidney Pkd1 protein levels at Day 22 in treated mice: A) schematic of the dosing strategy, B) Western Blot; and C) quantification of Pkd1 protein levels from the Western Blot. Gapdh protein was used as a loading standard for quantification.

FIG. 6A-B: Shows that mice treated with a higher dose (50 mg/kg) of compound ATXL322 have a greater increase in kidney Pkd1 protein level over a lower dose (15 mg/kg): A) Western Blot; and B) quantification of Pkd1 protein levels from the Western Blot. Tubulin protein was used as a loading standard for quantification.

FIG. 7A-B: Shows that compounds ATXL322 and ATXL551 can increase kidney Pkd1 protein levels in mice: A) Western Blot; and B) quantification of Pkd1 protein levels from the Western Blot. Actin protein was used as a loading standard for quantification.

FIG. 8A-B: Shows that various compounds can increase Pkd1 protein levels in mouse NIH3T3 cells 24 hours after transfection: A) Western Blot; and B) quantification of Pkd1 protein levels from the Western Blot. HSP90 protein was used as a loading standard for quantification.

FIG. 9A-B: Shows that various compounds can increase Pkd1 protein levels in mouse NIH3T3 cells 24 hours after transfection: A) Western Blot; and B) quantification of Pkd1 protein levels from the Western Blot. Hsp90 protein was used as a loading standard for quantification.

FIG. 10A-B: Shows that ACT-UP1 compound ATXL608 can increase PKD1 protein levels in human kidney HK-2 cells 24 hours after transfection: A) Western Blot; and B) quantification of PKD1 protein levels from the Western Blot. HSP90 protein was used as a loading standard for quantification.

FIG. 11A-B: Shows that ACT-UP1 compound ATXL608 can increase PKD1 protein levels in ADPKD patient-derived WT 9-7 cells 24 hours after transfection: A) Western Blot; and B) quantification of PKD1 protein levels from the Western Blot. HSP90 protein was used as a loading standard for quantification.

FIG. 12A-B: Shows that ACT-UP1 compound ATXL608 can increase PKD1 protein levels in ADPKD patient-derived WT 9-7 cells 72 hours after free uptake: A) Western Blot; and B) quantification of PKD1 protein levels from the Western Blot. HSP90 protein was used as a loading standard for quantification.

FIG. 13A-C: Shows that compounds ATXL607, ATXL608, and ATXL615 can increase kidney Pkd1 protein levels at Day 7 in treated mice: A) schematic of the dosing strategy, B) Western Blot; and C) quantification of Pkd1 protein levels from the Western Blot. Hsp90 protein was used as a loading standard for quantification.

FIG. 14A-C: Shows that ATXL608 can increase kidney Pkd1 protein levels in a dose-dependent manner at Day 7 in treated mice: A) schematic of the dosing strategy, B) Western Blot; and C) quantification of Pkd1 protein levels from the Western Blot. Hsp90 protein was used as a loading standard for quantification.

FIG. 15A-E: Shows a time course study of ATXL608 affecting Pkd1 protein levels in the kidney of treated mice: A) Schematic of the dosing strategy, B) Western Blot of Pkd1 protein levels at Day 10 after start of treatment; C) Western Blot of Pkd1 protein levels at Day 20 after start of treatment; D) Western Blot of Pkd1 protein levels at Day 30 after start of treatment; E) Graph of Pkd1 protein levels over time. Hsp90 protein was used as a loading standard for quantification.

FIG. 16A-C: Shows that ATXL608 treatment affects cAMP levels and proteins in the mTOR pathway in ADPKD patient-derived WT 9-7 cells: A) a chart of cAMP levels 72 hours after treatment; B) Western Blot of various proteins in the mTOR pathway 3 days after treatment; and C) quantification of the proteins from the Western Blot as ratios.

FIG. 17A-B: Shows that ATXL608 treatment affects cell survival and calcium (Ca²⁺) levels in ADPKD patient-derived WT 9-7 cells: A) graph showing cell survival by measuring cell numbers 2 or 3 days after treatment with ATXL608 at various concentrations; and B) chart showing Ca²⁺ levels 3 days after ATXL608 treatment at 0.1 μM or 1 μM.

FIG. 18A-B: Shows that ATXL608 treatment affects cyst formation in ADPKD patient-derived WT 9-7 cells: A) a representative image of cyst formation 14 days after treatment; and B) quantitation of cyst size in WT 9-7 cells treated with ATXL608 at various concentrations, indicating that ATXL608 treatment significantly decreased cyst size.

FIG. 19A-B: Shows that ATXL608 treatment decreases colony formation in ADPKD patient-derived WT 9-7 cells: A) representative image of colony formation 10 days after treatment with ATXL608 or control compound RGLS8429; and B) quantitation of colony formation in WT 9-7 cells treated with ATXL608 or control compound RGLS8429.

FIG. 20A-B: Shows that various compounds transfected at 10 nM and/or 20 nM can increase PKD1 protein levels 24 hours after treatment in ADPKD patient-derived WT 9-7 cells: A) Western Blot; and B) quantification of PKD1 protein levels from the Western Blot. HSP90 protein was used as a loading standard for quantification.

FIG. 21A-B: Shows that ACT-UP1 compounds ATXL814 and ATXL815 can increase PKD1 protein levels in ADPKD patient-derived WT 9-7 cells 48 hours after transfection: A) Western Blot; and B) quantification of PKD1 protein levels from the Western Blot. HSP90 protein was used as a loading standard for quantification.

FIG. 22A-D: Shows that ACT-UP1 compounds ATXL608, ATXL682, ATXL814 and ATXL815 can increase PKD1 protein levels in embryonic kidney explants: A) Western Blot of ATXL608 and ATXL682 treated samples; B) quantification of PKD1 protein levels from the Western Blot in panel A; C) Western Blot of ATXL814 and ATXL815 treated samples; and D) quantification of PKD1 protein levels from the Western Blot in panel C. Hsp90 protein was used as a loading standard for quantification.

FIG. 23A-C: Shows that ACT-UP1 compounds ATXL608 and ATXL682 can impede, decrease and/or reverse cyst formation in embryonic kidney explants: A) images of explants with cysts; B) Cyst Index Change based on the ratio of cyst area to the total kidney explant after treatment indicating that treatment can impede and decrease cyst formation; and C) per kidney Cyst Number Change indicating a reversal of cyst formation to below baseline (i.e., reversal) after treatment with ATXL608 or ATXL682.

FIG. 24A-C: Shows that ACT-UP1 compounds ATXL608, ATXL814 and ATXL815 can impede, decrease and/or reverse cyst formation in embryonic kidney explants: A) images of explants with cysts; B) Cyst Index Change based on the ratio of cyst area to the total kidney explant after treatment indicating that treatment can impede and decrease cyst formation; and C) per kidney Cyst Number Change indicating a reversal of cyst formation to below baseline (i.e., reversal) after treatment with ATXL608, ATXL814 and ATXL815.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject natter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Where permitted, all patents, applicator published applications and other publications, GENBANK Accession Numbers, and associated sequence information obtainable through databases such as National Center for Biotechnology Information (NCBI) and other data referred to throughout in the disclosure herein are incorporated-by-reference for the portions of the document discussed herein, as well as in their entirety.

Unless otherwise indicated, the following terms have the following meanings:
“2′-O-methoxyethyl” (also 2′-MOE and 2′-O(CH₂)₂—OCH₃) refers to an O-methoxy-ethyl modification at the 2′ position of a furanose ring. A 2′-O-methoxyethyl modified sugar is a modified sugar.
“2′-MOE nucleoside” (also 2′-O-methoxyethyl nucleoside) means a nucleoside comprising a 2′-MOE modified sugar moiety. “2′-MOE nucleotide” (also 2′-O-methoxyethyl nucleotide) means a nucleotide comprising a 2′-MOE modified sugar moiety.
“2′-O-methyl” (also 2′—OCH₃ and 2′-OMe) refers to an O-methyl modification at the 2′ position of a furanose ring. A 2′-O-methyl modified sugar is a modified sugar.
“2′-OMe nucleoside” (also 2′-O-methyl nucleoside) means a nucleoside comprising a 2′-OMe modified sugar moiety. “2′-OMe nucleotide” (also 2′-O-methyl nucleotide) means a nucleotide comprising a 2′-OMe modified sugar moiety.
“2′-substituted nucleoside” means a nucleoside comprising a substituent, i.e., a modification, at the 2′-position of the furanosyl ring other than H or OH. In certain embodiments, 2′-substituted nucleosides include nucleosides with a fluoro (2′-F), O-methyl (2′-OMe), O-methoxyethyl (2′-MOE), or bicyclic sugar modifications. A 2′-substituted nucleoside is a modified nucleoside.
“3′ target site” refers to the nucleotide of a target nucleic acid which is complementary to the 3′-most nucleotide of a particular antisense compound.
“5′ target site” refers to the nucleotide of a target nucleic acid which is complementary to the 5′-most nucleotide of a particular antisense compound.
“5-methylcytosine” means a cytosine modified with a methyl group attached to the 5 position. A 5-methylcytosine is a modified nucleobase and is part of a modified nucleoside. “5-methylcytidine” is the name of the nucleoside when the 5-methyl modified nucleobase is combined with a modified or unmodified sugar.
“About” as used herein means within ±10% of a measurable value. For example, if it is stated, “the compounds affected at least about 70% inhibition of mRNA”, it is implied that the mRNA levels are inhibited within a range of 63% and 77%. The term “about” as used herein when referring to an amount of a compound or agent of this invention, dose, time, temperature, and the like, is meant to encompass variations of ±10% of the specified amount. For sequence identity,
“about” as used for percent sequence identity encompasses variations of 5%. Unless otherwise indicated, all numbers expressing quantities of ingredients, properties such as reaction conditions, and so forth used in the specification and claims are to be understood as being modified in all instances by the term “about.”
“Animal” refers to a human or non-human animal, including, but not limited to, mice, rats, rabbits, dogs, cats, pigs, and non-human primates, including, but not limited to, monkeys and chimpanzees.
“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and F, region.
“Antisense compound” means an oligomeric compound that is capable of undergoing hybridization to a target nucleic acid through hydrogen bonding. Examples of antisense compounds include single-stranded and double-stranded compounds, such as, antisense oligonucleotides (ASOs), siRNAs, shRNAs, snoRNAs, miRNAs, and satellite repeats. Antisense compounds include ACT-UP1 compounds which comprise an antisense oligonucleotide (ASO) joined to a protein recruiting sequence (PRS). It is to be understood that antisense compounds include compounds targeting PKD1.
“Antisense oligonucleotide” or “ASO” means a single-stranded oligonucleotide having a nucleoside sequence comprising a nucleobase sequence that permits hybridization to a corresponding region or segment of a target nucleic acid. In certain embodiments, the ASO comprises one or more modified nucleosides. In certain embodiments, an ASO is a component of an ACT-UP1 compound, facilitating the binding of the compound to a target mRNA. As used herein, ASO does not refer to the protein recruiting sequence (PRS) in ACT-UP1 compounds.
“ACT-UP1” or “ASO Coupled Translation—Upregulation 1” refers to an antisense oligonucleotide (ASO) joined to a protein recruiting sequence (PRS), e.g., as described in WO2025165891, incorporated-by-reference herein. The ASO specifically hybridizes to a target mRNA sequence, bringing the PRS into close proximity to the target mRNA. The PRS, a short sequence of linked nucleosides, attracts translation regulatory proteins close to the target mRNA thereby increasing translation of the targeted mRNA. As used herein, the protein recruiting sequence (PRS), although it comprises a short linkage of nucleosides, is not an antisense oligonucleotide (ASO) as it does not hybridize with a target nucleic acid (i.e., the protein recruiting sequence is not antisense to a target nucleic acid). The ASO can be joined directly to the PRS or they can be joined by a linker. In some preferred aspects, the PRS is joined to the 5′ end of the ASO. In some aspects, the PRS is joined to the 3′ end of the ASO. Joined=covalently linked. It is to be understood that the ACT-UP1 compound may comprise additional sequences in addition to the ASO and PRS. For example, in some embodiments, the ACT-UP1 comprises an ASO and PRS and a linker for joining the ASO and PRS.
“ACT-UP1 activity” means any detectable or measurable activity attributable to the hybridization of an ACT-UP1 compound to its target nucleic acid. In certain embodiments, ACT-UP1 activity is an increase (i.e., up-regulation) in the amount of a target nucleic acid and/or expression of the protein encoded by such target nucleic acid.
“ACT-UP1 upregulation” or “ACT-UP1 enhancement” means increasing target protein levels in the presence of an ACT-UP1 compound compared to target protein levels in the absence of the ACT-UP1 compound.
“Average” as used herein may be mean, mode, or median for a group of measurements.

As used herein, the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art.
“Base complementarity” refers to the capacity for the base pairing of nucleobases of an oligonucleotide with corresponding nucleobases in a target nucleic acid (i.e., hybridization), and is mediated by Watson-Crick, Hoogsteen or reverse Hoogsteen hydrogen binding between corresponding nucleobases. Base complementarity also refers to canonical (e.g., A:U, A:T, or C:G) or non-canonical base pairings (e.g., A:G, A:U, G:U, I:U, I:A, or I:C).
“Bicyclic sugar” means a furanose ring modified by the bridging of two non-geminal carbon, atoms. A bicyclic sugar is a modified sugar.
“Cap structure” or “terminal cap moiety” means chemical modifications, which have been incorporated at either terminus of an antisense compound.
“cEt” or “constrained ethyl” means a bicyclic sugar moiety comprising a bridge connecting the 4′-carbon and the 2′-carbon, wherein the bridge has the formula: 4′-CH(CH₃)—O-2′.
“Constrained ethyl nucleoside” (also cEt nucleoside) means a nucleoside comprising a bicyclic sugar moiety comprising a 4′-CH(CH₃)—O-2′ bridge.
“Chemical modification” means a modification of molecular structure or element from naturally occurring molecules. For example, antisense oligonucleotides are composed of linked deoxyribonucleosides (also sometimes referred to herein as DNA nucleosides), therefore, substitution of a 2′-MOE nucleoside for a DNA nucleoside is considered a chemical modification of the antisense oligonucleotide. Examples of other chemical modifications may be found hereinbelow.
“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.
“Chemistry” when used in the context of a sequence refers to chemical modifications of the sequence, e.g., modifications of the nucleoside sugar, nucleoside base, and/or linkage, as further described herein. Thus, “sequence with chemistry” refers to the modifications of the nucleosides and their linkage, while “sequence” only describes the order of the nucleoside bases. However, neither the “sequence” nor “sequence with chemistry” refers to agent(s) attached to the sequences, such as a delivery agent.
“Complementarity” means the capacity for pairing between nucleobases of a first nucleic acid and a second nucleic acid.
“Comply” means the adherence to a recommended therapy by an individual.
“Comprise,” “comprises” and “comprising” will be understood to include a stated step or element or group of steps or elements but not the exclusion of any other step or element or group of steps or elements.
“Contiguous nucleobases” means nucleobases immediately adjacent to each other, for example to form a nucleobase sequence of a specified length.
“Contiguous nucleosides” means nucleosides immediately adjacent to each other, for example to form a nucleoside sequence of a specified length.
“Deoxyribonucleoside” means a nucleoside having a hydrogen at the 2′ position of the sugar portion of the nucleoside. A deoxyribonucleoside is sometimes referred to as DNA nucleoside, “D” or “d” herein. Deoxyribonucleosides may be modified with any of a variety of substituents and may be connected by covalent linkages other than naturally occurring phosphodiester such as phosphorothioate.
“Deoxyribonucleotide” means a nucleotide having a hydrogen at the 2′ position of the sugar portion of the nucleotide. A deoxyribonucleotide is sometimes referred to as DNA nucleotide, “D” or “d” herein. Deoxyribonucleotides may be modified with any of a variety of substituents and may be connected by covalent linkages other than naturally occurring phosphodiester such as phosphorothioate.
“Dual functional compound” or “dual functional ACT-UP1 compound” refers to a compound with an ACT-UP1 design plus a design for a second function or mechanism that is not ACT-UP1. For example, a dual functional compound has an ACT-UP1 design (e.g., binds to the 3′UTR of a target transcript and has a PRS) and targets the binding site of a miRNA. This dual functional compound could increase protein levels by both increasing mRNA levels (e.g., via blocking miRNA binding) and also increasing protein translation (via ACT-UP1).
“Efficacy” means the ability to produce a desired effect.
“Expression” includes all the functions by which a gene's coded information is converted into structures present and operating in a cell. Such structures include, but are not limited to, the products of translation.
“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.
“Fully modified” or “Fully chemically modified” refers to an antisense compound comprising a contiguous sequence of nucleosides wherein each nucleoside has a chemical modification.
“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.
“Immediately adjacent” means there are no intervening elements between the immediately adjacent elements.
“Individual” means a human or non-human animal selected for treatment or therapy.
“Induce”, “inhibit”, “potentiate”, “elevate”, “increase”, “decrease”, “enhance” or the like, generally denote quantitative differences between two states.
“Inhibiting the expression or activity” refers to a reduction, or blockade of the expression or activity and does not necessarily indicate a total elimination of expression or activity.
“Internucleoside linkage” or “linkage” refers to the chemical bond between nucleosides.

The 3′ position of a nucleoside is hereby linked to a 5′ position of a subsequent nucleoside via the internucleoside linkage.
“Isolated” means a state following one or more purifying steps but does not require absolute purity.
“Joined” ASO and PRS components of ACT-UP1 compound means that the ASO and PRS components are covalently linked to form the ACT-UP1 compound. It is to be understood that the ACT-UP1 compound may comprise additional sequences joined to the ASO and/or PRS. For example, in some embodiments, the ACT-UP1 comprises the ASO and PRS components and a linker for joining the ASO and PRS.
“Linked nucleosides” means adjacent nucleosides (e.g., A, G, C, T, or U) linked together by an internucleoside linkage. Examples of linked nucleosides include deoxyribonucleosides (sometimes referred to as DNA nucleosides herein) or ribonucleosides (sometimes referred to as RNA nucleosides herein).
“Linker” means a molecule that may function as a spacer between two components, e.g., between an antisense oligonucleotide and a delivery agent or between a PRS and ASO within an ACT-UP1 compound.
“Mismatch” or “non-complementary nucleobase” refers to the case when a nucleobase of a first nucleic acid is not capable of pairing with the corresponding nucleobase of a second or target nucleic acid through Watson-Crick base-pairing (e.g., A:T, A:U, or C:G). Merely by way of example, non-canonical basepair such as through Hoogsteen or reverse Hoogsteen may be considered a “mismatch” or “non-Complementary nucleobase.
“Modified internucleoside linkage” refers to a substitution or any change from a naturally occurring internucleoside bond (i.e. a phosphodiester internucleoside bond).
“Modified nucleobase” means any nucleobase other than adenine, cytosine, guanine, thymidine, or uracil. An “unmodified nucleobase” means the purine bases adenine (A) and guanine (G), and the pyrimidine bases thymine (f), cytosine (C), and uracil (U). In certain embodiments, an RNA nucleoside is considered modified when a DNA nucleoside is substituted for the RNA nucleoside. In certain embodiments, a DNA nucleoside is considered modified when an RNA nucleoside is substituted for the DNA nucleoside.
“Modified nucleoside” means a nucleoside having, independently, a modified sugar moiety and/or modified nucleobase.
“Modified nucleotide” means a nucleotide having, independently, a modified sugar moiety, modified internucleoside linkage, and/or a modified nucleobase.
“Modified oligonucleotide” means an oligonucleotide comprising at least one modified internucleoside linkage, a modified sugar, and/or a modified nucleobase.
“Modified sugar” means substitution and/or any change from a natural sugar moiety. Examples of a natural sugar moiety include ribose or deoxyribose.
“Moiety” means one of the portions into which something is divided i.e., a part or component of something. For example, a sugar moiety of a nucleotide is the sugar component of the nucleotide.
“Monomer” refers to a single unit of an oligomer. Monomers include, but are not limited to, nucleosides and nucleotides, whether naturally occurring or modified.
“Motif” means the pattern of unmodified and modified nucleosides in an antisense compound. Antisense compounds comprise motifs with various modified nucleobases, modified sugars, and/or internucleoside linkages in order to improve, among other characteristics, delivery, stability, specificity, safety and potency of the antisense compounds. The motif is independent of the nucleobase sequence of the antisense compound and identifies only the pattern of modifications.
“Natural sugar moiety” means a sugar moiety found in DNA (2′-H) or RNA (2′-OH).
“Naturally occurring internucleoside linkage” means a 3′ to 5′ phosphodiester linkage.
“Non-complementary nucleobase” refers to a pair of nucleobases that do not form hydrogen bonds with one another or otherwise support hybridization.
“Nucleic acid” refers to molecules composed of monomeric nucleotides. A nucleic acid includes, but is not limited to, ribonucleic acids (RNA e.g., mRNA), deoxyribonucleic acids (DNA), single-stranded nucleic acids (e.g., antisense oligonucleotides (ASOs) and microRNAs (miRNA)), double-stranded nucleic acids (e.g., small interfering ribonucleic acids (siRNAs) and short hairpin RNAs (shRNAs).
“Nucleobase” means a heterocyclic moiety capable of pairing with a base of another nucleic acid.
“Nucleobase complementarity” refers to a nucleobase that is capable of base pairing (also known as being complementary) with another nucleobase. If a nucleobase at a certain position of an oligomeric compound is capable of hydrogen bonding with a nucleobase at a certain position of a target nucleic acid, then the position of hydrogen bonding between the oligomeric compound and the target nucleic acid is considered to be complementary at that nucleobase pair. For example, in DNA, adenine (A) is complementary to thymine (T); in RNA, adenine (A) is complementary to uracil (U); and, Guanine (G) is complementary to cytosine (C) in both DNA and RNA. Base pairs, or complementary nucleobases, are usually canonical Watson-Crick base pairs (e.g., C:G, A:U, or A:T), but, non-canonical base pairs such as Hoogsteen base pairs (e.g., A:G, or A:U), Wobble base pairs (e.g., G:U, I:U, I:A, or I:C, wherein I is hypoxanthine) and the like are also included. Nucleobase complementarity facilitates hybridization of the oligomeric compounds described herein to their target nucleic acids;
“Nucleobase sequence” means the order of contiguous nucleobases independent of any sugar, linkage, and/or nucleobase modification.
“Nucleoside” means a nucleobase linked to a sugar.
“Nucleoside sequence” means the order of contiguous nucleosides independent of any sugar, linkage, and/or nucleobase modification.
“Nucleoside mimetic” includes those structures used to replace the sugar or the sugar and the base and not necessarily the linkage at one or more positions of an oligomeric compound, such as, for example, nucleoside mimetics having morpholino, cyclohexenyl, cyclohexyl, tetrahydropyranyl, bicyclo or tricyclo sugar mimetics, e.g., non-furanose sugar units. Nucleotide mimetics include those structures used to replace the nucleoside and the linkage at one or more positions of an oligomeric compound such as, for example, peptide nucleic acids or morpholinos (morpholinos linked by —N(H)—C(═O)—O- or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.
“Nucleotide” means a nucleoside having a linkage group (e.g., a phosphate (p) or phosphorothioate (PS) group) covalently linked to the sugar portion of the nucleoside. Nucleotides include ribonucleotides and deoxyribonucleotides. Ribonucleotides are the linked nucleotide units forming RNA. Deoxyribonucleotides are the linked nucleotide units forming DNA.
“Off-target effect” refers to an unwanted or deleterious biological effect associated with modulation of RNA or protein expression of a gene other than the intended target nucleic acid.
“Oligomeric compound” means a sequence of linked monomeric subunits that is capable of undergoing hybridization to at least a region of a target nucleic acid through hydrogen bonding. The monomeric subunits can be modified or unmodified nuclcobascs, nucleotides or nucleosides. Examples of oligomeric compounds include antisense compounds (e.g., antisense oligonucleotides (ASOs) or ACT-UP1 compounds comprising antisense oligonucleotides).
“Oligonucleotide” as used herein means a polymer of linked nucleosides, each of which can be modified or unmodified, independent from one another. Oligonucleotides can have a linking group other than a phosphate group (e.g., a phosphorothioate=thiophosphate group) used as a linking moiety between nucleosides.
“Substantially modified” or “substantially chemically modified” refers to an antisense compound comprising a contiguous sequence of nucleosides wherein the nucleosides are mostly, but, not fully, chemically modified. For example, the compound includes not more than about 5, 4, 3, 2, or 1 unmodified nucleoside.
“Phosphorothioate linkage” or “PS” means a linkage between nucleosides where the phosphodiester bond is modified by replacing one of the non-bridging oxygen atoms with a sulfur atom. A phosphorothioate (=thiophosphate or also known as thiophosphate) linkage is a modified internucleotide linkage.
“Protein recruiting sequence” or “PRS” is a short sequence of linked nucleosides that attracts and/or binds translation regulatory proteins. Merely by way of example, a translation regulatory protein can be an RNA binding protein (RBP) that regulates translation of mRNA into protein, which is well known in the art. The PRS is the component of the ACT-UP1 compound that interacts with RNA binding proteins, while the ASO is the component of the ACT-UP1 compound that hybridizes with a target nucleic acid; together, the ASO and PRS components of the ACT-UP1 compound enhance translation of an mRNA target. The protein recruiting sequence, although it comprises a short linkage of nucleosides, is not an antisense oligonucleotide (ASO) as it does not hybridize with a target nucleic acid (i.e., the protein recruiting sequence is not antisense to a target nucleic acid). For example, a PRS is/comprises a sequence having at least 80% identity to GGACU (SEQ ID NO: 4) as shown in Tables 3, 14, 23, or 25, GGACUGGACU (SEQ ID NO: 14) as shown in Table 3, GGACUGGAC (SEQ ID NO: 12) as shown in Tables 3, 14, 23, 25, or 28 and/or GGACTGGAC (SEQ ID NO: 13) as shown in Table 12, GGACUGGA (SEQ ID NO: 125), GAACUGAA (SEQ ID NO: 126).
“Portion” means a defined number of contiguous (i.e., linked)nucleosides of a nucleic acid. In certain embodiments, a portion is a defined number of contiguous nucleosides of a target nucleic acid. In certain embodiments, a portion is a defined number of contiguous nucleosides of an antisense compound.
“Region” is defined as a portion of the target nucleic acid having at least one identifiable structure, function, or characteristic.
“RNA” or “ribonucleic acid” consists of ribose nucleotides or ribonucleotides (nitrogenous bases attached to a ribose sugar) linked by phosphodiester bonds, forming strands of varying lengths. The nitrogenous bases in RNA are adenine, guanine, cytosine, and uracil.
“Ribonucleoside” means a nucleoside having a hydroxy at the 2′ position of the sugar portion of the nucleoside. A ribonucleoside is sometimes referred to as RNA nucleoside, “R” or “r” herein.
“Ribonucleotide” means a nucleotide having a hydroxy at the 2′ position of the sugar portion of the nucleotide. A ribonucleotide is sometimes referred to as RNA nucleotide, “R” or “r” herein.
“Segments” are defined as smaller or sub-portions of regions within a target nucleic acid.
“Sequence” as used herein refers to the order of nucleoside bases, without considering any modifications of the nucleosides or any additional agents coupled thereto.
“Sites,” as used herein, are defined as unique nucleoside positions within a target nucleic acid.
“Specifically hybridizable” refers to an antisense compound having a sufficient degree of complementarity between an antisense compound (e.g., ASO) and a target nucleic acid to induce a desired effect, while exhibiting minimal or no effects on non-target nucleic acids under conditions in which specific binding is desired, i.e., under physiological conditions in the case of in vivo assays and therapeutic treatments. Examples of sufficient degrees of complementarity are disclosed herein.
“Stringent hybridization conditions” or “stringent conditions” refer to conditions under which an antisense compound will hybridize to its target sequence, but to a minimal number of other sequences.
“Subject” means a human or non-human animal selected for treatment or therapy.
“Target” refers to a protein or nucleic acid sequence (e.g., mRNA), the modulation of which is desired. In certain embodiments, the modulation is an increase in expression of the target nucleic acid. In certain embodiments, the modulation is a decrease in expression of the target nucleic acid.
“Target gene” refers to a gene encoding a target.
“Targeting” means the process of design and selection of an antisense compound (e.g., ACT-UP1 compound) that will specifically hybridize to a target nucleic acid and induce a desired effect.
“Target nucleic acid,” “target RNA,” “target RNA transcript” and “nucleic acid target” all mean a nucleic acid capable of being targeted by antisense compounds.
“Target region” means a portion of a target nucleic acid to which one or more antisense compound is targeted.
“Target segment” means the sequence of nucleotides of a target nucleic acid to which an antisense compound is targeted. “5′ target site” refers to the 5′-most sequence of nucleotides of a target nucleic acid segment. “3′ target site” refers to the 3′-most sequence of nucleotides of a target nucleic acid segment. In an embodiment, a target segment is at least a 12-nucleoside portion (i.e., at least 12 consecutive nucleosides) of a target region to which an antisense compound is targeted.
“Therapeutic efficacy” refers to the effectiveness of a therapeutic compound such as an antisense compound. Therapeutic efficacy can be increased by improvements in delivery, stability, specificity, safety and potency of the therapeutic compound.
“Unmodified” RNA nucleosides mean the purines adenine (A) and guanine (G), and the pyrimidines cytosine (C) and uracil (U). “Unmodified” DNA nucleosides mean the purines adenine (A) and guanine (G), and the pyrimidines thymine (T) and cytosine (C). In certain embodiments, an unmodified RNA nucleoside is considered modified when a DNA nucleoside is substituted for the RNA nucleoside. In certain embodiments, an unmodified DNA nucleoside is considered modified when an RNA nucleoside is substituted for the DNA nucleoside.
“Unmodified nucleoside” means a nucleoside composed of commonly and naturally occurring nucleobases and sugar moieties. For example, an unmodified nucleoside is a DNA nucleoside if used in a DNA sequence, however, in such DNA sequence, any other nucleoside (e.g., RNA nucleoside, 2′-OMe, 2′-MOE, or 2′-F) is considered a modified nucleoside.
“Unmodified nucleotide” means a nucleotide composed of commonly and naturally occurring nucleobases, sugar moieties, and internucleoside linkages. For example, an unmodified nucleotide is a DNA nucleotide if used in a DNA sequence, however, in such DNA sequence, any other nucleotide (e.g., RNA nucleotide, 2′-OMe, 2′-MOE or 2′-F) is considered a modified nucleotide.
“Validated target segment” is defined as at least an 8-nucleoside portion (i.e. 8 consecutive nucleosides) of a target region to which an antisense compound is targeted.
“Wing segment” means a plurality of nucleosides at the 3′ and/or 5′ end of an antisense oligonucleotide, wherein the nucleosides are modified to impart to the antisense oligonucleotide properties such as, for example, enhanced activity, increased binding affinity for a target nucleic acid, and/or resistance to degradation by in vivo nucleases.

Disclosed herein are ARNATAR design modified oligomeric compounds targeting PKD1 that improve their effectiveness in enhancing PKD1 gene expression. In one embodiment, the oligomeric compound is an isolated or purified antisense oligonucleotide targeting the mRNA transcript of PKD1, wherein the compound enhances translation of PKD1 protein. In one embodiment, the oligomeric compound is an ACT-UP1 compound (e.g., as described in WO2025165891, incorporated-by-reference herein) targeting the mRNA transcript of PKD1, wherein the compound enhances translation of PKD1 protein. Specifically disclosed herein are antisense oligomeric compounds for enhancing expression of PKD1 in a cell, wherein the compound comprises any of the antisense oligomeric sequences targeting PKD1 in any one of, e.g., Tables 1, 3, 10, 12, 14, 23, 25 or 28. Further, disclosed herein are antisense compounds for enhancing expression of PKD1 in a cell as shown in any one of Tables 1, 3, 10, 12, 14, 23, or 25. Also contemplated are salt forms of the antisense compounds disclosed herein.

In certain embodiments, the, oligomeric compound targeting PKD1 comprises about 13 to 40, 13 to 39, 13 to 38, 13 to 37, 13 to 36, 13 to 35, 13 to 34, 13 to 33, 13 to 32, 13 to 31, 13 to 30, 13 to 29, 13 to 28, 13 to 27, 13 to 26, 13 to 25, 16 to 40, 16 to 35, 16 to 34, 16 to 33, 16 to 32, 16 to 31, 16 to 30, 16 to 29, 16 to 28:16 to 27, 16 to 26, 16 to 25, 19 to 40, 19 to 35, 19 to 34, 19 to 33, 19 to 32, 19 to 31, 19 to 30, 19 to 29, 19 to 28, 19 to 27, 19 to 26, 19 to 25, 22 to 40, 22 to 35, 22 to 34, 22 to 33,22 to 32, 22 to 31, 22 to 30, 22 to 29, 22 to 28, 22 to 27, 22 to 26, 22 to 25, 22 to 24, 22 to 23, 23 to 40, 23 to 35, 23 to 34, 23 to 33, 23 to 32, 23 to 31, 23 to 30, 23 to 29, 23 to 28, 23 to 27, 23 to 26, 23 to 25, 23 to 24, 24 to 40, 24 to 35, 24 to 34, 24 to 33, 24 to 32, 24 to 31, 24 to 30, 24 to 29, 24 to 28, 24 to 27, 24 to 26, 24 to 25, 25 to 40, 25 to 35, 25 to 34, 25 to 33, 25 to 32, 25 to 31, 25 to 30, 25 to 29, 25 to 28, or 25 to 27 linked subunits in length. In certain embodiments, the oligomeric compound targeting PKD1 comprises from 13 to 40, preferably 16 to 33, more preferably 19 to 25, and even more preferably 20 to 30, such as 22 to 28, linked subunits in length. In a preferred embodiment, the oligomeric compound is an ACT-UP1 compound.

In certain embodiments, the oligomeric compound targeting PKD1 comprises an ASO component comprising 13, 14, 15, 16, 17, 18, 19, 20, 21, 22, 23, 24, 25, 26, 27, 28, 29, 30, 31, 32, 33, 34, or 35 nucleosides in length and comprises no more than 6, no more than 5, no more than 4, no more than 3, no more than 2, or no more than 1 non-complementary nucleobase(s) relative to a PKD1 mRNA, or specified portion of the mRNA. In accordance with the practice of the invention, the no more than 6, no more than 5, no more than 4, no more than 3, no more than 2, or no more than 1 non-complementary nucleobase(s) relative to the target PKD1 mRNA, or specified portion thereof, can include zero (0) non-complementary nucleobase(s) relative to the target PKD1 mRNA, or specified portion thereof. In certain embodiments, the oligomeric compound targeting PKD1 comprises an ASO component comprising from 13 to 35, preferably 13 to 30, more preferably 13 to 17 nucleosides in length and comprises no more than 4, preferably no more than 3, more preferably no more than 2, non-complementary nucleobase(s) relative to a PKD1 mRNA, or specified portion of the mRNA. In a preferred embodiment, the oligomeric compound is an ACT-UP1 compound.

In certain embodiments, the oligomeric compound targeting PKD1 is an ACT-UP1 compound and comprises a PRS component comprising 5, 6, 7, 8, 9, 10, 11, 12, 13, 14, 15, 16, 17, 18, 19, 20, 21, 22, 23, 24, 25, 26, or 27 nucleosides in length. In certain embodiments, the oligomeric compound targeting PKD1 is an ACT-UP1 compound and comprises a PRS component comprising 5 to 27, preferably 6 to 20, more preferably 7 to 15, and even more preferably 7 to 13 nucleosides in length.

In one embodiment, the ACT-UP1 compound targeting PKD1 comprises: a PRS component about 5-20 nucleosides in length and an ASO component about 13-30 nucleosides in length with up to 3 non-complementary nucleobase(s) relative to a targeted PKD1 mRNA, or portion of the mRNA; preferably a PRS component about 5-15 nucleosides in length and an ASO component about 13-25 nucleosides in length with up to 3 non-complementary nucleobase(s) relative to a PKD1 mRNA, or portion of the mRNA; more preferably a PRS component about 7-13 nucleosides in length and an ASO component about 13-20 nucleosides in length with up to 3 non-complementary nucleobase(s) relative to a PKD1 mRNA, or portion of the mRNA; even more preferably a PRS component about 9-11 nucleosides in length and an ASO component about 13-20 nucleosides in length with up to 3 non-complementary nucleobase(s) relative to a PKD1 mRNA, or portion of the mRNA; such as a PRS component about 9-10 nucleosides in length and an ASO component about 13-20 nucleosides in length with up to 2 non-complementary nucleobase(s) relative to a PKD1 mRNA, or portion of the mRNA; a PRS component about 9-10 nucleosides in length and an ASO componentabout 13-20 nucleosides in length with up to I non-complementary nucleobase(s) relative to a PKD1 mRNA, or portion of the mRNA. In a most preferred embodiment, the compound targeting PKD1 comprises a PRS component 9 nucleosides in length and an ASO component 15 nucleosides in length with up to l non-complementary nucleobase(s) relative to a PKD1 mRNA, or portion of the mRNA. In accordance with the practice of the invention, the up 3 non-complementary nucleobase(s) relative to a PKD1 mRNA can include zero (0), 1, 2 or 3 non-complementary nucleobase(s) relative to the target PKD1 mRNA. In certain embodiments the PRS is or comprises a sequence having at least 80% identity to OGACU (SEQ ID NO: 4), GGACT (SEQ ID NO: 5), GGAUU (SEQ ID NO: 6), GAACU (SEQ ID NO: 7), AGACU (SEQ ID NO: 8), AAACU (SEQ ID NO: 9), GGACA (SEQ ID NO: 10), AAACA (SEQ ID NO: 11), GGACUGGAC (SEQ ID NO: 12), GGACTGGAC (SEQ ID NO: 13), GGACUGGACU (SEQ ID NO: 14), GGACUGGACUGGACU (SEQ ID NO: 15), ACGGACUUGGACU (SEQ ID NO: 16), AAACUAAACU (SEQ ID NO: 17), AAAAAAAAAAAA (SEQ ID NO: 18), AAAAAAAAAA (SEQ ID NO: 19), CCCCCCCCCC (SEQ ID NO: 20), GGGGGGGGGG (SEQ ID NO: 21), TITTIIITITT (SEQ ID NO: 22), UUUUUUUUUU (SEQ ID NO: 23), GGACUGGA (SEQ ID NO: 125), or GAACUGAA (SEQ. ID NO: 126. In certain embodiments the PRS is or comprises a sequence GGACU (SEQ ID NO: 4), GGACT (SEQ ID NO: 5), GGAUU (SEQ ID NO: 6), GAACU (SEQ ID NO: 7), AGACU (SEQ ID NO: 8), AAACU (SEQ ID NO: 9), GGACA (SEQ ID NO: 10), AAACA (SEQ ID NO: 11), GGACUGGAC (SEQ ID NO: 12), GGACTGGAC (SEQ ID NO: 13), GGACUGGACU (SEQ ID NO: 14), GGACUGGACUGGACU (SEQ ID NO: 15), ACGGACUUGGACU (SEQ ID NO: 16), AAACUAAACU (SEQ ID NO: 17), AAAAAAAAAAAA (SEQ ID NO: 18), AAAAAAAAAA (SEQ ID NO: 19), CCCCCCCCCC (SEQ ID NO: 20), GGGGGGGGGG (SEQ ID NO: 21), TITITITTT (SEQ ID NO: 22), UUUUUUUUUU (SEQ ID NO: 23), GGACUGGA (SEQ ID NO: 125), or GAACUGAA (SEQ ID NO: 126. The term “sequence” refers to the sequence of the bases in the linked nucleosides. The nucleosides may contain chemical modification(s) to natural nucleosides or their linkages as described below, which is preferable.

In certain embodiments, the ASO component comprises a sequence having at least 80% identity to GGACCCTGGGTCCTG (SEQ ID NO: 87) or GGACCCTGGGTCTTG (SEQ ID NO: 88). In certain embodiments, the ASO component comprises or consists of GGACCCTGGGTCCTG (SEQ ID NO: 87) or GGACCCTGGGTCTTG (SEQ ID NO: 88). The term “sequence” refers to the sequence of the bases in the linked nucleosides. The nucleosides may contain chemical modification(s) to natural nucleosides or their linkages as described below, which is preferable.

In certain embodiments, the ACT-UP1 compound targeting PKD1 further comprises an agent (e.g., a delivery, therapeutic or diagnostic agent) so as to form a conjugated compound. In certain embodiments, the conjugate agent (also referred to herein as moiety) can be selected from cholesterols, lipids, carbohydrates, phospholipids, biotin, phenazine, folate, phenanthridine, anthraquinone, acridine, fluoresceins, rhodamines, coumarins, peptides, antibodies, dyes, and tocopherol. In certain embodiments, the delivery agent comprises a conjugate moiety which may include one or more of HEG, FA-FI, and PALM.

In certain embodiments, the ACT-UP1 compound targeting PKD1 comprises at least one chemical modification. In certain embodiments, the compound targeting PKD1 is partially, substantially, or fully chemically modified. In certain embodiments, the compound targeting PKD1 is chemically modified such that the chemical modification is selected from one or more of 2′-O-methyl (2′-OMe), 2′-O-methoxyethyl (2′-MOE), 2′-fluoro (2′-F), constrained ethyl (cEt), unlocked nucleic acid (UNA), locked nucleic acid (LNA) and/or 5-methylcytosine base.

In certain embodiments, the ACT-UP1 compound targeting PKD1 comprises at least one modified internucleoside linkage; In certain embodiments, the at least one modified internucleoside linkage is a phosphorothioate internucleotide (PS) linkage.

In certain embodiments, the ACT-UP1 compound targeting PKD1 is about 13 to 40 linked nucleosides in length; includes at least one modified sugar, such as a bicyclic sugar, a 2′-O-methoxyethyl group (2′-MOE), a 2′-O-methyl group (2′-OMe), and/or a 4′-CH(CH₃)—O-2′ constrained ethyl (cEt) group; includes at least one modified internucleoside linkage, such as a phosphorothioate internucleoside linkage; and/or includes at least one modified nucleobase, such as a 5-methylcytidine. In certain embodiments, the ASO component of the ACT-UP1 compound targeting PKD1 is at least 70%, 75% 80%, 85%, 90% or 95% complementary to a target region of a PKD1 encoding mRNA (e.g., GENBANK Accession No. NM_001009944.3, SEQ ID NO: 1).

In certain embodiments, the ASO component of the ACT-UP1 compound targeting PKD1 is preferably at least 80%, and more preferably at least 90% complementary to a target region of a PKD1 encoding mRNA (e.g., GENBANK Accession No. NM_001009944.3, SEQ ID NO: 1).

Certain embodiments disclosed herein provide an ACT-UP1 compound, or salt thereof, for enhancing expression of PKD1 in a cell, wherein the compound comprises any of the sequences targeting PKD1 in Table 14. Certain embodiments disclosed herein provide an ACT-UP1 compound, or salt thereof, for enhancing expression of PKD1 in a cell, wherein the compound comprises any of the sequences targeting PKD1 in Table 14. In one embodiment, the ASO component of the ACT-UP1 compound comprises no more than 3, no more than 2, or no more than 1 non-complementary nucleobase(s) relative to the target PKD1 mRNA sequence. In accordance with the practice of the invention, the no more than 3, no more than 2, or no more than 1 non-complementary nucleobase(s) relative to the target PKD1 mRNA sequence can include zero (0) non-complementary nucleobase(s) relative to the target PKD1 sequence. In a preferred embodiment, the compound comprises the sequence of ATXL608 (SEQ ID NO: 77) or the chemistry and sequence of ATXL608 (SEQ ID NO: 43). In another preferred embodiment, the compound comprises the chemical structure of ATXL608 as shown below:

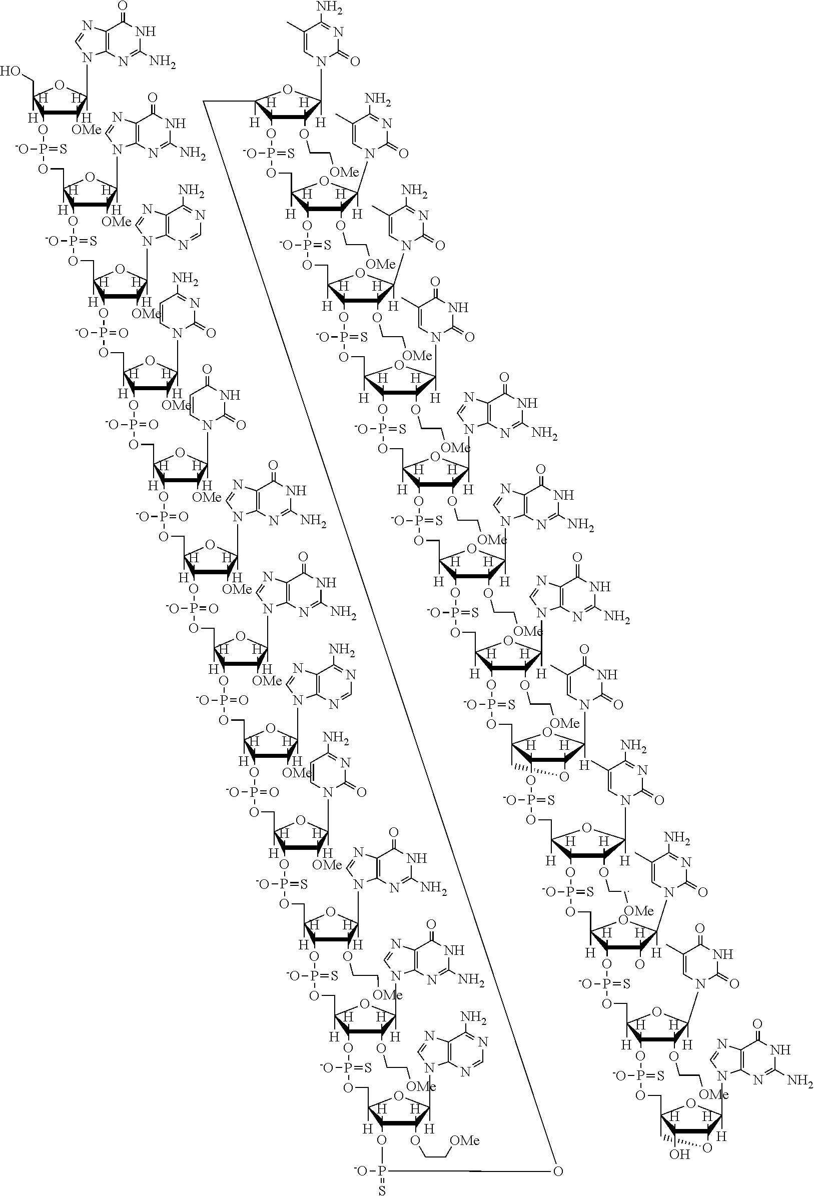

Certain embodiments disclosed herein provide an ACT-UP1 compound, or salt thereof, for enhancing expression of PKD1 in a cell, wherein the compound comprises any of the sequences targeting PKD1 in Table 23. Certain embodiments disclosed herein provide an ACT-UP1 compound, or salt thereof, for enhancing expression of PKD1 in a cell, wherein the compound comprises any of the sequences targeting PKD1 in Table 23. In one embodiment, the ASO component of the ACT-UP1 compound comprises no more than 3, no more than 2, or no more than 1 non-complementary nucleobase(s) relative to the target PKD1 mRNA sequence. In accordance with the practice of the invention, the no more than 3, no more than 2, or no more than 1 non-complementary nucleobase(s) relative to the target PKD1 mRNA sequence can include zero (0) non-complementary nucleobase(s) relative to the target PKD1 sequence. In a preferred embodiment, the compound comprises the sequence of ATXL682 (SEQ ID NO: 85) or the chemistry and sequence of ATXL682 (SEQ ID NO: 53). In another preferred embodiment, the compound comprises the chemical structure of ATXL682 as shown below:

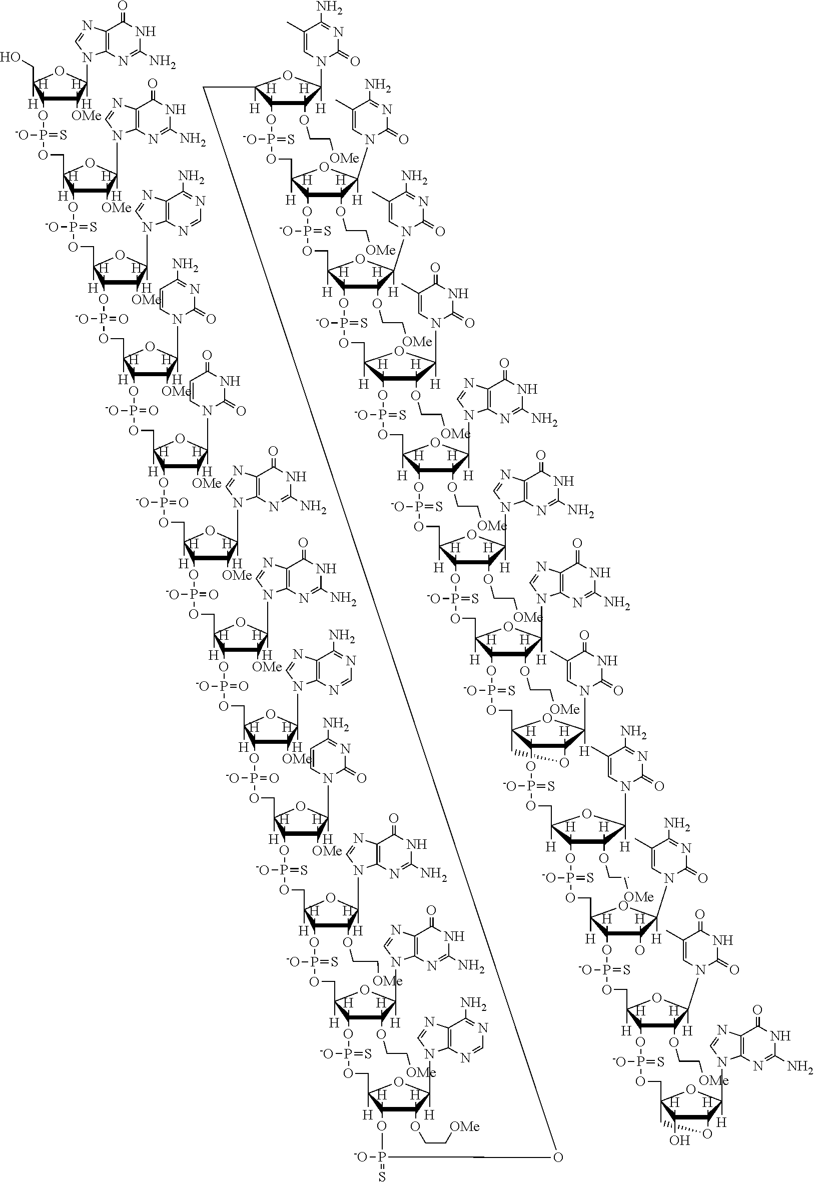

Certain embodiments disclosed herein provide an ACT-UP1 compound, or salt thereof, for enhancing expression of PKD1 in a cell, wherein the compound comprises any of the sequences targeting PKD1 in Table 23. Certain embodiments disclosed herein provide an ACT-UP1 compound, or salt thereof, for enhancing expression of PKD1 in a cell, wherein the compound comprises any of the sequences targeting PKD1 in Table 23. In one embodiment, the ASO component of the ACT-UP1 compound comprises no more than 3, no more than 2, or no more than 1 non-complementary nucleobase(s) relative to the target PKD1 mRNA sequence. In accordance with the practice of the invention, the no more than 3, no more than 2, or no more than 1 non-complementary nucleobase(s) relative to the target PKD1 mRNA sequence can include zero (0) non-complementary nucleobase(s) relative to the target PKD1 sequence. In a preferred embodiment, the compound comprises the sequence of ATXL686 (SEQ ID NO: 77) or the chemistry and sequence of ATXL686 (SEQ ID NO: 56). In another preferred embodiment, the compound comprises the chemical structure of ATXL686 as shown below:

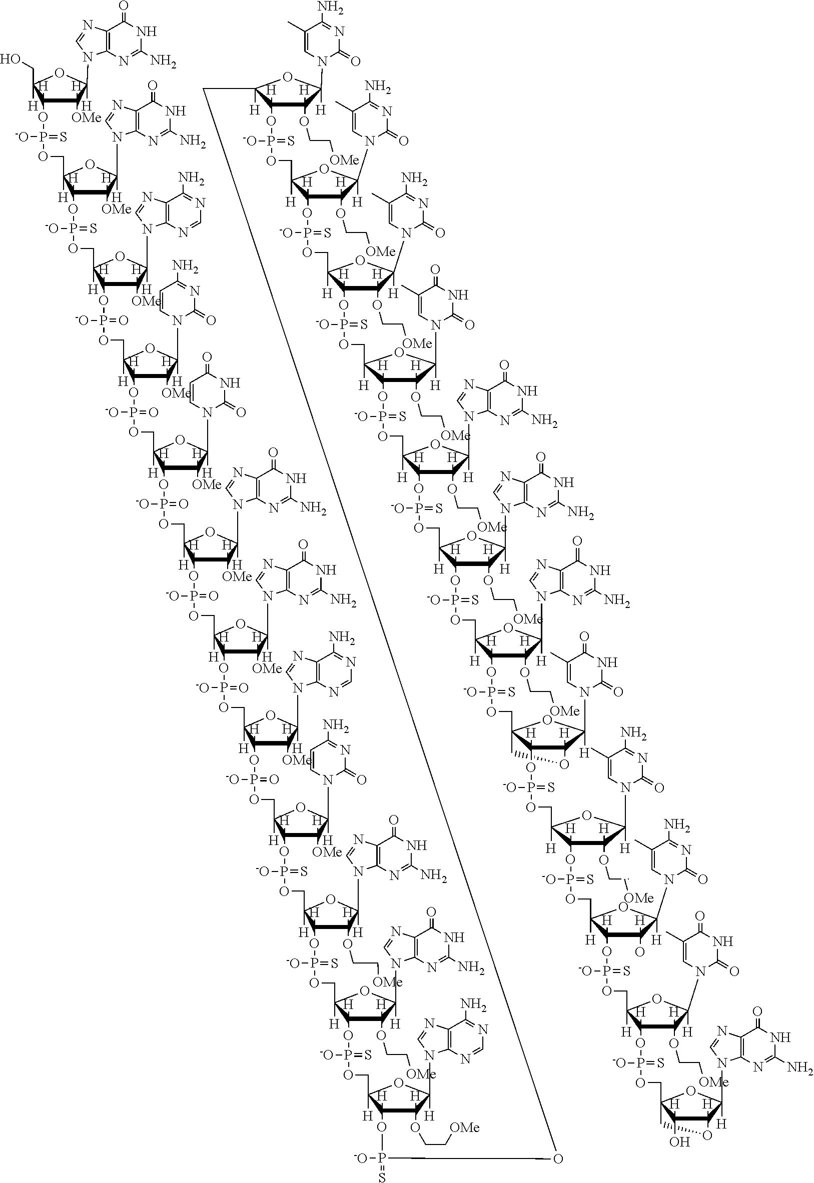

Certain embodiments disclosed herein provide an ACT-UP1 compound, or salt thereof, for enhancing expression of PKD1 in a cell, wherein the compound comprises any of the sequences targeting PKD1 in Table 25. Certain embodiments disclosed herein provide an ACT-UP1 compound, or salt thereof, for enhancing expression of PKD1 in a cell, wherein the compound comprises any of the sequences targeting PKD1 in Table 25. In one embodiment, the ASO component of the ACT-UP1 compound comprises no more than 3, no more than 2, or no more than 1 non-complementary nucleobase(s) relative to the target PKD1 mRNA sequence. In accordance with the practice of the invention, the no more than 3, no more than 2, or no more than 1 non-complementary nucleobase(s) relative to the target PKD1 mRNA sequence can include zero (0) non-complementary nucleobase(s) relative to the target PKD1 sequence. In a preferred embodiment, the compound comprises the sequence of ATXL814 (SEQ ID NO: 77) or the chemistry and sequence of ATXL814 (SEQ ID NO: 57). In another preferred embodiment, the compound comprises the chemical structure of ATXL814 as shown below:

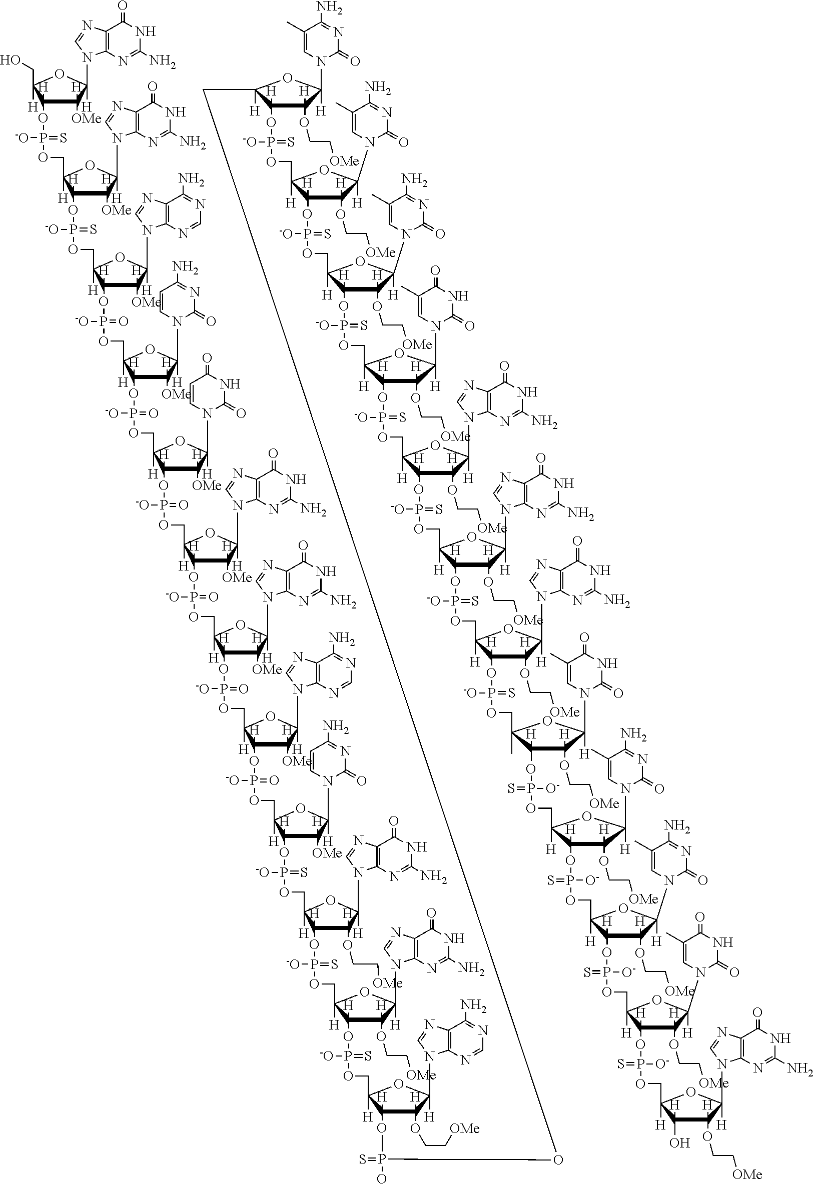

Certain embodiments disclosed herein provide an ACT-UP1 compound, or salt thereof, for enhancing expression of PKD1 in a cell, wherein the compound comprises any of the sequences targeting PKD1 in Table 25. Certain embodiments disclosed herein provide an ACT-UP1 compound, or salt thereof, for enhancing expression of PKD1 in a cell, wherein the compound comprises any of the sequences targeting PKD1 in Table 25. In one embodiment, the ASO component of the ACT-UP1 compound comprises no more than 3, no more than 2, or no more than I non-complementary nucleobase(s) relative to the target PKD1 mRNA sequence. In accordance with the practice of the invention, the no more than 3, no more than 2, or no more than 1 non-complementary nucleobase(s) relative to the target PKD1 mRNA sequence can include zero (0) non-complementary nucleobase(s) relative to the target PKD1 sequence. In a preferred embodiment, the compound comprises the sequence of ATXL815 (SEQ ID NO: 85) or the chemistry and sequence of ATXL815 (SEQ ID NO: 58). In another preferred embodiment, the compound comprises the chemical structure of ATXL815 as shown below:

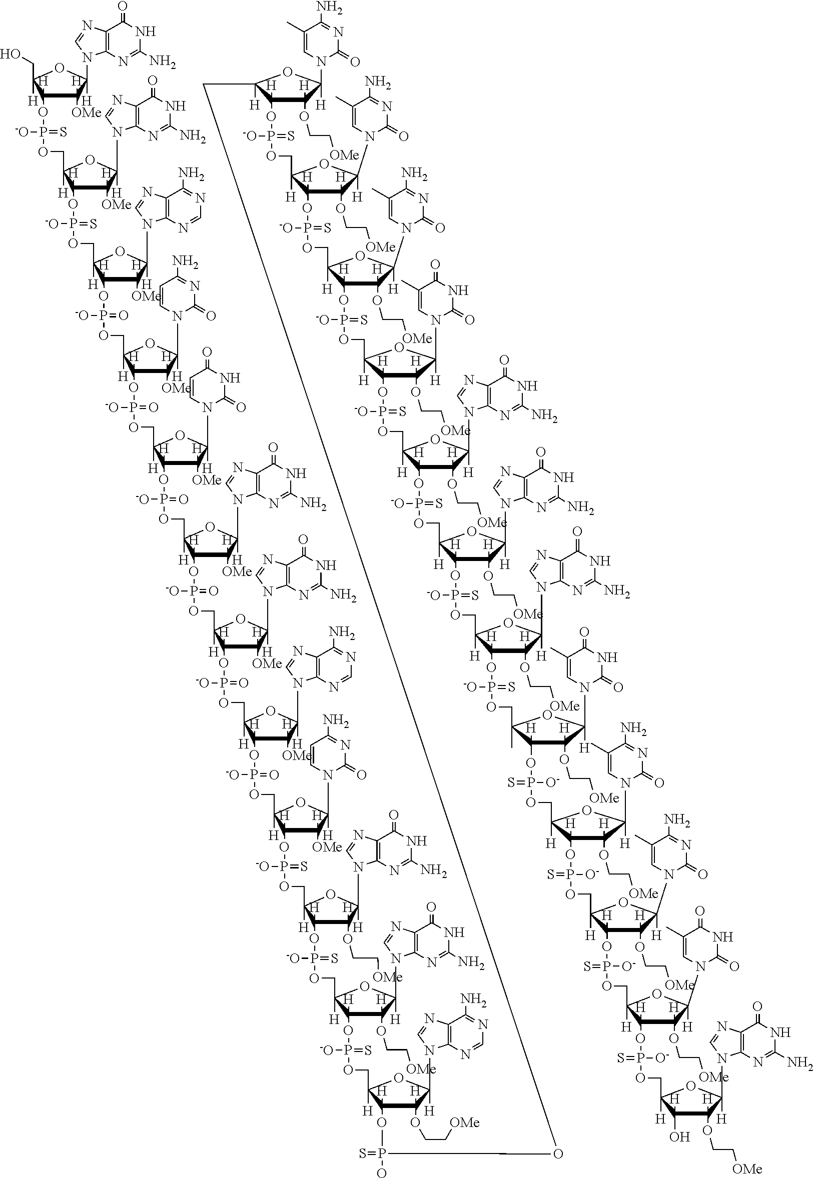

In certain embodiments, the compound, or salt thereof, targeting PKD11 mRNA increases expression of PKD1 protein in a cell by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300%. In a preferred embodiment, the compound, or salt thereof, increases expression of PKD1 by at least about 20%, 25%, 30%, 35%, 40%, 45%, 50%, 55%, or 60% in the cell. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

Certain embodiments disclosed herein provide a pharmaceutical composition comprising the compound targeting PKD1, or salt thereof, described herein, alone or in combination with a pharmaceutically acceptable carrier or excipient. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In certain embodiments, the pharmaceutical composition is administered to a subject in an amount sufficient to increase PKD1 expression by at least about 20%, 25%, 30%, 35%, 40%, 45%, 50%, 55%, or 60%, in the subject, wherein the increase in PKD1 protein expression treats a haploinsufficiency and/or loss-of-function disease, disorder and/or condition in the subject.

Certain embodiments disclosed herein provide a method for increasing translation of PKD1 mRNA in a cell comprising administering a compound, or salt thereof, targeting PKD1 to a cell, in an amount sufficient to increase translation of the PKD1 mRNA. The compound targeting PKD1 compound increases translation of the PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300%. In a preferred embodiment, the compound, or salt thereof, increases translation of PKD1 mRNA by at least about 20%, 25%, 30%, 35%, 40%, 45%, 50%, 55%, or 60% in the cell. In one embodiment, the compound, or salt thereof, comprises the sequence and/or chemistry of any one of the compounds targeting PKD1 in Tables 1, 3, 10, 12, 14,23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

In some embodiments, the invention provides methods for increasing translation of PKD1 mRNA in a cell comprising administering a compound, or salt thereof, targeting PKD1 to a cell so that the compound binds PKD11 mRNA, in an amount sufficient to increase translation of the PKD1 mRNA. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

Certain embodiments disclosed herein provide a method for increasing expression of PKD1 protein in a subject comprising administering a compound, or salt thereof, targeting PKD1 to a cell, in an amount sufficient to increase expression of PKD1 protein. The compound targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300%. In a preferred embodiment, the compound, or salt thereof, increases expression of PKD1 by at least about 20%, 25%, 30%, 35%, 40%, 45%, 50%, 55%, or 60% in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

Certain embodiments disclosed herein provide a method for increasing the level of PKD1 mRNA in a subject comprising administering a compound, or salt thereof, targeting PKD1 to a cell, in an amount sufficient to increase the level of PKD1 mRNA. The compound targeting PKD1 mRNA increases the level of PKD1 mRNA by at least about 10%, 15%, 20%, 25%, 30%, 35%, 40%, 45%. 50%, 55%, 60%, 65%, 70%, 75%, 80%, 85%, 90%, 95%, 100%, 150%, 200%, or 300%. In a preferred embodiment, the compound, or salt thereof, increases the level of PKD1 mRNA by at least about 20%, 25%, 30%, 35%, 40%, 45%, 50%, 55%, or 60% in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

In further embodiments, the invention provides methods for increasing expression of PKD1 protein in a subject comprising administering a compound, or salt thereof, targeting PKD1 to a cell so that the compound binds PKD1, in an amount sufficient to increase expression of PKD1 protein.

In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

In further embodiments, the invention provides methods for increasing the level of PKD1 mRNA in a subject comprising administering a compound, or salt thereof, targeting PKD1 to a cell so that the compound binds PKD1, in an amount sufficient to increase the level of PKD1 mRNA. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

Certain embodiments disclosed herein provide a method for treating a haploinsufficiency and/or loss-of-function disease, disorder and/or condition in a subject comprising administering a compound, or a salt thereof, targeting PKD1 to the subject, in an amount sufficient to treat the haploinsufficiency and/or loss-of-function disease, disorder and/or condition in the subject. The compound, or salt thereof, targeting PKD1 mRNA increases expression of PKM) protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to treat the haploinsufficiency and/or loss-of-function disease, disorder and/or condition in the subject. In certain embodiments, the haploinsufficiency and/or loss-of-function disease, disorder and/or condition is polycystic liver disease (PLD) and/or polycystic kidney disease (e.g., autosomal dominant polycystic kidney disease (ADPKD)). In a preferred embodiment, the compound, or salt thereof, increases expression of PKD1 by at least about 20%, 259%, 30%, 35%, 40%, 45%, 50%, 55%, or 60% in order to treat the haploinsufficiency and/or loss-of-function disease, disorder and/or condition in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

Certain embodiments disclosed herein provide a method for treating polycystic kidney disease (PKD) in a subject comprising administering a compound, or salt thereof, targeting PKD1 to the subject, in an amount sufficient to treat the subject. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to treat PKD in the subject. In a preferred embodiment, the compound, or salt thereof, increases expression of PKD1 by at least about 20%, 25%, 30%, 35%, 40%, 45%, 50%, 55%, or 60% in order to treat PKD1 in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In one embodiment, the polycystic kidney disease is autosomal dominant polycystic kidney disease (ADPKD).

Certain embodiments disclosed herein provide a method for treating polycystic liver disease (PLD) in a subject comprising administering a compound, or salt thereof, targeting PKD1 to the subject, in an amount sufficient to treat the subject. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to treat PLD in the subject. In a preferred embodiment, the compound, or salt thereof, increases expression of PKD1 by at least about 20%, 25%, 30%, 35%, 40%, 45%, 50%, 55%, or 60% in order to treat PLD in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

Certain embodiments disclosed herein provide a method for decreasing cyst size in an organ in a subject comprising administering a compound, or salt thereof, targeting PKD1 to the subject, in an amount sufficient to decrease cyst size in the subject. Decreasing cyst size can be assessed by measuring the cyst size in the organ after treatment of the subject with the compound, or salt thereof, in comparison to the cyst size in the organ of an untreated subject, or in comparison to the cyst size in the same organ before treatment. In a specific embodiment, the organ can be a liver and/or a kidney. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to decrease cyst size in the subject. In a preferred embodiment, the compound, or salt thereof, increases expression of PKD1 by at least about 20%, 25%, 30%, 35%, 40%, 45%, 50%, 55%, or 60% in order to decrease cyst size in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In one embodiment, the organ is a liver and/or kidney.

Certain embodiments disclosed herein provide a method for decreasing cyst formation (i.e., decreasing the number of cysts formed) in an organ in a subject comprising administering a compound, or salt thereof, targeting PKD1 to the subject, in an amount sufficient to decrease cyst formation in the subject. Decreasing cyst formation can be measured by, e.g., the number of cysts formed in the organ after treatment of the subject with the compound, or salt thereof, in comparison to the number of cysts formed in the organ of an untreated subject, or in comparison to the number of cysts formed in the same organ before treatment. In a specific embodiment, the organ can be a liver and/or a kidney. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to decrease cyst formation in the subject. In a preferred embodiment, the compound, or salt thereof, increases expression of PKD1 by at least about 20%, 25%, 30%, 35%, 40%, 45%, 50%, 55%, or 60% in order to decrease cyst formation in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In one embodiment, the organ is a liver and/or kidney.

Certain embodiments disclosed herein provide a method for impeding (i.e., inhibiting) cyst formation (i.e., impeding/inhibiting the development of cysts) in an organ in a subject comprising administering a compound, or salt thereof, targeting PKD1 to the subject, in an amount sufficient to impede cyst formation in the subject. Impeding cyst formation can be measured by, e.g., the number of cysts formed and/or the size of the cysts formed in the organ after treatment of the subject with the compound, or salt thereof, in comparison to number of cysts formed and/or the size of the cysts formed in the organ of an untreated subject, or in comparison to the number of cysts formed and/or the size of the cysts formed in the same organ before treatment. In a specific embodiment, the organ can be a liver and/or a kidney. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 1000, 150%, 200%, or 300% in order to impede cyst formation in the subject. In a preferred embodiment, the compound, or salt thereof, increases expression of PKD1 by at least about 20%, 25%, 30%, 35%, 40%, 45%, 50%, 55%, or 60% in order to impede cyst formation in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In one embodiment, the organ is a liver and/or kidney.

Certain embodiments disclosed herein provide a method for reversing cyst progression (i.e., reversing the progressive growth of cysts below the number and size of cysts present in the organ prior to administering the compound) in an organ in a subject comprising administering a compound, or salt thereof, targeting PKD1 to the subject, in an amount sufficient to reverse cyst formation in the subject. Reversing cyst progression can be measured by, e.g., the number of cysts formed and/or the size of the cysts formed in the organ after treatment of the subject with the compound, or salt thereof, in comparison to number of cysts formed and/or the size of the cysts formed in the organ of an untreated subject, or in comparison to the number of cysts formed and/or the size of the cysts formed in the same organ before treatment, wherein the number of cysts formed and/or the size of the cysts formed after treatment is less than the number and/or size of cysts in the organ before treatment. In a specific embodiment, the organ can be a liver and/or a kidney. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to reverse cyst progression in the subject. In a preferred embodiment, the compound, or salt thereof, increases expression of PKD1 by at least about 20%, 25%, 30%, 35%, 40%, 45%, 50%, 55%, or 60% in order to reverse cyst progression in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In one embodiment, the organ is a liver and/or kidney.

Certain embodiments disclosed herein provide a method for improving the function of an organ in a subject comprising administering a compound, or salt thereof, targeting PKD1 to the subject, in an amount sufficient to improve organ function in the subject. Organ function can be measured by various methods, including, but not limited to certain tests for: alanine transaminase (ALT), aspartate transaminase (AST), albumin, alkaline phosphatase (ALP), gamma-glutamyl transferase (GGT), bilirubin, blood urea nitrogen (BUN), serum creatinine, and estimated glomerular filtration rate (eGFR). The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to improve organ function in the subject. In a preferred embodiment, the compound, or salt thereof, increases expression of PKD1 by at least about 20%, 25%, 30%, 35%, 40%, 45%, 50%, 55%, or 60% in order to improve organ function in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In one embodiment, the organ is a liver and/or kidney.

Certain embodiments disclosed herein provide a method for reducing hypertension in a subject comprising administering a compound, or salt thereof, targeting PKD1 to the subject, in an amount sufficient to reduce hypertension in the subject. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to reduce hypertension in the subject. In a preferred embodiment, the compound, or salt thereof, increases expression of PKD1 by at least about 20%, 25%, 30%, 35%, 40%, 45%, 50%, 55%, or 60% in order to reduce hypertension in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28.

Certain embodiments disclosed herein provide a method for reducing fibrosis in an organ in a subject comprising administering a compound, or salt thereof, targeting PKD1 to the subject, in an amount sufficient to reduce organ fibrosis in the subject. The compound, or salt thereof, targeting PKD1 mRNA increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, or 300% in order to reduce organ fibrosis in the subject. In a preferred embodiment, the compound, or salt thereof, increases expression of PKD1 by at least about 20%, 25%, 30%, 35%, 40%, 45%, 50%, 55%, or 60% in order to reduce organ fibrosis in the subject. In one embodiment, the compound, or salt thereof, comprises the sequence and/or chemistry of any one of the compounds targeting PKD1 in Tables 1, 3, 10, 12, 14, 23, or 25. In a preferable embodiment, the compound, or salt thereof, comprises an ACT-UP1 compound with a sequence and/or chemistry in any one of Tables 3, 12, 14, 23, or 25. In certain embodiments, the sequence of the compound can differ up to 1, up to 2, up to 3, up to 4, up to 5, or up to 6 nucleobase positions from the sequence of any of the compounds disclosed in Tables 3, 12, 14, 23, 25, or 28. In one embodiment, the organ is a liver and/or kidney.

Certain embodiments disclosed herein provide the ACT-UP1 compound in any of the embodiments disclosed herein or the pharmaceutical composition in any of the embodiments disclosed herein for use in medicine. The use may be as disclosed for any of the methods above.

Certain embodiments disclosed herein provide the ACT-UP1 compound in any of the embodiments disclosed herein or the pharmaceutical composition in any of the embodiments disclosed herein for use in treating a haploinsufficiency and/or loss-of-function disease, disorder and/or condition in a subject comprising administering the compound or the pharmaceutical composition to the subject, in an amount sufficient to increase PKD1 expression by at least about 20%, wherein the increase in PKD1 protein expression treats the haploinsufficiency and/or loss-of-function disease, disorder and/or condition in the subject. The haploinsufficiency and/or loss-of-function disease, disorder and/or condition may be selected from polycystic kidney disease and/or polycystic liver disease,

Certain embodiments disclosed herein provide the ACT-UP1 compound in any of the embodiments disclosed herein or the pharmaceutical composition in any of the embodiments disclosed herein for use in decreasing cyst size in an organ of a subject comprising administering the compound or the pharmaceutical composition to the subject, in an amount sufficient to increase PKD1 expression by at least about 20%, wherein the increase in PKD1 protein expression decreases cyst size in the organ of the subject.

Certain embodiments disclosed herein provide the ACT-UP1 compound in any of the embodiments disclosed herein or the pharmaceutical composition in any of the embodiments disclosed herein for use in decreasing cyst formation in an organ of a subject comprising administering the compound or the pharmaceutical composition to the subject, in an amount sufficient to increase PKD1 expression by at least about 20%, wherein the increase in PKD1 protein expression decreases cyst formation in the organ of the subject.

Certain embodiments disclosed herein provide the ACT-UP1 compound in any of the embodiments disclosed herein or the pharmaceutical composition in any of the embodiments disclosed herein for use in impeding cyst formation in an organ of a subject comprising administering the compound or the pharmaceutical composition to the subject, in an amount sufficient to increase PKD1 expression by at least about 20%, wherein the increase in PKD1 protein expression impedes cyst formation in the organ of the subject.

Certain embodiments disclosed herein provide the ACT-UP1 compound in any of the embodiments disclosed herein or the pharmaceutical composition in any of the embodiments disclosed herein for use in reversing cyst progression in an organ of a subject comprising administering the compound or the pharmaceutical composition to the subject, in an amount sufficient to increase PKD1 expression by at least about 20%, wherein the increase in PKD1 protein expression reverses cyst progression in the organ of the subject.

In certain embodiments, the ACT-UP1 compound, or salt thereof, targeting PKD1 can be administered subcutaneously or intravenously to a subject.

Certain embodiments disclosed herein provide a process or methods for making a compound, or salt thereof, targeting PKD1 of the invention comprising synthesizing an oligonucleotide on a solid support using phosphoramidite chemistry thereby making the PKD1 targeting compound.

In one embodiment, a process for preparing a PKD1 targeting compound, or salt thereof, of the invention is provided, wherein the process comprises the steps of: a) preparing the compound by sequential coupling of modified and/or unmodified nucleotides and/or linkers via the phosphoramidite oligonucleotide synthesis on a conjugate modified or unmodified solid support; b) optionally, coupling a conjugate moiety to the compound on the solid support via the phosphoramidite oligonucleotide synthesis; c) detaching the compound from the solid support and removing the solid support; and (d) optionally, adding a conjugate post cleavage; and/or (e) optionally, further purifying the compound, optionally using chromatography.

In one embodiment, a process for preparing a PKD1 targeting compound, or salt thereof, of the invention is provided, wherein the process comprises the steps of: a) coupling a conjugate moiety to a solid support via the phosphoramidite oligonucleotide synthesis, b) coupling a modified and/or unmodified nucleotide via the phosphoramidite oligonucleotide synthesis to the conjugate moiety on the solid support; c) sequentially coupling additional modified and/or unmodified nucleotides via the phosphoramidite oligonucleotide synthesis to prepare the compound; d) detaching the compound from the solid support and removing the solid support; and e) optionally, further purifying the compound, optionally using chromatography.

### Antisense Compounds

An antisense compound is an oligomeric compound that is capable of undergoing hybridization with a target nucleic acid through hydrogen bonding. Examples of antisense compounds include single-stranded and double-stranded compounds such as antisense oligonucleotides (ASOs), siRNAs, shRNAs, snoRNAs, miRNAs, and satellite repeats. It is to be understood that antisense compounds include ACT-UP1 compounds, which comprise an antisense oligonucleotide (ASO) joined to a protein-recruiting sequence (PRS). Also contemplated are salt forms of the oligomeric compounds.

In certain embodiments, an antisense oligonucleotide comprises a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound is about 13 to 40, 13 to 39, 13 to 38, 13 to 37, 13 to 36, 13 to 35, 13 to 34, 13 to 33, 13 to 32, 13 to 31, 13 to 30, 13 to 29, 13 to 28, 13 to 27, 13 to 26, 13 to 25, 19 to 40, 19 to 35, 19 to 34, 19 to 33, 19 to 32, 19 to 31, 19 to 30, 19 to 29, 19 to 28, 19 to 27, 19 to 26, 19 to 25, 22 to 40, 22 to 35, 22 to 34, 22 to 33, 22 to 32, 22 to 31, 22 to 30, 22 to 29, 22 to 28, 22 to 27, 22 to 26, 22 to 25, 22 to 24, 22 to 23, 23 to 40, 23 to 35, 23 to 34, 23 to 33, 23 to 32, 23 to 31, 23 to 30, 23 to 29, 23 to 28, 23 to 27, 23 to 26, 23 to 25, 23 to 24, 24 to 40, 24 to 35, 24 to 34, 24 to 33, 24 to 32, 24 to 31, 24 to 30, 24 to 29, 24 to 28, 24 to 27, 24 to 26, 24 to 25, 25 to 40, 25 to 35, 25 to 34, 25 to 33, 25 to 32, 25 to 31, 25 to 30, 25 to 29, 25 to 28, or 25 to 27 linked subunits. In certain such embodiments, antisense compounds are about 13, 14, 15, 16, 17, 18, 19, 20, 21, 22, 23, 24, 25, 26, 27, 28, 29, 30, 31, 32, 33, 34, 35, 36, 37, 38, 39, or 40 linked subunits in length, or a range defined by any two of the above values. In certain embodiments, the antisense compound comprises from 13 to 40, preferably 16 to 33, more preferably 19 to 25, and even more preferably 20 to 30, such as 22 to 28, linked subunits in length.

In certain embodiments, an antisense compound is about 13 to 40 subunits in length and targets a PKD1 mRNA. In certain embodiments, the antisense compound is an ACT-UP1 compound targeting PKD1. In certain embodiments, the ASO component of the ACT-UP1 compound targeting PKD1 is at least about 70%, 75% 80%, 85%, 90% or 95% complementary to a PKD1 encoding mRNA (e.g., GENBANK Accession No. NM_001009944.3, SEQ ID NO: 1). In a preferred embodiment, the ASO component of an ACT-UP1 compound is about 13 to 35, 13 to 30, 13 to 25, 13 to 24, 13 to 23, 13 to 22, 13 to 21, 13 to 20, 13 to 19, 13 to 18, 13 to 17, 13 to 16, 13 to 15 or 13 to 14 linked subunits in length.

In certain embodiments, the ASO component comprises 13, 14, 15, 16, 17, 18, 19, 20, 21, 22, 23, 24, 25, 26, 27, 28, 29, 30, 31, 32, 33, 34 or 35 nucleosides in length and comprises no more than 6, no more than 5, no more than 4, no more than 3, no more than 2, or no more than I non-complementary nucleobase(s) relative to a PKD1 mRNA, or specified portion of the mRNA. In certain embodiments, the ASO component comprises from 13 to 35, preferably 13 to 30, more preferably 13 to 17 nucleosides in length. In certain embodiments, the sequence of the ASO component can differ up to 1, up to 2, up to 3, up to 4, up to 5, or up to 6 nucleobase positions from the sequence of any of the ASO components disclosed in herein and preferably at Tables 3, 12, 14, 23, 25, or 28.

In certain embodiments, the ACT-UP1 compound targeting PKD1 comprises a PRS component comprising 5, 6, 7, 8, 9, 10, 11, 12, 13, 14, 15, 16, 17, 18, 19, 20, 21, 22, 23, 24, 25, 26 or 27 nucleosides in length. In certain embodiments, the PRS component comprises 5 to 27, preferably 6 to 20, more preferably 7 to 15, and even more preferably 7 to 13 nucleosides in length. In certain embodiments, the sequence of the PRS component can differ up to 1, up to 2, up to 3, or up to 4 nucleobase positions from the sequence of any of the PRS components disclosed in herein and preferably at Tables 3, 12, 14, 23, 25, or 28.

In one embodiment, the ACT-UP1 compound targeting PKD1 comprises: a PRS component about 5-20 nucleosides in length and an ASO component about 13-30 nucleosides in length with up to 1, 2, or 3 non-complementary nucleobase(s) relative to a PKD1 mRNA, or portion of the mRNA; a PRS component about 5-15 nucleosides in length and an ASO component about 13-25 nucleosides in length with up to 1, 2, or 3 non-complementary nucleobase(s) relative to a PKD1 mRNA, or portion of the mRNA; a PRS component about 7-13 nucleosides in length and an ASO component about 13-20 nucleosides in length with up to 1, 2, or 3 non-complementary nucleobase(s) relative to a PKD1 mRNA, or portion of the mRNA; a PRS component about 9-11 nucleosides in length and an ASO component about 13-20 nucleosides in length with up to 1, 2, or 3 non-complementary nucleobase(s) relative to a PKD1 mRNA, or portion of the mRNA; a PRS component about 9-10 nucleosides in length and an ASO component about 13-20 nucleosides in length with up to 1, 2, or 3 non-complementary nucleobase(s) relative to a PKD1 mRNA, or portion of the mRNA; a PRS component about 9-10 nucleosides in length and an ASO component about 13-20 nucleosides in length with up to 1, 2, or 3 non-complementary nucleobase(s) relative to a PKD1 mRNA, or portion of the mRNA. In a preferred embodiment, the compound targeting PKD1 comprises a PRS component 9-10 nucleosides in length and an ASO component 15 nucleosides in length with up to 1, 2, or 3 non-complementary nucleobase(s) relative to a PKD1 mRNA, or portion of the mRNA.

It is possible to increase or decrease the length of an antisense compound, such as an antisense oligonucleotide, and/or introduce base mismatch(s) with the target without eliminating activity (U.S. Pat. No. 7,772,203, incorporated-by-reference herein). For example, it is possible to introduce non-canonical base pairings (e.g., A:G, A:C, G:U, I:U, I:A, or I:C) into an antisense oligonucleotide without eliminating activity. In certain embodiments, designing an antisense oligonucleotide with one or more non-canonical base pairings, i.e., mismatch(s), enhances the activity of the antisense compound.

### Antisense Oligonucleotide Motifs

A motif refers to a pattern of modification of an antisense compound. Various motifs have been described in the art and are incorporated-by-reference herein (e.g., U.S. Pat. Nos. 11,203,755; 10,870,849; EP Patent 1,532,248; U.S. Pat. Nos. 11,406,716; 10,668,170; 9,796,974; 8,754,201; 10,837,013; 7,732,593; 7,015,315; 7,750,144; 8,420,799; 8,809,516; 8,796,436; U.S. Pat. Nos. 8,859,749; 9,708,615; 10,233,448; 10,273,477; 10,612,024; 10,612,027; 10,669,544; 11,401,517; 9,260,471; 9,970,005; 11,193,126; 8,604,183; 9,150,605; 9,708,610; USSN 2020/0031862; and USSN 2016/0272970).

In certain embodiments, antisense compounds disclosed herein have chemically modified subunits arranged into motifs or patterns (i.e., chemical modification motifs/patterns) to confer on to the antisense compounds beneficial properties including, but not limited to: enhanced activity to increase potency; increased binding affinity to increase specificity for a target nucleic acid, thereby limiting off-target effects and increasing safety; or enhanced resistance to degradation by in vivo nucleases thereby increasing stability and durability.

Target mRNAs and Associated Gene Expression

Several embodiments are directed to methods of upregulating an mRNA and/or upregulating protein expression by a PKD1 targeting antisense compound. In certain embodiments, the PKD1 target is an mRNA transcript of PKD1. In a preferred embodiment, the PKD1 mRNA target is the sequence in GENBANK Accession No. NM_001009944.3 (SEQ ID NO: 1). In certain embodiments, a target region on the PKD1 mRNA is in the 5′ UTR or 3′UTR.

In certain embodiments, the target region on the mRNA is an upstream open reading frame (uORF) site. In certain embodiments, the target region on the mRNA is a microRNA (miRNA) binding site. In a preferred embodiment, the target region on the mRNA is a miR-17 binding site. In certain embodiments, the compound is a dual-functional compound that binds to (a) a region inthe 3′UTR of PKD1 and targets a miRNA binding site, or (b) a region in the 5′UTR of PKD1 and targets a uORF site.

In certain embodiments, the compound targets a region of the 3′UTR of PKD1 about 20 to 50, 40 to 70, 60 to 90, 80 to 110, 100 to 130, 120 to 150, 140 to 170, 160 to 190, 170 to 200, 180 to 210, 200 to 230, 220 to 250, 240 to 300, or 280 to 500 nucleotides downstream of its stop codon. In one embodiment, the compound targets the 3′UTR of PKD1 about 60 to 300, 100 to 260, 120 to 250, 140 to 220, 140 to 200, 170 to 200 or 175 to 195 nucleotides downstream of its stop codon. In a preferred embodiment, the compound targets the region of the 3′UTR of PKD1 about 170 to 200 nucleotides downstream of a stop codon. In a more preferred embodiment, the compound targets the region of the 3′UTR of PKD1 about 175 to 195 nucleotides downstream of a stop codon. In certain embodiments, the target mRNA is present in a eukaryotic cell or a prokaryotic cell. In certain embodiments, the target protein is expressed in a eukaryotic cell or a prokaryotic cell. In certain embodiments, the eukaryotic cell or a prokaryotic cell is a mammalian cell, a plant cell, a yeast cell, or a bacteria cell. In some embodiments, the mammalian cell includes cells from mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perissodactyla, including Equines (horses). In some aspects, the mammals are of the order Primates, Ceboids, or Simoids (monkeys) or of the order Anthropoids (humans and apes). In a preferred aspect, the mammalian cell is a human cell. In certain embodiments, the cell is in the form of a cultured cell line. In certain embodiments, the cell line is a primary cell line.

### Hybridization

In some embodiments, hybridization occurs between an antisense compound disclosed herein and a mRNA. The most common mechanism of hybridization involves hydrogen bonding (e.g., Watson-Crick, Hoogsteen or reverse Hoogsteen hydrogen bonding) between complementary nucleobases of the nucleic acid molecules.

In Watson-Crick canonical base pairings, adenine (A) is complementary to thymine (T) in DNA, adenine (A) is complementary to uracil (U) in RNA, and Guanine (G) is complementary to cytosine (C) in both DNA and RNA. Base pairs, or complementary nucleobases, are usually Watson-Crick base pairs (e.g., C:G, A:U, or A:T), but, non-canonical base pairs such as Hoogsteen base pairs (e.g., A:G or A:U), Wobble base pairs (e.g., G:U, I:U, I:A, or I:C, wherein I is hypoxanthine) and the like are also permitted during hybridization of the antisense compound to a target nucleic acid or target region. Wobble base pairs in RNAi agents have previously been described (see e.g., U.S. Pat. Nos. 7,732,593, 7,750,144).

Nucleobase complementarity facilitates hybridization of the antisense compounds described herein to their target nucleic acids, with the stronger the pairing (e.g., the more base pairs and/or the stronger the hydrogen bond), the stronger the hybridization of the antisense compound to the target. Hybridization can occur under varying conditions. Stringent conditions are sequence-dependent and are determined by the nature and composition of the antisense compound to be hybridized.

Methods of determining whether a sequence is specifically hybridizable to a target nucleic acid are well known in the art. In certain embodiments, the antisense compounds provided herein are specifically hybridizable with a target mRNA with little to no off-target binding.

### Complementarity

An antisense compound comprising an antisense oligonucleotide (ASO) is complementary to a target nucleic acid when a sufficient number of nucleobases of the antisense oligonucleotide can hydrogen bond with the corresponding nucleobases of the target nucleic acid, such that a desired effect will occur (e.g., upregulation of a target nucleic acid such as a PKD1 mRNA).

Non-complementary nucleobases between an antisense oligonucleotide and an mRNA nucleic acid may be tolerated provided that the antisense oligonucleotide remains able to specifically hybridize to a target nucleic acid. Moreover, an antisense oligonucleotide may hybridize to one or more segments of an mRNA nucleic acid such that intervening or adjacent segments are not involved in the hybridization event (e.g., a loop structure, mismatch, or hairpin structure).

In certain embodiments, for antisense compounds that comprise an antisense oligonucleotide, the antisense oligonucleotide portion is, or are at least, about 70%, 80%, 85%, 86%, 87%, 88%, 89%, 90%, 91%, 92%, 93%, 94%, 95%, 96%, 97%, 98%, 99%, or 100% complementary to an mRNA nucleic acid, a target region, target segment, or specified portion thereof. Percent complementarity of an antisense oligonucleotide with a target nucleic acid can be determined using routine methods.

For example, an antisense compound in which about 18 out of 20 nucleobases of the antisense oligonucleotide are complementary to a target region, and would therefore specifically hybridize to the target, would represent 90 percent complementarity. In this example, the remaining noncomplementary nucleobases of the antisense oligonucleotide may be clustered or interspersed with complementary nucleobases and need not be contiguous to each other or to complementary nucleobases. As such, an antisense oligonucleotide which is 18 nucleobases in length, having four noncomplementary nucleobases which are flanked by two regions of complete complementarity with the target nucleic acid, would have 77.8% overall complementarity with the target nucleic acid and would thus fall within the scope of the present invention. Percent complementarity of an antisense oligonucleotide with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649-656). Percent homology, sequence identity, or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense oligonucleotides provided herein, or specified portions thereof, are fully complementary (i.e., 100% complementary) to a target nucleic acid, or specified portion thereof. For example, an antisense oligonucleotide may be fully complementary to an mRNA nucleic acid, or a target region, or a target segment or a target sequence thereof. As used herein, “fully complementary” means each nucleobase of an antisense oligonucleotide is capable of precise base pairing with the corresponding nucleobases of a target nucleic acid. For example, a 20 nucleobase antisense oligonucleotide is fully complementary to a target sequence that is 400 nucleobases long, so long as there is a corresponding 20 nucleobase portion of the target nucleic acid that is fully complementary to the antisense oligonucleotide. Fully complementary can also be used in reference to a specified portion of the first and/or the second nucleic acid. For example, a 20 nucleobase portion of a 30 nucleobase antisense oligonucleotide can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense oligonucleotide. At the same time, the entire 30 nucleobase antisense oligonucleotide may or may not be fully complementary to the target sequence, depending on whether the remaining 10 nucleobases of the antisense oligonucleotide are also complementary to the target sequence.

The location of a non-complementary nucleobase may be at the 5′ end or 3′ end of the antisense oligonucleotide. Alternatively, the non-complementary nucleobase or nucleobases may be at an internal position of the antisense oligonucleotide. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense oligonucleotides that are, or are up to about 13, 14, 15, 16, 17, 18, 19, or 20 nucleosides in length comprise no more than 4, no more than 3, no more than 2, or no more than one (1) non-complementary nucleobase(s) relative to a target nucleic acid, such as an mRNA nucleic acid, or specified portion thereof. In accordance with the practice of the invention, the no more than 4, no more than 3, no more than 2, or no more than 1 non-complementary nucleobase(s) relative to a target nucleic acid, such as an mRNA nucleic acid, or specified portion thereof, can include zero (0) non-complementary nucleobase(s) relative to the target PKD1 sequence.

In certain embodiments, antisense oligonucleotides that are, or are up to about 13, 14, 15, 16, 17, 18, 19, 20, 21, 22, 23, 24, 25, 26, 27, 28, 29, or 30 nucleosides in length comprise no more than 6, no more than 5, no more than 4, no more than 3, no more than 2, or no more than 1 non-complementary nucleobase(s) relative to a target nucleic acid, such as an mRNA nucleic acid, or specified portion thereof. In accordance with the practice of the invention, the no more than 6, no more than 5, no more than 4, no more than 3, no more than 2, or no more than 1 non-complementary nucleobase(s) relative to a target nucleic acid, such as an mRNA nucleic acid, or specified portion thereof, can include zero (0) non-complementary nucleobase(s) relative to the target PKD1 sequence.

The antisense oligonucleotides provided also include those that are complementary to a portion of a target nucleic acid. As used herein, “portion” refers to a defined number of contiguous (i.e. linked) nucleosides within a region or segment of a target nucleic acid. A “portion” can also refer to a defined number of contiguous nucleosides of an antisense oligonucleotide. In certain embodiments, the antisense oligonucleotides are complementary to at least a 10 nucleoside portion of a target segment. In certain embodiments, the antisense oligonucleotides are complementary to at least an 11 nucleoside portion of a target segment. In certain embodiments, the antisense oligonucleotides are complementary to at least a 12 nucleoside portion of a target segment. In certain embodiments, the antisense oligonucleotides are complementary to at least a 13 nucleoside portion of a target segment. In certain embodiments, the antisense oligonucleotides are complementary to at least a 14 nucleoside portion of a target segment. In certain embodiments, the antisense oligonucleotides are complementary to at least a 15 nucleoside portion of a target segment. Also contemplated are antisense oligonucleotides that are complementary to at least an about 12, 13, 14, 15, 16, 17, 18, 19, 20, or more nucleobase portion of a target segment, or a range defined by any two of these values.

### Identity

The antisense oligonucleotides provided herein may also have a defined percent identity to a particular nucleotide sequence, SEQ ID NO, or compound represented by a specific identity number, or portion thereof. As used herein, an antisense oligonucleotide is identical to the sequence disclosed herein if it has the same nucleobase pairing ability. For example, an RNA that contains uracil in place of thymidine in a disclosed DNA sequence would be considered identical to the DNA sequence since both uracil and thymidine pair with adenine. Shortened and lengthened versions of the antisense oligonucleotide described herein as well as oligonucleotides having non-identical bases relative to the antisense oligonucleotides provided herein are also contemplated.

The non-identical bases may be adjacent to each other or dispersed throughout the antisense oligonucleotide. Percent identity of an antisense oligonucleotide is calculated according to the number of bases that have identical base pairing relative to the sequence to which it is being compared.

In certain embodiments, the antisense oligonucleotides, or portions thereof, are at least about 70%, 75%, 80%, 85%, 90%, 95%, 96%, 97%, 98%, 99%, or 100% identical to one or more of the antisense oligonucleotides or SEQ ID NOs, or a portion thereof, disclosed herein.

In certain embodiments, a portion of the antisense oligonucleotide is compared to an equal length portion of the target nucleic acid. In certain embodiments, an about 10, 11, 12, 13, 14, 15, 16, 17, 18, 19, 20, 21, 22, 23, 24, or 25 nucleoside portion is compared to an equal length portion of the target nucleic acid.

### Chemical Modifications

A nucleoside is a base-sugar combination. The nucleobase (also known as base) portion of the nucleoside is normally a heterocyclic base moiety. Nucleotides are nucleosides that further include a covalent linkage (e.g., phosphate group or a chemically modified linkage as described infra) to the sugar portion of the nucleoside. Oligonucleotides are formed through the covalent linkage of adjacent nucleotides to one another, to form a linear polymeric oligonucleotide. Within the oligonucleotide structure, the linkage groups are commonly referred to as forming the internucleoside linkages of the oligonucleotide.

Modifications to antisense compounds encompass substitutions or changes to nucleobases, internucleoside linkages or sugar moieties. Modified antisense compounds are often preferred over native or unmodified forms because of desirable properties such as, for example, enhanced delivery (e.g., increased cellular uptake), enhanced specificity or affinity for a nucleic acid target, increased stability in the presence of nucleases, enhanced safety (e.g., fewer side effects after administration of the compound to a subject) or increased potency (e.g., increased activity).

### Internucleoside Linkage Modifications

The naturally occurring internucleoside linkage of RNA and DNA is a 3′ to 5′ phosphodiester linkage. For nucleosides that include a pentofuranosyl sugar, the phosphate group can be linked to the 2′, 3′ or 5′ hydroxyl moiety of the sugar. Oligomeric compounds having one or more modified, i.e. non-naturally occurring, internucleoside linkages are often selected over oligomeric compounds having naturally occurring internucleoside linkages because of desirable properties such as, for example, enhanced cellular uptake, enhanced affinity for target nucleic acids, decreased toxicity, increased stability and durability, decreased degradation and other desirable features for an oligomeric compound. Modified internucleoside linkages and their advantages are well known in the art (Crooke, S. T., et al., 2021a Antisense technology: an overview and prospectus. Nat Rev Drug Discov 20:427-453; Crooke, S. T., et al., 2021b Antisense technology: A review. J Biol Chem 296: 100416).

Oligomeric compounds having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates (e.g., 5′-methylphosphonate (5′-MP)), phosphoramidate, phosphorothioates (e.g., phosphorodithioate Rp isomer (PS, Rp), phosphorodithioate Rp isomer (PS, Sp), or 5′-phosphorothioate (5′-PS)), methoxypropylphosphonate, (S)-5′-C-methyl with Phosphate, and 5′-(E)-vinylphosphonate. In certain aspects, the internucleoside linkage may be replaced with a peptide nucleic acid (PNA) linkage, a morpholino linkage, or Mesyl phosphoramidate linkage.

In certain embodiments, oligomeric compounds targeted to a nucleic acid comprise one or more modified internucleoside linkages. In certain embodiments, the modified internucleoside linkages are phosphorothioate (PS) linkages. In certain embodiments, one or more internucleoside linkage of an oligomeric compound is a phosphorothioate internucleoside linkage. In certain embodiments, each internucleoside linkage of an oligomeric compound is a phosphorothioate internucleoside linkage. In certain embodiments, each internucleoside linkage of an oligonucleotide is a phosphorothioate internucleoside linkage.

### Sugar Modifications

Oligomeric compounds provided herein can contain one or more nucleosides wherein the sugar group has been modified. Such sugar modified nucleosides may impart desirable features such as increased stability, increased durability (e.g., increased half-life), increased binding affinity, decreased off-target effects, decreased immunogenicity, decreased toxicity, increased potency, or some other beneficial biological property to the oligomeric compounds. Sugar modifications and their advantages are known in the art (Faria, M., and H. Ulrich, 2008 Sugar boost: when ribose modifications improve oligonucleotide performance. Curr Opin Mol Ther 10: 168-175; Crooke, S. T., et al., 2021b Antisense technology: A review. J Biol Chem 296: 100416; Egli, M., and M. Manoharan, 2023 Chemistry, structure and function of approved oligonucleotide therapeutics. Nucleic Acids Res 51: 2529-2573).

In certain embodiments, nucleosides comprise a chemically modified ribofuranose ring moiety. Examples of chemically modified ribofuranose rings can include, without limitation, addition of substituent groups (e.g., 5′ sugar modifications, or 2′ sugar modifications); bridging of non-geminal ring atoms to form bicyclic nucleic acids (BNA); replacement of the ribosyl ring oxygen atom with S, N(R), or C(R1)(R)2 (R=H, C₁-C₁₂ alkyl or a protecting group); nucleoside mimetic; and combinations thereof.

Examples of chemically modified sugars include, 2′-F-5′-methyl substituted nucleoside (see, e.g., PCT Publication WO2008101157 for other disclosed 5′, 2′-bis substituted nucleosides), replacement of the ribosyl ring oxygen atom with S with further substitution at the 2′-position (see, e.g., U.S. Publication US20050130923), or, alternatively, 5′-substitution of a BNA (see, PCT Publication WO2007134181 wherein LNA is substituted with, for example, a 5′-methyl or a 5′-vinyl group).

A 2′-modified sugar refers to a furanosyl sugar modified at the 2′ position. A 2′-modified nucleoside refers to a nucleoside comprising a sugar modified at the 2′ position of a furanose ring. In certain embodiments, such modifications include substituents selected from: a halide, including, but not limited to substituted and unsubstituted alkoxy, substituted and unsubstituted thioalkyl, substituted and unsubstituted aminoalkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, and a group for improving the pharmacodynamic properties of an oligomeric compound, and other substituents having similar properties.

Further examples of nucleosides having modified sugar moieties include, without limitation, nucleosides comprising 5′-vinyl, 5′-methyl (R or S), 2′-F-5′-methyl, 4′-S, 2′-deoxy-2′-fluoro (2′-F), 2′—OCH₃ (2′-O-methyl, 2′-OMe), 2′-O(CH₂)₂OCH₃ (2′-O-methoxyethyl, 2′-O-MOE, 2′-MOE), 2′-O-methyl-4-pyridine, phosphorodiamidate morpholino (PMO), tricyclo-DNA (tcDNA), 2′-arabino-fluoro, 2′-O-benzyl, glycol nucleic acid (GNA), and/or unlocked nucleic acid (UNA) substituent groups. The substituent at the 2′ position can also be selected from allyl, amino, azido, thio, O-allyl, O—C₁-C₁₀ alkyl, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C₁-C₁₀alkyl. 2′-OMe or 2′—OCH₃ or 2′-O-methyl each refers to a nucleoside comprising a sugar comprising an —OCH₃ group at the 2′ position of the sugar ring. 2′-F refers to a sugar comprising a fluoro group at the 2′ position. 2′-O-methoxyethyl or 2′-O-MOE or 2′-MOE each refers to a nucleoside comprising a sugar comprising an —O(CH₂)₂OCH₃ group at the 2′ position of the sugar ring.

BNAs refer to modified nucleosides comprising a bicyclic sugar moiety wherein a bridge connecting two carbon atoms of the sugar ring connects the 2′ carbon and another carbon of the sugar ring. Examples of bicyclic nucleosides include, without limitation, nucleosides comprising a bridge between the 4′ and the 2′ ribosyl ring atoms such as in locked nucleic acid (LNA). In certain embodiments, oligomeric compounds provided herein include one or more bicyclic nucleosides wherein the bridge comprises a 4′ to 2′ bicyclic nucleoside. LNAs and UNAs have been described by Campbell and Wengel (Chem Soc Rev, 2011, 40(12):5680-9) and are incorporated-by-reference herein.

In certain embodiments, oligomeric compounds comprise one or more nucleotides having modified sugar moieties. In certain embodiments, the modified sugar moiety is 2′-MOE. In certain embodiments, the modified sugar moiety has a 2′-OMe modification. In certain embodiments, the modified sugar moiety has a 2′-F modification. In certain embodiments, the modified sugar moiety is a cEt.

### Oligomeric Compound Delivery Systems

Oligomeric compounds require entry into target cells to become active. A variety of modalities have been used to traffic oligomeric compounds into target cells including viral delivery vectors, lipid-based delivery, polymer-based delivery, and conjugate-based delivery (Paunovska et al., Drug Delivery Systems for RNA Therapeutics, 2022, Nature Reviews Genetics, 23(5):265-280; Chen et al., 2022, Molecular Therapy, Nucleic Acids, 29:150-160).

Lipid-based particles can form specific structures such as micelles, liposomes, and lipid nanoparticles (LPNs) to carry oligomeric compounds-into cells. To form these particles. LPNs can-include one or more of a cationic or ionizable lipid (e.g., DLin-MC3-DMA, SM-102, or ALC-0315), cholesterol, a helper lipid, 1,2-distearoyl-sn-glycero-3-phosphocholine (DSPC), poly(ethylene glycol) (PEG) modified lipid (e.g., PEG-2000-C-DMG, PEG-2000-DMG, or ALC-0159), C12-200, cKK-E12, and the like. Different combinations of lipids can be formulated to affect the delivery of the oligomeric compound to different types of cells. In one example, therapeutic siRNA patisiran was formulated in cationic ionizable lipid DLin-MC3-DMA, cholesterol, polar phospholipid DSPC, and PEG-2000-C-DMG for delivery to hepatocytes.

Polymer-based particles are also used in oligomeric compound delivery systems. Such polymers include poly(lactic-co-glycolic acid) (PLGA), polyethylenimine (PEI), poly(l-lysine) (PLL), poly(beta-amino ester) (PBAE), dendrimers (e.g., poly(amidoamine) (PAMAM) or PLL), and other polymers or modified polymers thereof. The polymer composition can be varied depending on the traits desired for delivery of the oligomeric compound.

The oligomeric compounds, or a salt thereof, disclosed herein may be covalently linked to one or more moieties or conjugate agents which enhance the activity, cellular distribution or cellular uptake of the resulting compound. Conjugate agents can include cholesterols, lipids, carbohydrates, phospholipids, biotin, phenazine, folate, phenanthridine, anthraquinone, acridine, fluoresceins, rhodamines, coumarins, peptides, antibodies, dyes, tocopherol (Nishina et al., 2008, Molecular Therapy, 16(4):734-740), etc. Conjugate-based delivery can actively deliver oligomeric compounds to specific cell types.

In an example, N-Acetylgalactosamine (GalNAc) is conjugated to an oligomeric compound and delivers the compound into hepatocytes. Various Ga1NAc conjugate agents can be found in several publications including the following, all of which are incorporated-by-reference herein: Sharma et al., 2018, Bioconjugate Chem, 29:2478-2488; Nair et al., J. Am. Chem. Soc. 2014, 136(49):16958-16961; Keam, 2022, Drugs, 82:1419-1425; U.S. Pat. No. 10,087,208; Prakash et al., 2014, Nucleic Acids Res, 42(13):8796-807; Debacker et al., 2020, Molecular Therapy, 28(8):1759-1771; U.S. Pat. Nos. 11,110,174; 9,796,756; 9,181,549; 10,344,275; 10,570,169; 9,506,030; 7,582,744; and, WO2024137545.

### Oligomeric Compound Synthesis

Oligomeric compounds were designed, synthesized, and prepared using methods known in the art.

Solid phase syntheses of oligonucleotides were done on a MerMade™ 48× synthesizer (BioAutomation, LGC, Biosearch Technologies, Hoddesdon, UK), which can make up to 48 1 μMole or 5 μMole scale oligonucleotides per run using standard phosphoramidite chemistry.

Phosphoramidite synthesis of oligonucleotides on a solid support is well known in the art (e.g., Beaucage and Caruthers, 1981, Tetrahedron Letters, 22(20): 1859-1862; Roy and Caruthers, 2013, Molecules, 18:14268-14284; and Roy and Caruthers et al., 2021, Nature Communications, 12:2760). Solid support is controlled pore glass (500-1400 Å) loaded with universal linkers or loaded with 3′-GaINAc conjugates (AM Chemicals, Vista, CA, USA; Primetech ALC, Minsk, Belarus; Gene Link, Elmsford, NY, USA; or any GalNAc conjugate disclosed herein) or universal solid support (AM Chemicals, Vista, CA, USA). Ancillary synthesis reagents and standard 2′-cyanoethyl phosphoramidite monomers (2′-fluoro nucleosides, 2′-O-methyl nucleosides, RNA nucleosides, DNA nucleosides) were obtained from various sources (Hongene Biotech, Shanghai, China; Sigma-Aldrich, St. Louis, MO, USA; Glen Research, Sterling, VA, USA; ThermoFisher Scientific, Waltham, MA, USA; LGC Biosearch Technologies, Hoddesdon, UK).

Phosphoramidite mixtures were prepared in anhydrous acetonitrile or 30% DMF:acetonitrile and were coupled using 0.25M 4,5-dicyanoimidazole(DCI)(Sigma-Aldrich, St. Louis, MO, USA) with coupling times ranging from 120-360 seconds. Standard phosphodiester linkages were achieved using 0.02M iodine mixture in Tetrahydrofuran (THF), pyridine and water. Phosphorothiate linkages were generated using 0.05M sulfurizing Reagent II (3-((Dimethylamino-methylidene)amino)-3H-1,2,4-dithiazole-3-thione, DDTT) (40:60, Pyridine/Acetonitrile) (LGC Biosearch Technologies, Hoddesdon, UK) with an oxidation time of 6 minutes. All sequences were synthesized with Dimethoxy Trityl (DMT) protecting group removed.

Upon completion of solid phase synthesis, the oligonucleotides were cleaved from the solid support and deprotection of base labile groups performed by incubation in ammonium hydroxide at 55° C. for 6 hours. Ammonium hydroxide was removed using a centrifugal vacuum concentrator to dryness at room temperature. For sequences containing natural ribonucleotides (2′-OH) protected with tert-butyl dimethyl silyl (TBDMS), a second deprotection was performed using triethylamine; trihydrofluoride (TEA;3HF). To each TBDMS protected oligonucleotide 100 μL DMSO and 125 μL TEA:3HF were added and incubated at 65*C for 2.5 hours. After incubation, 25 μL of 3M sodium acetate was added to the solution which was subsequently precipitated in butanol at −20° C. for 30 minutes. The cloudy solution was centrifuged to a cake at which time the supernatant was carefully decanted with a pipette. The standard precipitation process was then completed with 75% ethanol:water then 100% ethanol as supernatant solutions. The oligonucleotide cake was dried for 30 minutes in a centrifugal vacuum concentrator.

Desalting without HPLC purification was performed after precipitation with 3M sodium acetate with a follow on G25 Sephadext column (Sigma-Aldrich, St. Louis, MO, USA) elution.

Purification of oligonucleotides was afforded by anion exchange chromatography on a Gilson GX271 prep HPLC system (Middleton, WI, USA) using BioWorks Q40 resin (Uppsala, Sweden).

The final desalting was performed using a Sephadex® G25 column. All oligonucleotides were analyzed by ion pairing reverse phase HPLC for purity on an Agilent 1200 analytical HPLC (Santa Clara, CA, USA), negative ion mass spectrometry for intact mass on an Agilent 6130 single quad mass spectrometer (Santa Clara, CA, USA), and A260 quantification by UV/Vis on a Tecan Infinite® M Plex plate reader (Zurich, Switzerland).

### In Vitro Testing of Oligomeric Compounds

Described herein are methods for the treatment of cells with oligomeric compounds such as compounds targeting PKD1.

Cells may be treated with oligomeric compounds when the cells reach approximately 60-80% confluency in culture.

Reagents commonly used to introduce oligomeric compounds into cultured cells include the cationic lipid transfection reagent Oligofectamine™ 2000 or Lipofectamine™ 2000 (ThermoFisher Scientific, Waltham, MA). In one example, oligomeric compounds may be mixed with Oligofectamine™ 2000 in OPTI-MEM (ThermoFisher Scientific, Waltham, MA) to achieve the desired final concentration of oligomeric compounds that may range from 0.001 to 300 nM oligomeric compounds in culture medium. Transfection procedures are done according to the manufacturer's recommended protocols.

Another technique used to introduce oligomeric compounds into cultured cells includes electroporation.

Oligomeric compounds conjugated with a delivery moiety can be introduced to cells through incubation of the conjugated compounds with cells without transfection reagents, referenced herein as “free uptake”.

Cells are treated with oligomeric compounds by routine methods. Cells may be harvested 4-144 hours after oligomeric compounds treatment, at which time mRNA (harvested at 4-144 hrs) or protein levels (extracted at 24-96 hrs) of target nucleic acids are measured by methods known in the art and described herein. In general, treatments are performed in multiple replicates, and the data are presented as the average of the replicate treatments plus the standard deviation.

The concentration of oligomeric compounds used varies from cell line to cell line and target to target. Methods to determine the optimal oligomeric compound concentration for a particular target in a particular cell line are well-known in the art. In general, cells are treated with oligomeric compounds in a dosgrdependent manner to allow for the calculation of the half-maximal inhibitory concentration value (IC50). Oligomeric compounds are typically used at concentrations ranging from 0.001 nM to 300 nM when transfected with Oligofectamine™ 2000. Oligomeric compounds are used at higher concentrations ranging from 7.5 to 20,000 nM when transfected using electroporation or free uptake.

### In Vivo Testing of Oligomeric Compounds

The oligomeric compounds of the invention, for example, compounds targeting, are tested in animals to assess their ability to modulate expression of a target protein and produce phenotypic changes, such as a change in one or more markers affected by the target nucleic acid. Also, the phenotypic change can be a decrease in a disease, disorder, condition, or symptom related to the target nucleic acid. Testing may be performed in normal animals, or in experimental disease models. For administration to animals, oligomeric compounds are formulated in a pharmaceutically acceptable diluent, such as phosphate-buffered saline (PBS). Administration includes parenteral routes of administration, such as intraperitoneal, intravenous, and subcutaneous. Calculation of dosage and dosing frequency depends upon factors such as route of administration and animal body weight. In one embodiment, following a period of treatment with oligomeric compounds of the invention, RNA encoding the target nucleic acid is isolated from liver tissue, and changes in the target nucleic acid expression are measured. Changes in protein levels expressed by the target nucleic acid can also be measured.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Thermo Fisher Scientific, Waltham, MA), Qiagen RNeasy kit (Qiagen, Hilden, Germany), or AcroPrep Advance 96-well Filter Plates (Pall Corporation, Port Washington, New York) using Qiagen's RLT, RWI, and RPE buffers. RNA extraction procedures are done according to the manufacturer's recommended protocols.

### Protein Isolation

Protein analysis can be conducted on total cell extracts or tissue lysates. Methods of cell extracts or tissue lysates are well known in the art. Cellular proteins are prepared using methods well known in the art, for example, using RIPA buffer (ThermoFisher Scientific, Waltham, MA) or other appropriate buffers. Tissue lysates are prepared in RIPA buffer, with tissue homogenizer. Levels of proteins can be analyzed using Western blotting, ELISA, or other approaches.

### Compositions and Methodsfor Formulating Pharmaceutical Compositions

The oligomeric compounds of the invention, such as compounds, or a salt thereof, targeting PKD1 described herein, can be combined with pharmaceutically acceptable active or inert substances, such as a diluent, excipient, or carrier, for the preparation of pharmaceutical compositions or formulations.

Compositions and methods for the formulation of pharmaceutical compositions are dependent upon a number of criteria, including, but not limited to, route of administration, extent of disease, or dose to be administered.

In certain embodiments, the pharmaceutical carrier or excipient is a pharmaceutically acceptable solvent, suspending agent, or any other pharmacologically inert vehicle for delivering one or more oligomeric compounds to an animal. The excipient can be liquid or solid and can be selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone and/or hydroxypropyl methylcellulose, etc.); fillers (e.g., lactose and other sugars, microcrystalline cellulose, pectin, gelatin, calcium sulfate, ethyl cellulose, polyacrylates and/or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, and/or sodium acetate, etc.); disintegrants (e.g., starch, and/or sodium starch glycolate, etc.); and wetting agents (e.g., sodium lauryl sulphate, etc.).

Pharmaceutically acceptable organic or inorganic excipients, which do not deleteriously react with nucleic acid compounds, suitable for parenteral or non-parenteral administration can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an oligomeric compound and a pharmaceutically acceptable diluent. In certain embodiments, the pharmaceutically acceptable diluent is PBS. In certain embodiments, the oligomeric compound is a PKD1 targeting compound.

Pharmaceutical compositions comprising oligomeric compounds such as compounds, or a salt thereof, targeting PKD1 can encompass any pharmaceutically acceptable salts, esters, or salts of such esters, which, upon administration to an animal, including a human, is capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts.

In certain embodiments, a pharmaceutical composition is prepared for administration by injection (e.g., intravenous, subcutaneous, and/or intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer (e.g., PBS). In certain embodiments, other ingredients are included (e.g., ingredients that aid in solubility or serve as preservatives). In certain embodiments, injectable suspensions are prepared using-appropriate liquid carriers, suspending agents and the like. Certain pharmaceutical compositions for injection are presented in unit dosage form, e.g., in ampoules or in multi-dose containers.

### Dosages

For purposes of the disclosure, the amount or dose of the active agent (i.e., oligomeric compound of the invention) administered should be sufficient to e.g., modulate the expression of a target protein in an animal. In the animal (e.g., human), dose will be determined by the efficacy of the particular active agent and the condition of the animal, as well as the body weight of the animal to be treated.

Many assays for determining an administered dose are known in the art.

The dose of the active agent of the present disclosure will also be determined by the existence, nature, and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated.

### Dosing

In certain embodiments, pharmaceutical compositions are administered according to a dosing regimen (e.g., dose, dose frequency, and duration) wherein the dosing regimen can be selected to achieve a desired effect. The desired effect can be, for example, reduction of a target nucleic acid or the prevention, reduction, amelioration or slowing the progression of a disease, disorder and/or condition, or symptom thereof, associated with the target nucleic acid. In certain embodiments, the variables of the dosing regimen are adjusted to result in a desired concentration of pharmaceutical composition in. a subject. “Concentration of pharmaceutical composition” as used with regard to dose regimen can refer to the oligomeric compound or active ingredient of the pharmaceutical composition. For example, in certain embodiments, dose and dose frequency are adjusted to provide a tissue concentration or plasma concentration of a pharmaceutical composition at an amount sufficient to achieve a desired effect.

Dosing is dependent on severity and responsiveness of the disease state to be treated, with the course of treatment lasting from several days to several months, or until a cure is effected or a diminution of the disease state is achieved. Dosing is also dependent on drug potency and metabolism. In certain embodiments, dosage is from about 0.01 sg to 50 mg per kg of body weight, 0.01 μg to 100 mg per kg of body weight, or within a range of about 0.001 mg to 1000 mg dosing, and may be given once or more daily, weekly, monthly, quarterly, or yearly, or even once every 2 to 20 years. Following successful treatment, it may be desirable to have the patient undergo maintenance therapy to prevent the recurrence of the disease state, wherein the oligomeric compound is administered in maintenance doses, ranging from about 0.01 μg to 100 mg per kg of body weight, once or more daily, once or more weekly, once or more monthly, once or more quarterly, once or more yearly, to once every 20 years or ranging from about 0.001 mg to 1000 mg dosing. In certain embodiments, it may be desirable to administer the oligomeric compound from at most once daily, once weekly, once monthly, once quarterly, once yearly, once every two years, once every three years, once every four years, once every five years, once every ten years, to once every 20 years.

In certain embodiments, the range of dosing is between any of about 1 mg-1500 mg, 100 mg-1400 mg, 100 mg-1300 mg, 100 mg-1200 mg, 100 mg-1100 mg, 100 mg-1000 mg, 100 mg-900 mg, 200 mg-800 mg, 300 mg-700 mg, 400 mg-600 mg, 100 mg-400 mg, 200 mg-500 mg, 300 mg-600 mg, and 400 mg-700 mg. In certain embodiments, a dose is about 100 mg, 150 mg, 200 mg, 250 mg, 300 mg, 350 mg, 400 mg, 450 mg, 500 mg, 550 mg, 600 mg, 650 mg, 700 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg.

In certain embodiments, the oligomeric compound is dosed at any of about 150 mg, 200 mg, 300 mg, 400 mg, 500 mg, 600 mg, 700 mg, 800 mg, or 900 mg twice a year. In certain embodiments, the oligomeric compound is dosed at about 100 mg, 150 mg, 200 mg, 300 mg, 400 mg, 500 mg, 600 mg, 700 mg, 800 mg, or 900 mg quarterly. In certain embodiments, the oligomeric compound is dosed at about 50 mg, 100 mg, 150 mg, 200 mg, 300 mg, 400 mg, 500 mg, 600 mg, 700 mg, 800 mg, or 900 mg once monthly or every two months. In certain embodiments, the oligomeric compound is dosed at about 10 mg, 15 mg, 20 mg, 25 mg, 50 mg, 100 mg, 150 mg, 200 mg, 300 mg, 400 mg, 500 mg, 600 mg, 700 mg, 800 mg, or 900 mg weekly or every two weeks.

### Administration

The oligomeric compounds, such as an antisense compound, or a salt thereof, targeting PKD1, or pharmaceutical compositions of the present invention can be administered in a number of ways depending upon whether local or systemic treatment is desired and upon the area to be treated. Administration can be oral, inhaled or parenteral.

In certain embodiments the compounds and compositions as described herein are administered parenterally. Parenteral administration includes intravenous, intra-arterial, subcutaneous, intraperitoneal or intramuscular injection or infusion; or intracranial, e.g., intrathecal or intraventricular, administration. In certain embodiments, parenteral administration is by infusion. Infusion can be chronic or continuous or short or intermittent. In certain embodiments, infused pharmaceutical agents are delivered with a pump.

In certain embodiments, parenteral administration is by injection. The injection can be delivered with a syringe or a pump. In certain embodiments, the injection is a bolus injection. In certain embodiments, the injection is administered directly to a tissue or organ.

In certain embodiments, formulations for parenteral, intrathecal or intraventricular administration can include sterile aqueous solutions which can also contain buffers, diluents and other suitable additives such as, but not limited to, penetration enhancers, carrier compounds and other pharmaceutically acceptable carriers or excipients.

In certain embodiments, formulations for oral administration of the compounds or compositions can include, but are not limited to, pharmaceutical carriers, excipients, powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In certain embodiments, oral formulations are those in which compounds provided herein are administered in conjunction with one or more penetration enhancers, surfactants and chelators.

### Kits of the Invention

According to another aspect of the invention, kits are provided. Kits according to the invention include package(s) comprising any of the compositions of the invention or oligomeric compound of the invention. In various aspects, the kit comprises any of the compositions of the invention as a unit dose. For purposes herein “unit dose” refers to a discrete amount dispersed in a suitable carrier.

The phrase “package” means any vessel containing compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes (including pre-filled syringes), bottles, and any packaging material suitable for a selected formulation and intended mode of administration and treatment.

The kit can also contain items that are not contained within the package but are attached to the outside of the package, for example, pipettes.

Kits may optionally contain instructions for administering compositions of the present invention to a subject having a condition in need of treatment. Kits may also comprise instructions for approved uses of components of the composition herein by regulatory agencies, such as the United States Food and Drug Administration. Kits may optionally contain labeling or product inserts for the present compositions. The package(s) and/or any product insert(s) may themselves be approved by regulatory agencies. The kits can include compositions in the solid phase or in a liquid phase (such as buffers provided) in a package. The kits can also include buffers for preparing solutions for conducting the methods, and pipettes for transferring liquids from one container to another.

The kit may also optionally contain one or more other compositions for use in combination therapies as described herein. In certain embodiments, the package(s) is a container for any of the means for administration such as intravitreal delivery, intraocular delivery, intratumoral delivery, peritumoral delivery, intraperitoneal delivery, intrathecal delivery, intramuscular injection, subcutaneous injection, intravenous delivery, intra-arterial delivery, intraventricular delivery, intrasternal delivery, intracranial delivery, or intradermal injection.

### Methods of Use

The invention provides methods for enhancing the expression of a target protein in a subject comprising administering an effective amount of an oligomeric compound of the invention, a salt thereof, or a pharmaceutical composition of the invention, so as to increase the expression of a target protein in the subject. I certain embodiments, the oligomeric compound is a PKD1 targeting compound.

The invention provides methods for enhancing the expression of a target protein in a eukaryotic cell or a prokaryotic cell comprising administering an effective amount of an oligomeric compound of the invention, a salt thereof, or a pharmaceutical composition of the invention, so as to increase the expression of a target protein in the subject. In certain embodiments, the oligomeric compound is a PKD1 targeting compound. In certain embodiments, eukaryotic cell or a prokaryotic cell is a mammalian cell, a plant cell, a yeast cell or a bacteria cell.

In certain embodiments, a method of enhancing a target gene expression in a cell comprises administering to the cell an oligomeric compound targeted to an mRNA transcript. In an embodiment, the oligomeric compound is a PKD1 compound.

In certain embodiments, an oligomeric compound of the invention increases expression of a protein by at least about 10%, 15%, 20%, 25%, 30%, 35%, 40%, 45%, 50%/o, 55%, 60%, 65%, 70%, 75%, 80%, 85%, 90%, 95%, or 100%. In an embodiment, the oligomeric compound is a PKD1 compound.

In certain embodiments, a method of enhancing PKD1 gene expression in a cell comprises administering to the cell an oligomeric compound, a salt thereof, targeted to a PKD1 mRNA transcript (GenBank Accession No: NM_001009944.3, SEQ ID NO: 1). In certain embodiments, enhancing PKD1 gene expression in a cell treats a subject suffering from a PKD1 related disease. In certain embodiments, the PKD1 related disease is related to a decreased level of PKD1 protein. In a preferred embodiment, the PKD1 related disease is ADPKD.

In certain embodiments, a method of enhancing PKD2 gene expression in a cell comprises administering to the cell an oligomeric compound targeted to a PKD2 mRNA transcript (GenBank No: NM_000297.4, SEQ ID NO: 2). In certain embodiments, enhancing PKD2 gene expression in a cell treats a subject suffering from a PKD2 related disease. In a preferred embodiment, the PKD2 related disease is ADPKD.

In certain embodiments, a method of enhancing PKD1 and PKD2 gene expression in a cell comprises administering to the cell an oligomeric compound targeted to both PKD1 and PKD2 mRNA transcript (GenBank Accession No: NM_001009944.3, SEQ ID NO: 1; GenBank Accession No: NM_000297.4, SEQ ID NO: 2). In certain embodiments, enhancing PKD1 and PKD2 gene expression in a cell treats a subject suffering from a PKD1 and PKD2 related disease. In a preferred embodiment, the PKD1 and PKD2 related disease is ADPKD.

In some embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perissodactyla, including Equines (horses). In some aspects, the mammals are of the order Primates, Ceboids, or Simoids (monkeys) or of the order Anthropoids (humans and apes). In a preferred aspect, the mammal is a human.

### In Vivo Testing of PKD1 Enhancing Compounds

In some embodiments of the present disclosure, PKD1 compounds, or a salt thereof, can be tested in subjects to assess their ability to enhance expression of a protein. In certain embodiments, PKD1 compounds can be tested in subjects to assess their ability to increase mRNA translation in order to increase protein production and/or produce phenotypic changes related to that protein. In certain embodiments, PKD1 compounds can be tested in subjects to assess their ability to treat a disease such as ADPKD associated with the protein. Testing may be performed in normal subjects, or in experimental disease models. For administration to subjects, PKD1 compounds are formulated in a pharmaceutically acceptable diluent, such as phosphate-buffered saline. Administration includes parenteral routes of administration, such as intraperitoneal, intravenous, and subcutaneous. Calculation of PKD1 compound dosage and dosing frequency depends upon factors such as route of administration and subjects body weight. In one embodiment, following a period of treatment with a PKD1 compound, the protein is isolated from a tissue and changes in protein expression are measured.

### In Vitro Assay to Identify Compounds Targeting PKD1 for Enhancing Protein Expression

In some embodiments of the present disclosure, in vitro assays to identify compounds targeting PKD1 for enhancing target protein expression are provided.

In one example, a test mRNA is chosen as a target to upregulate its protein expression. Cells in culture (eHg, HeLa, lepal-6, or HEK293), are seeded and grown for one day to ~70% confluency. The cells are then transfected with the compounds targeting PKD1 of interest at about 7.5 nM or 15 nM final concentrations using Lipofectamine™ 2000 Transfection Reagent (ThermoFisher Scientific, Waltham, MA), or are mock transfected as a control. Twenty-four (24) hr after transfection, cells are harvested and lysed, then the protein is extracted using RIPA Lysis and Extraction Buffer (ThermoFisher Scientific, Waltham, MA). The level of target protein can be determined by Western Blot or chemiluminescence, using a target protein specific antibody and optionally, a secondary antibody conjugated to either alkaline phosphatase or horseradish peroxidase. Western Blot images can be quantified using ImageJ (an open source software for processing and analyzing scientific images), and the results are quantified as percent protein levels relative to mock transfected cells following normalization to a loading control protein such as GAPDH and/or HSP90. As the control protein is not targeted by the compounds targeting PKD1 tested in the assay, the level of the control protein is not affected, showing the specificity of the compounds targeting PKD1 in modulating target protein levels.

### Certain Embodiments of the Invention

The invention is further illustrated by the following embodiments.

Embodiment I provides an ACT-UP1 compound, or salt thereof, for enhancing expression of PKD1 in a cell, wherein the compound, or salt thereof, comprises an oligomeric sequence comprising an antisense oligonucleotide (ASO) component targeting PKD1 and a protein recruiting sequence (PRS) component, wherein the ASO component comprises: (A) 13, 14, 15, 16, 17, 18, 19, 20, 21, 22, 23, 24, 25, 26, 27, 28, 29, 30, 31, 32, 33, 34, or 35 nucleosides, and (B) no more than 3, no more than 2, or no more than 1 non-complementary nucleobase(s) relative to a targeted PKD1 region of SEQ ID NO: 1.

Embodiment 2 provides aa; ACT-UP1 compound, or salt thereof, for enhancing expression of PKD1 in a cell, wherein the compound, or salt thereof, comprises an oligomeric sequence comprising an antisense oligonucleotide (ASO) component targeting PKD1 and a protein recruiting sequence (PRS) component, wherein the ASO component comprises: (A) from 13 to 35 nucleosides, and (B) no more than 3 non-complementary nucleobases relative to a targeted PKD1 region of SEQ ID NO: 1.

Embodiment 3 provides the compound of any preceding embodiment, wherein the PRS component is 5, 6, 7, 8, 9, 10, 11, 12, 13, 14, 15, 16, 17, 18, 19, 20, 21, 22, 23, 24, 25, 26, or 27 nucleosides in length.

Embodiment 4 provides the compound of any preceding embodiment, wherein the PRS component is from 5 to 20 nucleosides in length.

Embodiment 5 provides the compound of any preceding embodiment, wherein the PRS component comprises a sequence having at least 80% identity to GGACU (SEQ ID NO: 4), GGACUGGACU (SEQ ID NO: 14), GGACUGGAC (SEQ ID NO: 12), and/or GGACTGGAC (SEQ ID NO: 13).

Embodiment 6 provides the compound of any of embodiments 1-4, having a sequence in any one of Tables 3, 12, 14, 23, or 25.

Embodiment 7 provides the compound of any of embodiments 1 or 2, wherein the ASO component is from 14 to 17 nucleosides in length.

Embodiment 8 provides the compound of any of embodiments 1 or 2, wherein the ASO component is 15 nucleosides in length.

Embodiment 9 provides the compound of any of embodiments 1 or 2, wherein the ASO component comprises a sequence differing no more than 3 positions from the sequence of GGACCCTGGGTCCTG (SEQ ID NO: 87) or GGACCCTGGGTCTTG (SEQ ID NO: 88).

Embodiment 10 provides the compound of any preceding embodiment, wherein the PRS component is 9 nucleosides and the antisense oligonucleotide component is 15 nucleosides in length.

Embodiment 11 provides the compound of any preceding embodiment, wherein the PRS component is 10 nucleotides and the antisense oligonucleotide component is 16 nucleosides in length.

Embodiment 12 provides the compound of any preceding embodiment, wherein the compound targets PKD1 region at a 5′UTR, 3′UTR, uORF and/or miRNA binding site.

Embodiment 13 provides the compound of any preceding embodiment, wherein the compound targets the 3′UTR about 60 to 300, 100 to 260, 140 to 220, 170 to 200 or 175 to 195 nucleotides downstream of a stop codon.

Embodiment 14 provides the compound of any preceding embodiment, wherein the compound targets the 3′UTR about 120 to 250, 140 to 200, 170 to 200 or 175 to 195 nucleotides downstream of a stop codon.

Embodiment 15 provides the compound of any of embodiments 1 or 2, wherein the compound is a dual functional compound that binds to a region of the 3′UTR of PKD1 and targets a binding site of a miRNA.

Embodiment 16 provides the compound of any of embodiments 1 or 2, wherein the compound comprises at least one chemical modification.

Embodiment 17 provides the compound of embodiment 16, wherein the chemical modification can be selected from 2′-O-methyl (2′-OMe), 2′-O-methoxyethyl (2′-MOE), 2′-fluoro (2′-F), constrained ethyl (cEt), unlocked nucleic acid (UNA), locked nucleic acid (LNA) and/or 5-methylcytosine base.

Embodiment 18 provides the compound of any of embodiments 1 or 2, wherein the compound comprises at least one modified internucleoside linkage.

Embodiment 19 provides the compound of embodiment 18, wherein the at least one modified internucleoside linkage is a phosphorothioate internucleoside (PS) linkage.

Embodiment 20 provides the compound of any of embodiments 1 or 2, further comprising a conjugate moiety.

Embodiment 21 provides the compound of embodiment 20, wherein the conjugate moiety is selected from the group consisting of cholesterols, lipids, carbohydrates, phospholipids, biotin, phenazine, folate, phenanthridine, anthraquinone, acridine, fluoresceins, rhodamines, coumarins, peptides, antibodies, dyes, and tocopherol, and any combinations thereof.

Embodiment 22 provides the compound of embodiment 20, wherein the conjugate moiety is selected from the group consisting of HEG, FA-Fl, and PALM, and any combinations thereof.

Embodiment 23 provides the compound of any preceding embodiment, wherein the compound increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, 250%, or 300%.

Embodiment 24 provides the compound of any preceding embodiment, wherein the compound is a salt form.

Embodiment 25 provides the pharmaceutical composition comprising the compound, or salt thereof, of any preceding embodiment, alone or in combination with a pharmaceutically acceptable carrier and/or excipient.

Embodiment 26 provides a method for increasing translation of PKD1 mRNA in a cell comprising administering the compound of any of embodiments 1 to 24 or the pharmaceutical composition of embodiment 25 to the cell, in an amount sufficient to increase translation of PKD1 mRNA.

Embodiment 27 provides a method for increasing expression of PKD1 protein in a cell comprising administering the compound of any of embodiments 1 to 24 or the pharmaceutical composition of embodiment 25 to the cell, in an amount sufficient to increase expression of PKD1 protein.

Embodiment 28 provides a method for treating a haploinsufficiency and/or loss-of-function disease, disorder and/or condition in a subject comprising administering the compound of any of embodiments 1 to 24 or the pharmaceutical composition of embodiment 25 to the subject, in an amount sufficient to increase PKD1 expression by at least about 20%, 25%, 30%, 35%, 40%, 45%, 50%, 55%, or 60%, wherein the increase in PKD1 protein expression treats the haploinsufficiency and/or loss-of-function disease, disorder and/or condition in the subject.

Embodiment 29 provides the method of embodiment 28, wherein the haploinsufficiency and/or loss-of-function disease, disorder and/or condition is selected from polycystic kidney disease and/or polycystic liver disease.

Embodiment 30 provides a method for decreasing cyst size in an organ of a subject comprising administering the compound of any of embodiments 1 to 24 or the pharmaceutical composition of embodiment 25 to the subject, in an amount sufficient to increase PKD1 expression by at least about 20%, 25%, 30%, 35%, 40%, 45%, 50%, 55%, or 60%, wherein the increase in PKD1 protein expression decreases cyst size in the organ of the subject.

Embodiment 31 provides a method for decreasing cyst formation in an organ of a subject comprising administering the compound of any of embodiments 1 to 24 or the pharmaceutical composition of embodiment 25 to the subject, in an amount sufficient to increase PKD1 expression by at least about 20%, 25%, 30%, 35%, 40%, 45%, 50%, 55%, or 60%, wherein the increase in PKD1 protein expression decreases cyst formation in the organ of the subject.

Embodiment 32 provides a method for impeding cyst formation in an organ of a subject comprising administering the compound of any of embodiments 1 to 24 or the pharmaceutical composition of embodiment 25 to the subject, in an amount sufficient to increase PKD1 expression by at least about 20%, 25%, 30%, 35%, 40%, 45%, 50%, 55%, or 60%, wherein the increase in PKD1 protein expression impedes cyst formation in the organ of the subject.

Embodiment 33 provides a method for reversing cyst progression in an organ of a subject comprising administering the compound of any of embodiments 1 to 24 or the pharmaceutical composition of embodiment 25 to the subject, in an amount sufficient to increase PKD1 expression by at least about 20%, 25%, 30%, 35%, 40%, 45%, 50%, 55%, or 60%, wherein the increase in PKD1 protein expression reverses cyst progression in the organ of the subject.

Embodiment 34 provides the method of any of embodiments 26 to 33, where the compound or the pharmaceutical composition can be administered subcutaneously or intravenously to the subject.

Embodiment 35 provides a compound according to any of embodiments 1 to 24 or the pharmaceutical composition of embodiment 25 for use in medicine.

Embodiment 36 provides a compound according to any of embodiments 1 to 24 or the pharmaceutical composition of embodiment 26 for use in treating a haploinsufficiency and/or loss-of-function disease, disorder and/or condition in a subject comprising administering the compound or the pharmaceutical composition to the subject, in an amount sufficient to increase PKD1 expression by at least about 20%, wherein the increase in PKD1 protein expression treats the haploinsufficiency and/or loss-of-function disease, disorder and/or condition in the subject.

Embodiment 37 provides the compound or pharmaceutical composition for use of embodiment 36, wherein the haploinsufficiency and/or loss-of-function disease, disorder and/or condition is selected from polycystic kidney disease and/or polycystic liver disease.

Embodiment 38 provides a compound according to any of embodiments 1 to 24 or the pharmaceutical composition of embodiment 25 for use in decreasing cyst size in an organ of a subject comprising administering the compound or the pharmaceutical composition to the subject, in an amount sufficient to increase PKD1 expression by at least about 20%, wherein the increase in PKD1 protein expression decreases cyst size in the organ of the subject.

Embodiment 39 provides a compound according to any of embodiments 1 to 24 or the pharmaceutical composition-embodiment 25 for use in decreasing cyst formation in an organ of a subject comprising administering the compound or the pharmaceutical composition to the subject, in an amount sufficient to increase PKD1 expression by at least about 20%, wherein the increase in PKD1 protein expression decreases cyst formation in the organ of the subject.

Embodiment 40 provides a compound according to any of embodiments 1 to 24 or the pharmaceutical composition of embodiment 25 for use in impeding cyst formation in an organ of a subject comprising administering the compound or the pharmaceutical composition to the subject, in an amount sufficient to increase PKD1 expression by at least about 20%, wherein the increase in PKD1 protein expression impedes cyst formation in the organ of the subject.

Embodiment 41 provides a compound according to any of embodiments I to 24 or the pharmaceutical composition of embodiment 25 for use in reversing cyst progression in an organ of a subject comprising administering the compound or the pharmaceutical composition to the subject, in an amount sufficient to increase PKD1 expression by at least about 20%, wherein the increase in PKD1 protein expression reverses cyst progression in the organ of the subject.

Embodiment 42 provides the compound or pharmaceutical composition for use of any of embodiments 35 to 41, where the compound or the pharmaceutical composition is administered subcutaneously or intravenously to the subject.

Embodiment 43 provides a process for preparing compound of any one of embodiments 1-24, wherein the process comprises the steps of: (a) preparing the compound by sequential coupling of modified and/or unmodified nucleotides and/or linkers via the phosphoramidite oligonucleotide synthesis on a conjugate modified or unmodified solid support; (b) optionally, coupling a conjugate moiety to the compound on the solid support via the phosphoramidite oligonucleotide synthesis; (c) detaching the compound from the solid support and removing the solid support; and (d) optionally, adding a conjugate post cleavage; and/or (e) optionally, further purifying the compound, optionally using chromatography.

Embodiment 44 provides an ACT-UP1 compound comprising an oligonucleotide, or salt thereof, for enhancing expression of PKD1 in a cell, wherein the oligonucleotide comprises at least a 20 nucleobase portion of the sequence of SEQ ID NO: 77, or a sequence thereof which differs in up to 2 positions of SEQ ID NO: 77.

Embodiment 45 provides a compound of embodiment 44, wherein the sequence thereof which differs up to 2 positions of SEQ ID NO: 77 is changed at any of the nucleobases at position 10 to position 24.

Embodiment 46 provides a compound of embodiment 44, wherein the sequence thereof which differs up to 2 positions of SEQ ID NO: 77 is changed at any of the nucleobases at position 1 to position 9.

Embodiment 47 provides a compound of any of embodiments 45 or 46, wherein the change is a nucleobase change to either an A or T.

Embodiment 48 provides a compound of embodiment 44, wherein the compound with the sequence of SEQ ID NO: 77 is selected from the sequence and chemistry of ATXL608, ATXL681, ATXL685, ATXL686, or ATXL814.

Embodiment 49 provides a compound of embodiment 44, wherein the compound with up to 2 positions different from the sequence of SEQ ID NO: 77 is selected from ATXL501 (SEQ ID NO: 73), ATXL682 (SEQ ID NO: 85), ATXL683 (SEQ ID NO: 86), ATXL815 (SEQ ID NO: 85), ATXL-A (SEQ ID NO: 89), ATXL-B (SEQ ID NO: 90), ATXL-C(SEQ ID NO: 91), ATXL-E (SEQ ID NO: 93), ATXL-F (SEQ ID NO: 94), ATXL-L (SEQ ID NO: 100), ATXL-M (SEQ ID NO: 101), ATXL-D (SEQ ID NO: 92), ATXL-G (SEQ ID NO: 95), ATXL-H (SEQ ID NO: 96), ATXL-I (SEQ ID NO: 97), ATXL-J (SEQ ID NO: 98), ATXL-K (SEQ ID NO: 99), ATXL-N (SEQ ID NO: 102), ATXL-O (SEQ ID NO: 103), ATXL-P (SEQ ID NO: 104), ATXL-Q (SEQ ID NO: 105), ATXL-S(SEQ ID NO: 107), ATXL-T (SEQ ID NO: 108), ATXL-V (SEQ ID NO: 110), ATXL-WW (SEQ ID NO: 111), ATXL-Y (SEQ ID NO: 113), ATXL-Z (SEQ ID NO: 114), ATXL-BB (SEQ ID NO: 116), ATXL-CC (SEQ ID NO: 117), ATXL-EE (SEQ ID NO: 119), or ATXL-FF (SEQ ID NO: 120).

Embodiment 50 provides a compound of embodiment 44, wherein the compound comprises at least one chemical modification.

Embodiment 51 provides a compound of Embodiment 50, wherein the at least one chemical modification is selected from 2′-O-methyl (2′-OMe), 2′-O-(2-methoxyethyl) (2′-MOE), 2′-fluoro (2′-F), constrained ethyl (cEt), unlocked nucleic acid (UNA), locked nucleic acid (LNA), 2′-MOE modified T, and/or 5-methylcytosine base.

Embodiment 52 provides a compound of embodiment 44, wherein the compound is chemically modified at each nucleoside.

Embodiment 53 provides a compound of embodiment 52, wherein the chemical modifications are selected from 2′-O-methyl (2′-OMe), 2′-O-(2-methoxyethyl) (2′-MOE), 2 fluoro (2′-F), constrained ethyl (cEt), unlocked nucleic acid (UNA), locked nucleic acid (LNA), 2′-MOE modified T, and/or 5-methylcytosine base.

Embodiment 54 provides a compound of embodiment 44, wherein the compound further comprises a conjugate.

Embodiment 55 provides a compound of embodiment 44, wherein the compound comprises at least one modified internucleoside linkage.

Embodiment 56 provides a compound of embodiment 55, wherein the at least one modified internucleoside linkage is a phosphorothioate (PS) internucleoside linkage.

Embodiment 57 provides a compound of embodiment 44, wherein the compound comprises the sequence and chemical structure of ATXL608 (SEQ ID NO: 43) as shown below:

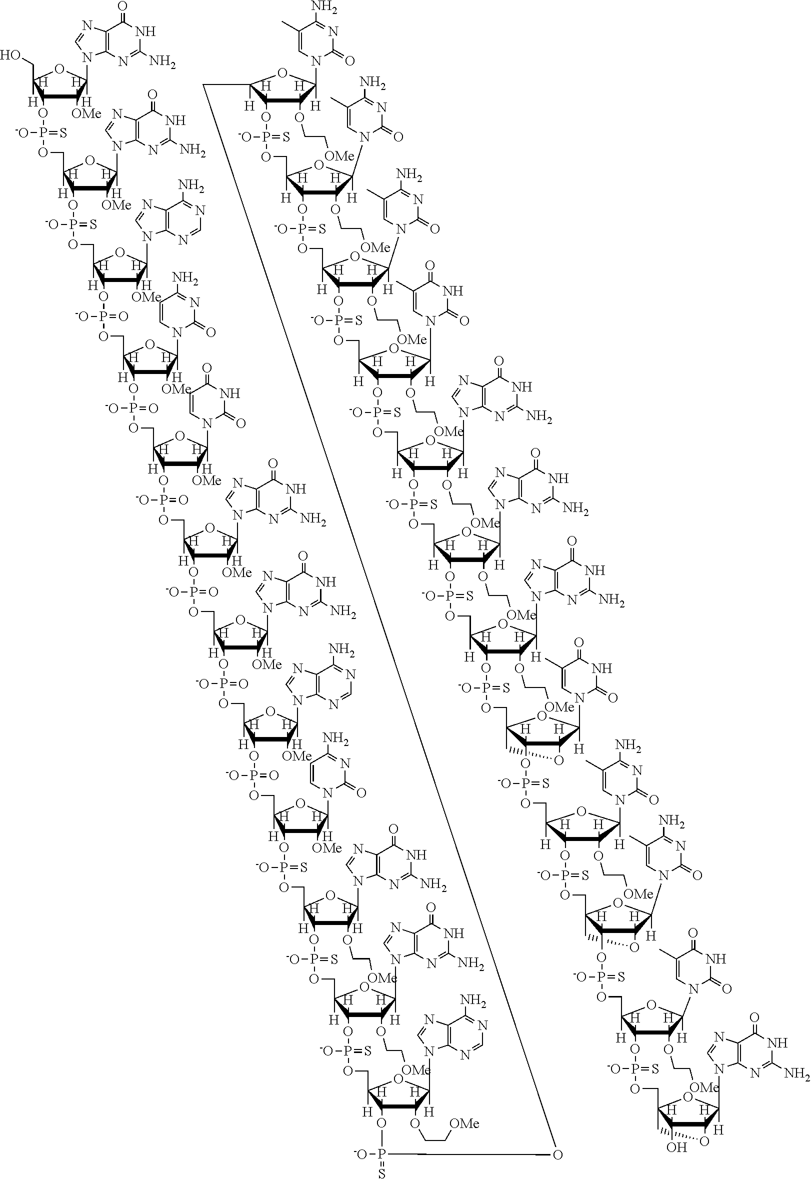

Embodiment 58 provides a compound of embodiment 44, wherein the compound comprises the sequence and chemical structure of ATXL682 (SEQ ID NO: 53) as shown below:

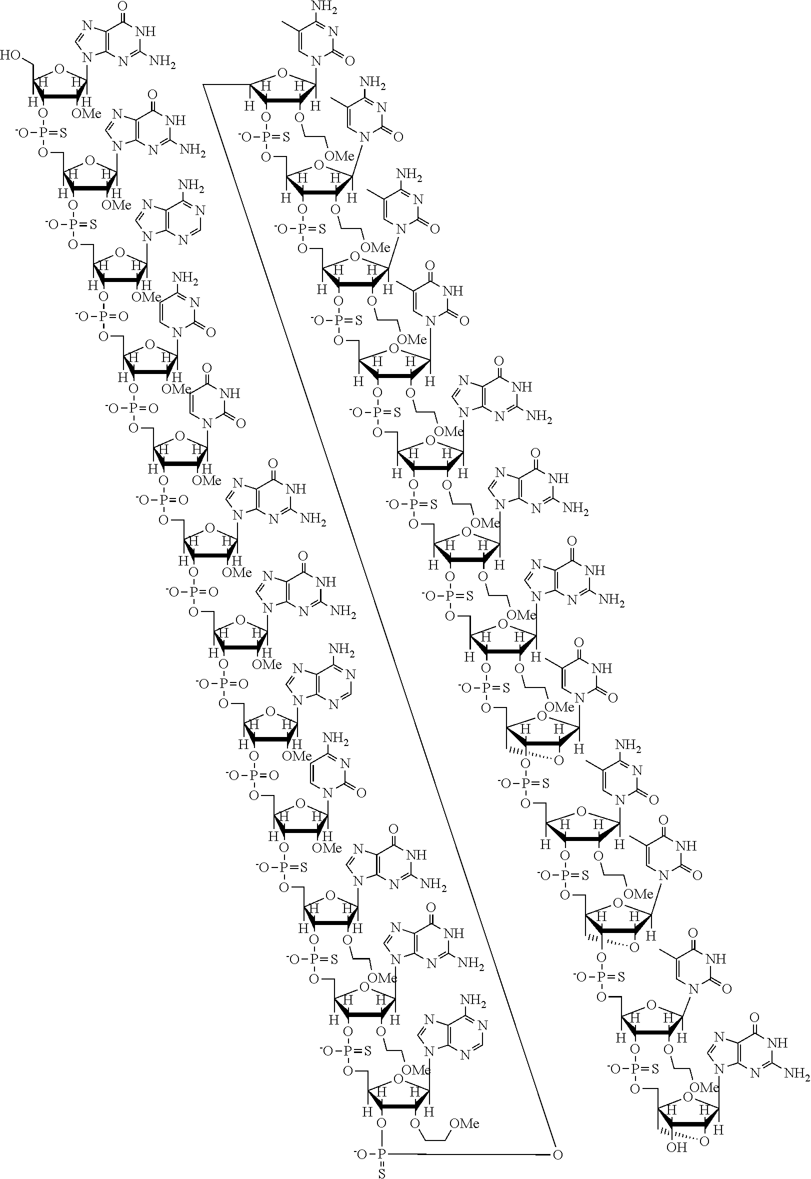

Embodiment 59 provides a compound of embodiment 44, wherein the compound comprises the sequence and chemical structure of ATXL686 (SEQ ID NO: 56) as shown below:

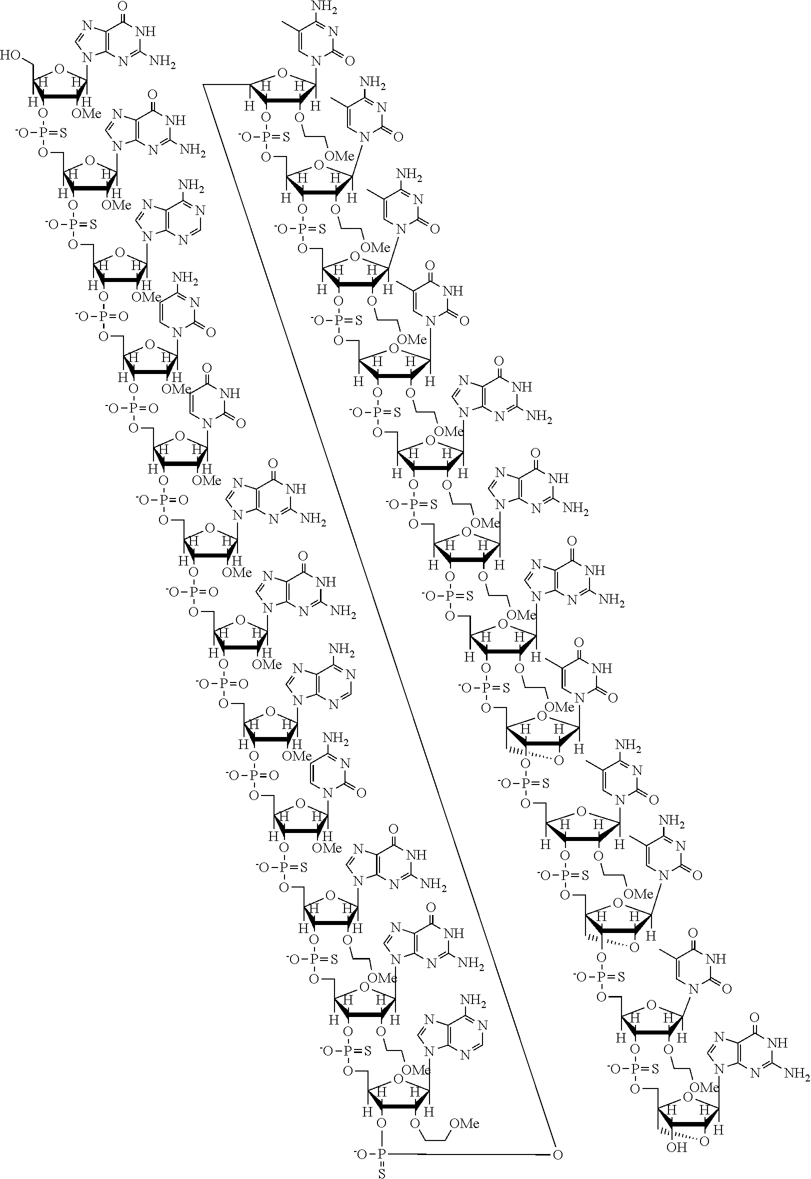

Embodiment 60 provides a compound of embodiment 44, wherein the compound comprises the sequence and chemical structure of ATXL814 (SEQ ID NO: 57) as shown below:

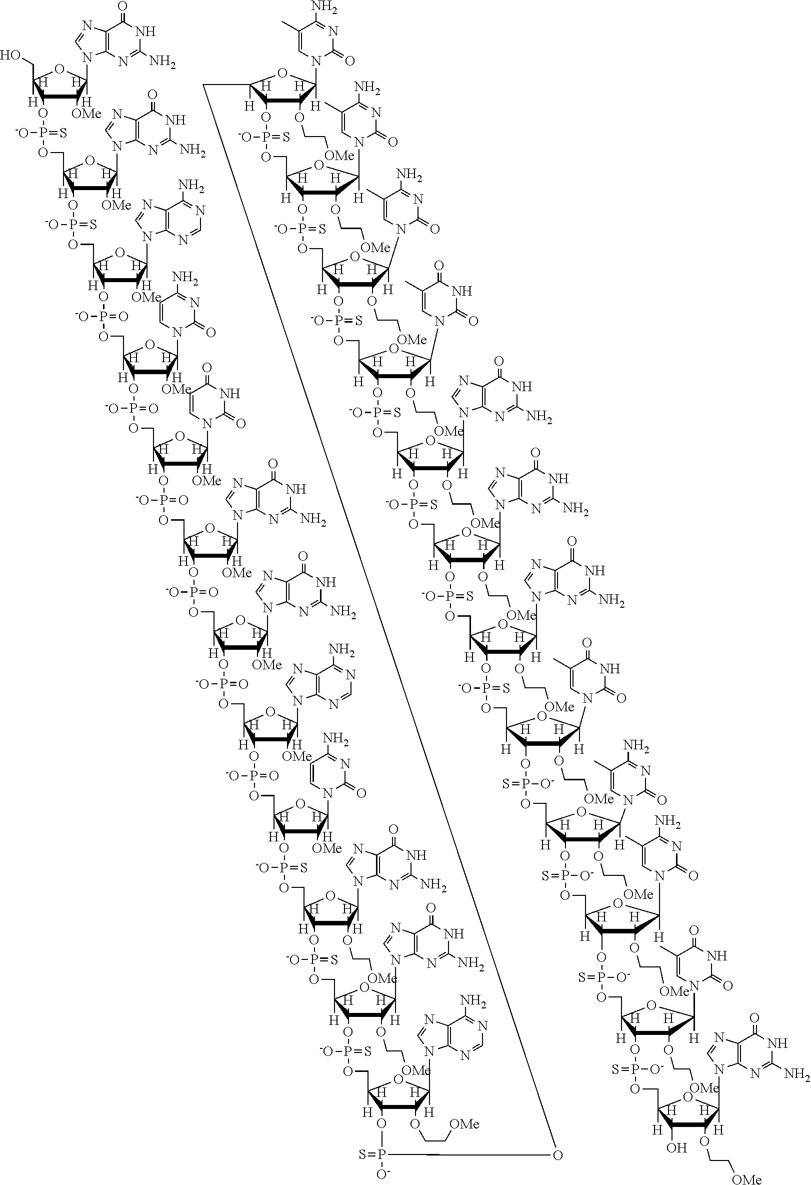

Embodiment 61 provides a compound of embodiment 44, wherein the compound comprises the sequence and chemical structure of ATXL815 (SEQ ID NO: 58) as shown below:

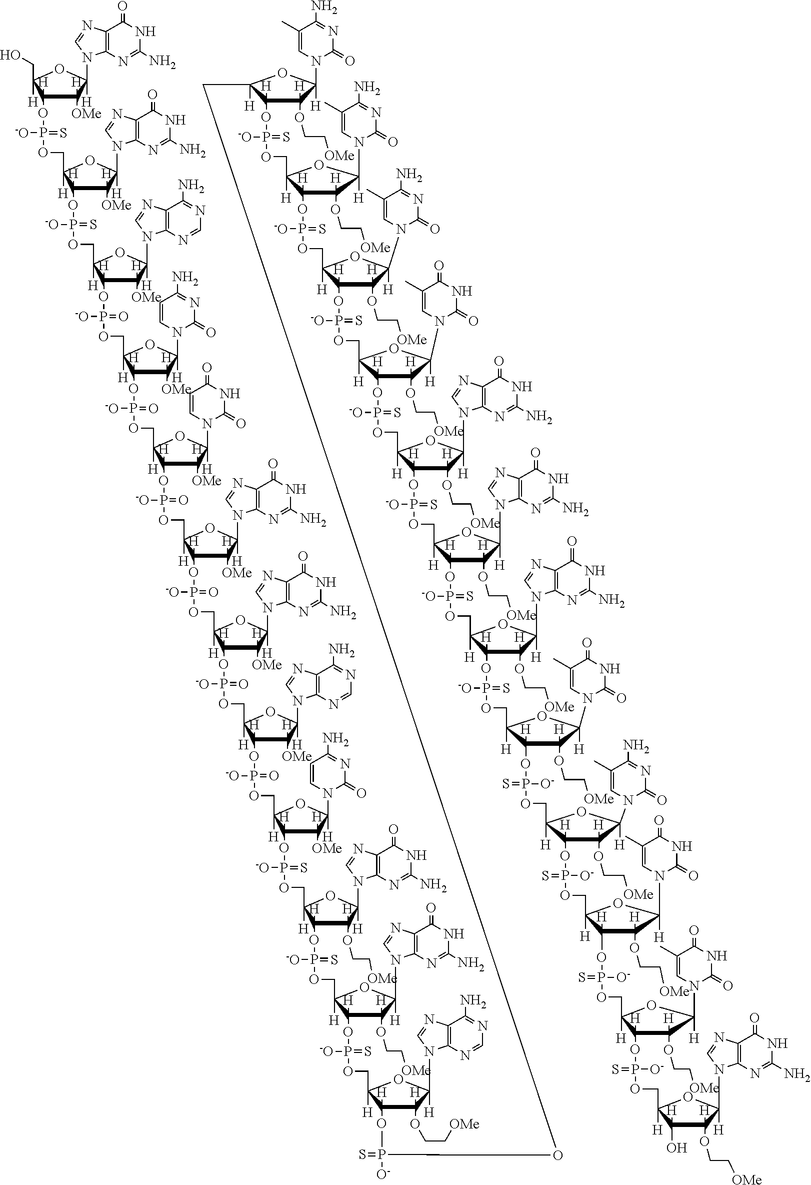

Embodiment 62 provides a compound of embodiment 44, wherein the compound increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, 250%, or 300%.

Embodiment 63 provides a pharmaceutical composition for enhancing the expression of PKD1 comprising the compound of embodiment 44, or salt thereof, alone or in combination with a pharmaceutically acceptable carrier and/or excipient.

Embodiment 64 provides a kit comprising the pharmaceutical composition of embodiment 63, and a label.

Embodiment 65 provides a method for increasing translation of PKD1 mRNA in a cell comprising administering the compound of embodiment 44 to the cell, in an amount sufficient to increase translation of PKD1 mRNA.

Embodiment 66 provides-a method for increasing expression of PKD1 protein in a cell comprising administering the compound of embodiment 44 to the cell, in an amount sufficient to increase expression of PKD1 protein.

Embodiment 67 provides a method for treating a haploinsufficiency and/or loss of function disease, disorder and/or condition in a subject comprising administering the compound of embodiment 44 to the subject, in an amount sufficient to increase PKD1 expression by at least about 20%, 25%, 30%, 35%, 40%, 45%, 50%, 55%, or 60%, wherein the increase in PKD1 protein expression treats the haploinsufficiency and/or loss of function disease, disorder and/or condition in the subject.

Embodiment 68 provides the method of embodiment 67, wherein the haploinsufficiency and/or loss of function disease, disorder and/or condition is selected from polycystic kidney disease and/or polycystic liver disease (PLD).

Embodiment 69 provides the method of embodiment 68, wherein the polycystic kidney disease is autosomal dominant polycystic kidney disease (ADPKD).

Embodiment 70 provides a method for decreasing cyst size in an organ of a subject comprising administering the compound of embodiment 44 to the subject, in an amount sufficient to increase PKD1 expression by at least about 20%, 25%, 30%, 35%, 40%, 45%, 50%, 55%, or 60%, wherein the increase in PKD1 protein expression decreases cyst size in the organ of the subject.

Embodiment 71 provides a method for decreasing cyst formation in an organ of a subject comprising administering the compound of embodiment 44 to the subject, in an amount sufficient to increase PKD1 expression by at least about 20%, 25%, 30%, 35%, 40%, 45%, 50%, 55%, or 60%, wherein the increase in PKD1 protein expression decreases cyst formation in the organ of the subject.

Embodiment 73 provides a method for impeding cyst formation in an organ of a subject comprising administering the compound of embodiment 44 to the subject, in an amount sufficient to increase PKD1 expression by at least about 20%, 25%, 30%, 35%, 40%, 45%, 50%, 55%, or 60%, wherein the increase in PKD1 protein expression impedes cyst formation in the organ of the subject.

Embodiment 74 provides a method for reversing cyst progression in an organ of a subject comprising administering the compound of embodiment 44 to the subject, in an amount sufficient to increase PKD1 expression by at least about 20%, 25%, 30%, 35%, 40%, 45%, 50%, 55%, or 60%, wherein the increase in PKD1 protein expression reverses cyst progression in the organ of the subject.

Embodiment 75 provides the method of embodiment 44, where the compound or the pharmaceutical composition is administered subcutaneously or intravenously to the subject.

Embodiment 76 provides a process for preparing compound of embodiment A, wherein the process comprises the steps of: (a) preparing the compound by sequential coupling of modified and/or unmodified nucleotides and/or linkers via the phosphoramidite oligonucleotide synthesis on a conjugate modified or unmodified solid support; (b) optionally, coupling a conjugate moiety to the compound on the solid support via the phosphoramidite oligonucleotide synthesis; and (c) detaching the compound from the solid support and removing the solid support.

Embodiment 77 provides an ACT-UP1 compound comprising an oligonucleotide, or salt thereof, for enhancing expression of PKD1 in a cell, wherein the oligonucleotide comprises at least a 20 nucleobase portion of the sequence of SEQ ID NO: 77, or a sequence thereof which differs in up to 2 positions of SEQ ID NO: 77.

Embodiment 78 provides a compound of embodiment 77, wherein the sequence thereof which differs up to 2 positions of SEQ ID NO: 77 is changed at any of the nucleobases at position 10 to position 24.

Embodiment 79 provides a compound of embodiment 77, wherein the sequence thereof which differs up to 2 positions of SEQ ID NO:77 is changed at any of the nucleobases at position 1 to position 9.

Embodiment 80 provides a compound of any of embodiments 78 or 79, wherein the change is a nucleobase change to either an A or T.

Embodiment 81 provides a compound of any preceding embodiment, wherein the compound with the sequence of SEQ ID NO:77 is selected from ATXL608, ATXL681, ATXL685, ATXL686, ATXL814.

Embodiment 82 provides a compound of any of embodiments 77 to 80, wherein the compound with up to 2 positions different from the sequence of SEQ ID NO:77 is selected from ATXL501 (SEQ ID NO: 73), ATXL682 (SEQ ID NO: 85), ATXL683 (SEQ ID NO: 86), ATXL815 (SEQ ID NO: 85), ATXL-A (SEQ ID NO: 89), ATXL-B (SEQ ID NO: 90), ATXL-C (SEQ ID NO: 91), ATXL-E (SEQ ID NO: 93), ATXL-F (SEQ ID NO: 94), ATXL-L (SEQ ID NO: 100), ATXL-M (SEQ ID NO: 101), ATXL-D (SEQ ID NO: 92), ATXL-G (SEQ ID NO: 95), ATXL-H (SEQ ID NO: 96), ATXL-I (SEQ ID NO: 97), ATXL-J (SEQ ID NO: 98), ATXL-K (SEQ ID NO: 99), ATXL-N(SEQ ID NO: 102), ATXL-O (SEQ ID NO: 103), ATXL-P (SEQ ID NO: 104), ATXL-Q (SEQ ID NG-105), ATXL-S(SEQ ID NO: 107), ATXL-T (SEQ ID NO: 108), ATXL-V (SEQ ID NO: 110), ATXL-WW (SEQ ID NO: 11l), ATXL-Y (SEQ ID NO: 113), ATXL-Z (SEQ ID NO: 114), ATXL-BB (SEQ ID NO: 116), ATXL-CC (SEQ ID NO: 117), ATXL-EE (SEQ ID NO: 119), and ATXL-FF (SEQ ID NO: 120).

Embodiment 83 provides a compound of any preceding embodiment, wherein the compound comprises at least one chemical modification, wherein the chemical modification is a modification of a nucleoside compared to a naturally occurring nucleoside.

Embodiment 84 provides a compound of Embodiment 83, wherein the at least one chemical modification is selected from 2′-O-methyl (2′-OMe), 2′-O-(2-methoxyethyl) (2′-MOE), 2′-fluoro (2′-F), constrained ethyl (cEt), unlocked nucleic acid (UNA), locked nucleic acid (LNA), 2′-MOE modified T, and/or 5-methylcytosine base.

Embodiment 85 provides a compound of any preceding embodiment, wherein the compound is chemically modified at each nucleoside.

Embodiment 86 provide a compound of embodiment 85, wherein the chemical modifications are selected from 2′-O-methyl (2′-OMe), 2′-O-(2-methoxyethyl) (2′-MOE), 2′ -fluoro (2′-F), constrained ethyl (cEt), unlocked nucleic acid (UNA), locked nucleic acid (LNA), 2′-MOE modified T, and/or 5-methylcytosine base.

Embodiment 87 provides a compound of any preceding embodiment, wherein the compound further comprises a conjugate.

Embodiment 88 provides a compound of any preceding embodiment, wherein the compound comprises at least one modified internucleotide linkage.

Embodiment 89 provides a compound of embodiment 88, wherein the at least one modified internucleotide linkage is a phosporothioate (PS) internucleoside linkage.

Embodiment 90 provides a compound of any preceding embodiment, wherein the compound comprises the sequence and chemical structure of ATXL608 (SEQ ID NO: 43) as shown below:

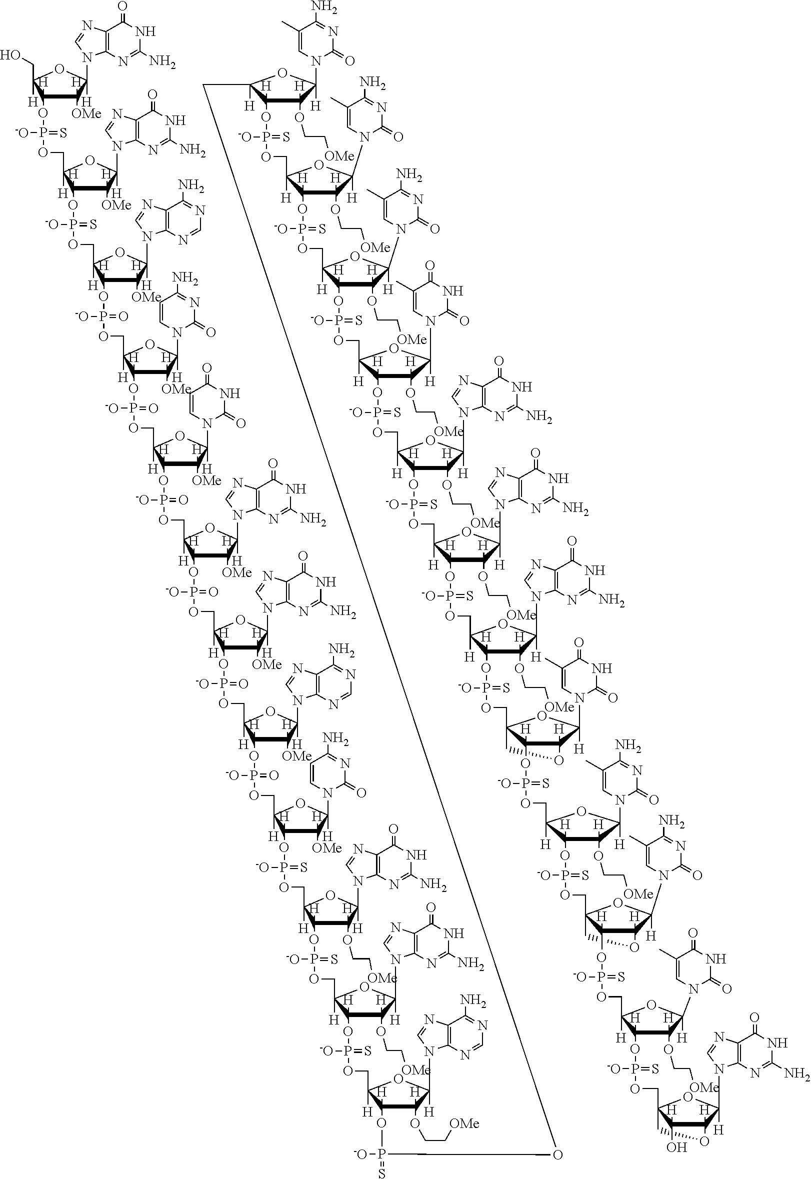

Embodiment 91 provides a compound of embodiment 77 to 89, wherein the compound comprises the sequence and chemical structure of ATXL682 (SEQ ID NO: 53) as shown below:

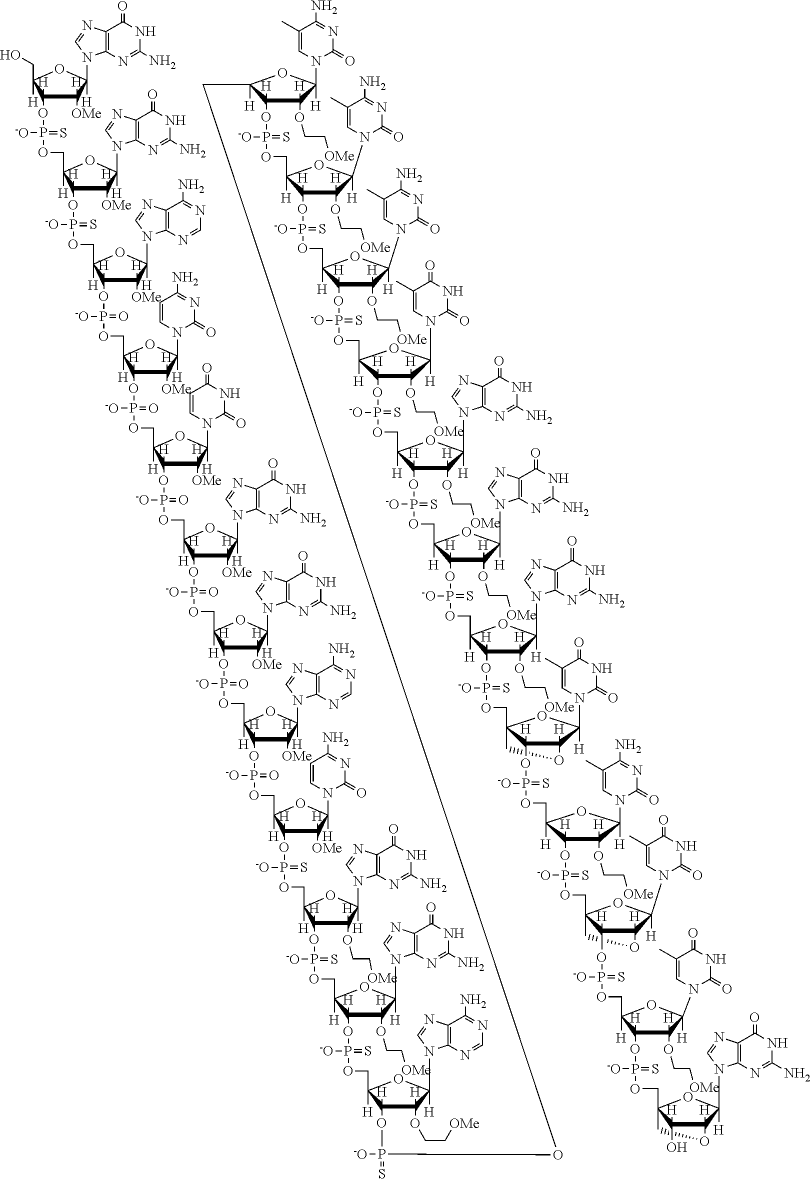

Embodiment 92 provides a compound of embodiment 77 to 89, wherein the compound comprises the sequence and chemical structure of ATXL686 (SEQ ID NO: 56) as shown below:

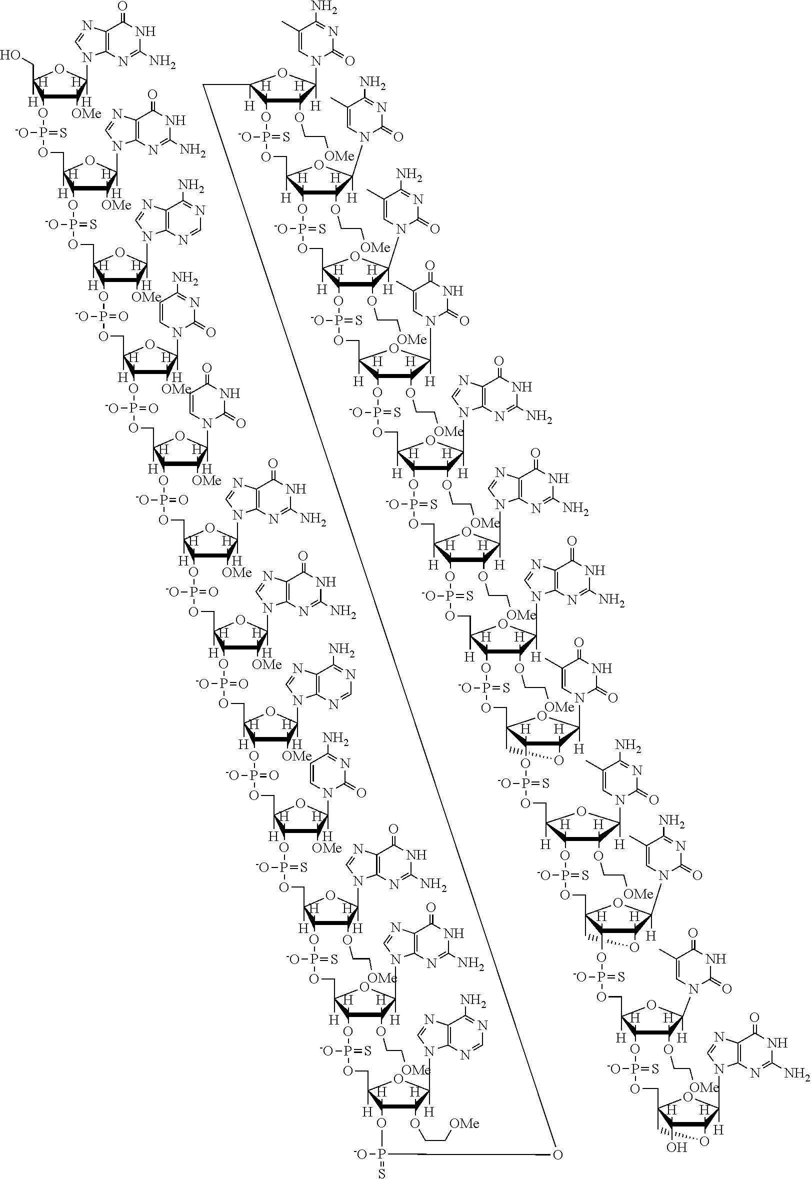

Embodiment 93 provides a compound of embodiment 77 to 89, wherein the compound comprises chemical D shown NO: 57) as below:

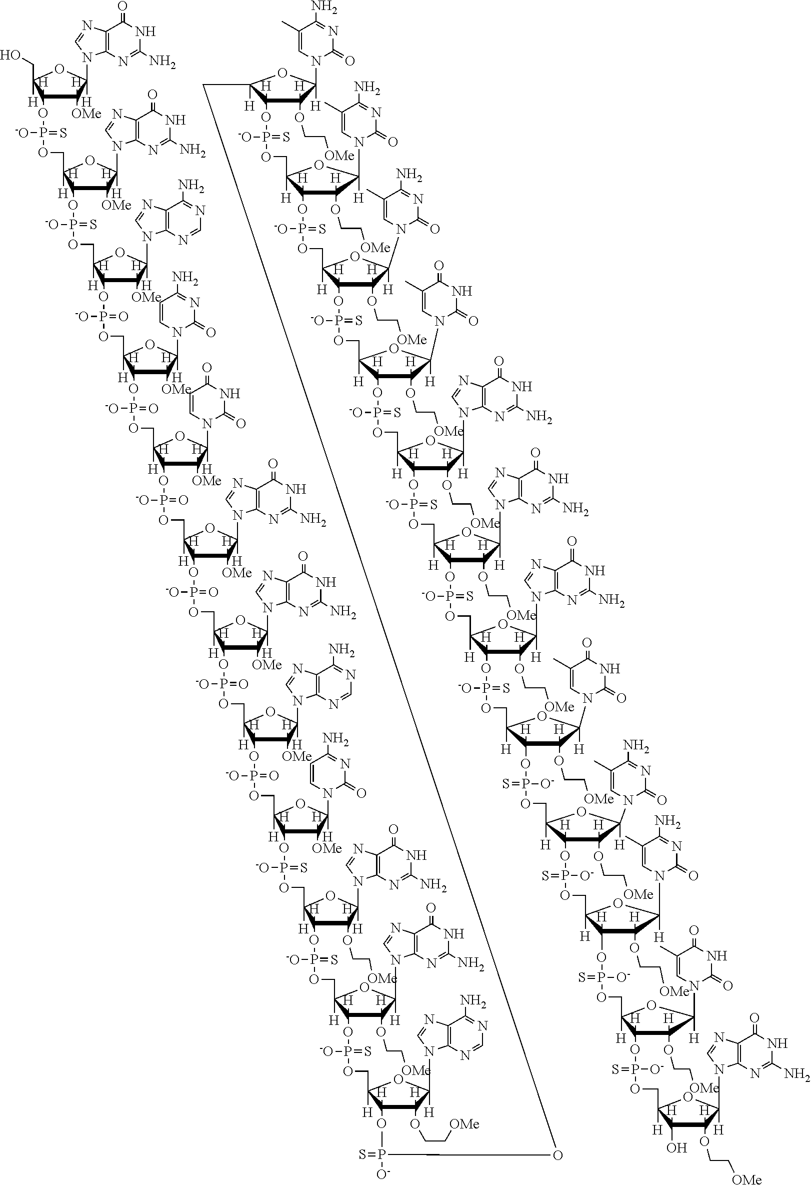

Embodiment 94 provides a compound of embodiment 77 to 89, wherein the compound comprises the sequence and chemical structure of ATXL815 (SEQ ID NO: 58) as shown below:

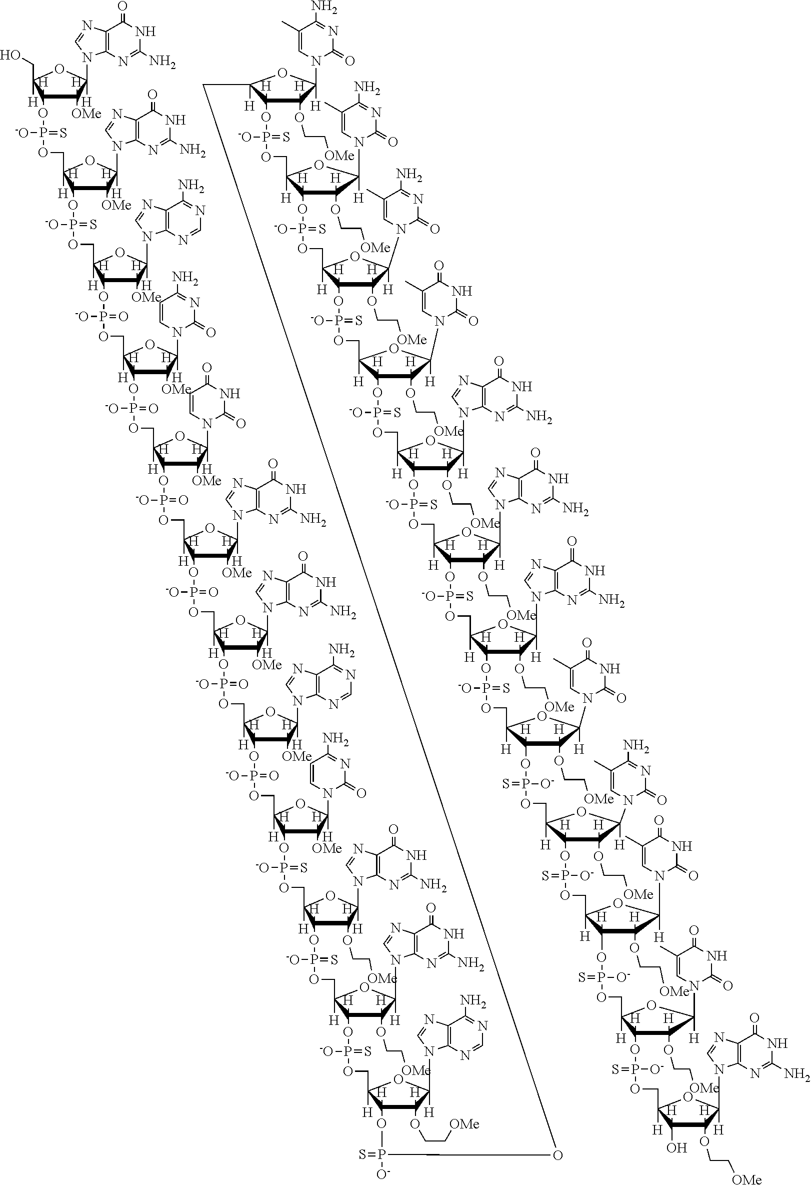

Embodiment 95 provides a compound of any preceding embodiment, wherein the compound increases expression of PKD1 protein by at least about 10%, 15%, 20%, 25%, 30%, 35%, 40%, 45%, 50%, 55%, 60%, 65%, 70%, 75%, 80%, 85%, 90%, 95%, 100%, 150%, 200%, 250%, or 300%.

Embodiment 96 provides a pharmaceutical composition comprising the compound of any preceding embodiment, or salt thereof, alone or in combination with a pharmaceutically acceptable carrier and/or excipient.

Embodiment 97 provides a kit comprising the pharmaceutical composition of embodiment 96, and a label.

Embodiment 98 provides a compound of any embodiment 77 to 94 for use in medicine.

Embodiment 99 provides a compound of any embodiment 77 to 94 for use in treating a haploinsufficiency and/or loss of function disease, disorder and/or condition in a subject comprising administering the compound to the subject, in an amount sufficient to increase PKD1 expression by at least about 20% wherein the increase in PKD1 protein expression treats the haploinsufficiency and/or loss of function disease, disorder and/or condition in the subject.

Embodiment 100 provides the compound for use of embodiment 99, wherein the haploinsufficiency and/or loss of function disease, disorder and/or condition is selected from polycystic kidney disease and/or polycystic liver disease (PLD).

Embodiment 101 provides the compound for use of embodiment 100, wherein the polycystic kidney disease is autosomal dominant polycystic kidney disease (ADPKD).

Embodiment 102 provides a compound of any embodiment 77 to 94 for use in decreasing cyst size in an organ of a subject comprising administering the compound to the subject, in an amount sufficient to increase PKD1 expression by at least about 20% wherein the increase in PKD1 protein expression decreases cyst size in the organ of the subject.

Embodiment 103 provides a compound of any embodiment 77 to 94 for use in decreasing cyst formation in an organ of a subject comprising administering the compound to the subject, in an amount sufficient to increase PKD1 expression by at least about 20%, wherein the increase in PKD1 protein expression decreases cyst formation in the organ of the subject.

Embodiment 104 provides a compound of any embodiment 77 to 94 for use in impeding cyst formation in an organ of a subject comprising administering the compound to the subject, in an amount sufficient to increase PKD1 expression by at least about 20%, wherein the increase in PKD1 protein expression impedes cyst formation in the organ of the subject.

Embodiment 105 provides a compound of any embodiment 77 to 94 for use in reversing cyst progression in an organ of a subject comprising administering the compound to the subject, in an amount sufficient to increase PKD1 expression by at least about 20%, wherein the increase in PKD1 protein expression reverses cyst progression in the organ of the subject.

Embodiment 106 provides the compound for use of embodiment 99 to 105, wherein the compound is administered subcutaneously or intravenously to the subject.

Embodiment 107 provides a process for preparing compound of any embodiment 77 to 94, wherein the process comprises the steps of: (a) preparing the compound by sequential coupling of modified and/or unmodified nucleotides and/or linkers via the phosphoramidite oligonucleotide synthesis on a conjugate modified or unmodified solid support; (b) optionally, coupling a conjugate moiety to the compound on the solid support via the phosphoramidite oligonucleotide synthesis; and (c) detaching the compound from the solid support and removing the solid support.

### EXAMPLES

### Non-Limiting Disclosure and Incorporation-by-Reference

While certain compounds, compositions and methods described herein have been described with specificity in accordance with certain embodiments, the following examples serve only to illustrate the compounds described herein and are not intended to limit the same. Each of the references recited in the present application is herein incorporated-by-reference in its entirety.

The following applies to all modified sequences disclosed herein.

A notation is made before or after each nucleoside indicating the type of chemical modification, if any, made to the nucleoside. If no modification notation is made before or after a letter designating a nucleoside, the nucleoside is a deoxyribonucleoside. Notations for the chemical modifications to the compounds can be found as follows:
“(5p)” before a nucleoside refers to a 5′-phosphate
“r” before a nucleoside refers to a ribonucleoside (ribonucleoside which has been substituted for a deoxyribonucleoside)
“d” (or no notation made before or after a nucleoside) before a nucleoside refers to a deoxyribonucleoside
“t” before a nucleoside, refers to a 2′-fluoro (also known as 2′-F) sugar modification
“m” before a nucleoside refers to a 2′—OCH₃ (also known as 2′-O-methyl or 2′-OMe) modification
“*[mCL]” specifically denotes an LNA 5-methyl cytidine 5′-thiophosphate
“e” before a nucleoside refers to a 2′—O(CH₂)₂OCH₃ (also known as 2′-O-methoxyethyl, 2′-O-MOE or 2′-MOE) modification (e.g., eG)
“cEt” before a nucleoside refers to a bicyclic sugar moiety comprising a bridge connecting the 4′-carbon and the 2′-carbon, wherein the bridge has the formula 4′-CH(CH₃)—O-2′ (e.g., cEt-A)
“eCm” refers to a 2′-MOE modified 5-methyl cytidine
“*” refers to a phosphorothioate (PS) linkage which has been substituted for a phosphate (PO) linkage
“gna” before a nucleoside refers to a glycol nucleic acid modification
“L” after a nucleoside refers to a locked nucleic acid (LNA) modification of the nucleoside (e.g., GL)
“AN-GaINAc” is a GalNAc moiety described in WO2024137545.
“HEG” refers to hexaethylene glycol (also known as spacer 18 or sp18):

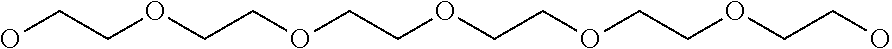 [SMILES: OCCOCCOCCOCCOCCOCCO]

“PALM” refers to a palmitate compound:

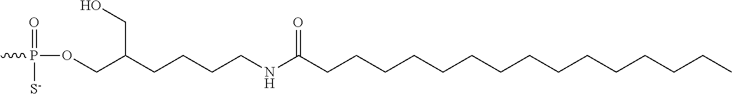 [SMILES: CCCCCCCCCCCCCCCC(=O)NCCCCC(CO)COP(C)(=O)[S-]]

“FA-FI” is a conjugate moiety wherein FA is folic acid:

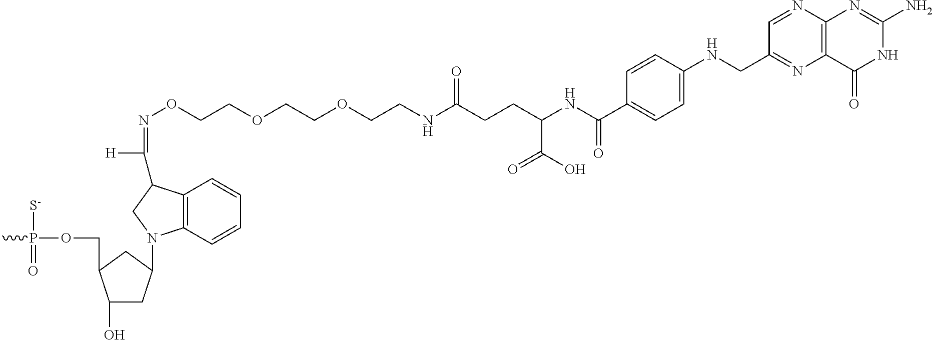 [SMILES: CP(=O)([S-])OCC1CC(N2CC(/C=N\OCCOCCOCCNC(=O)CCC(NC(=O)c3ccc(NCc4cnc5nc(N)[nH]c(=O)c5n4)cc3)C(=O)O)c3ccccc32)CC1O]

If more than one sequence is disclosed in one row of the tables, the SEQ ID NO applies to the modified sequence (“Sequence+Chemistry”).

### Example 1. Antisense Compounds Targeting Human PKD1 mRNA Increase PKD1 Protein Levels

Antisense compounds were designed to base-pair with different regions of the 5′ UTR or 3′ UTR of human PKD1 mRNA (GenBank Accession No: NM_001009944.3; SEQ ID NO: 1). It has previously been shown that antisense compounds can increase protein levels by targeting upstream open reading frames (uORFs) or structural inhibitory elements (Liang X H et al., Nat Biotechnol. 2016; 34(8):875-80); Liang X H et al., Nucleic Acids Res. 2017; 45(16):9528-9546). Compounds ATXL188a, ATXL189, ATXL190, and ATXL198 were designed to target the potential uORF or structural regions in the 5′ UTR of PKD1 mRNA. In addition, it has been demonstrated previously that miR-17 can target PKD1 mRNA and inhibit the expression of PKI)1 protein (Patel V., et al., Proc Natl Acad Sci USA. 2013; 110(26):10765-10770; Lee EC., et al., Nat Commun. 2019;10(1):4148; Lakhia R., et al., Nat Commun. 2022;13(1):4765), ATXL195 was designed to base-pair with the miR-17 binding site in the 3′ UTR of PKD1 mRNA, based on the predicted target position using online accessible server TargetScan (TargetScanHuman 8.0). As a control, an antisense compound not targeting PKD1, ATXL157, was designed to have a similar length and chemistry as the other compounds.

TABLE 1
Sequence and Chemistry of Antisense Compounds Targeting PKD1
to Increase Protein Levels

| | | | PKD1 | |
|---|---|---|---|---|
| Compound | Sequence and Chemistry | Sequence | Targeting | SEQ ID |
| ID | (5′ to 3′) | (5′ to 3′) | Design | NO |
| ATXL188a | mGmCmAmUmGmGmCmGmG | GCAUGGCGGGCG | 5′UTR | 24 |
| | mGmCmGmCmGmGmG | CGGG | uORF | |
| ATXL189 | mGmAmCmGmGmCmAmUmG | GACGGCAUGGCG | 5′UTR | 25 |
| | mGmCmGmGmGmCmG | GGCG | uORF | |
| ATXL190 | mCmAmUmGmGmCmCmCmC | CAUGGCCCCGCC | 5′UTR | 26 |
| | mGmCmCmGmUmCmC | GUCC | uORF | |
| ATXL195 | mCmAmCmAmGmCmCmUmC | CACAGCCUCUUU | 3′UTR | 27 |
| | mUmUmUmAmAmAmGmUmG | AAAGUGC | miR-17 target | |
| | mC | | site | |
| ATXL198 | mUmGmGmCmCmCmCmGmC | UGGCCCCGCCGU | 5′UTR | 28 |
| | mCmGmUmCmCmCmC | CCCC | uORF | |
| ATXL157 | mAmCmUmGmGmAmAmAmA | ACUGGAAAACAA | none | 29 |
| | mCmAmAmCmAmCmC | CACC | | |

Compounds targeting the mRNA of PKD1, described in Table 1. were assessed in human HeLa cells and human kidney cells HEK293 for their ability to increase PKD11 protein expression.

### In Vitro Assay

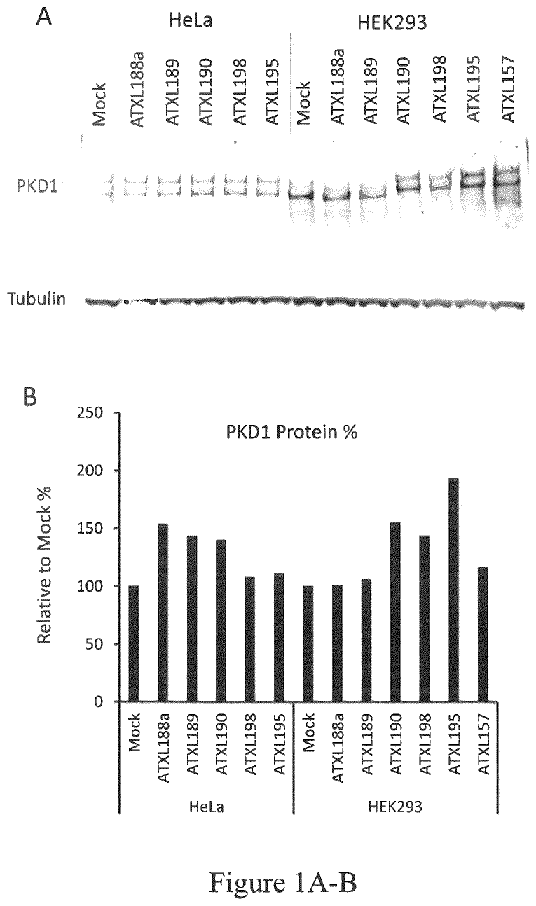

HeLa and HEK293 cells (ATCC, Manassas, VA, USA), seeded and grown in one day to ~70% confluency, were transfected with the compounds listed in Table I at 15 nM final concentrations using Lipofectamine™ 2000 Transfection Reagent (ThermoFisher Scientific, Waltham, MA, USA), or were mock transfected as a control. Twenty-four (24) hrs after transfection, cells were harvested and the protein was extracted using RIPA Lysis and Extraction Buffer (ThermoFisher Scientific, Waltham, MA, USA). The level of PKD1 protein was determined by Western Blotting the extracted proteins and probing the Western Blot using a PKD1 protein-specific antibody (Santa Cruz Biotech, Dallas, TX, USA; Catalog #sc-130554). The Western blotting result is shown in FIG. 1. The Western Blot image was quantified using ImageJ, and the results are shown in Table 2 as percent protein levels relative to mock transfected cells following normalization to a loading control protein, Tubulin (Abcam, Cambridge, UK; Catalog

TABLE 2
PKD1 Protein Levels in Cells Treated with Different Compounds

| Cell line | Compound | PKD1 Protein (%) |
|---|---|---|
| HeLa | Mock | 100.0 |
| | ATXL188a | 153.7 |
| | ATXL189 | 143.3 |
| | ATXL190 | 139.9 |
| | ATXL198 | 107.8 |
| | ATXL195 | 110.8 |
| HEK293 | Mock | 100.0 |
| | ATXL188a | 100.7 |
| | ATXL189 | 105.9 |
| | ATXL190 | 155.0 |
| | ATXL198 | 143.4 |
| | ATXL195 | 193.0 |
| | ATXL157 | 115.9 |

The results show that several compounds, such as ATXL188a, ATXL189, and ATXL190, can increase PKD1 protein levels in HeLa cells, whereas ATXL190, ATXL198, and ATXL195 can increase PKD1 protein levels in HEK293 cells, supporting that properly designed compounds can increase PKD1 protein level through different mechanisms. ATXL190 increased PKD1 protein levels in both HeLa and HEK293 cells. However, ATXL195 increased the protein level to approximately 190% in HEK293 cells; this compound only marginally increased the protein in HeLa cells, supporting that the protein expression may be differentially regulated in different cell types. HEK293 cells are more relevant to kidney, the target tissue for treating ADPKD.

### Example 2. Antisense Compounds Targeting PKD1 mRNA Increase PKD1 Protein Levels in HK-2 Cells

To further evaluate the possibility of increasing PKD1 protein expression using different mechanisms of action, three additional compounds were designed (Table 3) to target the 3′ UTR of PKD1 mRNA:
1) ATXL265 uses an antisense oligonucleotide (ASO) Coupled Translation—Upregulation 1 (ACT-UP1) compound;
2) ATXL322 targets the miR-17 binding site in PKD1 mRNA similar to ATXL195, supra, but with a different sequence and modification design; and
3) ATXL323 was designed with the same PKD1 mRNA binding sequence as ATXL322 and also contains an ACT-UP1 motif.

The ACT-UP1 compound may comprise an ASO component joined to a protein recruiting sequence (PRS) component (e.g., the PRS comprises GGACU, SEQ ID NO: 4, shown in Table 3 below), wherein the ASO component hybridizes to a target mRNA, the PRS recruits translation related proteins, and wherein the ACT-UP1 compound enhances expression of a target protein. The ACT-UP1 compound may further comprise a conjugate.

TABLE 3
Sequence and Chemistry of Antisense Compounds Targeting PKD1 mRNA

| | | | PKD1 | |
|---|---|---|---|---|
| Compound | Sequence and Chemistry | Sequence | Targeting | SEQ |
| ID | (5′ to 3′) | (S′ to 3′) | Design | ID NO |
| ATXL265 | mG*mG*mAmCmUmGmGmAmCmU | GGACUGGACU | 3′UTR | 30 |
| | mCmCmAmUmUmCmUmGmCmCmU | CCAUUCUGCCUG | ACT-UP1 | |
| | mGmGmCmCmC | GCCC | | |
| ATXL322 | mU*mU*mA*mA*mA*mG*mU*m | UUAAAGUGCUGA | 3′UTR | 31 |
| | G*mC*mU*mG* [AL] *mA* | AGCC | miR-17 | |
| | [GL] *mC* [mCL] | | target site | |
| ATXL323 | mG*mG*mAmCmUmGmGmAmCmU | GGACUGGAC | 3′UTR | 32 |
| | *mU*mA*mA*mA*mG*mU*mG* | UUAAAGUGCUGA | ACT-UP1 & | |
| | mC*mU*mG* [AL] *mA* [GL] * | AGCC | miR-17 | |
| | mC* [mCL] | | | |

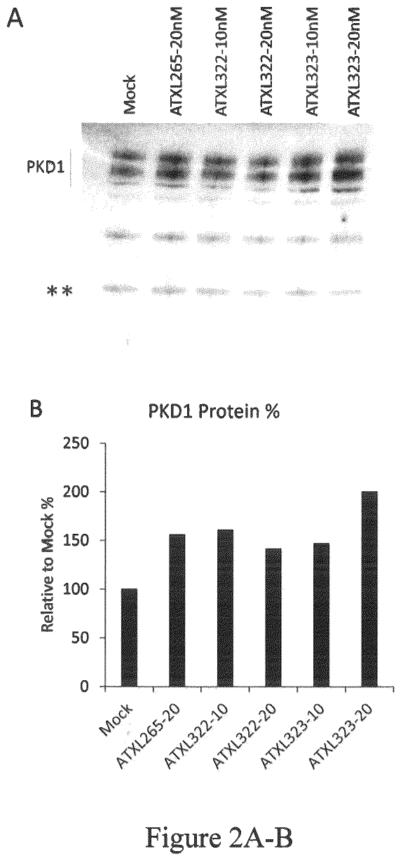

The compounds in Table 3 were transfected into human HK-2 cells (ATCC, Manassas. VA, USA), a proximal tubular cell line derived from the kidney. Cells seeded and grown in one day to ~70% confluency were transfected with the compounds at 10 or 20 nM final concentration using Lipofectamine™ 2000 Transfection Reagent (ThermoFisher Scientific, Waltham, MA), or were mock transfected as a control. Twenty-four (24) hrs after transfection, cells were harvested and the protein was extracted using RIPA Lysis and Extraction Buffer (ThermoFisher Scientific, Waltham, MA, USA). The level of PKD1 protein was determined by Western Blotting the extracted proteins and probing the Western Blot using a PKD1 protein specific antibody (Santa Cruz Biotech, Dallas, TX, USA; Catalog #sc-130554). The Western blotting result is shown in FIG. 2. The Western Blot image was quantified using ImageJ, and the results are shown in Table 4 as percent protein levels relative to mock transfected cells following normalization to a non-specific protein band detected with the same PKD1 protein antibody.

TABLE 4
PKDI Protein Levels in HK-2 Cells Treated with
Antisense Compounds

| Compound | Mock | ATXL265 | ATXL322 | | ATXL323 | |
|---|---|---|---|---|---|---|
| (nM) | 0 nM | 20 nM | 10 nM | 20 nM | 10 nM | 20 nM |
| PKD1 | 100.00 | 155.85 | 160.72 | 141.31 | 146.86 | 199.95 |
| Protein % | | | | | | |

Table 4. PKD1 Protein Levels in HK-2 Cells Treated with Antisense Compounds Mock ATXL265 ATXL322 ATXL323 Compound (nM) 0 nM 20 nM 10 nM 20 nM 10 nM 20 nM PKD1 Protein % 100.00 155.85 160.72 141,31 146.86 199.95 The results show that the 3 compounds can increase PKD1 protein levels in HK-2 cells by 40-100%. ATXL265 and ATXL322 showed similar protein increases; however, under this condition, ATXL323 can cause a higher protein increase compared to ATXL322 at 20 nM. Since ATXL322 and ATXL323 both can bind to the miR-17 binding sites, these compounds can inhibit the suppression effect of miR-17, thus increasing the protein levels. Moreover, ATXL323 also contains an ACT-UP1 motif, which can enhance translation and cause greater protein increase in HK-2 cells at the higher treatment dose.

### Example 3. Antisense Compounds Targeting PKD1 mRNA Increase Pkd1 Protein Levels in Mouse NIH3T3 Cells

As the ATXL265 and ATXL322, ATXL323 binding sites in human PKD1 mRNA are largely conserved between human and mouse, and these compounds only have one mismatch with mouse Pkd1 mRNA (GenBank Accession No: NM_013630.3; SEQ ID NO: 3), the activity of these compounds in mouse NIH3T3 cells (ATCC, Manassas, VA, USA) was evaluated.

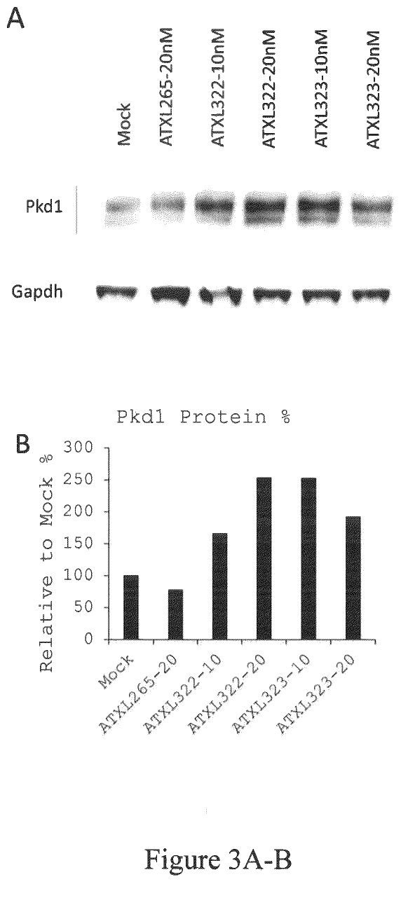

Cells seeded and grown in one day to ~70% confluency were transfected with the compounds at 10 or 20 nM final concentration using Lipofectaminer™ 2000 Transfection Reagent (ThermoFisher Scientific, Waltham, MA), or were mock transfected as a control. Twenty-four (24) hrs after transfection, cells were harvested and the protein was extracted using RIPA Lysis and Extraction Buffer (ThermoFisher Scientific, Waltham, MA, USA). The level of mouse Pkd1 protein was determined by Western Blotting the extracted proteins and probing the Western Blot using a PKD1 protein specific antibody that also recognizes mouse Pkd1 protein (Santa Cruz Biotech, Dallas, TX, USA; Catalog #sc-130554). The Western blotting result is shown in FIG. 3. The Western Blot image was quantified using ImageJ, and the results are shown in Table 5 as percent protein levels relative to mock transfected cells following normalization to mouse Gadph detected using a GAPDH specific antibody (Santa Cruz Biotech, Dallas, TX, USA; Catalog #sc-47724).

TABLE 5
Pkd1 Protein Levels in
NIH3T3 Cells Treated with Antisense Compounds

| Compound | Mock | ATXL265 | ATXL322 | | ATXL323 | |
|---|---|---|---|---|---|---|
| (nM) | 0 | 20 | 10 | 20 | 10 | 20 |
| Pkd1 | 100.00 | 77.62 | 166.11 | 253.53 | 252.67 | 192.14 |
| protein % | | | | | | |

The results showed that, like in human cells, ATXL322 and ATXL323 both increased Pkd1 protein levels, supporting that the regulation of PKD1 mRNA expression with miR-17 is conserved between humans and mice. However, ATXL265 did not increase Pkd1 protein level in NIH3T3 cells, likely due to different regulations in different species or accessibility to the target site in mouse Pkd1 mRNA, which differs from human mRNA. Nevertheless, both ATXL322 and ATXL323 could increase Pkd1 protein levels in mouse cells.

### Example 4. Activity of Antisense Compounds Targeting Pkd1 in Mouse Kidney

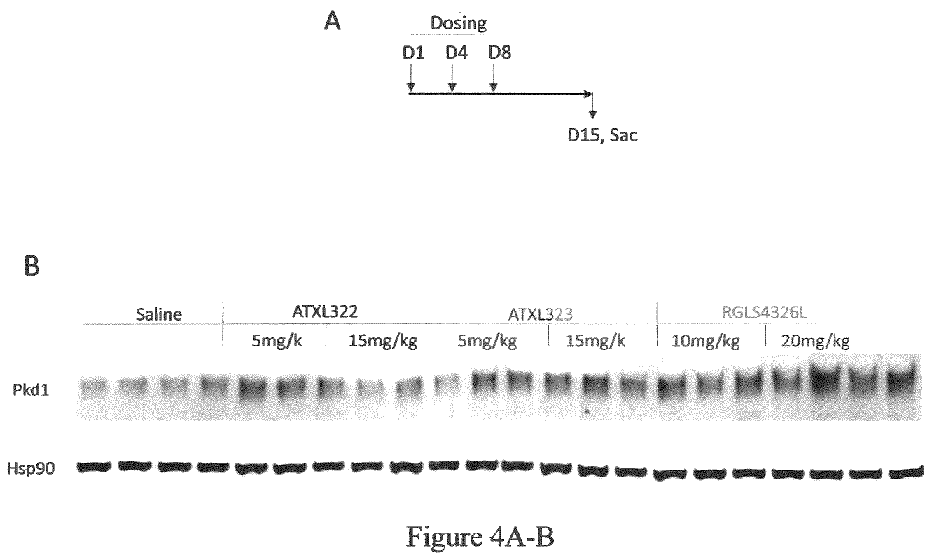

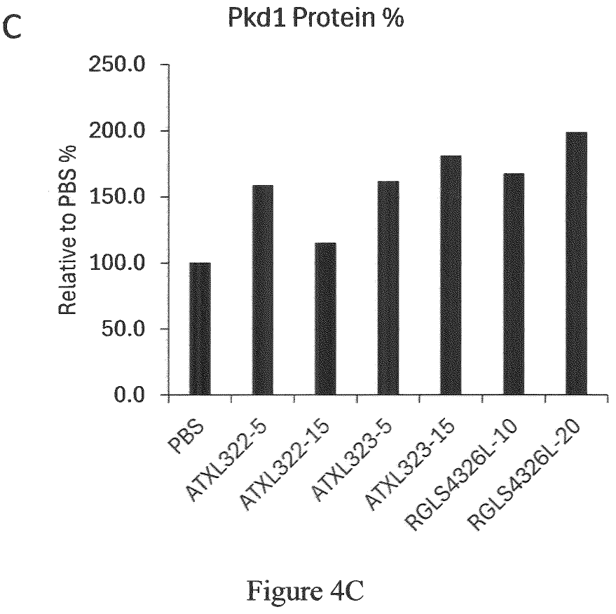

To determine if ATXL322 and ATXL323 can increase Pkd1 protein levels in mouse kidneys in vivo, they were subcutaneously injected at 5 or 15 mg/kg every 4 days, 3 times, in adult (7-8 weeks) male BALB/c mice (N=3). A RGLS compound has been shown previously to increase PKD1 and PKD2 protein levels by inhibiting miR-17 (Lakhia R., et al., 2022, Nature Comm.). As a positive control, a compound similar to RGLS4326 (RGLS4326-Like or RGLS4325-L) was made (Table 6) and was dosed similarly at 10 and 20 mg/kg (N=3). Phosphate-buffered saline (PBS) was injected as a negative control. A schematic of the dosing regimen is shown in FIG. 4.

Mice were sacrificed one week after the last dosing, 15 days after the first dosing, and kidneys were collected. Total protein from the kidney tissue samples was extracted using RIPA Lysis and Extraction Buffer (ThermoFisher Scientific, Waltham, MA, USA) and a tissue homogenizer, and separated by SDS-PAGE. The level of mouse Pkd1 protein was determined by Western Blotting the extracted proteins and probing the Western Blot using a PKD1 protein specific antibody (Santa Cruz Biotech, Dallas, TX, USA; Catalog #sc-130554). The Western blotting result is shown in FIG. 4. The level of Pkd1 on the Western Blot image was quantified using ImageJ, and the results are shown in Table 7 as percent protein levels relative to PBS-treated control mice group following normalization to HSP90, which was detected using an HSP90-specific antibody that recognizes human and mouse protein (ProteinTech, Rosemont, IL, USA; Catalog #13171-1-AP).

TABLE 6
Sequence and Chemistry of RGLS4326-L Targeting Pkd1 mRNA

| | Sequence and Chemistry | Sequence | Targeting | SEQ |
|---|---|---|---|---|
| Compound ID | (5′ to 3′) | (S′ to 3′) | Design | ID NO |
| RGLS4326-L | [AL]*[GL]*mCfAfCfUmU*[TL]* | AGCACUUTG | Targets | 33 |
| | [GL] | | miR-17 | |

TABLE 7
Pkd1 Protein Levels in Mice Kidneys One Week after Last Dose

| Compound | | ATXL322 | ATXL322 | ATXL323 | ATXL323 | RGLS4326 | RGLS4326 |
|---|---|---|---|---|---|---|---|
| ID | PBS | 5 mg/kg | 15 mg/kg | 5 mg/kg | 15 mg/kg | 10 mg/kg | 20 mg/kg |
| Pkd1 | 100.0 | 158.5 | 115.0 | 161.6 | 180.7 | 167.5 | 198.6 |
| protein % | | | | | | | |

The results show that one week after dosing, ATXL322 increased Pkd1 protein levels by about 58%, whereas ATXL323 increased Pkd1 protein by approximately 80%, supporting that both compounds can increase the protein in the kidney. The positive control, RGLS4326-L, also increased Pkd1 protein level as expected.

### Example 5. Activity of Antisense Compounds Targeting Pkd1 in Mouse Kidney for Longer Duration

To determine if ATXL322 and ATXL323 can increase Pkd1 protein levels in mouse kidneys at later time, they were subcutaneously injected at 5 or 15 mg/kg every 4 days, 3 times, to adult (7-8 weeks) male BALB/c mice (N=3). As a positive control, previously described RGLS4326-L (Table 6) was dosed at 10 and 20 mg/kg (N=3). Phosphate-buffered saline (PBS) was injected as a negative control.

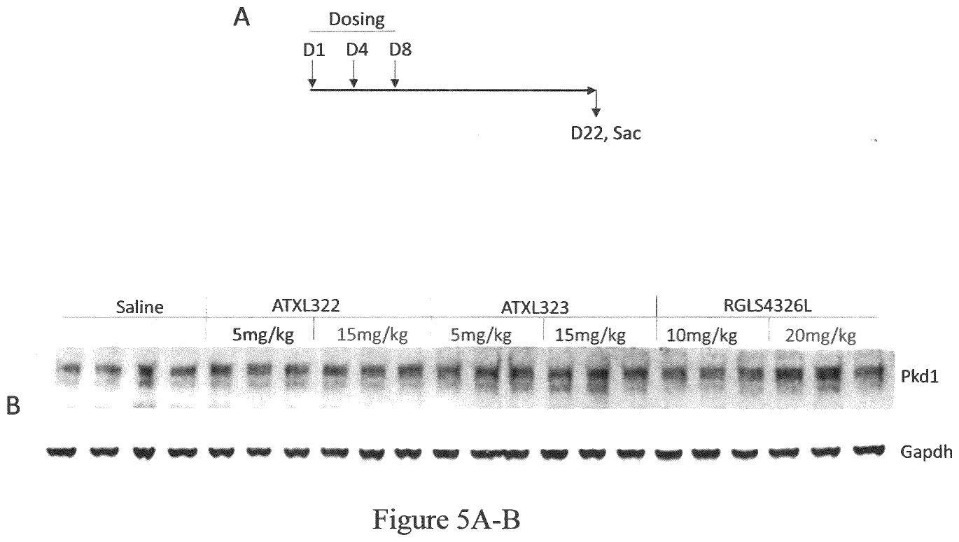

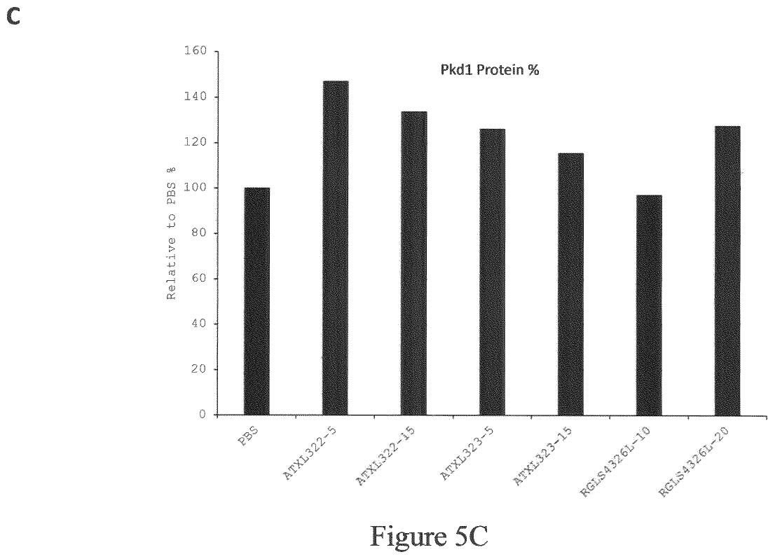

Mice were sacrificed two weeks after the last dosing, 22 days after the first dosing, and kidneys were collected. A schematic of the dosing regimen is shown in FIG. 5. Total protein from the kidney tissue samples was extracted using RIPA Lysis and Extraction Buffer (ThermoFisher Scientific, Waltham, MA, USA) and a tissue homogenizer, and separated by SDS-PAGE. The level of Pkd1 protein was determined by Western Blotting the extracted proteins and probing the Western Blot using a PKD1 protein-specific antibody (Santa Cruz Biotech, Dallas, TX, USA; Catalog #sc-130554). The Western blotting result is shown in FIG. 5. The level of Pkd1 on the Western Blot image was quantified using ImageJ, and the results are shown in Table 8 as percent protein levels relative to PBS-treated control mice group following normalization to GAPDH detected using a GAPDH-specific antibody (Santa Cruz Biotech, Dallas, TX, USA; Catalog #sc-47724).

TABLE 8
Pkd1 Protein Levels in Kidneys of Mice Two Weeks after Last Dose

| Compound | | ATXL322 | ATXL322 | ATXL323 | ATXL323 | RGLS4326-L | RGLS4326-L |
|---|---|---|---|---|---|---|---|
| ID | PBS | 5 mg/kg | 15 mg/kg | 5 mg/kg | 15 mg/kg | 10 mg/kg | 20 mg/kg |
| Pkd1 | 100.0 | 147.0 | 133.6 | 126.1 | 115.5 | 97.0 | 127.5 |
| Protein % | | | | | | | |

The results show that, two weeks after the last dose, ATXL322 increased Pkd1 protein levels by about 47%, whereas ATXL323 increased Pkd1 protein by approximately 26%, supporting that both compounds can increase the protein in the kidney, yet ATXL322 has better activity. The positive control. RGLS4326-L, also modestly increased Pkd1 protein level to a lesser extent than ATXL322. Thus, it appears that ATXL322 has a higher activity than ATXL323 and the positive control RGLS4326-L at a longer time after dosing.

Without being bound by any particular theory, it has been shown previously that shorter antisense compounds may have better delivery to kidney (Khvorova A., Watts J. K., Nat Biotechnol. 2017; 35(3):238-248; Zanardi Z. A., et al., 2012, J. Phar. & Exp. Ther. 343 (2) 489-496). RGLS4326-L is the shortest compound tested (9nt) and its short length allows faster uptake by kidney cells than the longer ATXL322 and ATXL323 compounds, hence the high activity of RGLS4326-L seen in FIG. 4. However, at the longer time point, the longer compounds may be more stable than the shorter compounds. Further, ATXL322 and ATXL323 target the same site in the Pkd1 3′ UTR, however, ATXL323 is longer than ATXL322, which may reduce the uptake of ATXL323 to the kidney compared to ATXL322 as shown by the relative activity levels in Table 8.

### Example 6. ATXL322 Activity in Mouse Kidney at a Higher Dose

To evaluate if increasing the dose can further increase Pkd1 protein levels, ATXL322 was subcutaneously injected at 15 or 50 mg/kg every 4 days, for 2 times, to adult (7-8 weeks) male BALB/c mice (N=4). As a control, PBS was also dosed similarly.

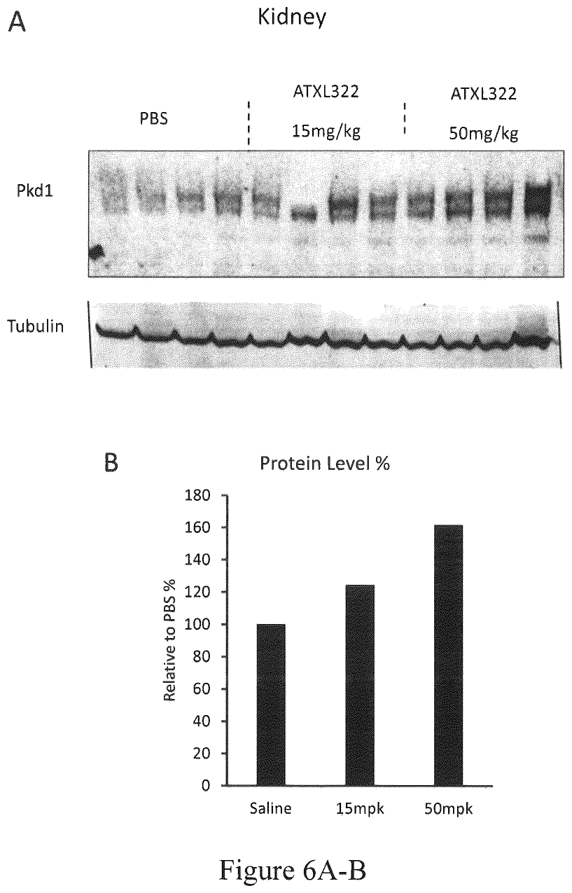

One week after the last dosing, mice were sacrificed, and kidneys were collected. Total protein from the kidney tissue samples was extracted using RIPA Lysis and Extraction Buffer (ThermoFisher Scientific, Waltham, MA, USA) and a tissue homogenizer, and separated by SDS-PAGE. The level of Pkd1 protein was determined by Western Blotting the extracted proteins and probing the Western Blot using a PKD1 protein-specific antibody (Santa Cruz Biotech, Dallas, TX, USA; Catalog #sc-130554). The Western blotting result is shown in FIG. 6. The level of Pkd1 on the Western Blot image was quantified using ImageJ, and the results are shown in Table 9 as percent protein levels relative to PBS-treated control mice group following normalization to a loading control protein detected using a Tubulin-specific antibody (Abcam, Cambridge, UK; Catalog #ab7291).

TABLE 9
Pkd1 Protein Levels in Kidneys of Mice Treated with ATXL322

| Compound ID | Pkd1 Protein level % |
|---|---|
| Saline | 100 |
| ATXL322 (15 mg/kg) | 124.3 |
| ATXL322 (50 mg/kg) | 161.4 |

The results show that increasing the dose level of ATXL322 can increase the upregulation of Pkd1 protein, leading to higher protein levels in mouse kidneys.

### Example 7. Effects of Antisense Compound Conjugation on Pkd1 Protein Levels in Kidney

To determine whether uptake of an ACT-UP1 compound by kidney cells can be increased, a Pkd1 targeting compound was conjugated with a cholesterol moiety (Ostergaard ME., et al., Nucleic Acids Res., 2019, 47(12)6045-6058). A compound, ATXL551, was designed that has the same sequence as ATXL322 but with 2′-O-Methoxyethyl (2′-MOE) modifications and conjugated at the 3′ end to a cholesterol moiety (HG-CHOL) (Table 10).

TABLE 10
Chemistry and Sequence of ATXL551

| Compound | Sequence and Chemistry | Sequence | SEQ ID |
|---|---|---|---|
| ID | (5′ to 3′) | (5′ to 3′) | NO |
| ATXL551 | [eT] * [eT] * [eA] * [eA] * [eA] * [eG] * | TTAAAGTGC | 34 |
| | [eT] * [eG] * [eCm] * [eT] * [eG] * [AL] * | TGAAGCC | |
| | [eA] * [GL] * [eCm] * [mCL] [HG-CHOL] | | |

ATXL551 and ATXL322 were subcutaneously injected at 5 or 15 mg/kg every 4 days, 2 times to adult (7-8 weeks) male BALB/c mice (N=1-3 in different groups). As a control, PBS was also dosed similarly (N=2).

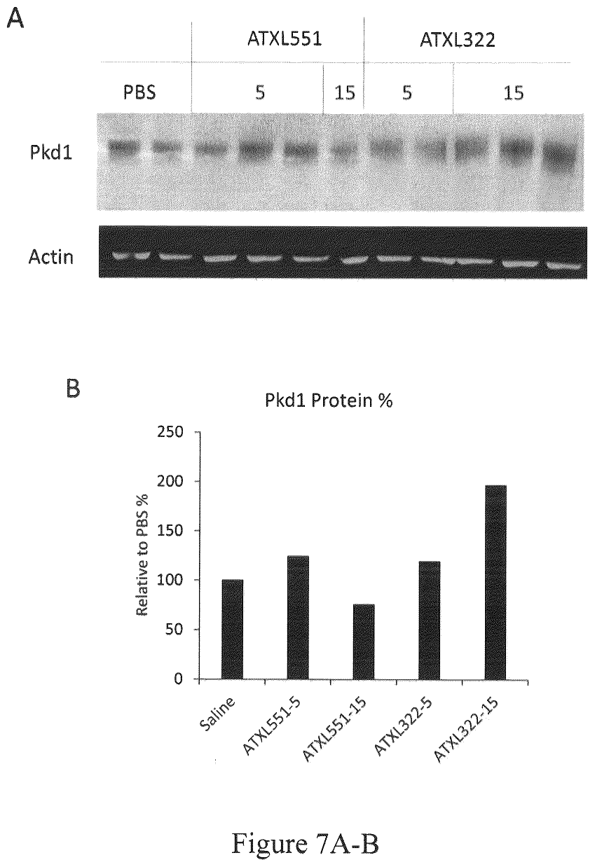

One week after the last dosing, mice were sacrificed, and kidneys were collected. Total protein from the kidney tissue samples were extracted using RIPA Lysis and Extraction Buffer (ThermoFisher Scientific, Waltham, MA, USA) and a tissue homogenizer, and separated by SDS-PAGE. The level of Pkd1 protein was determined by Western Blotting the extracted proteins and probing the Western Blot using a-PKD1 protein-specific antibody (Santa Cruz Biotech, Dallas, TX, USA; Catalog #sc-130554). The Western blotting result is shown in FIG. 7. The level of Pkd1 on the Western Blot image was quantified using ImageJ, and the results are shown in Table 11 as percent protein levels relative to PBS-treated control mice group following normalization to

TABLE 11
Pkd1 Protein Levels in the Kidney of Mice Treated with ATXL322

| | | ATXL551 | ATXL551 | ATXL322 | ATXL322 |
|---|---|---|---|---|---|
| Compound ID | Saline | 5 mg/kg | 15 mg/kg | 5 mg/kg | 15 mg/kg |
| Pkd1 Protein | 100.0 | 124.3 | 75.6 | 119.2 | 196.5 |
| % | | | | | |

The results show that the cholesterol conjugate does not enhance activity much in the kidney, whereas, ATXL322 at 15 mg/kg significantly increased Pkd1 protein levels compared to PBS group. (P=:0.0157).

### Example 8. Additional Antisense Compounds Targeting Pkd1 mRNA Can Increase Pkd1 Protein Levels in Mouse NIH3T3 Cells

New compounds were made with improved designs to enhance PKD1 protein expression.

These new compounds include:
1) three compounds targeting the 5′ UTR region of PKD1 (ATXL494, ATXL512, ATXL513);
2) four compounds derived from previously described ATXL322, supra, but with different 3′ conjugate moieties linked to the oligonucleotide by a phosphate bond (ATXL586 has a HEG conjugate moiety, ATXL587 has HEG+PALM, ATXL594 has HEG+FA-FI, ATXL595 has HEG+FA-FI+PALM);
3) an RNase H1-active compound (ATXL591) that can reduce the Pkd1 protein levels and is used as a control to validate antibody detection of Pkd1 protein level changes after treatment; and.
4) an ACT-UP1 compound (ATXL501) targeting 3′ UTR.

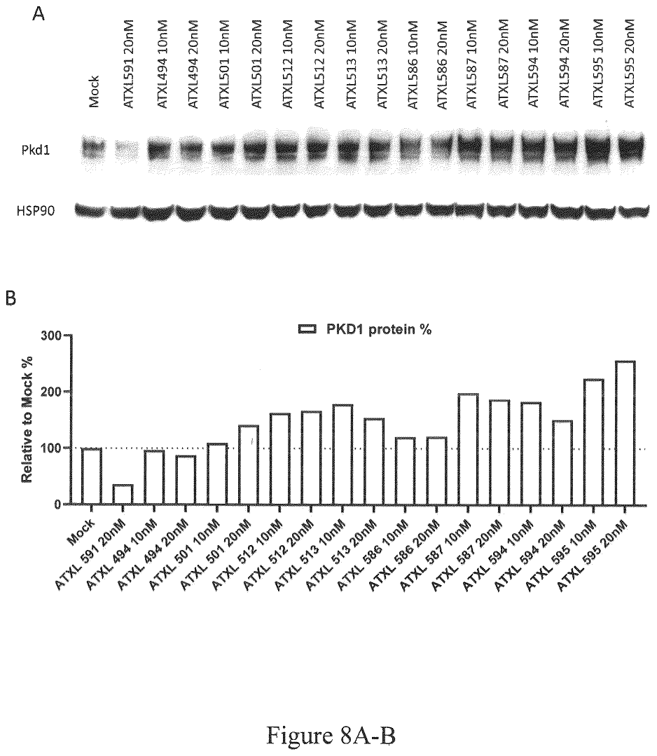

These compounds are listed in Table 12 and their activity were evaluated in mouse NIH3T3 cells (ATCC, Manassas, VA, USA). Cells seeded and grown in one day to ~70% confluency were transfected with the compounds at 10 or 20 nM final concentration using Lipofectamine™ 2000 Transfection Reagent (ThermoFisher Scientific, Waltham, MA), or were mock transfected as a control. Twenty-four (24) hrs after transfection, cells were harvested, and the protein was extracted using RIPA Lysis and Extraction Buffer (ThermoFisher Scientific Waltham, MA, USA). The level of Pkd1 protein was determined by Western Blotting using a PKD1 protein specific antibody (Santa Cruz Biotech, Dallas, TX, USA; Catalog #sc-130554). The Western blotting result is shown in FIG. 8. The Western Blot image was quantified using ImageJ, and the results are shown in Table 12 as percent protein levels relative to mock transfected cells following normalization to HSP90, which was detected using an HSP90-specific antibody (ProteinTech, Rosemont, IL, USA; Catalog #13171-1-AP).

TABLE 12
Sequence and Chemistry of New Compounds Targeting Pkd1 mRNA

| | | | Pkd1 | |
|---|---|---|---|---|
| Compound | Sequence and Chemistry | Sequence | Targeting | SEQ ID |
| ID | (5′ to 3′) | (5′ to 3′) | Design | NO |
| ATXL494 | mG*mG*mC*mC*mC*mC*mG*mC | GGCCCCGCC | 5′UTR | 35 |
| | *mC*mG*mU* [mCL] *mC* [mCL] | GUCCCCA | | |
| | *mC* [AL] | | | |
| ATXL512 | [eT] * [eG] * [eG] * [eCm] * [e | TGGCCCCGC | 5′UTR | 36 |
| | Cm] * [eCm] * [eCm] * [eG] * [e | CGTCC | | |
| | Cm] * [eCm] * [eG] * [eT] * [eC | | | |
| | m] * [eCm] | | | |
| ATXL513 | [eT] * [eG] * [eG] * [eCm] *[e | TGGCCCCGC | 5′UTR | 37 |
| | Cm] * [eCm] * [eCm] * [eG] * [e | CGTCC | | |
| | Cm] * [mCL] * [eG] * [TL] * [eC | | | |
| | m] * [mCL] | | | |
| ATXLS86 | mU*mU*mA*mA*mA*mG*mU*mG | UUAAAGUGC | miR-17 | 31 |
| | *mC *mU*mG* [AL] *mA* [GL] * | UGAAGCC | target site | |
| | mC* [mCL] [HEG] | | 3′UTR | |
| ATXL587 | mU*mU*mA*mA*mA*mG*mU*mG | UUAAAGUGC | miR-17 | 31 |
| | *mC*mU*mG* [AL] *mA* [GL] * | UGAAGCC | target site | |
| | mC* [mCL] [HEG] * [PALM] | | 3′UTR | |
| ATXL594 | mU*mU*mA*mA*mA*mG*mU*mG | UUAAAGUGC | miR-17 | 31 |
| | *mC*mU*mG* [AL] *mA* [GL] * | UGAAGCC | target site | |
| | mC* [mCL] [HEG] * [FA-FI] | | 3′UTR | |
| ATXL595 | mU*mU*mA*mA*mA*mG*mU*mG | UUAAAGUGC | miR-17 | 31 |
| | *mC*mU*mG* [AL] *mA* [GL] * | UGAAGCC | target site | |
| | mC* [mCL] [HEG] * [FA- | | 3′UTR | |
| | FI] * [PALM] | | | |
| ATXL591 | [GL] * [AL] * [AL] *G*A*G*A* | GAAGAGATG | RNAse H | 38 |
| | T*G*T*T*G*T* [TL] * [GL] * [GL] | TTGTTGG | sensitive | |
| ATXL501 | [eG] * [eG] * [eA] * [eCm]*[eT] | GGACTGGAC | ACT-UP1 & | 39 |
| | * [eG] * [eG] * [eA] * [eCm] | GGACCCTGG | 3′UTR | |
| | * [eG ] * [eG] * [eA] * [eCm] * | GTCCTGG | | |
| | [eCm] *[eCm]*[eT] * [eG] * | | | |
| | [eG] * [eG] * [eT] * [eCm] * | | | |
| | [eCm] * [eT] * [eG] * [eG] | | | |

TABLE 13
Pkd1 Protein Levels in Mouse NIH3T3 Cells After Treatment

| Compound ID | Pkd1 Protein % |
|---|---|
| Mock | 100 |
| ATXL591 20 nM | 36.43 |
| ATXL494 10 nM | 96.66 |
| ATXL494 20 nM | 87.56 |
| ATXL501 10 nM | 109.81 |
| ATXL501 20 nM | 141.89 |
| ATXL512 10 nM | 163.37 |
| ATXL512 20 nM | 167.16 |
| ATXL513 10 nM | 179.39 |
| ATXL513 20 nM | 154.69 |
| ATXL586 10 nM | 121.09 |
| ATXL586 20 nM | 121.78 |
| ATXL587 10 nM | 198.92 |
| ATXL587 20 nM | 188.46 |
| ATXL594 10 nM | 183.64 |
| ATXL594 20 nM | 151.47 |
| ATXL595 10 nM | 224.81 |
| ATXL595 20 nM | 257.32 |

The results show that, the RNase H1-dependent compound ATXL591 reduced the level of Pkd1 protein, validating the ability of the PKD1 antibody to detect changes in Pkd1 expression after treatment. Pkd1 protein levels were increased ~150-180% by compounds ATXL512 and ATXL513 that target the 5′ UTR. The ACT-UP1 compound, ATXL501, increased the protein level to ~140%, supporting that Pkd1 protein levels can be increased through different mechanisms. Consistent with the observation for ATXL322 in Example 3, compounds with the same sequence and chemistry, but, with different conjugation at the 3′ end, increased Pkd1 protein levels.

### Example 9. Additional Design of Compounds Targeting the PKD1

Previous studies showed that compounds targeting 5′UTR, compounds targeting 3′ UTR, or compounds with ACT-UP1 design can increase PKD1 protein levels. Additional compounds were designed to enhance the potency of PKD1 protein expression: 1) ACT-UP1 compounds (the protein recruiting sequence (PRS) is bolded and underlined in the table); 2) compounds targeting the 5′UTR targeting site of ATXL512; 3) compounds targeting a miR-17 target site in the 3′UTR, and, 4) a mouse-specific compound designed based on the homologous site of ATXL512. The sequence and chemistry of the new compounds are listed in Table 14.

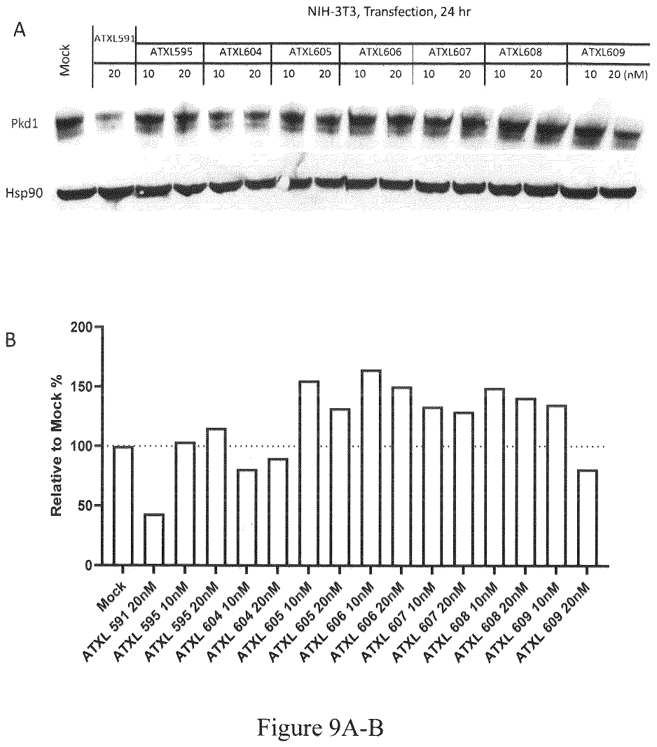

The activities of select compounds from Tables 12 and 14 were evaluated in mouse NIH3T3 cells (ATCC, Manassas, VA, USA). Cells seeded and grown in one day to ~70% confluency were transfected with the compounds at 10 or 20 nM final concentration using Lipofectamine™ 2000 Transfection Reagent (ThermoFisher Scientific, Waltham, MA), or were mock transfected as a control. Twenty-four (24) hrs after transfection, cells were harvested, and the protein was extracted using RIPA Lysis and Extraction Buffer (ThermoFisher Scientific, Waltham, MA, USA). The level of Pkd1 protein was determined by Western Blotting using a PKD1 protein specific antibody (Santa Cruz Biotech, Dallas, TX, USA; Catalog #sc-130554). The Western blotting result is shown in FIG. 9. The Western Blot image was quantified using ImageJ, and the results are shown in Table 15 as percent protein levels relative to mock transfected cells following normalization to Hsp90, which was detected using an HSP90-specific antibody (ProteinTech, Rosemont, IL, USA; Catalog #13171-1-AP).

TABLE 14
Sequence and Chemistry of New Compounds Targeting PKD1 mRNA

| | | | PKD1 Targeting | |
|---|---|---|---|---|
| | | | Design | |
| Compound | Sequence and Chemistry | Sequence | (Parental | SEQ ID |
| ID | (5′ to 3′) | (5′ to 3′) | Compound) | NO |
| ATXL604 | [eT] * [eG] * [eG] * [eCm] * [e | TGGCCCCGC | 5′UTR | 40 |
| | Cm] * [eCm] * [eCm] * [eG] *[e | CGTCCCC | (ATXL512) | |
| | Cm] * [eCm] * [eG] * [eT] * [eC | | | |
| | m]* [eCm] * [eCm] * [eCm] | | | |
| ATXL605 | mU*mU*mA*mA*mA*mG*mU*mG | UUAAAGUGC | 3′UTR, miR-17 | 41 |
| | *mC*mU*mG* [GL] *mA* [GL] * | UGGAGCC | target site | |
| | mC* [mCL] | | (ATXL322) | |
| ATXL606 | [eT] * [eT] * [eA] * [eA] * [eA] | TTAAAGTGC | 3′UTR, miR-17 | 34 |
| | * [eG] * [e]] * [eG] * [eCm] * | TGAAGCC | target site | |
| | [eT] * [eG] * [AL] * [eA] * [GL] | | (ATXL322) | |
| | * [eCm] * [mCL] | | | |
| ATXL607 | mG*mG*mAmCmUmGmGmAmC* [e | GGACUGGAC | ACT-UP1 & | 42 |
| | Cm] * [eCm] * [eA] * [eT] * [eT] | CCATTCTGC | 3′UTR | |
| | * [eCm] * [eT] * [eG] * [eCm] | CTGGCC | (ATXL265) | |
| | * [eCm] * [TL] * [eG] * [GL] * | | | |
| | [eCm] * [mCL] | | | |
| ATXL608 | mG*mG*mAmCmUmGmGmAmC* [e | GGACUGGAC | ACT-UP1 & | 43 |
| | G] * [eG] * [eA] * [eCm] * [eCm] | GGACCOTGG | 3′UTR | |
| | * [ eCm] * [eT] * [eG] * [eG] * | GTCCTG | (ATXL501) | |
| | [eG] * [TL] * [eCm] * [mCL] * | | | |
| | [eT] * [GL] | | | |
| ATXL609 | mG*mG*mAmCmUmGmGmAmC* [e | GGACUGGAC | ACT-UP1 & | 44 |
| | Cm] * [eCm] * [eA] * [eA] * [eT] | CCAATACTG | 3′UTR | |
| | * [eA] * [eCm] * [eT] * [eG] * | CTGTGT | | |
| | [eCm] * [TL] * [eG] * [TL] *[e | | | |
| | G] * [TL] | | | |
| ATXL610 | [eG] * [eG] * [eCm] * [eCm] * | GGCCCCGCC | 5′UTR | 45 |
| | [eCm] * [eCm] * [eG] * [eCm] * | GTCCCCA | (ATXL494) | |
| | [eCm] * [eG] * [eT] * [eCm] * [e | | | |
| | Cm] * [eCm] * [eCm] * [eA] | | | |
| ATXL611 | [eG] * [eG] * [eCm] * [eCm] * | GGCCCCGCC | 5′UTR | 46 |
| | [eCm] * [eCm] * [eG] * [eCm] * | GTCCCCA | (ATXL512) | |
| | [eCm] *[eG] * [e]] * [eCm] * [e | | | |
| | Cm] * [eCm] * [mCL] * [AL] | | | |
| ATXL612 | [eG] * [eG] * [eCm] * [eCm] * | GGCCCCGCC | 5 UTR | 47 |
| | [eCm]*[eCm] *[eG] * [eCm]* | GTCCCC | (ATXL512) | |
| | [eCm]* [eG] *[eT] *[eCm] *[e | | | |
| | Cm] * [eCm] * [eCm] | | | |
| ATXL613 | [eG] * [eG] * [eCm] * [eCm] * | GGCCCCGCC | 5′UTR | 48 |
| | [eCm] * [eCm] * [eG] * [eCm] * | GTCCC | (ATXL512) | |
| | [eCm] * [eG] * [eT] * [eCm] * [m | | | |
| | CL ] * [mCL] | | | |
| ATXL614 | [eG] * [eCm] * [eCm] * [eCm] * | GCCCCGCCG | 5′UTR | 49 |
| | [eCm] * [eG] * [eCm] * [eCm] * | TCCCC | (ATXL512) | |
| | [eG] * [e]] * [eCm] * [eCm] * | | | |
| | [mCL] * [mCL] | | | |
| ATXL615 | [eG] * [eG] * [eCm] * [eCm] * | GGCCCCGGT | 5′UTR | 50 |
| | [eCm] * [eCm] * [eG] * [eG] * [e | TCCCCG | (mouse-sequence | |
| | T] * [eT] * [eCm] * [eCm] * [eC | | of ATXLS12 | |
| | m] * [mCL] * [GL] | | site) | |

TABLE 15
Pkd1 Protein Levels in NIH3T3 Cells 24 Hours After Transfection

| Compound ID | Pkd1 Protein Level % |
|---|---|
| Mock | 100.0 |
| ATXL591 (20 nM) | 43.5 |
| ATXL595 (10 nM) | 103.7 |
| ATXL595 (20 nM) | 115.4 |
| ATXL604 (10 nM) | 81.0 |
| ATXL604 (20 nM) | 90.3 |
| ATXL605 (10 nM) | 155.5 |
| ATXL605 (20 nM) | 132.1 |
| ATXL606 (10 nM) | 164.7 |
| ATXL606 (20 nM) | 150.5 |
| ATXL607 (10 nM) | 133.6 |
| ATXL607 (20 nM) | 129.4 |
| ATXL608 (10 nM) | 149.3 |
| ATXL608 (20 nM) | 141.1 |
| ATXL609 (10 nM) | 135.2 |
| ATXL609 (20 nM) | 80.9 |

The results showed that compound ATXL604 targeting the 5′UTR did not increase Pkd1 protein level. The compounds targeting miR-17 binding sites (also referred to herein as target sites) in the 3′UTR of Pkd1 mRNA, ATXL595, ATXL605 and ATXL606, increased Pkd1 protein levels. In addition, three ACT-UP1 compounds that target the 3′ UTR of Pkd1 mRNA, ATXL607, ATXL608, and ATXL609, also increased Pkd1 protein levels.

### Example 10. An ACT-UP1 Compound Increases PKD1 Protein Levels in Human Cells

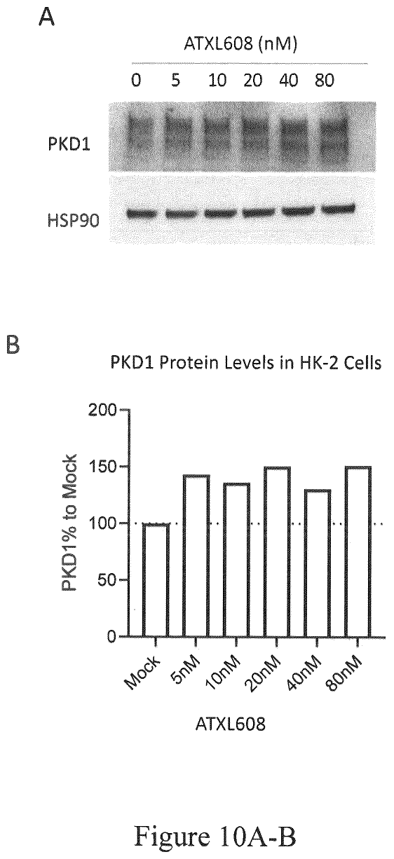

Since several ACT-UP1 compounds from the previous example target a conserved site in PKD1 mRNA, the activity of one compound, ATXL608, was tested in human HK-2 cells (ATCC, Manassas, VA, USA), epithelial cells derived from kidney proximal tubule. Cells seeded and grown in one day to ~70% confluency were transfected with the ATXL608 compound at 5, 10, 20, 40, or 80 nM final concentrations using Lipofectamine™ 2000 Transfection Reagent (ThermoFisher Scientific, Waltham, MA), or were mock transfected as a control. Twenty-four (24) hrs after transfection, cells were harvested and the protein was extracted using RIPA Lysis and Extraction Buffer (ThermoFisher Scientific, Waltham, MA, USA). The level of PKD1 protein was determined by Western Blotting using a PKD1 protein specific antibody (Santa Cruz Biotech, Dallas, TX, USA; Catalog #sc-130554). The Western blotting result is shown in FIG. 10. The Western Blot image was quantified using ImageJ, and the results are shown in Table 16 as percent protein levels relative to mock transfected cells following normalization to HSP90, which was detected using an HSP90-specific antibody (ProteinTech, Rosemont, IL, USA; Catalog #13171-1-AP).

TABLE 16
Antisense Compound Activity in Human HK-2 Cells

| ATXL608 | PKD1 Protein Level % |
|---|---|
| 0 (Mock) | 100.0 |

| | | |
|---|---|---|
| 5 | nM | 143.2 |
| 10 | nM | 136.0 |
| 20 | nM | 150.6 |
| 40 | nM | 130.6 |
| 80 | nM | 151.1 |

The results showed that ATXL608 increased PKD1 protein expression by about 30-50% in HK-2 human cell line under all the concentrations tested.

### Example 11. An ACT-UP1 Compound Increases PKD1 Protein Levels in ADPKD Patient-Derived Cells After Transfection

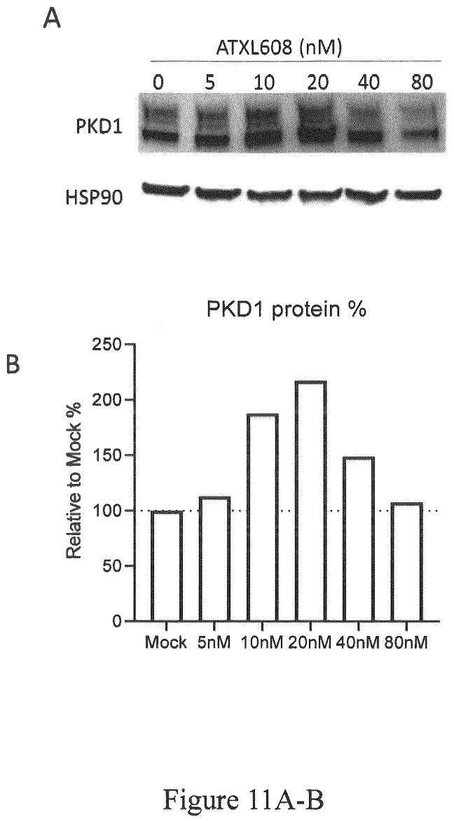

To evaluate if the ATXL608-mediated protein increase seen in the previous example can also be achieved in a disease setting, a WT 9-7 cell line derived from an ADPKD patient and containing a heterozygous mutation of the PKD1 gene (ATCC, Manassas, VA, USA; catalog #CRL-2830) was transfected with the compound. Cells seeded and grown in one day to ~70% confluency were transfected with the compound at 5, 10, 20, 40, or 80 nM final concentrations using Lipofectamine™ 2000 Transfection Reagent (ThermoFisher Scientific, Waltham, MA), or were mock transfected as a control. Twenty-four (24) hrs after transfection, cells were harvested and the protein was extracted using RIPA Lysis and Extraction Buffer (ThermoFisher Scientific, Waltham, MA, USA). The level of PKD1 protein was determined by Western Blotting using a PKD1 protein specific antibody (Santa Cruz Biotech, Dallas, TX, USA; Catalog #sc-130554). The Western blotting result is shown in FIG. 11. The Western Blot image was quantified using ImageJ, and the results are shown in Table 17 as percent protein levels relative to mock transfected cells following normalization to HSP90, which was detected using an HSP90-specific antibody (ProteinTech, Rosemont, IL, USA; Catalog #13171-1-AP).

TABLE 17
Activity in Patient-Derived WT 9-7 Cells by Transfection

| | |
|---|---|
| ATXL608 | PKD1 Protein Level % |

| | | |
|---|---|---|
| 0 | nM (Mock) | 100.0 |
| 5 | nM | 113.0 |
| 10 | nM | 187.6 |
| 20 | nM | 217.3 |
| 40 | nM | 148.9 |
| 80 | nM | 107.5 |

The results show that PKD1 protein levels were increased up to about 217% after transfection with ATXL608 in WT 9-7 patient-derived cell line.

### Example 12. An ACT-UP1 Compounds Increase PKD1 Protein Levels in ADPKD Patient-Derived Ceils After Free Uptake

To mimic an in vivo situation, the activity of ACT-UP1 compound ATXL608 was evaluated in WT 9-7 cells by free uptake, i.e., entry of the compound into cells in the absence of transfection reagents. As a positive control, RGLS8429 (also known as RG-NG-1015 in WO2024215846) was made (Table 18) and also assessed in WT 9-7 cells.

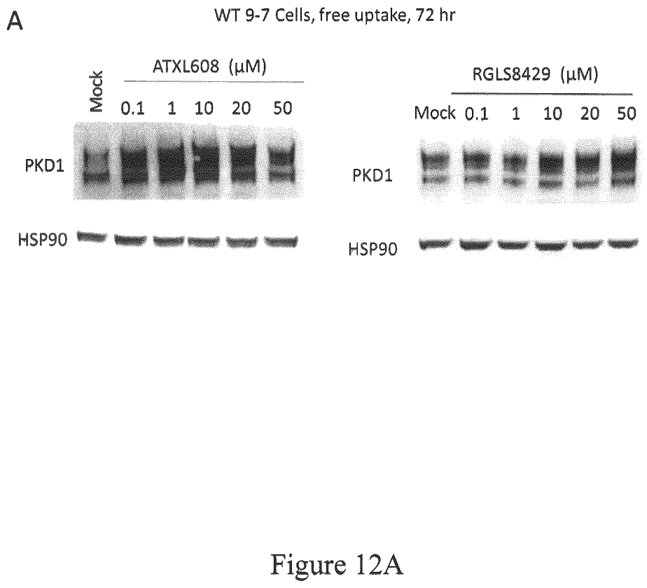

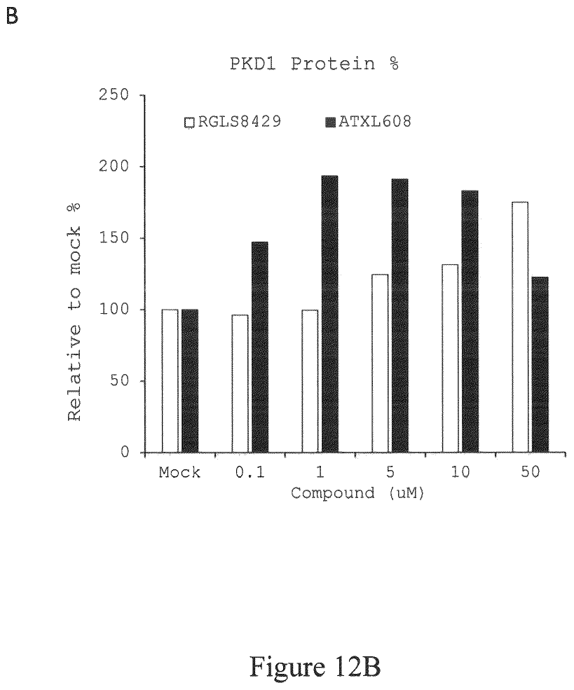

Cells seeded and grown in one day to ~60% confluency were incubated with the compound at 0, 1, 1, 10, 20, and 50 μM final concentrations. Seventy-two (72) hrs after incubation, cells were harvested, and the protein was extracted using RIPA Lysis and Extraction Buffer (ThermoFisher Scientific. Waltham, MA, USA). The level of PKD1 protein was determined by Western Blotting using a PKD1 protein specific antibody (Santa Cruz Biotech, Dallas, TX, USA; Catalog #sc-130554). The Western blotting result is shown in FIG. 12. The Western Blot image was quantified using ImageJ, and the results are shown in Table 19 as percent protein levels relative to mock incubated cells following normalization to HSP90, which was detected using an HSP90-specific antibody (ProteinTech, Rosemont. IL, USA; Catalog #13171-1-AP).

TABLE 18
Sequence and Chemistry of RGLS8429 Targeting Pkd1 mRNA

| | Sequence and Chemistry | Sequence | Targeting | SEQ |
|---|---|---|---|---|
| Compound ID | (S′ to 3′) | (5′ to 3′) | Design | ID NO |
| RGLS8429 | [cEt-A] * [cEt- | AGCACUUUA | Targets | 51 |
| | G] * [mC] * [fA] * [fC] * [fU] * | | miR-17 | |
| | [mU] * [cEt-U] * [cEt-A] | | | |

TABLE 19
Activity in Patient-Derived WT 9-7 Cells by Free Uptake

PKD1 Protein Level %

| Compound (μM) | ATXL608 | RGLS8429 |
|---|---|---|
| 0 (Mock) | 100.0 | 100.0 |
| 0.1 | 147.3 | 96.2 |
| 1 | 193.5 | 99.5 |
| 10 | 191.2 | 124.5 |
| 20 | 182.9 | 131.4 |
| 50 | 122.4 | 175.0 |

The results showed that PKD1 protein can be substantially increased nearly two-fold in these patient-derived cells when ATXL608 was delivered by free uptake, a situation that is more relevant to in vivo settings.

### Example 13. Antisense Compounds Increase Pkd1 Protein Levels In Vivo

Select antisense compounds were assessed to determine whether they can increase mouse Pkd1 protein levels in vivo. Compounds ATXL607, ATXL608, and ATXL615 were subcutaneously injected with a dosing regimen of 10 mg/kg every 3 days, for 2 times to adult (7-8 weeks) male BALB/c mice (N=3). As a control, PBS was also dosed similarly.

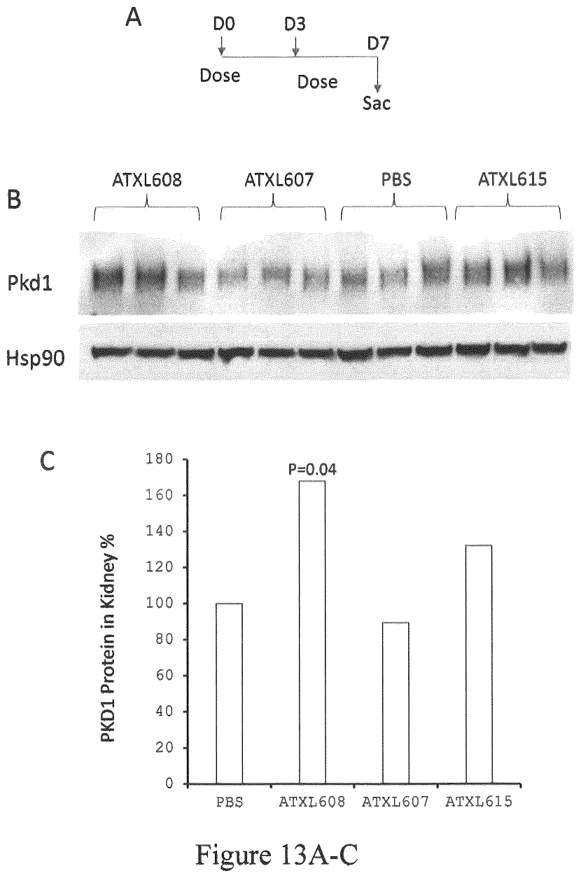

One week ater the first dosing, mice were sacrificed, and kidneys were collected. Total protein from the kidney tissue samples was extracted using RIPA Lysis and Extraction Buffer (ThermoFisher Scientific, Waltham, MA, USA) and a tissue homogenizer, and separated by SDS-PAGE. The level of Pkd1 protein was determined by Western Blotting the extracted proteins and probing the Western Blot using a PKD1 protein-specific antibody (Santa Cruz Biotech, Dallas, TX, USA; Catalog #sc-130554). The Western blotting result is shown in FIG. 13. The level of Pkd1 on the Western Blot image was quantified using ImageJ, and the results are shown in Table 20 as percent protein levels relative to PBS-treated control mice group following normalization to a loading control protein, Hsp90, which was detected using an HSP90-specific antibody (ProteinTech, Rosemont, IL, USA; Catalog #13171-1-AP).

TABLE 20
Activity of Pkd1 in Mouse Kidney

| Compound ID | Pkd1 Protein Level in Kidney % |
|---|---|
| PBS | 100.0 |
| ATXL608-10 mg/kg | 168.0 |
| ATXL607-10 mg/kg | 89.3 |
| ATXL615-10 mg/kg | 132.0 |

The results showed that the ACT-UP1 compound ATXL608 significantly increased Pkd1 protein level (P=0.015) in mouse kidneys. Another 5′ UTR targeting compound, ATXL615, also increased Pkd1 in the kidney after subcutaneous injection.

### Example 14. ACT-UP1 Compound ATXL608 Increases Pkd1 Protein Levels In Vivo

To evaluate whether ACT-UP1 compound ATXL608 would enhance Pkd1 protein expression in a dose-responsive manner in vivo, it was subcutaneously injected with a dosing regimen of 7.5 or 15 mg/kg every 3 days, for 2 times into adult (7-8 weeks) male BALB/c mice (N=3). As a control, PBS was also dosed similarly.

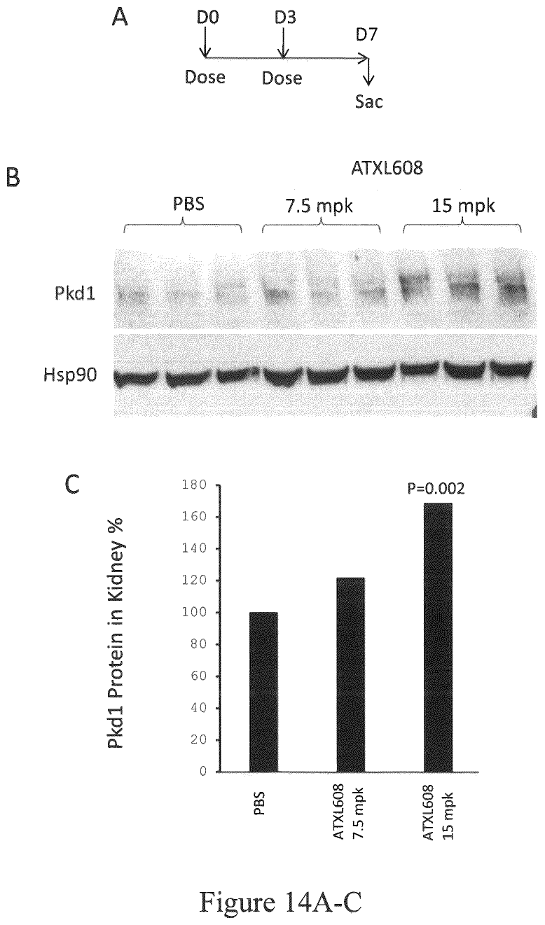

One week after the first dosing, mice were sacrificed, and kidneys were collected. Total protein from the kidney tissue samples was extracted using RIPA Lysis and Extraction Buffer (ThermoFisher Scientific, Waltham, MA, USA) and a tissue homogenizer, and separated by SDS-PAGE. The level of Pkd1 protein was determined by Western Blotting the extracted proteins and probing the Western Blot using a PKD1 protein-specific antibody (Santa Cruz Biotech, Dallas, TX, USA; Catalog #sc-130554). The Western blotting result is shown in FIG. 14. The level of Pkd1 on the Western Blot image was quantified using ImageJ, and the results are shown in Table 21 as percent protein levels relative to PBS-treated control mice group following normalization to a loading control protein, Hsp90, which was detected using an HSP90-specific antibody (ProteinTech, Rosemont, IL, USA; Catalog #13171-1-AP).

TABLE 21
Dose-Dependent Expression of Pkd1 in Mouse Kidney

| Compound ID | Pkd1 Protein Level in Kidney % |
|---|---|
| PBS | 100.0 |
| ATXL608-7.5 mg/kg | 121.8 |
| ATXL608-15 mg/kg | 168.7 |

The results indicate that a modest Pkd1 protein increase in the kidney was observed at 7.5 mg/kg, whereas a significant Pkd1 protein increase was detected at 15 mg/kg, supporting a dose-dependent effect. This suggests that a dosage of 10-15 mg/kg could achieve a significant Pkd1 increase in vivo.

### Example 15. In Vivo Durability of ACT-UP1 Compound ATXL608

To determine the durability of the ACT-UP1 compound ATXL608 in increasing Pkd1 protein levels in vivo, it was subcutaneously injected with a dosing regimen of 15 mg/kg every 3 days, for 2 times into adult (7-8 weeks) male BALB/c mice (N=3). As a control, PBS was also dosed similarly.

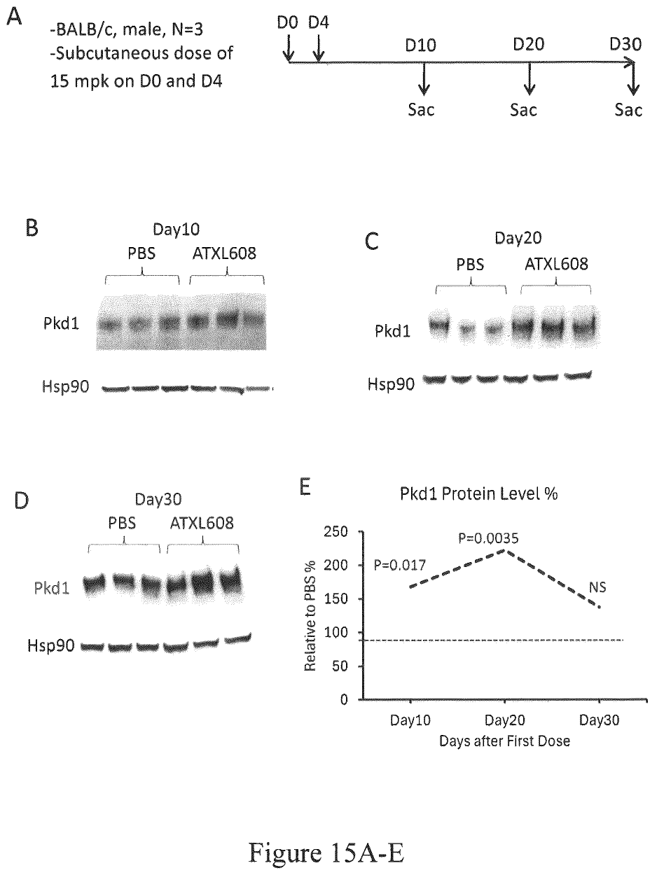

Mice were sacrificed at days 10, 20, or 30 after the first dosing. At sacrifice, kidneys were collected, and total protein from the kidney tissue samples was extracted using RIPA Lysis and Extraction Buffer (ThermoFisher Scientific, Waltham, MA, USA) and a tissue homogenizer, and separated by SDS-PAGE. The level of Pkd1 protein was determined by Western Blotting the extracted proteins and probing the Western Blot using a PKD1 protein-specific antibody (Santa Cruz Biotech, Dallas, TX, USA; Catalog #sc-130554). The Western blotting result is shown in FIG. 15. The level of Pkd1 on the Western Blot image was quantified using ImageJ, and the results are shown in Table 22 as percent protein levels relative to PBS-treated control mice group following normalization to a loading control protein, HSP90, which was detected using an HSP90-specific antibody (ProteinTech, Rosemont, IL, USA; Catalog #13171-1-AP).

TABLE 22
In Vivo Expression of Pkd1 at Different Times After Dosing

| Compound ID | Day 10 | Day 20 | Day 30 |
|---|---|---|---|
| PBS | 100.0 | 100.0 | 100.0 |
| ATXL608 (15 mg/kg) | 167.7 | 222.7 | 137.4 |

The results showed significant increases in Pkd1 protein levels in mouse kidneys, which were observed 10 days and 20 days after dosing. Even 30 days after the initial treatment, approximately a 37% increase was still observed, supporting the long-lasting effect of treatment by ATXL608.

### Example 16. Characterization of PKD1 Proteins Elicited by an ACT-UP1 Compound

In ADPKD patients with a PKD1 mutation, the Ca²⁺ signaling pathway was disrupted, leading to a reduction in cellular Ca²⁺ levels, an increase in cyclic AMP (cAMP) levels, activation of the mTOR pathway, and enhancement of cell proliferation (Niloofar Nobakht et al., 2020 Kidney Medicine, 2(2):196-208). Previous examples showed that ACT-UP1 compounds increased PKD1 protein levels. In order to assess whether the increased PKD1 protein elicited by an ACT-UP1 compound is active, the effects of ATXL608 treatment in ADPKD patient-derived WT 9-7 cells were characterized.

ATXL608 Treatment Reduced cAMP Levels in ADPKD Patient-Derived WT 9-7 Cells

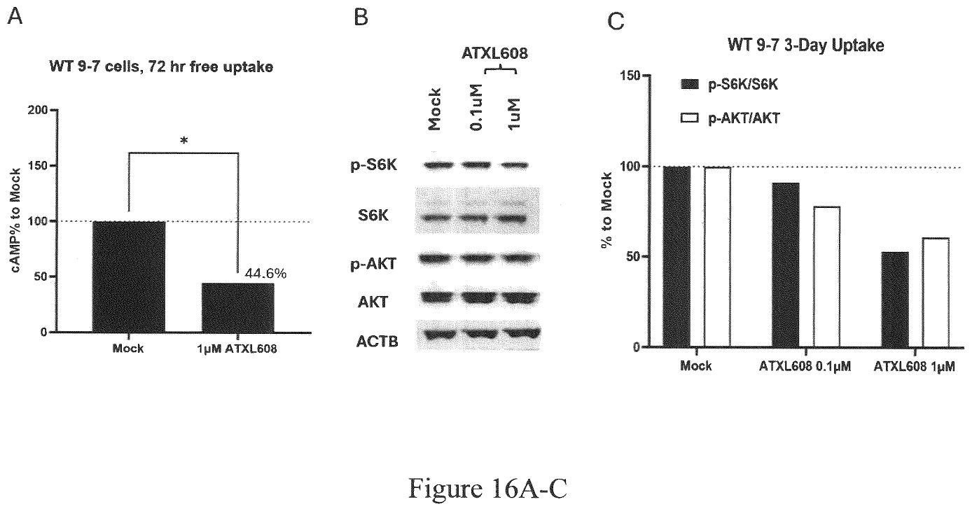

To assess whether ACT-UP1 compound treatment affected cAMP levels, WT 9-7 cells were treated with 1 μM ATXL608, by free uptake, or treated with PBS as mock control. 72 hr after treatment, cellular cAMP levels were determined using a cAMP Parameter Assay Kit (R&D Systems. Minneapolis, MN, USA, Catalog #KGE002B), based on the manufacturer's instructions. Briefly, cells were collected and washed 3 times with cold PBS, and resuspended in the cell lysis buffer provided in the kit. After two rounds of freeze-thaw, lysed cells were cleared by centrifugation at 600×g, and supernatants were analyzed. 100 pl samples were added to microplates that were preincubated with Antibody Solution, and then cAMP conjugated with standard or samples. After 2 hr incubation, the wells were washed four times using Wash Buffer. Next, 200 μl Substrate Solution was added. After 30 minutes of incubation, 100 μl Stop Solution was added. and the plates were read at 450 nM within 30 minutes. The cAMP levels were then calculated based on the standard curve. The results are shown in FIG. 16A and indicate a significant (55.4%) cAMP reduction in cells treated with ATXL608 compared with PBS mock treatment.

ATXL608 Treatment Reduced the Activity of the mTOR Pathway in WT 9-7 Cells To assess whether ACT-UP1 compound treatment affected the mTOR pathway, WT 9-7 cells were treated with 1 μM ATXL608, or treated with PBS as mock control. 72 hr after treatment, total cellular proteins were isolated as described previously and analyzed using SDS-PAGE. The levels of S6K protein, phosphorylated S6K, and AKT, phosphorylated AKT were determined by Western Blotting using an S6K protein-specific antibody (Cell Signaling Technology, Danvers, MA, USA: Catalog #9202S), a p-S6K specific antibody (Cell Signaling Technology, Danvers, MA, USA: Catalog #9206S), a p-AKT specific antibody (Cell Signaling Technology, Danvers, MA, USA: Catalog #4060L), a AKT specific antibody (Cell Signaling Technology, Danvers, MA, USA: Catalog #2920S). Beta-ACTIN (Cell Signaling Technology, Danvers, MA, USA: Catalog #3700S) was used as loading control. The Western Blotting result is shown in FIG. 16B, and the quantification data using ImageJ is shown in FIG. 16C, after normalization of the levels of phosphor-protein to total protein. The results showed that the ratios of p-S6K/total S6K and p-AKT/total AKT were reduced after ATXL608 treatment, supporting reduced activity of mTOR pathway. Together, these results indicate that PKD1 protein increased by treatment with ATXL608 is functionally active.

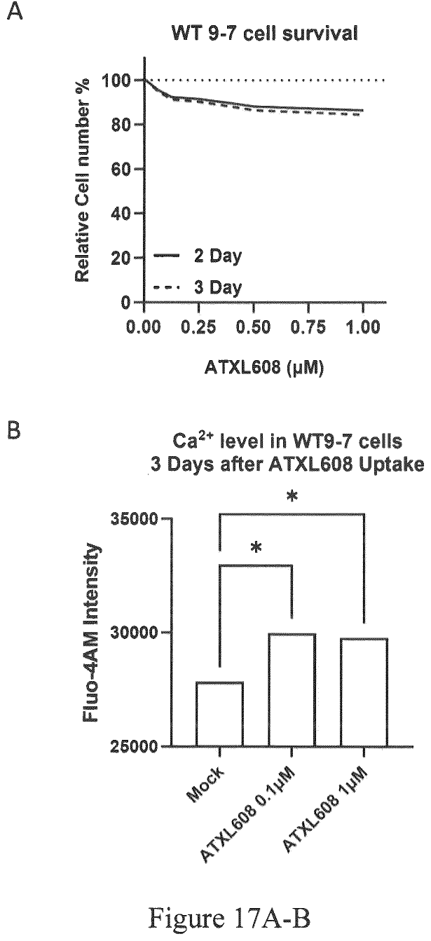

ATXL608 Treatment Reduced Cell Proliferation in WT 9-7 Cells To assess whether ATXL608 treatment influences cell proliferation, WT 9-7 cells were seeded into 96-well plates at 5,000 cells/well and cultured in 100 μl DMEM medium supplemented with 10% FBS and 1% penicillin/streptomycin for 24 hours in 5% CO₂ at 37° C. Subsequently, diluted ATXL608 (in total a volume of 10 μL) was added to cells for final concentrations at 0, 0.25, 0.5, 0.75, and 1 μM. Cells were treated in triplicate. Cells were counted 2 and 3 days after treatment using a WST-8 kit (Abeam, Cambridge, UK; Catalog #ab228554), based on the manufacturer's instructions. The results show that ATXL608 treatment reduced cell numbers compared with the mock-treated cells at both time points (FIG. 17A).

ATXL608 Treatment Increased Cellular Ca²⁺ Levels in WT 9-7 Cells To assess whether ATXL 608 treatment influences cellular Ca²⁺ levels, WT 9-7 cells were seeded in 6-well plates at 100,000 cells/well and cultured in 2 ml DMEM medium supplemented with 10% FBS and 1% penicillin/streptomycin for 24 hours in 5% CO₂ at 37° C. Subsequently, diluted ATXL608 (in a total volume of 20 μL) was added to the cells for final concentrations of 0, 0.1, or 1 μM. After 72 hrs of incubation, cells were then collected using trypsin. Cells (~1×10⁶ cells) were centrifuged (3,000×g, 5 mins, 4° C.) and washed with PBS. Cells were then spun down again and resuspended in 0.5 mL PBS with 1 μM of Fluo-4 AM stain (MedChemExpress, Monmouth Junction, NJ, USA, Catalog #HY-101896) followed by 1 hr of incubation at 37° C. Subsequently, stained cells were washed with PBS three times. Finally, cells were resuspended in 1 ml PBS, transferred to Nunc Lab Tek Chamber Slides (ThermoFisher Scientific, Waltham, MA, USA; Catalog #154526), and imaged by Echo Revolve fluorescent microscope. The fluorescence signal (integrated intensity per cell) was quantified using ImageJ. The results show that Ca²⁺ level was significantly increased after ATXL608 treatment compared to mock treated control cells (FIG. 17B).

### ATXL608 Treatment Decreases Cyst Size in WT 9-7 Cells

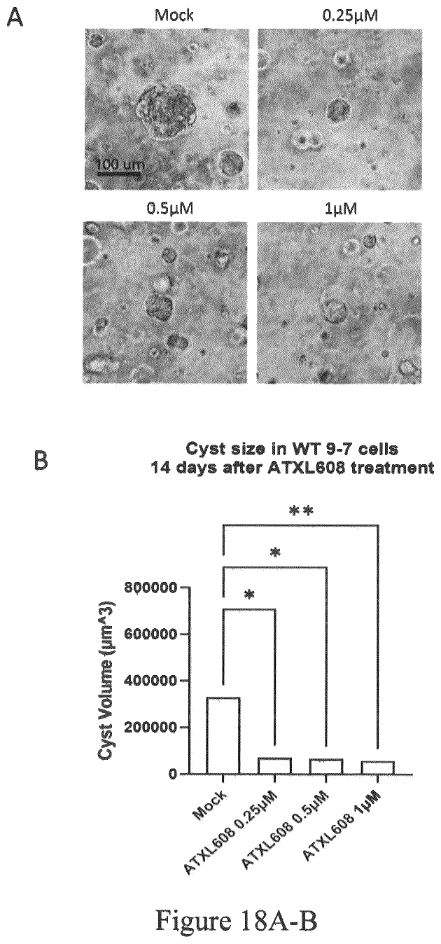

To determine the effects of ATXL608 treatment on cyst development, healthy growing WT 9-7 cells were treated with trypsin and re-suspended in DMEM media containing 10% FBS and counted. The cells were mixed with 70 μl of basement Matrigel® (Corning Life Sciences, Tewksbury, MA, USA; Catalog #354230) at a concentration of 1.5×10⁵/ml, plated into 96-well plates in triplicate, and incubated for 30 minutes at 37° C. to facilitate gel formation. Next, 100 μl of DMEM media supplemented with 10% FBS was added to each well, and cells were returned to cell incubator for 24 hours to allow, them to recover. After 24 hours, the media was aspirated and replaced with fresh media (DMEM+10% FBS) supplemented with either vehicle (mock) or ATXL608 at 0.25, 0.5, and 1 μM. Medium and ATXL608 were replaced every 3 days. The cells were photographed on day 14, and cysts were visualized under a microscope; representative images are shown in FIG. 18A. The cyst sizes (volume of ellipsoid) were measured using ImageJ, and the mean of the largest cyst size per view is shown in FIG. 18B. The results showed that ATXL608 treatment significantly reduced the cyst size compared with mock treatment.

### ATXL608 Treatment Decreases Colony Formation in WT 9-7 Cells

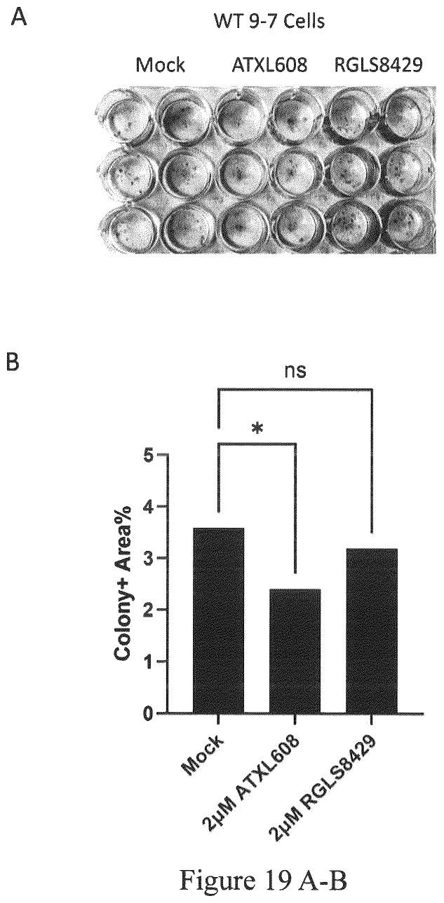

To determine the effects of increasing PKD1 protein in cell colony formation, WT 9-7 cells were treated with ATXL608. As a positive control, RGLS8429 (also known as RG-NG-1015 in WO2024215846) previously described above in Table 18 was also assessed in WT 9-7 cells. WT 9-7 cells at 80% confluence were treated with trypsin and plated in a 24-well plate at 100 cells/well density with 500 μL culture media. H₂O (Mock), ATXL608, or RGLS8429 was added to each well to reach working concentration at 2 μM. H₂O (Mock), ATXL608, or RGLS8429 was replenished at Day 5. After 10 days, cell colonies were rinsed with PBS, fixed with methanol for 15 mins, and stained with 0.5% crystal violet for 20 mins. The plate was rinsed with deionized water and let dry overnight. Finally, colony-occupied areas in each well were quantified by ImageJ and plotted (FIG. 19). One-way ANOVA analysis was used for statistical analysis, while significance was defined by p-value <0.05. The results show that ATXL608 treatment significantly decreases WT 9-7 colony formation.

Altogether, the results described herein show that ATXL608 treatment reduced cellular cAMP levels, decreased mTOR pathway activity, reduced cell proliferation, increased cellular Ca²⁺ levels, reduced cyst size, and decreased colony formation. The findings indicate that the PKD1 protein elicited by ATXL68 treatment is functional and reverts pathogenic phenotypes of ADPKD patient-derived cells.

### Example 17. Design of New Compounds to Increase PKD1 Protein Levels

To further examine the effects of sequence and chemical modification on the activity of ACT-UP1 compounds in increasing PKD1 protein levels, new compounds were designed based on ATXL608 that either change one nucleotide sequence, the position of the number of locked nucleic acid (LNA) modifications, and/or the number and position of phosphorothioate (PS) backbone modifications. The protein recruiting sequence (PRS) is bolded and underlined in the table for the ACT-UP1 compounds. The newly designed compounds are described in Table 23.

TABLE 23
ACT-UP1 Compounds Designed to Test Effects of
Sequence and Chemical Modification on Activity

| | | Sequence | Targeting | SEQ |
|---|---|---|---|---|
| Compound ID | Sequence and Chemistry (5′ to 3′) | (5′ to 3′) | Design | ID NO |
| ATXL681 | mG*mG*mAmCmUmGmGmAmC* [eG] * [eG] | GGACUGGACGG | ACT-UP1 | 52 |
| | *[eA] * [eCm] * [eCm] * [eCm] * | ACCCTGGGTCC | & 3′UTR | |
| | [eT] * [eG] * [eG] * [eG] * [eT] | TG | | |
| | [eCm] * [eCm] * [TL] [GL] | | | |
| ATXL682 | mG*mG*mAmCmUmGmGmAmC* [eG] * [eG] | GGACUGGACGG | ACT-UP1 | 53 |
| | *[eA] * [eCm] * [eCm] * [eCm] * | ACCCTGGGTCT | & 3′UTR | |
| | [eT] * [eG] * [eG] * [eG] * [TL] | TG | | |
| | * [eCm] * [TL] * [eT] * [GL] | | | |
| ATXL683 | mG*mG*mAmCmUmGmGmAmC* [eG] * [eG] | GGACUGGACGG | ACT-UP1 | 54 |
| | * [eA] * [eCm] * [eCm] * [eCm] * | ACCCTGGGTTC | & 3′UTR | |
| | [eT] * [eG] * [eG] * [eG] * [TL] | TG | | |
| | * [eT] * [mCL] * [eT] * [GL] | | | |
| ATXL685 | mG*mG*mAmCmUmGmGmAmC* [eG] * [eG] | GGACUGGACGG | ACT-UP1 | 55 |
| | * [eA] * [eCm] * [eCm] * [eCm] * | ACCCTGGGTCC | & 3′UTR | |
| | [eT] * [eG] * [eG] * [eG] * [TL] | TG | | |
| | [eCm] * [mCL] * [eT] * [GL] | | | |
| ATXL686 | mG*mG*mAmCmUmGmGmAmC* [eG] * [eG] | GGACUGGACGG | ACT-UP1 | 56 |
| | * [eA] * [eCm] * [eCm] * [eCm] * | ACCCTGGGTCC | & 3′UTR | |
| | [eT] * [eG] * [eG] * [eG] * [TL] | TG | | |
| | [eCm] * [mCL] * [eT] * [GL] | | | |

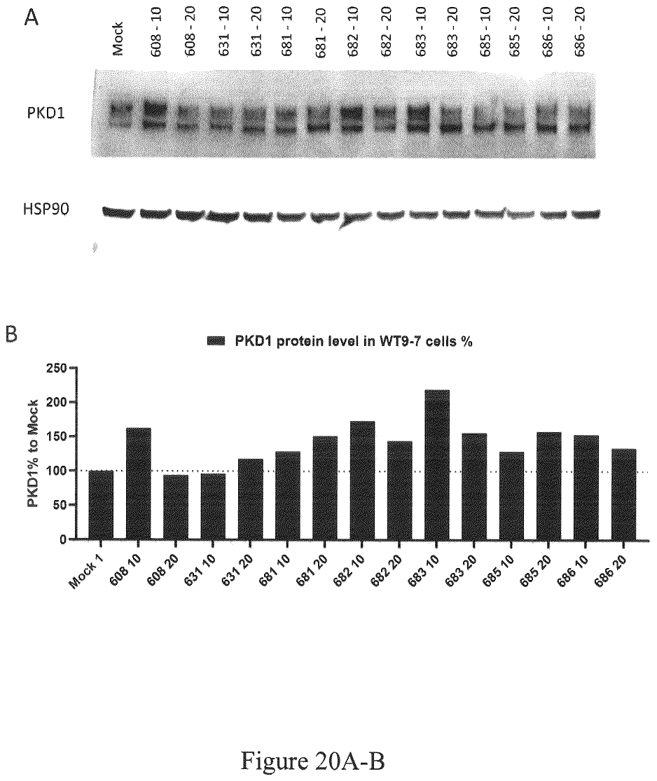

In Vitro Activity of the New Compounds in WT9-7 Cells To evaluate the efficacy of the new antisense compounds in upregulating PKD1 protein expression, WT 9-7 cells derived from an ADPKD patient were seeded and cultured to approximately 70% confluency within one day. Cells were then transfected with the compounds at final concentrations of 10 or 20 nM using Lipofectamine™ 2000 Transfection Reagent (Thermo Fisher Scientific, Waltham, MA, USA). A mock transfection served as the negative control. At 24 hours post-transfection, cells were harvested and lysed using RIPA Lysis and Extraction Buffer (Thermo Fisher Scientific, Waltham, MA, USA) to extract total protein. PKD1I protein levels were assessed via Western Blot using a PKD1-specific antibody (Santa Cruz Biotechnology, Dallas, TX, USA; Catalog #sc-130554). The resulting Western Blot image is presented in FIG. 20. Quantification was performed using ImageJ software, and the relative PKD1 protein levels, normalized to HSP90 as a loading control, are summarized in Table 24. HSP90 was detected using an HSP90-specific antibody (ProteinTech, Rosemont, IL, USA; Catalog #13171-1-AP).

TABLE 24
Activity of Compounds in WT 9-7 Cells 24 hr After Transfection

| Compound ID | PKD1 protein level % |
|---|---|
| Mock | 100.0 |
| ATXL608-10 | 162.3 |
| ATXL608-20 | 94.0 |
| ATXL631-10 | 96.4 |
| ATXL631-20 | 118.1 |
| ATXL681-10 | 128.9 |
| ATXL681-20 | 151.4 |
| ATXL682-10 | 173.3 |
| ATXL682-20 | 144.6 |
| ATXL683-10 | 219.4 |
| ATXL683-20 | 155.9 |
| ATXL685-10 | 129.1 |
| ATXL685-20 | 157.9 |
| ATXL686-10 | 153.2 |
| ATXL686-20 | 133.3 |

The results show that several newly designed compounds can also increase PKD1 protein levels like ATXL608 in WT 9-7 cells.

### Example 18. Design of Additional ACT-UP1 Compounds to Increase PKD1 Protein Levels

To further examine the effects of chemical modification on the activity of ACT-UP1 compounds in increasing PKD1 protein levels, new compound ATXL814 was designed based on the sequence of previously described ACT-UP1 compound ATXL608 with new chemistry, and new compound ATXL815 was designed based on the sequence of previously described ACT-UP1 compound ATXL682 with new chemistry. The PRS is bolded and underlined in the table for the ACT-UP1 compounds. The newly designed compounds are described in Table 25.

TABLE 25
ACT-UP1 Compounds Designed to Test Effects of
Sequence and Chemical Modification on Activity

| | | Sequence | Targeting | SEQ |
|---|---|---|---|---|
| Compound ID | Sequence and Chemistry (5′ to 3′) | (S′ to 3′) | Design | ID NO |
| ATXL814 | mG*mG*mAmCmUmGmGmAmC* [eG] * [eG] | GGACUGGACGG | ACT-UP1 | 57 |
| | * [eA] * [eCm] * [eCm] * [eCm] * | ACCCTGGGTCC | & 3′UTR | |
| | [eT] * [eG] * [eG] * [eG] * [eT] | TG | | |
| | * [eCm] * [eCm] * [eT] * [eG] | | | |
| ATXL815 | mG*mG*mAmCmUmGmGmAmC* [eG] * [eG] | GGACUGGACGG | ACT-UP1 | 58 |
| | * [eA] * [eCm] * [eCm] * [eCm] * | ACCCTGGGTCT | & 3′UTR | |
| | [eT] * [eG] * [eG] * [eG] * [eT] | TG | | |
| | * [eCm] * [eT] * [eT] * [eG] | | | |

### In Vitro Activity of the New Compounds in WT 9-7 Cells

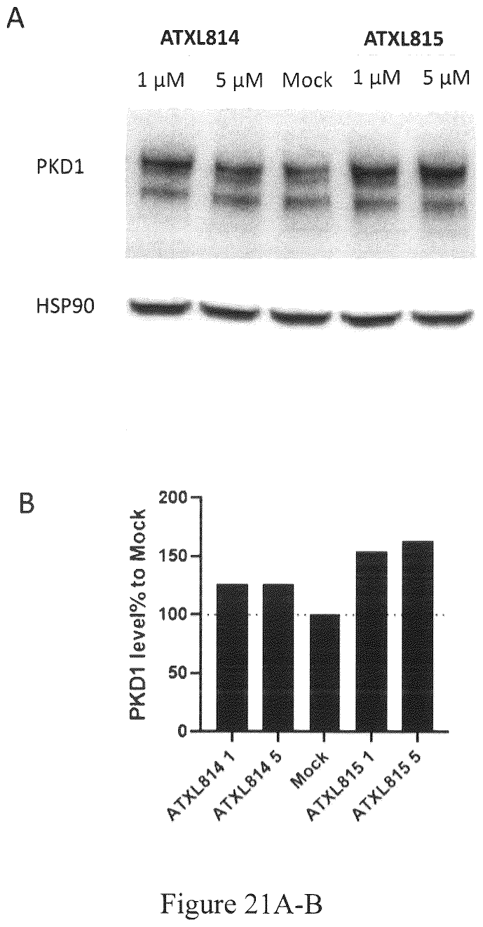

To evaluate the efficacy of the new antisense compounds in upregulating PKD1 protein expression, WT 9-7 cells derived from an ADPKD patient were seeded and cultured to approximately 70% confluency within one day. Cells were incubated with ATXL814 or ATXL815 at 1 μM or 5 μM final concentrations. Forty-eight (48) hrs after incubation, cells were harvested, and the protein was extracted using RIPA Lysis and Extraction Buffer (ThermoFisher Scientific, Waltham, MA, USA). The level of PKD1 protein was determined by Western Blotting using a PKD1 protein-specific antibody (Santa Cruz Biotech, Dallas, TX, USA; Catalog #sc-130554). The Western blotting result is shown in FIG. 21. The Western Blot image was quantified using ImageJ, and the results are shown in Table 26 as percent protein levels relative to mock incubated cells following normalization to HSP90, which was detected using an HSP90-specific antibody (ProteinTech, Rosemont, IL. USA; Catalog #13171-1-AP).

TABLE 26
Activity of Compounds in WT 9-7 Cells 48 hr After Incubation

| Compound ID | PKD1 protein level % |
|---|---|
| Mock | 100.0 |
| ATXL814 (1 μM) | 126 |
| ATXL814 (5 μM) | 126 |
| ATXL815 (1 μM) | 154 |
| ATXL815 (5 μM) | 164 |

The results show that newly designed compounds ATXL814 and ATXL815 can also increase PKD1 protein levels in WT 9-7 cells.

### Example 19. Impeding, Preventing, and/or Reversing Embryonic Cyst Formation After Treatment with PKD1 Compounds

To evaluate the effects of PKD1 upregulation in kidney cyst formation, selected PKD1 compounds that were previously found to be effective in enhancing PKD1 expression were assessed in an embryonic cyst formation assay.

Isolation, Culture. and Cyst Induction in Embryonic Kidney Explants Embryonic kidney tissues 14.5 days after fertilization (E14.5) were isolated from C57BL6 mice and kept in sterile cold PBS buffer. Millicell® inserts (0.4 pm pore)(Sigma-Aldrich, St. Louis, MO, USA) were placed in each well of 6-well plates containing 2 ml of culture media (DMEM/F12 media supplemented with 10 mM HEPES, 2 mM glutamine, 1× Insulin-Transferrin-Selenium (ITS) supplement and 1% penicillin/streptomycin). Subsequently, explanted kidneys were randomized and transferred to Millicell® inserts, followed by addition of 10 μM forskolin (MedChemExpress, Monmouth Junction, NJ, USA; Cat #HY-15371) for cyst induction. Explants were cultured at 37° C. for 5 days to allow cyst development. Explants were imaged for cysts using dissection microscope, prior to treatment to provide a baseline level of cysts.

### Free-Uptake Treatment

The explants were treated with 5 μM of compounds ATXL-Mock, ATXL608, ATXL682, ATXL814 or ATXL815 by incubating the explants in culture media containing the compounds for 3 days to allow free-uptake of the compounds into the explant. ATXL-Mock is a compound that does not target PKD1 and was included as a negative control. Additionally, the explants were treated with a mock reagent that did not contain any compound. After 3 days of treatment, cystic explants were imaged again followed by rinsing with PBS, snap-frozen and stored at −80° C.

### Western Blot Analysis of PKD1 Protein

To assess whether the compounds could enhance PKD1 expression in embryonic kidney explants, explant samples were assessed. Samples from each treatment or placebo condition were pooled and homogenized using RIPA buffer (ThermoFisher Scientific, Waltham, MA). Total proteins were prepared through centrifugation (12000×g, 4° C., 30 minutes) to separate the protein into the supernatant. The supernatant was transferred to clean tubes, and protein concentration was determined using Bicinchoninic acid (BCA) assay (Pierce™ Dilution-Free™ Rapid Gold BCA Protein Assay; Catalog #A55860; ThermoFisher Scientific, Waltham, MA, USA).

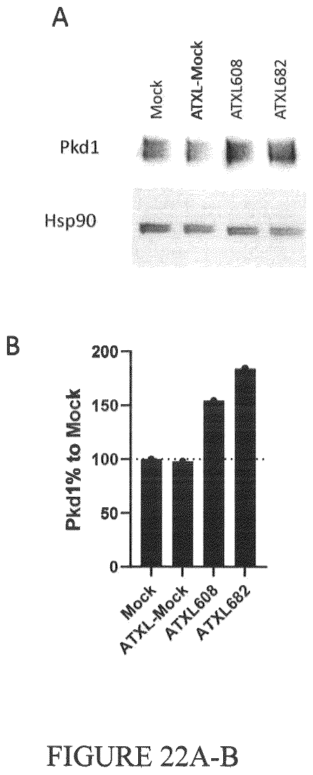

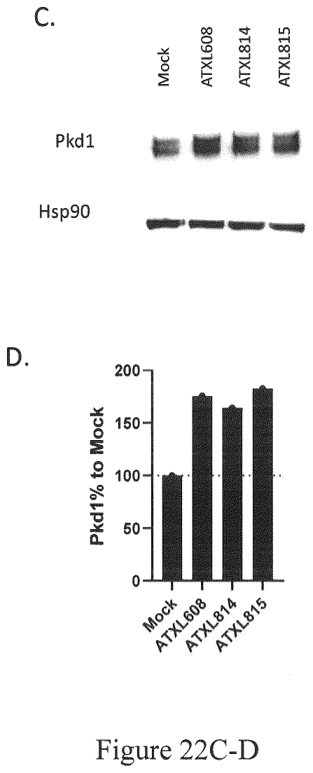

For Western Blot, 20 μg total protein was loaded onto a NuPAGE™ 4-12% Bis-Tris gel (ThermoFisher Scientific, Waltham, MA, USA), and separated by electrophoresis in NuPAGE™ MOPS SDS running buffer (ThermoFisher Scientific, Waltham, MA, USA) at room temperature for 2 hours. Proteins were transferred for 9 minutes onto the membrane using an iBlot™ 2 semi-dry system (ThermoFisher Scientific, Waltham, MA, USA). The membrane was blocked with 5% non-fat dry milk at room temperature for 30 minutes, and antibodies against Pkd1 (1:1000 dilution; Santa Cruz Biotech, Dallas, TX, USA; Catalog #sc-130554) and Hsp90 (1:5000 dilution; ProteinTech, Rosemont, IL, USA; Catalog #13171-1-AP) were incubated overnight with the membrane at 4° C. The next day, after washing the membrane 3 times with wash buffer (1× PBS +0.01% Tween-20), the membrane was incubated for 1.5 hours at room temperature with HRP conjugated anti-mouse secondary antibody (R&D Systems, Minneapolis, MN, USA; Catalog #HAF007) for Pkd1 detection, and AF647 conjugated anti-rabbit secondary antibody (Abcam, Cambridge, UK; Catalog #ab150083) for Hsp90 detection, respectively (1:2000 dilution). After washing 3 times with wash buffer, the protein was detected using HRP substrate (Millipore Sigma™ Immobilon™ Western Chemiluminescent HRP Substrate (ECL); Catalog #WBKLS0500; Sigma-Aldrich, St. Louis, MO, USA) and visualized through a ChemiDoc™ imager (Bio-Rad, Hercules, CA, USA). Protein band intensities were quantified with ImageJ. The ratio of Pkd1 to Hsp90 was calculated and subsequently plotted using GraphPad Prism. The Western Blots and associated charts showing the quantity of Pkd1 are shown in FIG. 22. The quantified values are shown in Table 27.

TABLE 27
Activity of Compounds in Embryonic Kidney
Cells 3 Days After Incubation

| | Compound ID | Pkd1 protein level % |
|---|---|---|
| FIG. 22 | Mock | 100 |
| Panel A | ATXL-M (5 μM) | 98.2 |
| | ATXL608 (5 μM) | 154.6 |
| | ATXL682 (5 μM) | 184.7 |
| FIG. 22 | Mock | 100.0 |
| Panel C | ATXL608 (5 μM) | 175.5 |
| | ATXL814 (5 μM) | 164.3 |
| | ATXL815 (5 μM) | 182.7 |

The results show that PKD1 compounds ATXL608, ATXL682, ATXL814 and ATXL815 can increase Pkd1 protein levels in explanted embryonic kidney cells.

### Kidney Cyst Analysis

Randomly selected kidney explants were selected for cyst analysis after PKD1 compound treatment to assess cyst formation in the explants. Baseline assessment of cyst formation was done at day 14.5 after fertilization and before treatment with PKD1 compounds. After 3 days incubation with each of the compounds, the explants were assessed for cyst formation.

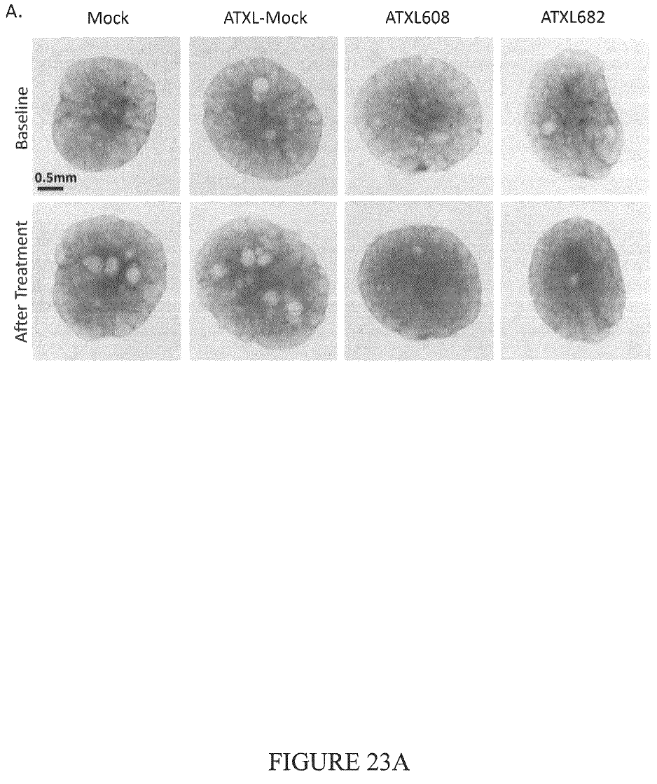

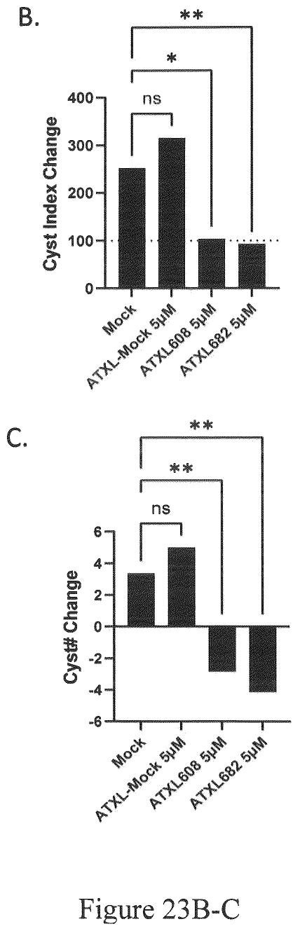

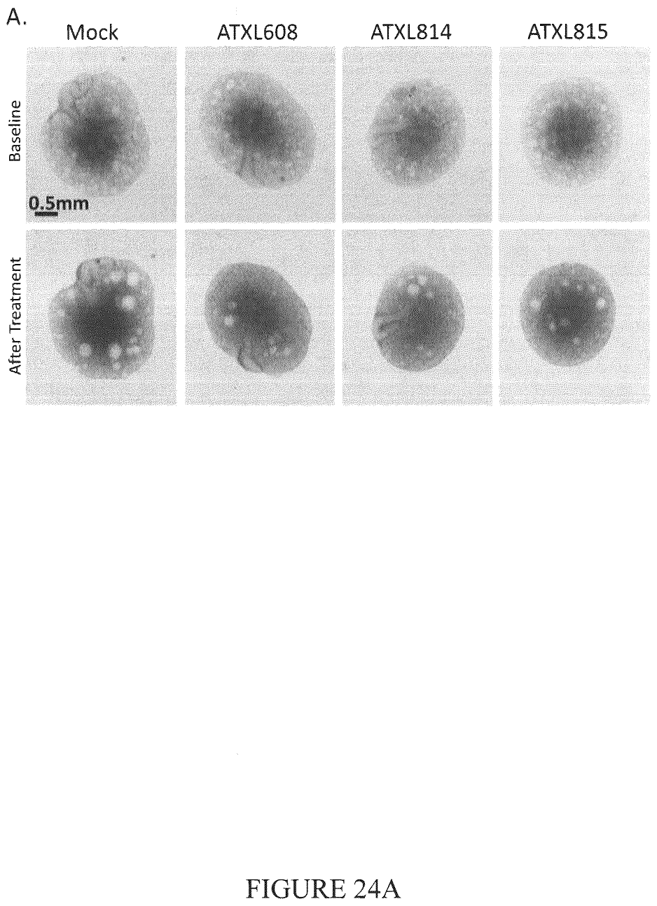

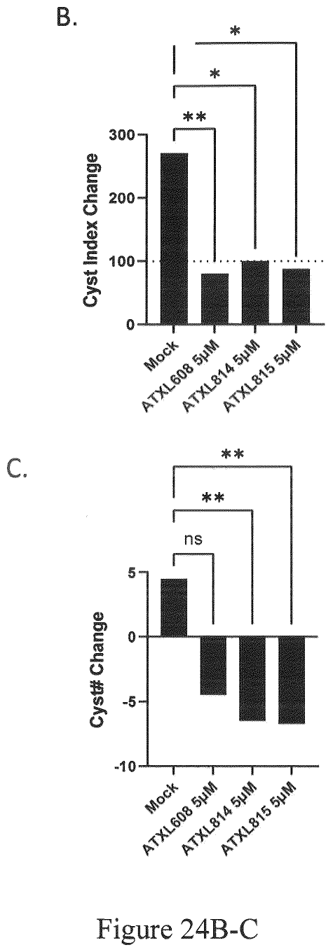

Assessment of cyst formation was performed by measuring the area occupied by the kidney explants and cysts. The measurement was performed by manually outlining the area occupied by kidney explants and cysts and measuring the outlined areas using ImageJ software (FIGS. 23A and 24A). The Cyst Index (in percentage) was defined as the ratio of cyst area to the total kidney explant area. The Cyst Index change was calculated as the Cyst Index after treatment divided by the Cyst Index at baseline, expressed as a percentage (FIGS. 23B and 24B). The Cyst Number Change was defined as cyst number post treatment subtracting cyst number before treatment in the same kidney explant and measured manually (FIGS. 23C and 24C). Statistical significance was assessed using a one-way ANOVA test.

The results show that treatment with PKD1 compounds ATXL608, ATXL682, ATXL814 and ATXL815 can impede and decrease cyst formation (FIGS. 23B-24B) as well as reverse cyst formation (FIGS. 23C-24C) in kidney explants when compared to mock treated kidney explants.

### Example 20. Additional ACT-UP1 Compounds to Increase PKD1 Protein Expression

The compounds ATXL608, ATXL681, ATXL685, ATXL686, and ATXL814 comprising the same sequence (SEQ ID NO: 77), but, having different chemical modifications were disclosed in prior examples. Compounds that differ up to 2 nucleobase positions from the sequence of SEQ ID NO: 77 were also disclosed in prior examples: ATXL501 (SEQ II) NO: 73), ATXL682 (SEQ ID NO: 85), ATXL683 (SEQ ID NO: 86). and ATXL815 (SEQ ID NO: 85).

Additional compounds, ATXL-A to ATXL-JJ (SEQ ID NOs: 89-124) that differ by up to 3 nucleobase positions from the sequence of SEQ ID NO:77 are shown in Table 28 below.

Compounds that differ from the sequence of SEQ ID NO: 77 by 1 nucleobase position include ATXL-A (SEQ ID NO: 89), ATXL-B (SEQ ID NO: 90), ATXL-C(SEQ ID NO: 91), ATXL-E (SEQ ID NO: 93), ATXL-F (SEQ ID NO: 94). ATXL-L (SEQ ID NO: 100), and ATXL-M (SEQ ID NO: 101).

Compounds that differ from the sequence of SEQ ID NO: 77 by 2 nucleobase positions include ATXL-D (SEQ ID NO: 92), ATXL-G (SEQ ID NO: 95), ATXL-H (SEQ ID NO: 96), ATXL-I (SEQ ID NO: 97), ATXL-J (SEQ ID NO: 98), ATXL-K (SEQ ID NO: 99), ATXL-N (SEQ ID NO: 102), ATXL-O (SEQ ID NO: 103), ATXL-P (SEQ ID NO: 104), ATXL-Q (SEQ ID NO: 105), ATXL-S(SEQ ID NO: 107), ATXL-T (SEQ ID NO: 108), ATXL-V (SEQ ID NO: 110), ATXL-WW (SEQ ID NO: 111), ATXL-Y (SEQ ID NO: 113), ATXL-Z (SEQ ID NO: 114), ATXL-BB (SEQ ID NO: 116), ATXL-CC (SEQ ID NO: 117), ATXL-EE (SEQ ID NO: 119), and ATXL-FF (SEQ ID NO: 120).

Compounds that differ from the sequence of SEQ ID NO: 77 by 3 nucleobase positions ATXL-R (SEQ ID NO: 106), ATXL-U (SEQ ID NO: 109), ATXL-X (SEQ ID NO: 112), ATXL-AA (SEQ ID NO: 115), ATXL-DD (SEQ ID NO: 118), ATXL-GG (SEQ ID NO: 121), ATXL-HH (SEQ ID NO: 122), ATXL-II (SEQ ID NO: 123), and ATXL-JJ (SEQ iD NO: 124).

The PRS is bolded and underlined. Any nucleobase differences to SEQ ID NO: 77 are shown in bolded italicized lowercase letters.

TABLE 28
Sequence of Antisense Compounds Targeting
PKD1 mRNA Based on SEQ ID NO: 77

| | | SEQ |
|---|---|---|
| Compound ID | Sequence (5′ to 3′) | ID NO |
| ATXL608 | GGACUGGACGGACCCTGGGTCCTG | 77 |
| ATXL681 | GGACUGGACGGACCCTGGGTCCTG | 77 |
| ATXL685 | GGACUGGACGGACCCTGGGTCCTG | 77 |
| ATXL686 | GGACUGGACGGACCCTGGGTCCTG | 77 |
| ATXL814 | GGACUGGACGGACCCTGGGTCCTG | 77 |
| ATXL501 | GGACtGGACGGACCCTGGGTCCTGg | 73 |
| ATXL682 | GGACUGGACGGACCCTGGGTCtTG | 85 |
| ATXL815 | GGACUGGACGGACCCTGGGTCtTG | 85 |
| ATXL683 | GGACUGGACGGACCCTGGGTtCTG | 86 |
| ATXL-A | GGACUGGACGGACtCTGGGTCCTG | 89 |
| ATXL-B | GGACUGGACGGAtCCTGGGTCCTG | 90 |
| ATXL-C | GGACUGGACGGACCtTGGGTCCTG | 91 |
| ATXL-D | GGACUGGACGGACtCTGGGTtCTG | 92 |
| ATXL-E | GGACtGGACGGACCCTGGGTCCTG | 93 |
| ATXL-F | GGACUGGACGGACCCTGGGTCCTGg | 94 |
| ATXL-G | GGACtGGACGGACCCTGGGTCtTG | 95 |
| ATXL-H | GGACtGGACGGACCCTGGGTtCTG | 96 |
| ATXL-I | GGACtGGACGGACtCTGGGTCCTG | 97 |
| ATXL-J | GGACtGGACGGAtCCTGGGTCCTG | 98 |
| ATXL-K | GGACtGGACGGACCtTGGGTCCTG | 99 |
| ATXL-L | GGACUGGACuGGACCCTGGGTCCTG | 100 |
| ATXL-M | GGACUGGACtGGACCCTGGGTCCTG | 101 |
| ATXL-N | GGACtGGACuGGACCCTGGGTCCTG | 102 |
| ATXL-O | GGACtGGACtGGACCCTGGGTCCTG | 103 |
| ATXL-P | GGACUGGACuGGACCCTGGGTCCTGg | 104 |
| ATXL-Q | GGACUGGACtGGACCCTGGGTCCTGg | 105 |
| ATXL-R | GGACtGGACuGGACCCTGGGTCCTGg | 106 |
| ATXL-S | GGACUGGACuGGACCCTGGGTCtTG | 107 |
| ATXL-T | GGACUGGACtGGACCCTGGGTCtTG | 108 |
| ATXL-U | GGACtGGACuGGACCCTGGGTCtTG | 109 |
| ATXL-V | GGACUGGACuGGACCCTGGGTtCTG | 110 |
| ATXL-W | GGACUGGACtGGACCCTGGGTtCTG | 111 |
| ATXL-X | GGACtGGACuGGACCCTGGGTtCTG | 112 |
| ATXL-Y | GGACUGGACuGGACtCTGGGTCCTG | 113 |
| ATXL-Z | GGACUGGACtGGACtCTGGGTCCTG | 114 |
| ATXL-AA | GGACtGGACuGGACtCTGGGTCCTG | 115 |
| ATXL-BB | GGACUGGACuGGAtCCTGGGTCCTG | 116 |
| ATXL-CC | GGACUGGACtGGAtCCTGGGTCCTG | 117 |
| ATXL-DD | GGACtGGACuGGAtCCTGGGTCCTG | 118 |
| ATXL-BE | GGACUGGACuGGACCtTGGGTCCTG | 119 |
| ATXL-FF | GGACUGGACtGGACCtTGGGTCCTG | 120 |
| ATXL-GG | GGACtGGACuGGACCtTGGGTCCTG | 121 |
| ATXL-HH | GGACUGGACuGGACtCTGGGTtCTG | 122 |
| ATXL-II | GGACUGGACtGGACtCTGGGTtCTG | 123 |
| ATXL-JJ | GGACtGGACGGACtCTGGGTtCTG | 124 |

TABLE 29A
Sequence Listing of GenBank Sequences

| SEQ ID NO | Sequence | Name/Description |
|---|---|---|
| 1 | gcactgcagcgccagcgtccgagcgggcggccgagctcccggag | Human PKD1 mRNA |
| | cggcctggccccgagccccgagcgggcgtcgctcagcagcaggt | GenBank Accession |
| | cgcggccgcagccccatccagccccgcgcccgccatgccgtccg | No. NM_001009944.3 |
| | cgggccccgcctgagctgcggcctccgcgcgcgggcgggcctgg | |
| | ggacggcggggccatgcgcgcgctgccctaacgatgccgcccgc | |
| | cgcgcccgcccgcctggcgctggccctgggcctgggcctgtggc | |
| | tcggggcgctggcggggggccccgggcgcggctgcgggccctgc | |
| | gagcccccctgcctctgcggcccagcgcccggcgccgcctgccg | |
| | cgtcaactgctcgggccgcgggctgcggacgctcggtcccgcgc | |
| | tgcgcatccccgcggacgccacagcgctagacgtctcccacaac | |
| | ctgctccgggcgctggacgttgggctcctggcgaacctctcggc | |
| | gctggcagagctggatataagcaacaacaagatttctacgttag | |
| | aagaaggaatatttgctaatttatttaatttaagtgaaataaac | |
| | ctgagtgggaacccgtttgagtgtgactgtggcctggcgtggct | |
| | gccgcgatgggcggaggagcagcaggtgcgggtggtgcagcccg | |
| | aggcagccacgtgtgctgggcctggctccctggctggccagcct | |
| | ctgcttggcatccccttgctggacagtggctgtggtgaggagta | |
| | tgtcgcctgcctccctgacaacagctcaggcaccgtggcagcag | |
| | tgtccttttcagctgcccacgaaggcctgcttcagccagaggcc | |
| | tgcagcgccttctgcttctccaccggccagggcctcgcagccct | |
| | ctcggagcagggctggtgcctgtgtggggcggcccagccctcca | |
| | gtgcctcctttgcctgcctgtccctctgctccggccccccgcca | |
| | cctcctgcccccacctgtaggggccccaccctcctccagcacgt | |
| | cttccctgcctccccaggggccaccctggtggggccccacggac | |
| | ctctggcctctggccagctagcagccttccacatcgctgccccg | |
| | ctccctgtcactgccacacgctgggacttcggagacggctccgc | |
| | cgaggtggatgccgctgggccggctgcctcgcatcgctatgtgc | |
| | tgcctgggcgctatcacgtgacggccgtgctggccctgggggcc | |
| | ggctcagccctgctggggacagacgtgcaggtggaagcggcacc | |
| | tgccgccctggagctcgtgtgcccgtcctcggtgcagagtgacg | |
| | agagcctcgacctcagcatccagaaccgcggtggttcaggcctg | |
| | gaggccgcctacagcatcgtggccctgggcgaggagccggcccg | |
| | agcggtgcacccgctctgcccctcggacacggagatcttccctg | |
| | gcaacgggcactgctaccgcctggtggtggagaaggcggcctgg | |
| | ctgcaggcgcaggagcagtgtcaggcctgggccggggccgccct | |
| | ggcaatggtggacagtcccgccgtgcagcgcttcctggtctccc | |
| | gggtcaccaggagcctagacgtgtggatcggcttctcgactgtg | |
| | cagggggtggaggtgggcccagcgccgcagggcgaggccttcag | |
| | cctggagagctgccagaactggctgcccggggagccacacccag | |
| | ccacagccgagcactgcgtccggctcgggcccaccgggtggtgt | |
| | aacaccgacctgtgctcagcgccgcacagctacgtctgcgagct | |
| | gcagcccggaggcccagtgcaggatgccgagaacctcctcgtgg | |
| | gagcgcccagtggggacctgcagggacccctgacgcctctggca | |
| | cagcaggacggcctctcagccccgcacgagcccgtggaggtcat | |
| | ggtattcccgggcctgcgtctgagccgtgaagccttcctcacca | |
| | cggccgaatttgggacccaggagctccggcggcccgcccagctg | |
| | cggctgcaggtgtaccggctcctcagcacagcagggaccccgga | |
| | gaacggcagcgagcctgagagcaggtccccggacaacaggaccc | |
| | agctggcccccgcgtgcatgccagggggacgctggtgccctgga | |
| | gccaacatctgcttgccgctggacgcctcctgccacccccaggc | |
| | ctgcgccaatggctgcacgtcagggccagggctacccggggccc | |
| | cctatgcgctatggagagagttcctcttctccgttcccgcgggg | |
| | ccccccgcgcagtactcggtcaccctccacggccaggatgtcct | |
| | catgctccctggtgacctcgttggcttgcagcacgacgctggcc | |
| | ctggcgccctcctgcactgctcgccggctcccggccaccctggt | |
| | ccccgggccccgtacctctccgccaacgcctcgtcatggctgcc | |
| | ccacttgccagcccagctggagggcacttgggcctgccctgcct | |
| | gtgccctgcggctgcttgcagccacggaacagctcaccgtgctg | |
| | ctgggcttgaggcccaaccctggactgcggctgcctgggcgcta | |
| | tgaggtccgggcagaggtgggcaatggcgtgtccaggcacaacc | |
| | tctcctgcagctttgacgtggtctccccagtggctgggctgcgg | |
| | gtcatctaccctgccccccgcgacggccgcctctacgtgcccac | |
| | caacggctcagccttggtgctccaggtggactctggtgccaacg | |
| | ccacggccacggctcgctggcctgggggcagtgtcagcgcccgc | |
| | tttgagaatgtctgccctgccctggtggccaccttcgtgcccgg | |
| | ctgcccctgggagaccaacgataccctgttctcagtggtagcac | |
| | tgccgtggctcagtgagggggagcacgtggtggacgtggtggtg | |
| | gaaaacagcgccagccgggccaacctcagcctgcgggtgacggc | |
| | ggaggagcccatctgtggcctccgcgccacgcccagccccgagg | |
| | cccgtgtactgcagggagtcctagtgaggtacagccccgtggtg | |
| | gaggccggctcggacatggtcttccggtggaccatcaacgacaa | |
| | gcagtccctgaccttccagaacgtggtcttcaatgtcatttatc | |
| | agagcgcggcggtcttcaagctctcactgacggcctccaaccac | |
| | gtgagcaacgtcaccgtgaactacaacgtaaccgtggagcggat | |
| | gaacaggatgcagggtctgcaggtctccacagtgccggccgtgc | |
| | tgtcccccaatgccacgctagcactgacggcgggcgtgctggtg | |
| | gactcggccgtggaggtggccttcctgtggacctttggggatgg | |
| | ggagcaggccctccaccagttccagcctccgtacaacgagtcct | |
| | tcccggttccagacccctcggtggcccaggtgctggtggagcac | |
| | aatgtcatgcacacctacgctgccccaggtgagtacctcctgac | |
| | cgtgctggcatctaatgccttcgagaacctgacgcagcaggtgc | |
| | ctgtgagcgtgcgcgcctccctgccctccgtggctgtgggtgtg | |
| | agtgacggcgtcctggtggccggccggcccgtcaccttctaccc | |
| | gcacccgctgccctcgcctgggggtgttctttacacgtgggact | |
| | tcggggacggctcccctgtcctgacccagagccagccggctgcc | |
| | aaccacacctatgcctcgaggggcacctaccacgtgcgcctgga | |
| | ggtcaacaacacggtgagcggtgcggcggcccaggcggatgtgc | |
| | gcgtctttgaggagctccgcggactcagcgtggacatgagcctg | |
| | gccgtggagcagggcgcccccgtggtggtcagcgccgcggtgca | |
| | gacgggcgacaacatcacgtggaccttcgacatgggggacggca | |
| | ccgtgctgtcgggcccggaggcaacagtggagcatgtgtacctg | |
| | cgggcacagaactgcacagtgaccgtgggtgcggccagccccgc | |
| | cggccacctggcccggagcctgcacgtgctggtcttcgtcctgg | |
| | aggtgctgcgcgttgaacccgccgcctgcatccccacgcagcct | |
| | gacgcgcggctcacggcctacgtcaccgggaacccggcccacta | |
| | cctcttcgactggaccttcggggatggctcctccaacacgaccg | |
| | tgcgggggtgcccgacggtgacacacaacttcacgcggagcggc | |
| | acgttccccctggcgctggtgctgtccagccgcgtgaacagggc | |
| | gcattacttcaccagcatctgcgtggagccagaggtgggcaacg | |
| | tcaccctgcagccagagaggcagtttgtgcagctcggggacgag | |
| | gcctggctggtggcatgtgcctggcccccgttcccctaccgcta | |
| | cacctgggactttggcaccgaggaagccgcccccacccgtgcca | |
| | ggggccctgaggtgacgttcatctaccgagacccaggctcctat | |
| | cttgtgacagtcaccgcgtccaacaacatctctgctgccaatga | |
| | ctcagccctggtggaggtgcaggagcccgtgctggtcaccagca | |
| | tcaaggtcaatggctcccttgggctggagctgcagcagccgtac | |
| | ctgttctctgctgtgggccgtgggcgccccgccagctacctgtg | |
| | ggatctgggggacggtgggtggctcgagggtccggaggtcaccc | |
| | acgcttacaacagcacaggtgacttcacccttagggtggccggc | |
| | tggaatgaggtgagccgcagcgaggcctggctcaatgtgacggt | |
| | gaagcggcgcgtgcgggggctcgtcgtcaatgcaagccgcacgg | |
| | tggtgcccctgaatgggagcgtgagcttcagcacgtcgctggag | |
| | gccggcagtgatgtgcgctattcctgggtgctctgtgaccgctg | |
| | cacgcccatccctgggggtcctaccatctcttacaccttccgct | |
| | ccgtgggcaccttcaatatcatcgtcacggctgagaacgaggtg | |
| | ggctccgcccaggacagcatcttcgtctatgtcctgcagctcat | |
| | agaggggctgcaggtggtgggcggtggccgctacttccccacca | |
| | accacacggtacagctgcaggccgtggttagggatggcaccaac | |
| | gtctcctacagctggactgcctggagggacaggggcccggccct | |
| | ggccggcagcggcaaaggcttctcgctcaccgtgctcgaggccg | |
| | gcacctaccatgtgcagctgcgggccaccaacatgctgggcagc | |
| | gcctgggccgactgcaccatggacttcgtggagcctgtggggtg | |
| | gctgatggtggccgcctccccgaacccagctgccgtcaacacaa | |
| | gcgtcaccctcagtgccgagctggctggtggcagtggtgtcgta | |
| | tacacttggtccttggaggaggggctgagctgggagacctccga | |
| | gccatttaccacccatagcttccccacacccggcctgcacttgg | |
| | tcaccatgacggcagggaacccgctgggctcagccaacgccacc | |
| | gtggaagtggatgtgcaggtgcctgtgagtggcctcagcatcag | |
| | ggccagcgagcccggaggcagcttcgtggcggccgggtcctctg | |
| | tgcccttttgggggcagctggccacgggcaccaatgtgagctgg | |
| | tgctgggctgtgcccggcggcagcagcaagcgtggccctcatgt | |
| | caccatggtcttcccggatgctggcaccttctccatccggctca | |
| | atgcctccaacgcagtcagctgggtctcagccacgtacaacctc | |
| | acggcggaggagcccatcgtgggcctggtgctgtgggccagcag | |
| | caaggtggtggcgcccgggcagctggtccattttcagatcctgc | |
| | tggctgccggctcagctgtcaccttccgcctgcaggtcggcggg | |
| | gccaaccccgaggtgctccccgggccccgtttctcccacagctt | |
| | cccccgcgtcggagaccacgtggtgagcgtgcggggcaaaaacc | |
| | acgtgagctgggcccaggcgcaggtgcgcatcgtggtgctggag | |
| | gccgtgagtgggctgcaggtgcccaactgctgcgagcctggcat | |
| | cgccacgggcactgagaggaacttcacagcccgcgtgcagcgcg | |
| | gctctcgggtcgcctacgcctggtacttctcgctgcagaaggtc | |
| | cagggcgactcgctggtcatcctgtcgggccgcgacgtcaccta | |
| | cacgcccgtggccgcggggctgttggagatccaggtgcgcgcct | |
| | tcaacgccctgggcagtgagaaccgcacgctggtgctggaggtt | |
| | caggacgccgtccagtatgtggccctgcagagcggcccctgctt | |
| | caccaaccgctcggcgcagtttgaggccgccaccagccccagcc | |
| | cccggcgtgtggcctaccactgggactttggggatgggtcgcca | |
| | gggcaggacacagatgagcccagggccgagcactcctacctgag | |
| | gcctggggactaccgcgtgcaggtgaacgcctccaacctggtga | |
| | gcttcttcgtggcgcaggccacggtgaccgtccaggtgctggcc | |
| | tgccgggagccggaggtggacgtggtcctgcccctgcaggtgct | |
| | gatgcggcgatcacagcgcaactacttggaggcccacgttgacc | |
| | tgcgcgactgcgtcacctaccagactgagtaccgctgggaggtg | |
| | tatcgcaccgccagctgccagcggccggggcgcccagcgcgtgt | |
| | ggccctgcccggcgtggacgtgagccggcctcggctggtgctgc | |
| | cgcggctggcgctgcctgtggggcactactgctttgtgtttgtc | |
| | gtgtcatttggggacacgccactgacacagagcatccaggccaa | |
| | tgtgacggtggcccccgagcgcctggtgcccatcattgagggtg | |
| | gctcataccgcgtgtggtcagacacacgggacctggtgctggat | |
| | gggagcgagtcctacgaccccaacctggaggacggcgaccagac | |
| | gccgctcagtttccactgggcctgtgtggcttcgacacagaggg | |
| | aggctggcgggtgtgcgctgaactttgggccccgcgggagcagc | |
| | acggtcaccattccacgggagcggctggcggctggcgtggagta | |
| | caccttcagcctgaccgtgtggaaggccggccgcaaggaggagg | |
| | ccaccaaccagacggtgctgatccggagtggccgggtgcccatt | |
| | gtgtccttggagtgtgtgtcctgcaaggcacaggccgtgtacga | |
| | agtgagccgcagctcctacgtgtacttggagggccgctgcctca | |
| | attgcagcagcggctccaagcgagggcggtgggctgcacgtacg | |
| | ttcagcaacaagacgctggtgctggatgagaccaccacatccac | |
| | gggcagtgcaggcatgcgactggtgctgcggcggggcgtgctgc | |
| | gggacggcgagggatacaccttcacgctcacggtgctgggccgc | |
| | tctggcgaggaggagggctgcgcctccatccgcctgtcccccaa | |
| | ccgcccgccgctggggggctcttgccgcctcttcccactgggcg | |
| | ctgtgcacgccctcaccaccaaggtgcacttcgaatgcacgggc | |
| | tggcatgacgcggaggatgctggcgccccgctggtgtacgccct | |
| | gctgctgcggcgctgtcgccagggccactgcgaggagttctgtg | |
| | tctacaagggcagcctctccagctacggagccgtgctgcccccg | |
| | ggtttcaggccacacttcgaggtgggcctggccgtggtggtgca | |
| | ggaccagctgggagccgctgtggtcgccctcaacaggtctttgg | |
| | ccatcaccctcccagagcccaacggcagcgcaacggggctcaca | |
| | gtctggctgcacgggctcaccgctagtgtgctcccagggctgct | |
| | gcggcaggccgatccccagcacgtcatcgagtactcgttggccc | |
| | tggtcaccgtgctgaacgagtacgagcgggccctggacgtggcg | |
| | gcagagcccaagcacgagcggcagcaccgagcccagatacgcaa | |
| | gaacatcacggagactctggtgtccctgagggtccacactgtgg | |
| | atgacatccagcagatcgctgctgcgctggcccagtgcatgggg | |
| | cccagcagggagctcgtatgccgctcgtgcctgaagcagacgct | |
| | gcacaagctggaggccatgatgctcatcctgcaggcagagacca | |
| | ccgcgggcaccgtgacgcccaccgccatcggagacagcatcctc | |
| | aacatcacaggagacctcatccacctggccagctcggacgtgcg | |
| | ggcaccacagccctcagagctgggagccgagtcaccatctcgga | |
| | tggtggcgtcccaggcctacaacctgacctctgccctcatgcgc | |
| | atcctcatgcgctcccgcgtgctcaacgaggagcccctgacgct | |
| | ggcgggcgaggagatcgtggcccagggcaagcgctcggacccgc | |
| | ggagcctgctgtgctatggcggcgccccagggcctggctgccac | |
| | ttctccatccccgaggctttcagcggggccctggccaacctcag | |
| | tgacgtggtgcagctcatctttctggtggactccaatccctttc | |
| | cctttggctatatcagcaactacaccgtctccaccaaggtggcc | |
| | tcgatggcattccagacacaggccggcgcccagatccccatcga | |
| | gcggctggcctcagagcgcgccatcaccgtgaaggtgcccaaca | |
| | actcggactgggctgcccggggccaccgcagctccgccaactcc | |
| | gccaactccgttgtggtccagccccaggcctccgtcggtgctgt | |
| | ggtcaccctggacagcagcaaccctgcggccgggctgcatctgc | |
| | agctcaactatacgctgctggacggccactacctgtctgaggaa | |
| | cctgagccctacctggcagtctacctacactcggagccccggcc | |
| | caatgagcacaactgctcggctagcaggaggatccgcccagagt | |
| | cactccagggtgctgaccaccggccctacaccttcttcatttcc | |
| | ccggggagcagagacccagcggggagttaccatctgaacctctc | |
| | cagccacttccgctggtcggcgctgcaggtgtccgtgggcctgt | |
| | acacgtccctgtgccagtacttcagcgaggaggacatggtgtgg | |
| | cggacagaggggctgctgcccctggaggagacctcgccccgcca | |
| | ggccgtctgcctcacccgccacctcaccgccttcggcgccagcc | |
| | tcttcgtgcccccaagccatgtccgctttgtgtttcctgagccg | |
| | acagcggatgtaaactacatcgtcatgctgacatgtgctgtgtg | |
| | cctggtgacctacatggtcatggccgccatcctgcacaagctgg | |
| | accagttggatgccagccggggccgcgccatccctttctgtggg | |
| | cagcggggccgcttcaagtacgagatcctcgtcaagacaggctg | |
| | gggccggggctcaggtaccacggcccacgtgggcatcatgctgt | |
| | atggggtggacagccggagcggccaccggcacctggacggcgac | |
| | agagccttccaccgcaacagcctggacatcttccggatcgccac | |
| | cccgcacagcctgggtagcgtgtggaagatccgagtgtggcacg | |
| | acaacaaagggctcagccctgcctggttcctgcagcacgtcatc | |
| | gtcagggacctgcagacggcacgcagcgccttcttcctggtcaa | |
| | tgactggctttcggtggagacggaggccaacgggggcctggtgg | |
| | agaaggaggtgctggccgcgagcgacgcagcccttttgcgcttc | |
| | cggcgcctgctggtggctgagctgcagcgtggcttctttgacaa | |
| | gcacatctggctctccatatgggaccggccgcctcgtagccgtt | |
| | tcactcgcatccagagggccacctgctgcgttctcctcatctgc | |
| | ctcttcctgggcgccaacgccgtgtggtacggggctgttggcga | |
| | ctctgcctacagcacggggcatgtgtccaggctgagcccgctga | |
| | gcgtcgacacagtcgctgttggcctggtgtccagcgtggttgtc | |
| | tatcccgtctacctggccatcctttttctcttccggatgtcccg | |
| | gagcaaggtggctgggagcccgagccccacacctgccgggcagc | |
| | aggtgctggacatcgacagctgcctggactcgtccgtgctggac | |
| | agctccttcctcacgttctcaggcctccacgctgagcaggcctt | |
| | tgttggacagatgaagagtgacttgtttctggatgattctaaga | |
| | gtctggtgtgctggccctccggcgagggaacgctcagttggccg | |
| | gacctgctcagtgacccgtccattgtgggtagcaatctgcggca | |
| | gctggcacggggccaggcgggccatgggctgggcccagaggagg | |
| | acggcttctccctggccagcccctactcgcctgccaaatccttc | |
| | tcagcatcagatgaagacctgatccagcaggtccttgccgaggg | |
| | ggtcagcagcccagcccctacccaagacacccacatggaaacgg | |
| | acctgctcagcagcctgtccagcactcctggggagaagacagag | |
| | acgctggcgctgcagaggctgggggagctggggccacccagccc | |
| | aggcctgaactgggaacagccccaggcagcgaggctgtccagga | |
| | caggactggtggagggtctgcggaagcgcctgctgccggcctgg | |
| | tgtgcctccctggcccacgggctcagcctgctcctggtggctgt | |
| | ggctgtggctgtctcagggtgggtgggtgcgagcttccccccgg | |
| | gcgtgagtgttgcgtggctcctgtccagcagcgccagcttcctg | |
| | gcctcattcctcggctgggagccactgaaggtcttgctggaagc | |
| | cctgtacttctcactggtggccaagcggctgcacccggatgaag | |
| | atgacaccctggtagagagcccggctgtgacgcctgtgagcgca | |
| | cgtgtgccccgcgtacggccaccccacggctttgcactcttcct | |
| | ggccaaggaagaagcccgcaaggtcaagaggctacatggcatgc | |
| | tgcggagcctcctggtgtacatgctttttctgctggtgaccctg | |
| | ctggccagctatggggatgcctcatgccatgggcacgcctaccg | |
| | tctgcaaagcgccatcaagcaggagctgcacagccgggccttcc | |
| | tggccatcacgcggtctgaggagctctggccatggatggcccac | |
| | gtgctgctgccctacgtccacgggaaccagtccagcccagagct | |
| | ggggcccccacggctgcggcaggtgcggctgcaggaagcactct | |
| | acccagaccctcccggccccagggtccacacgtgctcggccgca | |
| | ggaggcttcagcaccagcgattacgacgttggctgggagagtcc | |
| | tcacaatggctcggggacgtgggcctattcagcgccggatctgc | |
| | tgggggcatggtcctggggctcctgtgccgtgtatgacagcggg | |
| | ggctacgtgcaggagctgggcctgagcctggaggagagccgcga | |
| | ccggctgcgcttcctgcagctgcacaactggctggacaacagga | |
| | gccgcgctgtgttcctggagctcacgcgctacagcccggccgtg | |
| | gggctgcacgccgccgtcacgctgcgcctcgagttcccggcggc | |
| | cggccgcgccctggccgccctcagcgtccgcccctttgcgctgc | |
| | gccgcctcagcgcgggcctctcgctgcctctgctcacctcggtg | |
| | tgcctgctgctgttcgccgtgcacttcgccgtggccgaggcccg | |
| | tacttggcacagggaagggcgctggcgcgtgctgcggctcggag | |
| | cctgggcgcggtggctgctggtggcgctgacggcggccacggca | |
| | ctggtacgcctcgcccagctgggtgccgctgaccgccagtggac | |
| | ccgtttcgtgcgcggccgcccgcgccgcttcactagcttcgacc | |
| | aggtggcgcagctgagctccgcagcccgtggcctggcggcctcg | |
| | ctgctcttcctgcttttggtcaaggctgcccagcagctacgctt | |
| | cgtgcgccagtggtccgtctttggcaagacattatgccgagctc | |
| | tgccagagctcctgggggtcaccttgggcctggtggtgctcggg | |
| | gtagcctacgcccagctggccatcctgctcgtgtcttcctgtgt | |
| | ggactccctctggagcgtggcccaggccctgttggtgctgtgcc | |
| | ctgggactgggctctctaccctgtgtcctgccgagtcctggcac | |
| | ctgtcacccctgctgtgtgtggggctctgggcactgcggctgtg | |
| | gggcgccctacggctgggggctgttattctccgctggcgctacc | |
| | acgccttgcgtggagagctgtaccggccggcctgggagccccag | |
| | gactacgagatggtggagttgttcctgcgcaggctgcgcctctg | |
| | gatgggcctcagcaaggtcaaggagttccgccacaaagtccgct | |
| | ttgaagggatggagccgctgccctctcgctcctccaggggctcc | |
| | aaggtatccccggatgtgcccccacccagcgctggctccgatgc | |
| | ctcgcacccctccacctcctccagccagctggatgggctgagcg | |
| | tgagcctgggccggctggggacaaggtgtgagcctgagccctcc | |
| | cgcctccaagccgtgttcgaggccctgctcacccagtttgaccg | |
| | actcaaccaggccacagaggacgtctaccagctggagcagcagc | |
| | tgcacagcctgcaaggccgcaggagcagccgggcgcccgccgga | |
| | tcttcccgtggcccatccccgggcctgcggccagcactgcccag | |
| | ccgccttgcccgggccagtcggggtgtggacctggccactggcc | |
| | ccagcaggacaccccttcgggccaagaacaaggtccaccccagc | |
| | agcacttagtcctccttcctggcgggggtgggccgtggagtcgg | |
| | agtggacaccgctcagtattactttctgccgctgtcaaggccga | |
| | gggccaggcagaatggctgcacgtaggttccccagagagcaggc | |
| | aggggcatctgtctgtctgtgggcttcagcactttaaagaggct | |
| | gtgtggccaaccaggacccagggtcccctccccagctcccttgg | |
| | gaaggacacagcagtattggacggtttctagcctctgagatgct | |
| | aatttatttccccgagtcctcaggtacagcgggctgtgcccggc | |
| | cccaccccctgggcagatgtcccccactgctaaggctgctggct | |
| | tcagggagggttagcctgcaccgccgccaccctgcccctaagtt | |
| | attacctctccagttcctaccgtactccctgcaccgtctcactg | |
| | tgtgtctcgtgtcagtaatttatatggtgttaaaatgtgtatat | |
| | ttttgtatgtcactattttcactagggctgaggggcctgcgccc | |
| | agagctggcctcccccaacacctgctgcgcttggtaggtgtggt | |
| | ggcgttatggcagcccggctgctgcttggatgcgagcttggcct | |
| | tgggccggtgctgggggcacagctgtctgccaggcactctcatc | |
| | accccagaggccttgtcatcctcccttgccccaggccaggtagc | |
| | aagagagcagcgcccaggcctgctggcatcaggtctgggcaagt | |
| | agcaggactaggcatgtcagaggaccccagggtggttagaggaa | |
| | aagactcctcctgggggctggctcccagggtggaggaaggtgac | |
| | tgtgtgtgtgtgtgtgtgcgcgcgcgcacgcgcgagtgtgctgt | |
| | atggcccaggcagcctcaaggccctcggagctggctgtgcctgc | |
| | ttctgtgtaccacttctgtgggcatggccgcttctagagcctcg | |
| | acacccccccaacccccgcaccaagcagacaaagtcaataaaag | |
| | agctgtctgactgcaa | |
| 2 | aggggcggcgggcgccgggaagaaaggaacatggctcctgagg | Human PKD2 mRNA |
| | cgcacagcgccgagcgcggcgccgcgcacccgcgcgccggacgc | GenBank Accession |
| | cagtgaccgcgatggtgaactccagtcgcgtgcagcctcagcag | No. NM_000297.4 |
| | cccggggacgccaagcggccgcccgcgccccgcgcgccggaccc | |
| | gggccggctgatggctggctgcgcggccgtgggcgccagcctcg | |
| | ccgccccgggcggcctctgcgagcagcggggcctggagatcgag | |
| | atgcagcgcatccggcaggcggccgcgcgggaccccccggccgg | |
| | agccgcggcctccccttctcctccgctctcgtcgtgctcccggc | |
| | aggcgtggagccgcgataaccccggcttcgaggccgaggaggag | |
| | gaggaggaggtggaaggggaagaaggcggaatggtggtggagat | |
| | ggacgtagagtggcgcccgggcagccggaggtcggccgcctcct | |
| | cggccgtgagctccgtgggcgcgcggagccgggggcttgggggc | |
| | taccacggcgcgggccacccgagcgggaggcggcgccggcgaga | |
| | ggaccagggcccgccgtgccccagcccagtcggcggcggggacc | |
| | cgctgcatcgccacctccccctggaagggcagccgccccgagtg | |
| | gcctgggcggagaggctggttcgcgggctgcgaggtctctgggg | |
| | aacaagactcatggaggaaagcagcactaaccgagagaaatacc | |
| | ttaaaagtgttttacgggaactggtcacatacctcctttttctc | |
| | atagtcttgtgcatcttgacctacggcatgatgagctccaatgt | |
| | gtactactacacccggatgatgtcacagctcttcctagacaccc | |
| | ccgtgtccaaaacggagaaaactaactttaaaactctgtcttcc | |
| | atggaagacttctggaagttcacagaaggctccttattggatgg | |
| | gctgtactggaagatgcagcccagcaaccagactgaagctgaca | |
| | accgaagtttcatcttctatgagaacctgctgttaggggttcca | |
| | cgaatacggcaactccgagtcagaaatggatcctgctctatccc | |
| | ccaggacttgagagatgaaattaaagagtgctatgatgtctact | |
| | ctgtcagtagtgaagatagggctccctttgggccccgaaatgga | |
| | accgcttggatctacacaagtgaaaaagacttgaatggtagtag | |
| | ccactggggaatcattgcaacttatagtggagctggctattatc | |
| | tggatttgtcaagaacaagagaggaaacagctgcacaagttgct | |
| | agcctcaagaaaaatgtctggctggaccgaggaaccagggcaac | |
| | ttttattgacttctcagtgtacaacgccaacattaacctgttct | |
| | gtgtggtcaggttattggttgaattcccagcaacaggtggtgtg | |
| | attccatcttggcaatttcagcctttaaagctgatccgatatgt | |
| | cacaacttttgatttcttcctggcagcctgtgagattatctttt | |
| | gtttctttatcttttactatgtggtggaagagatattggaaatt | |
| | cgcattcacaaactacactatttcaggagtttctggaattgtct | |
| | ggatgttgtgatcgttgtgctgtcagtggtagctataggaatta | |
| | acatatacagaacatcaaatgtggaggtgctactacagtttctg | |
| | gaagatcaaaatactttccccaactttgagcatctggcatattg | |
| | gcagatacagttcaacaatatagctgctgtcacagtattttttg | |
| | tctggattaagctcttcaaattcatcaattttaacaggaccatg | |
| | agccagctctcgacaaccatgtctcgatgtgccaaagacctgtt | |
| | tggctttgctattatgttcttcattattttcctagcgtatgctc | |
| | agttggcataccttgtctttggcactcaggtcgatgacttcagt | |
| | actttccaagagtgtatcttcactcaattccgtatcattttggg | |
| | cgatatcaactttgcagagattgaggaagctaatcgagttttgg | |
| | gaccaatttatttcactacatttgtgttctttatgttcttcatt | |
| | cttttgaatatgtttttggctatcatcaatgatacttactctga | |
| | agtgaaatctgacttggcacagcagaaagctgaaatggaactct | |
| | cagatcttatcagaaagggctaccataaagctttggtcaaacta | |
| | aaactgaaaaaaaataccgtggatgacatttcagagagtctgcg | |
| | gcaaggaggaggcaagttaaactttgacgaacttcgacaagatc | |
| | tcaaagggaagggccatactgatgcagagattgaggcaatattc | |
| | acaaagtacgaccaagatggagaccaagaactgaccgaacatga | |
| | acatcagcagatgagagacgacttggagaaagagagggaggacc | |
| | tggatttggatcacagttctttaccacgtcccatgagcagccga | |
| | agtttccctcgaagcctggatgactctgaggaggatgacgatga | |
| | agatagcggacatagctccagaaggaggggaagcatttctagtg | |
| | gcgtttcttacgaagagtttcaagtcctggtgagacgagtggac | |
| | cggatggagcattccatcggcagcatagtgtccaagattgacgc | |
| | cgtgatcgtgaagctagagattatggagcgagccaaactgaaga | |
| | ggagggaggtgctgggaaggctgttggatggggtggccgaggat | |
| | gaaaggctgggtcgtgacagtgaaatccatagggaacagatgga | |
| | acggctagtacgtgaagagttggaacgctgggaatccgatgatg | |
| | cagcttcccagatcagtcatggtttaggcacgccagtgggacta | |
| | aatggtcaacctcgccccagaagctcccgcccatcttcctccca | |
| | atctacagaaggcatggaaggtgcaggtggaaatgggagttcta | |
| | atgtccacgtatgatatgtgtgtttcagtatgtgtgtttctaat | |
| | aagtgaggaagtggctgtcctgaattgctgtaacaagcacacta | |
| | tttatatgccctgaccaccataggatgctagtctttgtgaccga | |
| | ttgctaatcttctgcactttaatttattttatataaactttacc | |
| | catggttcaaagatttttttttctttttctcatataagaaatct | |
| | aggtgtaaatattgagtacagaaaaaaaatcttcatgatgtgta | |
| | ttgagcggtacgcccagttgccaccatgactgagtcttctcagt | |
| | tgacaatgaagtagccttttaaagctagaaaactgtcaaagggc | |
| | ttctgagtttcatttccagtcacaaaaatcagtattgttatttt | |
| | tttccaagagtgtgaaggaaaatggggcattcctttccactctg | |
| | gcatagttcatgagcttaatacatagctttcttttaagaaagga | |
| | gccttttttttcaactagcttcctggggtaaacttttctaaaag | |
| | ataaaatggaaaggaactccaaactatgatagaatctgtgtgaa | |
| | tggttaagatgaatgttaaatactatgcttttttgtaagttgat | |
| | cgtatctgatgtctgtgggactaactgtatcacttaatttttac | |
| | cttattttggctctaatttgaataagctgagtaaaaccaccaaa | |
| | gatcagttataggataaaatggcatctctaaccataacacagga | |
| | gaattggaaggagccctaagttgtcactcagtttaatttctttt | |
| | aatggttagtttagcctaaagatttatctgcatattctttttcc | |
| | catgtggctctactcatttgcaactgaatttaatgttataactc | |
| | atctagtgagaccaacttactaaatttttagtatgcactgaaag | |
| | tttttatccaacaattatgttcattttaagcaaaattttaagaa | |
| | agttttgaaattcataaagcatttggttttaaactattttaaga | |
| | atatagtactcggtcaggtatgacggctcacgcctgtaatccca | |
| | gcactttgggaggccgaaacaggcgaatcacttgagcccaggag | |
| | ttcaagaccaacatgggcaatgtggcgaaactccatctctacaa | |
| | aaaatgcaaaaataaaaaatatagtactcaagtattcttgatcc | |
| | tgtgtttcaaaactagaatttgtaatgcaaatggagctcagtct | |
| | aataaaaaagaggttttggtattaaaagttcatacattagacag | |
| | tatcagccaaaatttgagttagcaacactgttttctttacgaga | |
| | gggtctcacccaaatttatggggagaaatctatttctcaaaaaa | |
| | aaaaatcttcttttacagaaatgttgagtaaggtgacattttga | |
| | gcgctaataagcaaaagagcatgcagtgctgttgaataaccctc | |
| | acttggagaaccaagagaatcctgtcgtttaatgctatatttta | |
| | atttcacaagttgttcatttaactggtagaatgtcagtccaatc | |
| | tccaatgagaacatgagcaaatagacctttccaggttgaaagtg | |
| | aaacatactgggtttctgtaagtttttcctcatggcttcatctc | |
| | tatctttactttctcttgaatatgctacacaaagttctttatta | |
| | ctacatactaaagtttgcattccagggatattgactgtacatat | |
| | ttatgtatatgtaccatgttgttacatgtaaacaaacttcaatt | |
| | tgaagtgcagctattatgtggtatccatgtgtatcgaccatgtg | |
| | ccatatatcaattatggtcactagaaagtctctttatgatactt | |
| | tttattgtactgtttttcatttcacttgcaaaattttgcagaat | |
| | tcctcctttctacccataaattacatatatttttcttctttagt | |
| | catggagaactccccccctcatctcttccctattatctttccct | |
| | gtgtactggtattattaaaaagacattacatacgcaagtctttc | |
| | tcgacaatcaagaatgttattaatgtgtaatactgagcacttta | |
| | cttcttaataaaaacttgatatagtagca | |
| 3 | agtgcagcgcgaatgcgcgagcaggcggccaaggccctgaggtg | Mouse Pkd1 mRNA |
| | cggcctgccccagagcgctgagcagctgtcgcaccgcagacggg | GenBank Accession |
| | ccacggccgccgcggcattcagccccgtgccgcaccatgaggtc | No. NM_013630.3 |
| | cgcgggcccctcctgaactgcggctgccggcgcaaggggcctcg | |
| | gggaaccggggccatgcgcgggctgcgctgacgatgccgctcgg | |
| | cgcgcctgctctcctggcgctggccctaggcctgggcctgtggc | |
| | tcggagcgctggccggagaccctgggcgcggctgcgggccttgc | |
| | ccgctcccctgcttttgcggccctgcgcccgacgccgcctgccg | |
| | cgtcaattgctccggccgctggctgcagacgctagggccgagtc | |
| | tgcgcatcccggctgacgccaccgcgctagacctgtcccacaac | |
| | ctacttcagacgctggacatagggctcctggtgaacctttcagc | |
| | actggtggagctggacttaagcaacaataggatttctacattag | |
| | aagaaggcgtgtttgctaatttatttaatttaagtgaaataaac | |
| | ctgagtgggaacccatttgagtgcaactgtggtctggcgtggct | |
| | gccacgctgggcaaaggaacatcaggtgcatgtggtacagtctg | |
| | aggcaaccacgtgcagggggcccattccactggccggccaacct | |
| | ctcctcagtatccccttattggacaatgcctgtggtgaggaata | |
| | tgtcgcctgcctccctgacaacagctcaggtgctgtggcagcag | |
| | tacccttctactttgcccatgagggaccactggagactgaggcc | |
| | tgcagtgccttctgcttctcagctggtgagggccttgcagccct | |
| | ctcggagcagaatcaatgcctctgtggggcaggccaggcctcta | |
| | actcttctgcagcctgctcatcctggtgttccagcatctcactg | |
| | tccctcaactctgcctgtgggggccccaccttgctccagcacac | |
| | ctttcccgcctccccaggggctaccctggtggggccccatggac | |
| | ccctggcctctggccagccagcagacttccatattacttcctcc | |
| | ctgcccattagctcaacacgctggaactttggagatggctcacc | |
| | tgaggtggacatggctagccctgctgccacccacttctatgtgc | |
| | tacctgggagctatcacatgacagttgtgctggccttgggggct | |
| | ggctcagctctgctggagacagaggtgcaagtagaagcaacacc | |
| | tactgtcctggagcttgtgtgcccatcctttgtgcacagtaacg | |
| | agagccttgaacttggcatacggcatcgtggaggctcagccctt | |
| | gaggtcacctatagcatcctggctttggataaagagcctgccca | |
| | agtggtccacccactctgccccttggacacagagatcttccctg | |
| | gcaatgggcactgctaccgcctggtggcagagaaggcaccctgg | |
| | ctgcaagcacaggagcaatgtcggacttgggctggagctgcctt | |
| | ggctatggtggacagtcctgccatccagcacttcctagtctcca | |
| | aggttaccaggagcctggatgtgtggatcggcttctcatctgtg | |
| | gaggggacagaaggcctggatccccggggtgaggccttcagcct | |
| | ggaaagctgccagaactggctgcctggggagccacacccagcca | |
| | cagcagagcattgtgtgcggttggggcctgctgggcaatgcaac | |
| | acagacctgtgttcagcaccacatagttatgtctgcgagctgcg | |
| | gcctggagggcctgtgtgggatactgagaactttgtcatgggaa | |
| | tgtctggtggaggcctgtctggacctctgcatccattggcacag | |
| | caggaaactgtccagggcccgctgcgacctgtggaggtcatggt | |
| | gttccctggcctgagccctagccgggaagcctttctcactgcag | |
| | cagagttttctacccagaagcttgaagagcctgcccagatgcgg | |
| | ctgcaggtgtaccggccctcaggaggagcagcagcggtcccaga | |
| | aggcagcagtgagcctgacaacaggactgagccggcccccaagt | |
| | gtgtgcctgaggaactctggtgtccaggagccaatgtctgcata | |
| | ccttttgatgcttcgtgtaactctcatgtctgcatcaatgggtc | |
| | tgtgtcaaggctgggactctctagggccagctacacactatgga | |
| | aggagttctttttttctgtgcctgcagggcctcccacacagtac | |
| | ttggtcaccctccacagccaggatgttcctatgctccctggtga | |
| | cctcattggcttacagcatgatgctggcccaggtactctcctac | |
| | agtgtcccctggcttccagctgtcctggccaagccctgtacctt | |
| | tcaaccaatgcctcagattggatgaccaacctgcctgtccatct | |
| | ggaagaagcttgggctggccctgtctgctccctacagctgcttt | |
| | tggtcacagagcggcttacaccactgctgggccttgggcctaac | |
| | cctggactacagcatcctgggcactatgaggtccgggctacagt | |
| | gggcaacagcgtatccaggcaaaacctgtcctgcagcttcagtg | |
| | tggtctctccaatagctgggctgagagtcattcaccccatcccc | |
| | cttgatggccacatctatgtgccaaccaacggctcagtcttggt | |
| | gcttcaggtagattctggtgctaatgccactgccacagctcagt | |
| | ggtttgggggcaatattagtgctcccttcgaggatgcctgccct | |
| | cctgaggtggattttctcaagcaagattgcacagaagaggccaa | |
| | tggcaccctgttctcagtgttgatgttgcccaggctcaaggagg | |
| | gggatcacacagtggagattgtggcgcaaaatggagccagccag | |
| | gccaacctcagcctgagagtgacagctgaggagcccatctgtgg | |
| | cctccgtgctgtgccaagccctgaggcccgagtgctacagggca | |
| | tcctggtgcggtacagccccatggtagaggctggctcagacgta | |
| | gccttccggtggaccattgatgataagcagtctctgactttcca | |
| | caacactgtcttcaatgtaatataccagagtgctgccatcttca | |
| | agctctcgctgacagcctctaaccatgtgagcaacatcaccgtg | |
| | aactacaatgtcactgtggagcggatgaacaagatgcatgggct | |
| | gtgggtatctgcagtaccgactgtgttaccccccaatgccacac | |
| | tggcactgacaggtggtgtgctggtggattcagctgtggaggtg | |
| | gccttcctgtggaactttggggatggggagcaggtactccgcca | |
| | gttcaagcctccatacgacgagtccttccaggttccagacccca | |
| | cagtggcgcaggtgctggtggaacacaataccacgcatatttac | |
| | actaccccaggtgagtacaacttgaccgtccttgtgtccaatac | |
| | ctatgagaatctgacgcagcaggtgacagtaagtgtgcgcactg | |
| | ttcttcccaacgtggctattggcatgagcagcaacgtcctggtg | |
| | gccggccaacccatcaccttttccccatatccgctgccctcaac | |
| | tgatggtgtcctatatacgtgggactttggggatggctcacctg | |
| | tcctaatacagagccagccagtactcaaccacacctacagcatg | |
| | acgggcgcttacagaatcaccttggaggttaacaacacagtgag | |
| | cagcgtgacagcacatgcagacatccgtgtcttccaggagttgc | |
| | atggcctgactgtgtatttgagcccatctgtggagcagggagcc | |
| | cccatggtagtcagtgcttctgtggagtcaggtgacaacattac | |
| | atggacatttgacatgggagatggcacagtattcacaggccctg | |
| | aggctacagtacaacatgtatacctgcgggcccagaacttcaca | |
| | gtgacagtagaagcagccaaccctgctggccacttgtcccagag | |
| | cctgcatgtgcaagtgtttgtcctggaggtattacacatagagc | |
| | cctctacctgcatccccacacagcccagtgcccagcttatggcc | |
| | catgtcactggggaccctgtccattacctctttgattggacttt | |
| | tggagatggctcctccaatgtgactgtccatgggcatccatcag | |
| | tgacacacaacttcactaggagtggcatattcccactggcattg | |
| | gttttgtcaagccatgtgaacaaggcacattattttaccagcat | |
| | ctgtgtggaacctgagatacgtaacatcaccctgcagcctgaga | |
| | ggcagtttgtgaagctgggggatgaagcccggctggttgcctat | |
| | tcttggcccccattcccgtaccgatatacctgggactttggcac | |
| | tgaagacactacccacacccaaactgggggctctgaagtgaaat | |
| | ttatctaccgagaaccaggctcctacttggtgatagtcactgtg | |
| | tccaacaacatctcttctaccaatgactcagcttttgtggaggt | |
| | ccaggagcctgtgttagtcactggcatcaggatcaatggctccc | |
| | atgtgttagagctgcagcagccttacctgttgtcagccatgggc | |
| | agtgggagccctgccacctacctatgggaactgggagacggcag | |
| | ccagagtgagggccctgaggtcacccatatctacagcagcaccg | |
| | gtgatttcactgtcagggtgtctggctggaatgaggtgagccgt | |
| | agtgaggcccaactcaacatcaccgtaaagcagcgtgtgcgggg | |
| | cctcaccatcaatgccagccgcacagtggtgcccctgaatggca | |
| | gtgtgagctttagtaccttgttggaggtagggagtgatgtgcat | |
| | tactcctgggtgctctgtgacagatgcacacccattccaggcgg | |
| | acccactatttcctacaccttccgctctgtgggcaccttcaata | |
| | ttattgtcacagctgagaatgaggtgggttctgcccaggacagc | |
| | atcttcatctatgttctccagttcattgaggggctgcaggtggc | |
| | agggggtgataacggctgctgcttccctaccaactacacactgc | |
| | agctgcaagctgcagttagggatggcaccaacatctcttatagc | |
| | tggacagcgcagcaggagggcagcctcatcacactcttcggcag | |
| | tggcaagtgcttttctctcacttcactcaaagctagcacctact | |
| | atgtccatcttagggccactaacatgttgggtagtgccgcagcc | |
| | aaccgtactatagactttgtggaacctgtggagagtctaatcct | |
| | atctgcatcccctaatccagctgctgtcaacatgagtctcaccc | |
| | tttgtgctgaattggctggtggcagtggtgttgtttacacttgg | |
| | tacctggaggaaggactgagttggaagacctccatgccatctac | |
| | cacccacacctttgctgcacctggcctgcacttggtcagagtca | |
| | cagctgagaaccagttgggctcagtcaatgccacagttgaagtg | |
| | gccatacaggtgcctgtgggtggtctgagtatcagaaccagtga | |
| | gccagatagcatctttgtggcagctggctccactctacccttct | |
| | ggggtcagctggctgaaggtaccaatgtgacttggtgctggacc | |
| | ttgccaggtgggagtaaggacagccagtacattgctgtgcgctt | |
| | ctccacggctggcagtttctctctgcagctcaatgcttccaatg | |
| | cagtcagttgggtctcagccatgtacaacctcacagtagaggaa | |
| | cccattgtgaacctgatgctgtgggccagcagcaaggtggtggc | |
| | gcctgggcagccagtccactttgaaatcctgctggcagctggct | |
| | ctgctcttactttccgacttcaggttggtgggtctgtccctgaa | |
| | gtgctccctagcccccacttctcccacagcttctttcgggttgg | |
| | agaccatctggtgaatgtgcaggctgagaaccacgtgagtcatg | |
| | cccaggcacaggttcgcatcctagtgctggaggctgttgttggg | |
| | ctgcaggtacccaactgctgtgagccaggcatggccacaggcac | |
| | tgagaagaacttcacagcccgggtgcagcgaggttcccgagttg | |
| | cctatgcctggtatttctccttgcaaaaggttcagggtgactca | |
| | ttggttattttatctggccgtgatgtcacctacacacctgtggc | |
| | tgcagggttactggagatccatgtgcgtgcctttaatgaactgg | |
| | gtggtgtgaacctcacactcatggtggaagtccaggatatcatc | |
| | cagtatgtaacacttcagagtggccgctgctttaccaaccgctc | |
| | agccaggtttgaggctgctacaagccccagtccacggcgtgtaa | |
| | cctaccactgggactttggggatgggactccggtacagaaaaca | |
| | gaggaattctgggctgatcactactacctgcgtcctggggacta | |
| | ccatgtagaggtgaatgctacaaacctagtgagtttttttgtgg | |
| | cacaggccacagtgactgtccaggtgctggcctgtagggagcct | |
| | gaagtggaggtagcactgcctctgcaggtgctgatgcggcgttc | |
| | ccagcgcaactacctggaggcccatgttgatctacgcaactgtg | |
| | tctcctaccagactgaatatcggtgggagatatatcgtactgcc | |
| | agctgccaacggccaggacgtatggctcaaatggtactgcctgg | |
| | tgtggatgtgagccgaccccagctggtggtgccacggctggcac | |
| | tacctgtgggccactactgctttgtgtttgtggtttcatttggg | |
| | gacacaccattggcacggagcatccaggccaatgtgacagtggc | |
| | tgctgagcgtctggtacccatcattgaaggtggctcataccggg | |
| | tatggtcagacacgcaggacctggtgctggatgggagcaagtcc | |
| | tatgaccctaatttagaggatggtgatcagacaccgctcaactt | |
| | ccattgggcctgcgtggcttcaacacagagtgagacaggtggct | |
| | gcgtgctaaactttgggccccgtgggagcagtgtggtcaccatc | |
| | cccctggaacgcctagaagctggtgtagaatataccttcaacct | |
| | catagtgtggaaagcaggtcggaaggaagaagccaccaaccaga | |
| | cagtactgatccgaagtgggcgagtccccattgtgtcccttgag | |
| | tgtgtgtcctgcaaggcacaagcagtgtatgaagtgagccgaag | |
| | ttcctatgtgtacttggagggtcactgccacaactgcagcagag | |
| | gctacaagcaagggtgttgggccgctcgcactttcagcaataag | |
| | actctggtgctgaacgagacgacaacatccacgggtagcaccgg | |
| | catgaatctggtagtgcggccaggtgcactgcgtgatggggagg | |
| | gctacatcttcacactcactgtgctgggccactcaggggaagaa | |
| | gagggctgtgcctcaatccgcctctcacccaaccgccccccact | |
| | tggaggctcctgccgcctattcccattggattctgtgcgtggcc | |
| | tcaccactaaggttcacttcgagtgcacaggttggcgtgatgca | |
| | gaagatggtggagccccactggtatatgctctgttgctgaagcg | |
| | ctgtcgtcagagctactgtgagaatttctgcatctataagggta | |
| | gtctttccacctatggggctgtcctgcctcctggattccagccc | |
| | ctttttgtggtgagcctagctgtcgttgtacaagaccagctgcg | |
| | tgctgctgttgttgcactcaacaggtctctgaccattgtcctac | |
| | cagaacccagtggaaatcctgcagacctcgtaccctggctgcac | |
| | agcctgactgccagtgtgctgcctgggctcctgaagcaggctga | |
| | ccctcagcatgtcatcgagtactctttagccctcattactgtgc | |
| | tcaatgagtatgagcaggccccagatgtgtcagagcccaacgtt | |
| | gagcagcagctgagagcccaaatgcgtaagaacatcacggagac | |
| | cctgatctccctgcgggtcaacactgtggatgacatccagcaga | |
| | ttactgctgcgttagcccagtgcatggtgtccagcagggagctc | |
| | atgtgccgctcctgcctgaagaagatgctgcagaagctggaggg | |
| | catgatgcgcatcttgcaagctgaaaccactgagggcaccttga | |
| | cacccaccaccattgctgacagcattctcaatatcacaggtgac | |
| | ctcatccacctggccagcttggacatgcagggcccacagccctt | |
| | ggagctgggggtggagccgccctccctgatggtggcttctaagg | |
| | cctacaacctctcgtcggccctcatgcgtatcctcatgcgttcc | |
| | cgagtgctcaatgaggagcctctgactctggctggcgaggagat | |
| | tgtggccctaggcaagcgctcagacccactcagtcttctgtgtt | |
| | atggcaaagccttggggcccagctgccacttctccattcctgag | |
| | gccttcagtggggccctgtcgaacctcagtgatgtggtgcagct | |
| | catcttccttgtggattccaaccccttcccctttggctacatta | |
| | gcaactacactgtctccaccaaagtggcatctatggcattccag | |
| | acacagactggtacccagatccctattgagcagttggctgcaga | |
| | gcgtgccatcacagtgaaggtgccaaacaactcagaccaggctg | |
| | cccagagctctcacaatcctgtgggttctaccattgtacagccc | |
| | cagacctctgtcagcgctgtggtcactgcagacaacagtaaccc | |
| | tcaagctggcctgcatctgaggatcacttacacagtgcttaatg | |
| | agcgctatctgtccgcggagcctgagccctacctggctgtgtac | |
| | ctgcactcagtgtcccagcccaatgagtacaactgctcggccag | |
| | caggaggatcagcctggaggtgctggagggtgctgaccacaggc | |
| | tctacaccttcttcattgccccaggaactgggaccttggacagg | |
| | agttactacctgaacctgacgagccatttccactggtctgccct | |
| | ggaggtgtctgtgggcctctatacatccctgtgccagtacttca | |
| | gtgaggagatgatgatgtggagaactgagggcattgtacccctg | |
| | gaggagacctcacccagccaggctgtctgcctcacccgccacct | |
| | cactgccttcggtgccagcctttttgtgcctcccagccatgtcc | |
| | agttcatctttcctgaaccatctgcaagcatcaactacattgtc | |
| | ctcctgacgtgtgtcatatgcctggtgacctatgtggtcatggc | |
| | tatgatcctgcgcaagctggaccagctggatgttagccgtgtcc | |
| | gtgtcatcccgttctgtgggaaggggggccgcttcaaatatgaa | |
| | atacttgttaagaccggctggagccgaggttcaggtaccactgc | |
| | tcacgtcggcatcatgctgtatggagaggacaaccgtagtggtc | |
| | accggcacttagatggggacagagcctttcatcgaaacagcctg | |
| | gacatcttccaaattgccaccccgcacagcctgggcagcgtgtg | |
| | gaagatccgagtgtggcatgataacaaagggctcagccctgcct | |
| | ggttcctgcaacacattatcgtccgggacctacagagtgcccgc | |
| | agcaccttctttttggtcaatgactggctgtctgtggagactga | |
| | ggccaacgggggcctagtggagaaggaggtgcttgcagcaaatg | |
| | aagctgcgctgtggcagttccagcggctcctcgtggctgagttg | |
| | cagagaggcttctttgacaagcacatctggctctccatatggga | |
| | ccggccgcctcgtagccgctttactagagtccagagggttacct | |
| | gctgtgttctcctcctctgcctcttcctggctgccaatgctgtg | |
| | tggtacggagttgtgagagacaccacctacagcatgggacctgt | |
| | gtccagtctgatctctcctggtgttgacacagttgctattggcc | |
| | ttgtgtccagtgtggtagtctaccctgtctatttggctgtcctg | |
| | ttcctcttccggatgtctcggagtaaggtgtctggggaccaaaa | |
| | ccccacacccacagggcagcaggccctagatgttgacagctacc | |
| | tggacccatctgtgctagacagctccctcctcacactctctggc | |
| | ctcacagaggccttcgctgggcaagtgaagaatgacttgtttct | |
| | ggaagatgctaaaagcctggtgtgctggccgtccagtgagggta | |
| | ctcttagttggccagacctgctcagtgacccatctgttgtgagc | |
| | agcacactgcagcgattgacccagggcaggcctggctgcatgtt | |
| | gggctcagaggaagatggtgcctctctggttagcccttccttgc | |
| | ctgccaaatacttgtcggcctcagatgaagacctgatccatcag | |
| | gtactggctgatggggccaacaacctggttcctacccaggacac | |
| | cctcctagaaacagacctgcttactagcctgtccagtgttcctg | |
| | gagagaaaacagaaactctgatactgcagacagtgggggaggag | |
| | agaccagccagcatgggcctcagttgggaacagtccccggtgac | |
| | aaggctttccaggacaggactggtggaaggttttcagaagcgac | |
| | tgctgcctgcctggtgtgccccactggcccatgggctcagcctg | |
| | ctcttggtagctgtagctgtagcagtttcagggtggattggtgc | |
| | cagcttccctcccagcgtgagtgtcatgtggcttctctcaagca | |
| | gctccagcttcctggcctcctttcttggctgggagccccttaag | |
| | gtcctgctggaagccctctacttctcccttgtggccaagcggct | |
| | tcaccctgatgaagatgacactctggtagagagcccagctgtaa | |
| | cccctgtaagtgagcgtgtgccccgtgtgcggcccccacatggt | |
| | tttgcactcttcttggcaaaggaggaagctcgaaaggtcaagag | |
| | gctccatgacatgttgaagagactcttggtgtacatgcttttct | |
| | tactggtgacgctgctggccaactatggggatgcttcttgccat | |
| | ggtcacgcctatcgcctgcagagcgccatcaagcaggagctgga | |
| | tagccaagcctttctggccatcacccggtctgatgagttctggc | |
| | catggatgtcacatgtgttcttgccctatgtccatgggaaccag | |
| | tctagccctgagctgggccctccacggctacggcaggtgcgact | |
| | gcaggaagcattctgcccagacccttctagctcggagcacatgt | |
| | gttcagcggcaggaagcttgagcaccagtgactatggcattggc | |
| | tggcagagtgtggtgcaaaatggctctgagacatgggcctactc | |
| | tgcaccggacctgctgggcgcctggtactggggctactgtgcag | |
| | tgtatgacagtgggggctacatacaggagttaggactgagtctg | |
| | gaggagagccgtgcacgactgggcttcctgcagctgcacaactg | |
| | gcttgacagcaggagccgtgcagtgtttgtagaactcacccgct | |
| | acagccctgctgtggggctccatgctgctgttacactgcgcctc | |
| | gagttccctgtagccggccatgcactggctgccttcagtgtccg | |
| | cccatttgcgttacggcgactcagcactggcctgtctctaccac | |
| | tgctcacctcggtgtgcttgctgctgtttgccctatacttttcc | |
| | atggccgaggtgcagacctggcgcaaagatgggtgtgcgtgcac | |
| | agcaaggcctgacacctgggcacggtgcctactggtgatactga | |
| | cagcagctacagggcttgtacgcctggcccagctgggcattgct | |
| | gatcgccagtggacccattttgtgcaggaccatccacgccactt | |
| | cactagcttcgaccaagtggctcagctgggctctgtggcccgtg | |
| | gcctggcagcttcactactcttcctgcttttggtcaaggctgcc | |
| | cagcagctgcggtttgtgcgccagtggtctgtttttggcaagac | |
| | attgtgtcgggccttgcccgagctcatgggagccaccttgggcc | |
| | tggtgctgcttggtgtggcctatgcacagatggccatcctgctt | |
| | atttcctctggtgctgacactctctacaacatggcccgtgcctt | |
| | cctggtgctgtgccctggggcgagggtccccactctgtgccctt | |
| | cagagtcctggtacctgtcccctctgctgtgtgtggggctctgg | |
| | gctctgcgagtgtggggtgccctgaggttaggggctatcctcct | |
| | gcgttggcggtaccacgccttgcgtggcgagctctaccgcccag | |
| | catgggaaccccaggactatgagatggtggagcttttcctgcgt | |
| | aggcttcggctctggatgggcttcagcaaggtcaaggagttccg | |
| | ccacaaagtccgctttgaaggaatggatccactgccttcccgct | |
| | catccaggggctccaagtcatccccagttgtgctcccacctagc | |
| | tcaggctcagaagcttcacacccatccacctcgtccagccaacc | |
| | agacgggccgagcgccagcttaagccgctcgacgctgaagctgg | |
| | aaccagagccctctcgcctccatgctgtgtttgaaagtctgctt | |
| | gtccagtttgaccgactcaaccaggccacagaggacgtctacca | |
| | gctggagcaacaactccagagccttcaaggccatgggcacaatg | |
| | gacctccttcctctccctcccctggctgcttcccaggctctcag | |
| | ccagctttgcccagccgcctttctcgggccagtcaggggctgga | |
| | tcagactgtaggccccaacagggtgtccctgtggcctaataaca | |
| | aggtccaccccagcagcacttaggccctaggggtcttggccatt | |
| | cccttccctgggaatgcctgagcttcacactggcctctcagagc | |
| | cagggtggacaccactcagtattaccttctgctgccctctagct | |
| | tgggccaggcagaacggctgcatgccagttcttttgggtacagg | |
| | tattgctgccttccttacctgtccacatatggggcttctgcact | |
| | ttaaaaaggctgtgtggccagccaggacccagggtcccctcccc | |
| | acaggaggacacagcagtattggaccaagtgagtacccagcctc | |
| | caagatgctaatttattcgccccccctccccccaagtcctcagg | |
| | ttcagcgggctgcgcccagctctacccctaggtggccatctcct | |
| | cttgctaaaaatctaagctggagggagggctaagctacacttct | |
| | cctttatccgtcccctaagttattacctctcttgtttctgcagc | |
| | atacttgccctctagctaccatctgctttctatgtccaccatca | |
| | ataatttatatggggttaaaatgtatatatttttgtatgtcagt | |
| | attttctacttgggctgaaaatgggtccattgttacccagggca | |
| | ggggtacaggggaagctgcagctattactggatccgagtctggc | |
| | ctcgggcaggtgctggacagcaattggggccaccgtggccttgt | |
| | tttcctcccttgtctggggccaggcagcacgcagatctgctggt | |
| | ttcagatctgagcaagggcaggactactgtgggatgaaagatgc | |
| | caagaagttactggacaagcccaccaatggggcttgcccccatg | |
| | gcgggtgggggaaagagaatgtatgtgatggcacctgcctgctt | |
| | ctgtgggctcatctgcagctctagcctggattctgccccaaccc | |
| | caaacagacaagacaaagtcaaataaagaagctgtctgactgc | |

TABLE 29B
Sequence Listing of PRSs

| Sequence | SEQ ID NO |
|---|---|
| GGACU | 4 |
| GGACT | 5 |
| GGAUU | 6 |
| GAACU | 7 |
| AGACU | 8 |
| AAACU | 9 |
| GGACA | 10 |
| AAACA | 11 |
| GGACUGGAC | 12 |
| GGACTGGAC | 13 |
| GGACUGGACU | 14 |
| GGACUGGACUGGACU | 15 |
| ACGGACUUGGACU | 16 |
| AAACUAAACU | 17 |
| AAAAAAAAAAAA | 18 |
| AAAAAAAAAA | 19 |
| CCCCCCCCCC | 20 |
| GGGGGGGGGG | 21 |
| TTTTTTTTTT | 22 |
| UUUUUUUUUU | 23 |
| GGACCCTGGGTCCTG | 87 |
| GGACCCTGGGTCTTG | 88 |
| GGACUGGA | 125 |
| GAACUGAA | 126 |

TABLE 29C
Sequence Listing of Compound Sequence with Chemistry

| Compound ID | Sequence + Chemistry | SEQ ID NO |
|---|---|---|
| ATXL188a | mGmCmAmUmGmGmCmGmGmGmCmGmCmGmGmG | 24 |
| ATXL189 | mGmAmCmGmGmCmAmUmGmGmCmGmGmGmCmG | 25 |
| ATXL190 | mCmAmUmGmGmCmCmCmCmGmCmCmGmUmCmC | 26 |
| ATXL195 | mCmAmCmAmGmCmCmUmCmUmUmUmAmAmAmGmUmGmC | 27 |
| ATXL198 | mUmGmGmCmCmCmCmGmCmCmGmUmCmCmCmC | 28 |
| ATXL157 | mAmCmUmGmGmAmAmAmAmCmAmAmCmAmCmC | 29 |
| ATXL265 | mG*mG*mAmCmUmGmGmAmCmUmCmCmAmUmUmCmUmGmCm | 30 |
| | CmUmGmGmCmCmC | |
| ATXL322 | mU*mU*mA*mA*mA*mG*mU*mG*mC*mU*mG* [AL] *mA* | 31 |
| | [GL] *mC* [mCL] | |
| ATXL323 | mG*mG*mAmCmUmGmGmAmCmU*mU*mA*mA*mA*mG*mU* | 32 |
| | mG*mC*mU*mG* [AL] *mA* [GL] *mC* [mCL] | |
| RGLS4326-L | [AL] * [GL] *mCfAfCfUmU* [TL] * [GL] | 33 |
| ATXL551 | [eT] * [eT] * [eA] * [eA] *[eA] * [eG] * [eT] * [eG] | 34 |
| | * [eCm] * [eT] * [eG] [AL] * [eA] * [GL] * [eCm] * | |
| | [mCL] * [HG-CHOL] | |
| ATXL494 | mG*mG*mC*mC*mC*mC*mG*mC*mC*mG*mU* [mCL] *mC | 35 |
| | * [mCL] *mC* [AL] | |
| ATXL512 | [eT] * [eG] * [eG] * [eCm] * [eCm] * [eCm] * [eCm] | 36 |
| | * [eG] * [eCm] * [eCm] * [eG] * [eT] * [eCm] * [eCm] | |
| ATXL513 | [eT] * [eG] * [eG] * [eCm] * [eCm] * [eCm] * [eCm] | 37 |
| | * [eG] * [eCm] * [mCL] * [eG] * [TL] * [eCm] * [mCL] | |
| ATXL586 | mU*mU*mA*mA*mA*mG*mU*mG*mC*mU*mG* [AL] *mA* | 31 |
| | [GL] *mC* [mCL] [HEG] | |
| ATXL587 | mU*mU*mA*mA*mA*mG*mU*mG*mC*mU*mG* [AL] *mA* | 31 |
| | [GL] *mC* [mCL] [HEG] * [PALM] | |
| ATXL594 | mU*mU*mA*mA*mA*mG*mU*mG*mC*mU*mG* [AL] *mA* | 31 |
| | [GL] *mC* [mCL] [HEG] * [FA-FI] | |
| ATXL595 | mU*mU*mA*mA*mA*mG*mU*mG*mC*mU*mG* [AL] *mA* | 31 |
| | [GL] *mC* [mCL] [HEG] * [FA-FI] * [PALM] | |
| ATXL591 | [GL] * [AL] * [AL] * G*A*G*A*T*G*T*T*G*T* [TL] | 38 |
| | [GL] * [GL] | |
| ATXL501 | [eG] * [eG] * [eA] * [eCm] * [eT] * [eG] * [eG] * | 39 |
| | [eA] * [eCm] * [eG] * [eG] * [eA] * [eCm] * [eCm] * | |
| | [eCm] * [eT] * [eG] * [eG] * [eG] * [eT] * [eCm] * | |
| | [eCm] * [eT] * [eG] * [eG] | |
| ATXL604 | [eT] * [eG] * [eG] * [eCm] * [eCm] * [eCm] * [eCm] * | 40 |
| | [eG] * [eCm] * [eCm] * [eG] * [eT] * [eCm] * [eCm] * | |
| | [eCm] * [eCm] | |
| ATXL605 | mU*mU*mA*mA*mA*mG*mU*mG*mC*mU*mG* [GL] *mA* [GL] | 41 |
| | *mC* [mCL] | |
| ATXL606 | [eT] * [eT] * [eA] * [eA] * [eA] * [eG] * [eT] * | 34 |
| | [eG] * [eCm] * [eT] * [eG] * [AL] * [eA] * [GL] * | |
| | [eCm] * [mCL] | |
| ATXL607 | mG*mG*mAmCmUmGmGmAmC* [eCm] * [eCm] * [eA] * [eT] * | 42 |
| | [eT] * [eCm] * [eT] * [eG] * [eCm] * [eCm] * [TL] * | |
| | [eG] * [GL] * [eCm] * [mCL] | |
| ATXL608 | mG*mG*mAmCmUmGmGmAmC* [eG] * [eG] * [eA] * [eCm] * | 43 |
| | [eCm] * [eCm] * [eT] * [eG] * [eG] * [eG] * [TL] * | |
| | [eCm] * [mCL] [eT] * [GL] | |
| ATXL609 | mG*mG*mAmCmUmGmGmAmC* [eCm] * [eCm] * [eA] * [eA] * | 44 |
| | [eT] * [eA] * [eCm] * [eT] * [eG] * [eCm] * [TL] * | |
| | [eG] * [TL] * [eG] * [TL] | |
| ATXL610 | [eG] * [eG] * [eCm] * [eCm] * [eCm] * [eCm] * [eG] | 45 |
| | * [eCm] * [eCm] * [eG] * [eT] * [eCm] * [eCm] * | |
| | [eCm] * [eCm] * [eA] | |
| ATXL611 | [eG] * [eG] * [eCm] * [eCm] * [eCm] * [eCm] * [eG] | 46 |
| | * [eCm] * [eCm] * [eG] * [eT] * [eCm] * [eCm] * | |
| | [eCm] * [mCL] * [AL] | |
| ATXL612 | [eG] * [eG] * [eCm] * [eCm] * [eCm] * [eCm] * [eG] | 47 |
| | * [eCm] * [eCm] * [eG] * [eT] * [eCm] * [eCm] * | |
| | [eCm] * [eCm] | |
| ATXL613 | [eG] * [eG] * [eCm] * [eCm] * [eCm] * [eCm] * [eG] | 48 |
| | * [eCm] * [eCm] * [eG] * [eT] * [eCm] * [mCL] * | |
| | [mCL] | |
| ATXL614 | [eG] * [eCm] * [eCm] * [eCm] * [eCm] * [eG] * [eCm] | 49 |
| | [eCm] * [eG] * [eT] * [eCm] * [eCm] * [mCL] * [mCL] | |
| ATXL615 | [eG] * [eG] * [eCm] * [eCm] * [eCm] * [eCm] * [eG] | 50 |
| | [eG] * [eT] * [eT] * [eCm] * [eCm] * [eCm] * [mCL] | |
| | * [GL] | |
| RGLS8429 | [cEt-A] * [cEt-G] * [mC] * [fA] * [fC] * [fU] * | 51 |
| | [mU] * [cEt-U] * [cEt-A] | |
| ATXL681 | mG*mG*mAmCmUmGmGmAmC* [eG] * [eG] * [eA] * [eCm] * | 52 |
| | [eCm] * [eCm] * [eT] * [eG] * [eG] * [eG] * [eT] * | |
| | [eCm] * [eCm] * [TL] * [GL] | |
| ATXL682 | mG*mG*mAmCmUmGmGmAmC* [eG] * [eG] * [eA] * [eCm] * | 53 |
| | [eCm] * [eCm] * [eT] * [eG] * [eG] * [eG] * [TL] * | |
| | [eCm] * [TL] * [eT] * [GL] | |
| ATXL683 | mG*mG*mAmCmUmGmGmAmC* [eG] * [eG] * [eA] * [eCm] * | 54 |
| | [eCm] * [eCm] * [eT] * [eG] * [eG] * [eG] * [TL] * | |
| | [eT] * [mCL] * [eT] * [GL] | |
| ATXL685 | mG*mG*mAmCmUmGmGmAmC* [eG] * [eG] * [eA] * [eCm] * | 55 |
| | [eCm] * [eCm] * [eT] * [eG] * [eG] * [eG] * [TL] * | |
| | [eCm] * [mCL] * [eT] * [GL] | |
| ATXL686 | mG*mG*mAmCmUmGmGmAmC* [eG] * [eG] * [eA] * [eCm] * | 56 |
| | [eCm] * [eCm] * [eT] * [eG] * [eG] * [eG] * [TL] * | |
| | [eCm] * [mCL] * [eT] * [GL] | |
| ATXL814 | mG*mG*mAmCmUmGmGmAmC* [eG] * [eG] * [eA] * [eCm] * | 57 |
| | [eCm] * [eCm] * [eT] * [eG] * [eG] * [eG] * [eT] * | |
| | [eCm] * [eCm] * [eT] * [eG] | |
| ATXL815 | mG*mG*mAmCmUmGmGmAmC* [eG] * [eG] * [eA] * [eCm] * | 58 |
| | [eCm] * [eCm] * [eT] * [eG] * [eG] * [eG] * [eT] * | |
| | [eCm] * [eT] * [eT] * [eG] | |

TABLE 29D
Sequence Listing of Compound
Sequence without Chemistry

| | | SEQ |
|---|---|---|
| Compound ID | Sequence | ID NO |
| ATXL188a | GCAUGGCGGGCGCGGG | 59 |
| ATXL189 | GACGGCAUGGGGGGCG | 60 |
| ATXL190 | CAUGGCCCCGCCGUCC | 61 |
| ATXL195 | CACAGCCUCUUUAAAGUGC | 62 |
| ATXL198 | UGGCCCCGCCGUCCCC | 63 |
| ATXL157 | ACUGGAAAACAACACC | 64 |
| ATXL265 | GGACUGGACUCCAUUCUGCCUGGCCC | 65 |
| ATXL322 | UUAAAGUGCUGAAGCC | 66 |
| ATXL323 | GGACUGGACUUAAAGUGCUGAAGCC | 67 |
| RGLS4326-L | AGCACUUTG | 68 |
| ATXL551 | TTAAAGTGCTGAAGCC | 69 |
| ATXL494 | GGCCCCGCCGUCCCCA | 70 |
| ATXL512 | TGGCCCCGCCGTCC | 71 |
| ATXL513 | TGGCCCCGCCGTCC | 71 |
| ATXL586 | UUAAAGUGCUGAAGCC | 66 |
| ATXL587 | UUAAAGUGCUGAAGCC | 66 |
| ATXL594 | UUAAAGUGCUGAAGCC | 66 |
| ATXL595 | UUAAAGUGCUGAAGCC | 66 |
| ATXL591 | GAAGAGATGTTGTTGG | 72 |
| ATXL501 | GGACTGGACGGACCCTGGGTCCTGG | 73 |
| ATXL604 | TGGCCCCGCCGTCCCC | 74 |
| ATXL605 | UUAAAGUGCUGGAGCC | 75 |
| ATXL606 | TTAAAGTGCTGAAGCC | 69 |
| ATXL607 | GGACUGGACCCATTCTGCCTGGCC | 76 |
| ATXL608 | GGACUGGACGGACCCTGGGTCCTG | 77 |
| ATXL609 | GGACUGGACCCAATACTGCTGTGT | 78 |
| ATXL610 | GGCCCCGCCGTCCCCA | 79 |
| ATXL611 | GGCCCCGCCGTCCCCA | 79 |
| ATXL612 | GGCCCCGCCGTCCCC | 80 |
| ATXL613 | GGCCCCGCCGTTCCC | 81 |
| ATXL614 | GCCCCGCCGTCCCC | 82 |
| ATXL615 | GGCCCCGGTTTCCCCG | 83 |
| RGLS8429 | AGCACUUUA | 84 |
| ATXL681 | GGACUGGACGGACCCTGGGTCCTG | 77 |
| ATXL682 | GGACUGGACGGACCCTGGGTCTTG | 85 |
| ATXL683 | GGACUGGACGGACCCTGGGTTCTG | 86 |
| ATXL685 | GGACUGGACGGACCCTGGGTCCTG | 77 |
| ATXL686 | GGACUGGACGGACCCTGGGTCCTG | 77 |
| ATXL814 | GGACUGGACGGACCCTGGGTCCTG | 77 |
| ATXL815 | GGACUGGACGGACCCTGGGTCTTG | 85 |
| ATXL-A | GGACUGGACGGACTCTGGGTCCTG | 89 |
| ATXL-B | GGACUGGACGGATCCTGGGTCCTG | 90 |
| ATXL-C | GGACUGGACGGACCTTGGGTCCTG | 91 |
| ATXL-D | GGACUGGACGGACTCTGGGTTCTG | 92 |
| ATXL-E | GGACTGGACGGACCCTGGGTCCTG | 93 |
| ATXL-F | GGACUGGACGGACCCTGGGTCCTGG | 94 |
| ATXL-G | GGACTGGACGGACCCTGGGTCTTG | 95 |
| ATXL-H | GGACTGGACGGACCCTGGGTTCTG | 96 |
| ATXL-I | GGACTGGACGGACTCTGGGTCCTG | 97 |
| ATXL-J | GGACTGGACGGATCCTGGGTCCTG | 98 |
| ATXL-K | GGACTGGACGGACCTTGGGTCCTG | 99 |
| ATXL-L | GGACUGGACUGGACCCTGGGTCCTG | 100 |
| ATXL-M | GGACUGGACTGGACCCTGGGTCCTG | 101 |
| ATXL-N | GGACTGGACUGGACCCTGGGTCCTG | 102 |
| ATXL-O | GGACTGGACTGGACCCTGGGTCCTG | 103 |
| ATXL-P | GGACUGGACUGGACCCTGGGTCCTGG | 104 |
| ATXL-Q | GGACUGGACTGGACCCTGGGTCCTGG | 105 |
| ATXL-R | GGACTGGACUGGACCCTGGGTCCTGG | 106 |
| ATXL-S | GGACUGGACUGGACCCTGGGTCTTG | 107 |
| ATXL-T | GGACUGGACTGGACCCTGGGTCTTG | 108 |
| ATXL-U | GGACTGGACUGGACCCTGGGTCTTG | 109 |
| ATXL-V | GGACUGGACUGGACCCTGGGTTCTG | 110 |
| ATXL-W | GGAGUGGACTGGACCCTGGGTTCTG | 111 |
| ATXL-X | GGACTGGACUGGACCCTGGGTTCTG | 112 |
| ATXL-Y | GGAGUGGACUGGACTCTGGGTCCTG | 113 |
| ATXL-Z | GGACUGGACTGGACTCTGGGTCCTG | 114 |
| ATXL-AA | GGACTGGACUGGACTCTGGGTCCTG | 115 |
| ATXL-BB | GGACUGGACUGGATCCTGGGTCCTG | 116 |
| ATXL-CC | GGACUGGACTGGATCCTGGGTCCTG | 117 |
| ATXL-DD | GGACTGGACUGGATCCTGGGTCCTG | 118 |
| ATXL-EE | GGACUGGACUGGACCTTGGGTCCTG | 119 |
| ATXL-FF | GGACUGGACTGGACCTTGGGTCCTG | 120 |
| ATXL-GG | GGACTGGACUGGACCTTGGGTCCTG | 121 |
| ATXL-HH | GGACUGGACUGGACTCTGGGTTCTG | 122 |
| ATXL-II | GGACUGGACTGGACTCTGGGTTCTG | 123 |
| ATXL-JJ | GGACTGGACGGACTCTGGGTTCTG | 124 |

1. (canceled)

2-28. (canceled)

29. A compound comprising an oligonucleotide, or salt thereof, for enhancing expression of PKD1 in a cell, wherein the oligonucleotide comprises at least a 17 nucleobase portion of the sequence of SEQ ID NO: 77, or a sequence thereof which differs up to 2 positions of SEQ ID NO: 77.

30. The compound of claim 29, wherein the compound with the sequence of SEQ ID NO:77 is selected from ATXL608, ATXL681, ATXL685, ATXL686, ATXL814.

31. The compound of claim 29, wherein the compound with up to 2 positions different from the sequence of SEQ ID NO:77 is selected from ATXL501 (SEQ ID NO: 73), ATXL682 (SEQ ID NO: 85), ATXL683 (SEQ ID NO: 86), ATXL815 (SEQ ID NO: 85), ATXL-A (SEQ ID NO: 89), ATXL-B (SEQ ID NO: 90), ATXL-C(SEQ ID NO: 91), ATXL-E (SEQ ID NO: 93), ATXL-F (SEQ ID NO: 94), ATXL-L (SEQ ID NO: 100), ATXL-M (SEQ ID NO: 101), ATXL-D (SEQ ID NO: 92), ATXL-G (SEQ ID NO: 95), ATXL-H (SEQ ID NO: 96), ATXL-I (SEQ ID NO: 97), ATXL-J (SEQ ID NO: 98), ATXL-K (SEQ ID NO: 99), ATXL-N(SEQ ID NO: 102), ATXL-O (SEQ ID NO: 103), ATXL-P (SEQ ID NO: 104), ATXL-Q (SEQ ID NO: 105), ATXL-S(SEQ ID NO: 107), ATXL-T (SEQ ID NO: 108), ATXL-V (SEQ ID NO: 110), ATXL-WW (SEQ ID NO: 111), ATXL-Y (SEQ ID NO: 113), ATXL-Z (SEQ ID NO: 114), ATXL-BB (SEQ ID NO: 116), ATXL-CC (SEQ ID NO: 117), ATXL-EE (SEQ ID NO: 119), and ATXL-FF (SEQ ID NO: 120).

32. The compound of claim 29, wherein the compound comprises at least one chemical modification.

33. The compound of claim 30, wherein the at least one chemical modification can be selected from 2′-O-methyl (2′-OMe), 2′-O-(2-methoxyethyl) (2′-MOE), 2′-fluoro (2′-F), constrained ethyl (cEt), unlocked nucleic acid (UNA), locked nucleic acid (LNA), 2′-MOE modified T, and/or 5-methylcytosine base.

34. The compound of claim 29, wherein the compound is fully chemically modified.

35. The compound of claim 34, wherein the chemical modifications can be selected from 2′-O-methyl (2′-OMe), 2′-O-(2-methoxyethyl) (2′-MOE), 2′-fluoro (2′-F), constrained ethyl (cEt), unlocked nucleic acid (UNA), locked nucleic acid (LNA), 2′-MOE modified T, and/or 5-methylcytosine base.

36. The compound of claim 29, wherein the compound further comprises a conjugate.

37. The compound of claim 29, wherein the compound comprises at least one modified internucleoside linkage.

38. The compound of claim 37, wherein the at least one modified internucleoside linkage is a phosphorothioate (PS) internucleoside linkage.

39. The compound of claim 29, wherein the compound comprises the sequence and chemistry of ATXL608 (SEQ ID NO: 43) as shown in the chemical structure:

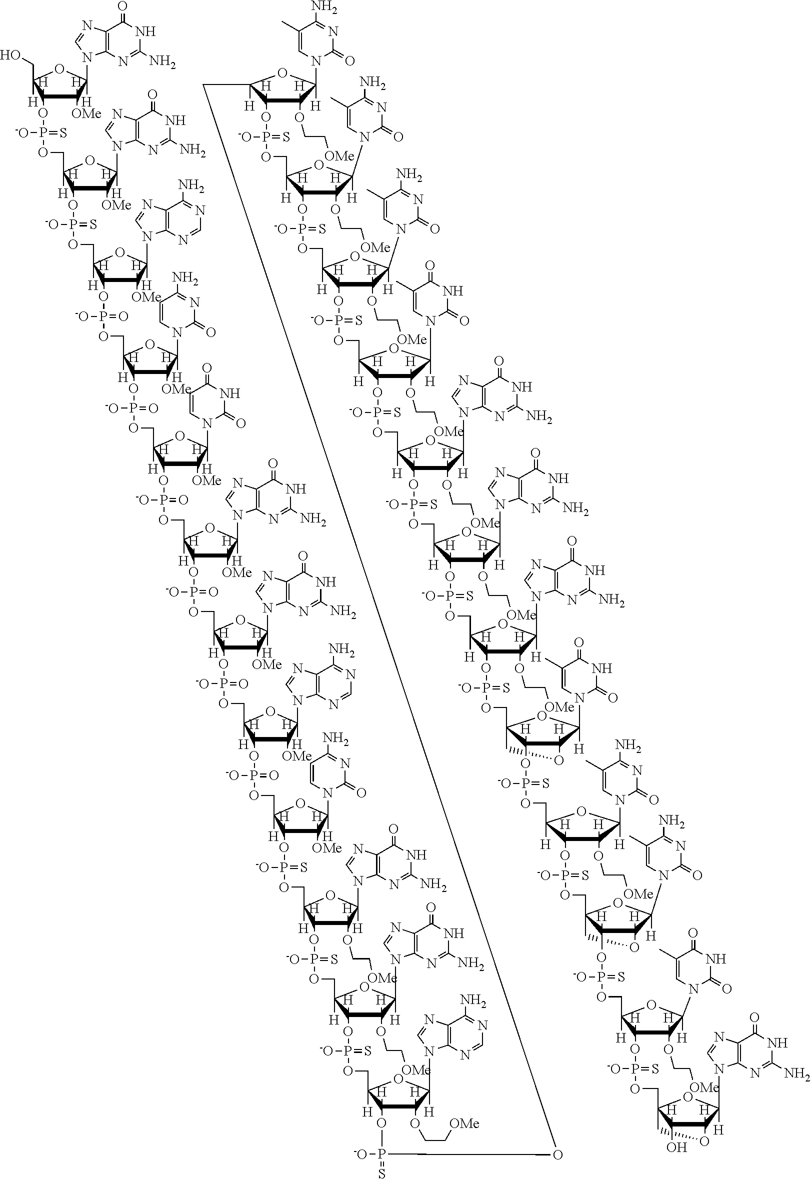

40. The compound of claim 29, wherein the compound comprises the sequence and chemistry of ATXL682 (SEQ ID NO: 53) as shown in the chemical structure:

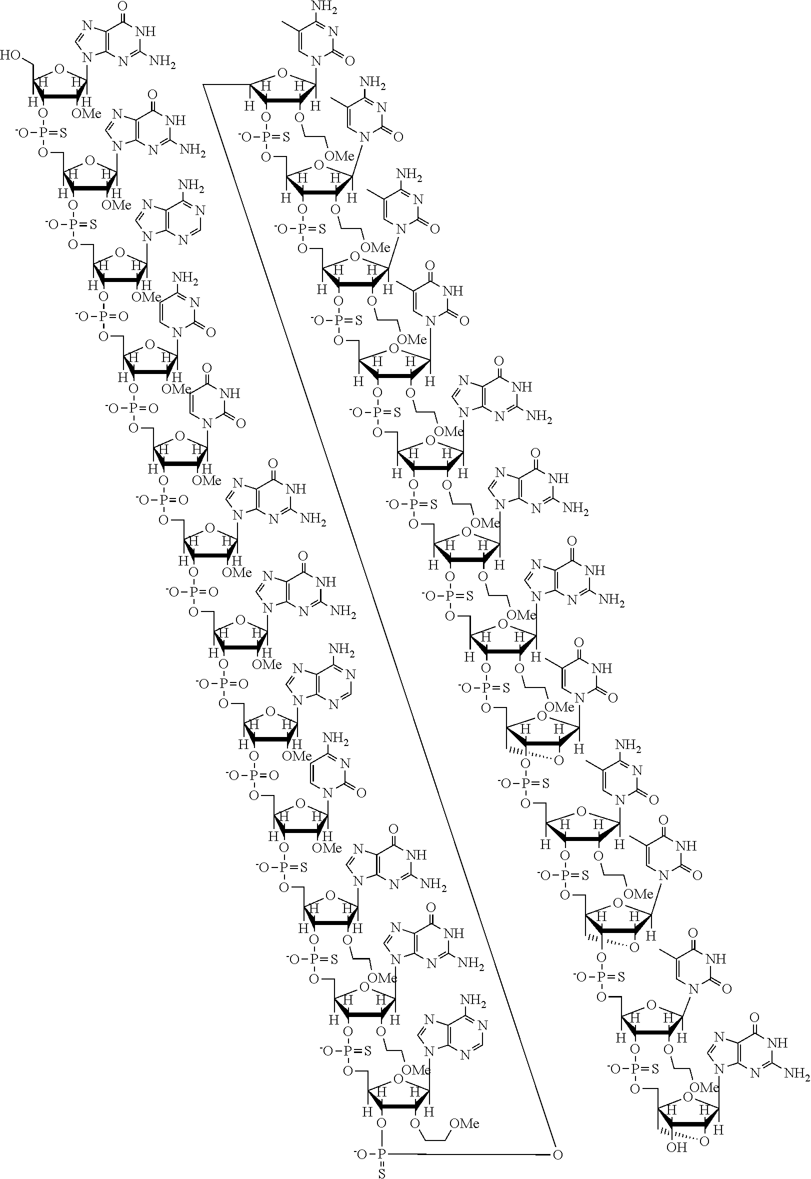

41. The compound of claim 29, wherein the compound comprises the sequence and chemistry of ATXL686 (SEQ ID NO: 56) as shown in the chemical structure:

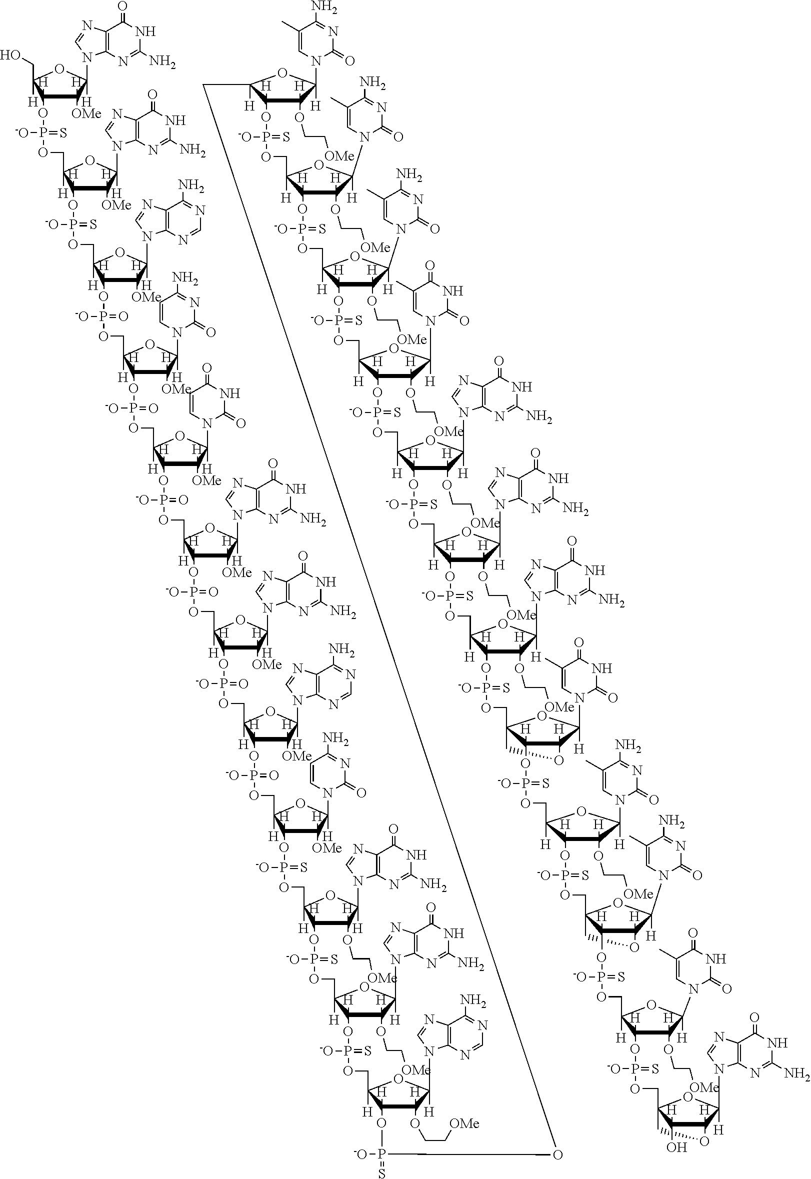

42. The compound of claim 29, wherein the compound comprises the sequence and chemistry of ATXL814 (SEQ ID NO: 57) as shown in the chemical structure:

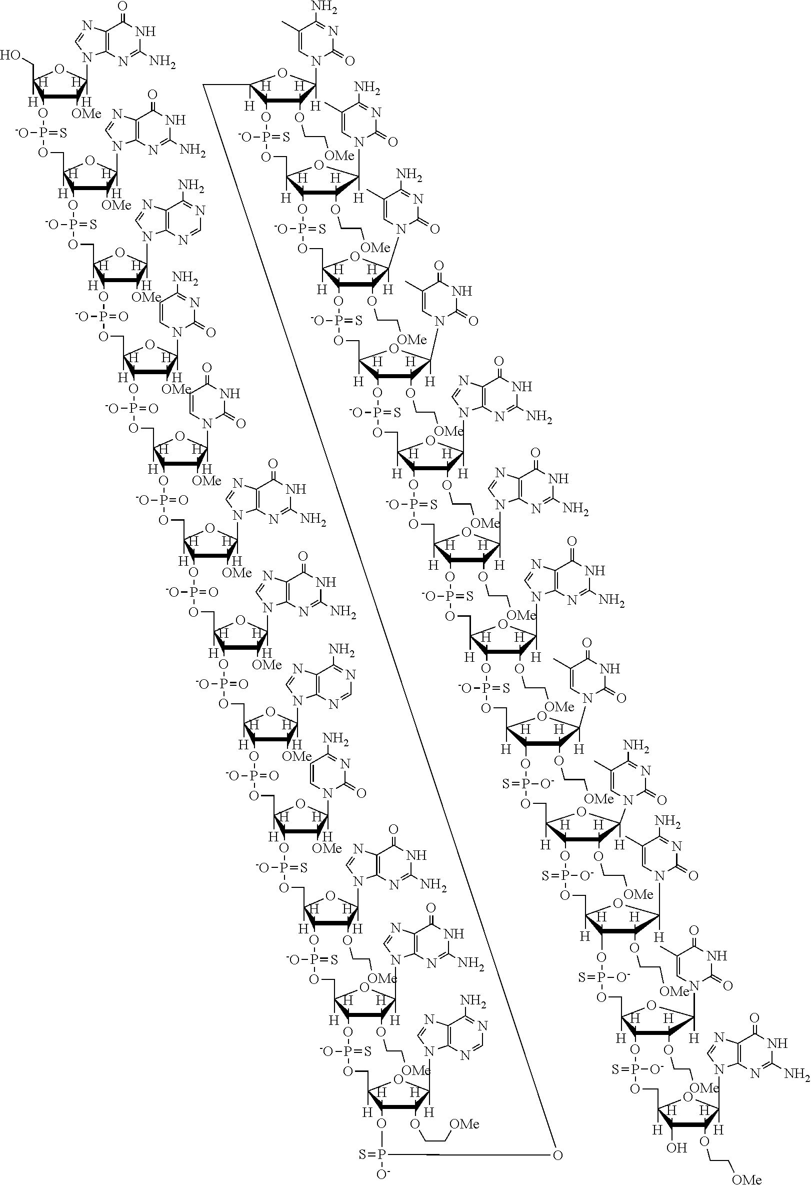

43. The compound of claim 29, wherein the compound comprises the sequence and chemistry of ATXL815 (SEQ ID NO: 58) as shown in the chemical structure:

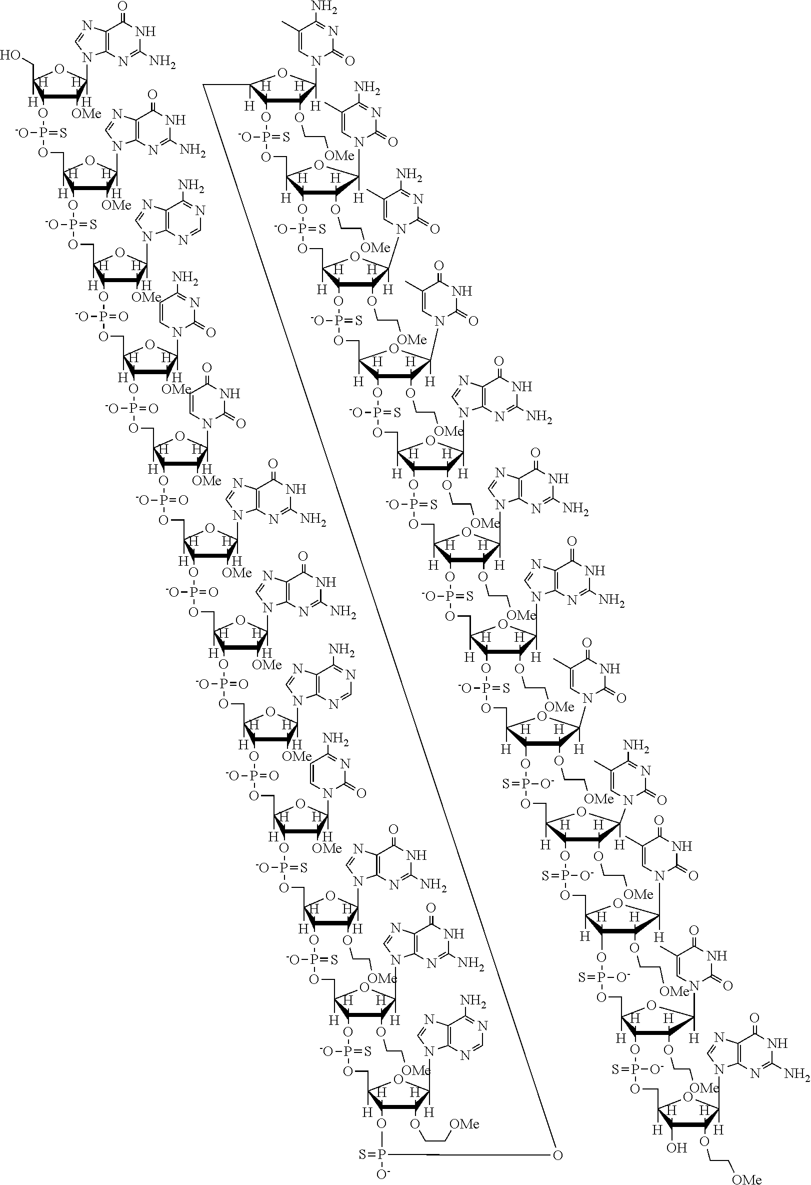

44. The compound of claim 29, wherein the compound increases expression of PKD1 protein by at least about 10%, 15%, 20⁰/, 25%, 30%, 35%, 40%, 45%, 50%, 55%, 60%, 65%, 70%, 75%, 80%, 85%, 90%, 95%, 100%, 150%, 200%, 250%, or 300%.

45. A pharmaceutical composition for enhancing the expression of PKD1 comprising the compound of claim 29, or salt thereof, alone or in combination with a pharmaceutically acceptable carrier and/or excipient.

46. A kit comprising the pharmaceutical composition of claim 45, and a label.

47. A method for increasing translation of PKD1 mRNA in a cell comprising administering the compound of claim 29 to the cell, in an amount sufficient to increase translation of PKD1 mRNA.

48. A method for increasing expression of PKD1 protein in a cell comprising administering the compound of claim 29 to the cell, in an amount sufficient to increase expression of PKD1 protein.

49. A method for treating a haploinsufficiency and/or loss of function disease, disorder and/or condition in a subject comprising administering the compound of claim 29 to the subject, in an amount sufficient to increase PKD1 expression by at least about 20%, 25%, 30%, 35%, 40%, 45%, 50%, 55%, or 60%, wherein the increase in PKD1 protein expression treats the haploinsufficiency and/or loss of function disease, disorder and/or condition in the subject.

50. The method of claim 50, wherein the haploinsufficiency and/or loss of function disease, disorder and/or condition is selected from polycystic kidney disease and/or polycystic liver disease (PLD).

51. The method of claim 50, wherein the polycystic kidney disease is autosomal dominant polycystic kidney disease (ADPKD).

52. A method for decreasing cyst size in an organ of a subject comprising administering the compound of claim 29 to the subject, in an amount sufficient to increase PKD1 expression by at least about 20%, 25%, 30%, 35%, 40%, 45%, 50%, 55%, or 60%, wherein the increase in PKD1 protein expression decreases cyst size in the organ of the subject.

53. A method for decreasing cyst formation in an organ of a subject comprising administering the compound of claim 29 to the subject, in an amount sufficient to increase PKD1 expression by at least about 20%, 25%, 30%, 35%, 40%, 45%, 50%, 55%, or 60%, wherein the increase in PKD1 protein expression decreases cyst formation in the organ of the subject.

54. A method for impeding cyst formation in an organ of a subject comprising administering the compound of claim 29 to the subject, in an amount sufficient to increase PKD1 expression by at least about 20%, 25%, 30%, 35%, 40%, 45%, 50%, 55%, or 60%, wherein the increase in PKD1 protein expression impedes cyst formation in the organ of the subject.

55. A method for reversing cyst progression in an organ of a subject comprising administering the compound of any of claim 29 to the subject, in an amount sufficient to increase PKD1 expression by at least about 20%, 25%, 30%, 35%, 40%, 45%, 50%, 55%, or 60%, wherein the increase in PKD1 protein expression reverses cyst progression in the organ of the subject.

56. The method of any of claim 29, where the compound or the pharmaceutical composition can be administered subcutaneously or intravenously to the subject.

57. A process for preparing compound of claim 29, wherein
the process comprises the steps of:
a. preparing the compound by sequential coupling of modified and/or unmodified nucleotides and/or linkers via the phosphoramidite oligonucleotide synthesis on a conjugate modified or unmodified solid support;
b. optionally, coupling a conjugate moiety to the compound on the solid support via the phosphoramidite oligonucleotide synthesis; and
c. detaching the compound from the solid support and removing the solid support.


SEQUENCE LISTING (124 sequences)

SEQ ID NO: 1 - DNA, 14140 bp, Homo sapiens
  source 1..14140: mol_type = other DNA
gcactgcagcgccagcgtccgagcgggcggccgagctcccggagcggcctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaacgatgccgcccgccgcgcccgcccgcctggcgctggccctgggcctgggcctgtggctcggggcgctggcggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagcctcgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccgggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttagtcctccttcctggcgggggtgggccgtggagtcggagtggacaccgctcagtattactttctgccgctgtcaaggccgagggccaggcagaatggctgcacgtaggttccccagagagcaggcaggggcatctgtctgtctgtgggcttcagcactttaaagaggctgtgtggccaaccaggacccagggtcccctccccagctcccttgggaaggacacagcagtattggacggtttctagcctctgagatgctaatttatttccccgagtcctcaggtacagcgggctgtgcccggccccaccccctgggcagatgtcccccactgctaaggctgctggcttcagggagggttagcctgcaccgccgccaccctgcccctaagttattacctctccagttcctaccgtactccctgcaccgtctcactgtgtgtctcgtgtcagtaatttatatggtgttaaaatgtgtatatttttgtatgtcactattttcactagggctgaggggcctgcgcccagagctggcctcccccaacacctgctgcgcttggtaggtgtggtggcgttatggcagcccggctgctgcttggatgcgagcttggccttgggccggtgctgggggcacagctgtctgccaggcactctcatcaccccagaggccttgtcatcctcccttgccccaggccaggtagcaagagagcagcgcccaggcctgctggcatcaggtctgggcaagtagcaggactaggcatgtcagaggaccccagggtggttagaggaaaagactcctcctgggggctggctcccagggtggaggaaggtgactgtgtgtgtgtgtgtgtgcgcgcgcgcacgcgcgagtgtgctgtatggcccaggcagcctcaaggccctcggagctggctgtgcctgcttctgtgtaccacttctgtgggcatggccgcttctagagcctcgacacccccccaacccccgcaccaagcagacaaagtcaataaaagagctgtctgactgcaa

SEQ ID NO: 2 - DNA, 5089 bp, Homo sapiens
  source 1..5089: mol_type = other DNA
aggcggcggcgggcgccgggaagaaaggaacatggctcctgaggcgcacagcgccgagcgcggcgccgcgcacccgcgcgccggacgccagtgaccgcgatggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttacccatggttcaaagatttttttttctttttctcatataagaaatctaggtgtaaatattgagtacagaaaaaaaatcttcatgatgtgtattgagcggtacgcccagttgccaccatgactgagtcttctcagttgacaatgaagtagccttttaaagctagaaaactgtcaaagggcttctgagtttcatttccagtcacaaaaatcagtattgttatttttttccaagagtgtgaaggaaaatggggcattcctttccactctggcatagttcatgagcttaatacatagctttcttttaagaaaggagccttttttttcaactagcttcctggggtaaacttttctaaaagataaaatggaaaggaactccaaactatgatagaatctgtgtgaatggttaagatgaatgttaaatactatgcttttttgtaagttgatcgtatctgatgtctgtgggactaactgtatcacttaatttttaccttattttggctctaatttgaataagctgagtaaaaccaccaaagatcagttataggataaaatggcatctctaaccataacacaggagaattggaaggagccctaagttgtcactcagtttaatttcttttaatggttagtttagcctaaagatttatctgcatattctttttcccatgtggctctactcatttgcaactgaatttaatgttataactcatctagtgagaccaacttactaaatttttagtatgcactgaaagtttttatccaacaattatgttcattttaagcaaaattttaagaaagttttgaaattcataaagcatttggttttaaactattttaagaatatagtactcggtcaggtatgacggctcacgcctgtaatcccagcactttgggaggccgaaacaggcgaatcacttgagcccaggagttcaagaccaacatgggcaatgtggcgaaactccatctctacaaaaaatgcaaaaataaaaaatatagtactcaagtattcttgatcctgtgtttcaaaactagaatttgtaatgcaaatggagctcagtctaataaaaaagaggttttggtattaaaagttcatacattagacagtatcagccaaaatttgagttagcaacactgttttctttacgagagggtctcacccaaatttatggggagaaatctatttctcaaaaaaaaaaatcttcttttacagaaatgttgagtaaggtgacattttgagcgctaataagcaaaagagcatgcagtgctgttgaataaccctcacttggagaaccaagagaatcctgtcgtttaatgctatattttaatttcacaagttgttcatttaactggtagaatgtcagtccaatctccaatgagaacatgagcaaatagacctttccaggttgaaagtgaaacatactgggtttctgtaagtttttcctcatggcttcatctctatctttactttctcttgaatatgctacacaaagttctttattactacatactaaagtttgcattccagggatattgactgtacatatttatgtatatgtaccatgttgttacatgtaaacaaacttcaatttgaagtgcagctattatgtggtatccatgtgtatcgaccatgtgccatatatcaattatggtcactagaaagtctctttatgatactttttattgtactgtttttcatttcacttgcaaaattttgcagaattcctcctttctacccataaattacatatatttttcttctttagtcatggagaactccccccctcatctcttccctattatctttccctgtgtactggtattattaaaaagacattacatacgcaagtctttctcgacaatcaagaatgttattaatgtgtaatactgagcactttacttcttaataaaaacttgatatagtagca

SEQ ID NO: 3 - DNA, 14035 bp, Mus musculus
  source 1..14035: mol_type = other DNA
agtgcagcgcgaatgcgcgagcaggcggccaaggccctgaggtgcggcctgccccagagcgctgagcagctgtcgcaccgcagacgggccacggccgccgcggcattcagccccgtgccgcaccatgaggtccgcgggcccctcctgaactgcggctgccggcgcaaggggcctcggggaaccggggccatgcgcgggctgcgctgacgatgccgctcggcgcgcctgctctcctggcgctggccctaggcctgggcctgtggctcggagcgctggccggagaccctgggcgcggctgcgggccttgcccgctcccctgcttttgcggccctgcgcccgacgccgcctgccgcgtcaattgctccggccgctggctgcagacgctagggccgagtctgcgcatcccggctgacgccaccgcgctagacctgtcccacaacctacttcagacgctggacatagggctcctggtgaacctttcagcactggtggagctggacttaagcaacaataggatttctacattagaagaaggcgtgtttgctaatttatttaatttaagtgaaataaacctgagtgggaacccatttgagtgcaactgtggtctggcgtggctgccacgctgggcaaaggaacatcaggtgcatgtggtacagtctgaggcaaccacgtgcagggggcccattccactggccggccaacctctcctcagtatccccttattggacaatgcctgtggtgaggaatatgtcgcctgcctccctgacaacagctcaggtgctgtggcagcagtacccttctactttgcccatgagggaccactggagactgaggcctgcagtgccttctgcttctcagctggtgagggccttgcagccctctcggagcagaatcaatgcctctgtggggcaggccaggcctctaactcttctgcagcctgctcatcctggtgttccagcatctcactgtccctcaactctgcctgtgggggccccaccttgctccagcacacctttcccgcctccccaggggctaccctggtggggccccatggacccctggcctctggccagccagcagacttccatattacttcctccctgcccattagctcaacacgctggaactttggagatggctcacctgaggtggacatggctagccctgctgccacccacttctatgtgctacctgggagctatcacatgacagttgtgctggccttgggggctggctcagctctgctggagacagaggtgcaagtagaagcaacacctactgtcctggagcttgtgtgcccatcctttgtgcacagtaacgagagccttgaacttggcatacggcatcgtggaggctcagcccttgaggtcacctatagcatcctggctttggataaagagcctgcccaagtggtccacccactctgccccttggacacagagatcttccctggcaatgggcactgctaccgcctggtggcagagaaggcaccctggctgcaagcacaggagcaatgtcggacttgggctggagctgccttggctatggtggacagtcctgccatccagcacttcctagtctccaaggttaccaggagcctggatgtgtggatcggcttctcatctgtggaggggacagaaggcctggatccccggggtgaggccttcagcctggaaagctgccagaactggctgcctggggagccacacccagccacagcagagcattgtgtgcggttggggcctgctgggcaatgcaacacagacctgtgttcagcaccacatagttatgtctgcgagctgcggcctggagggcctgtgtgggatactgagaactttgtcatgggaatgtctggtggaggcctgtctggacctctgcatccattggcacagcaggaaactgtccagggcccgctgcgacctgtggaggtcatggtgttccctggcctgagccctagccgggaagcctttctcactgcagcagagttttctacccagaagcttgaagagcctgcccagatgcggctgcaggtgtaccggccctcaggaggagcagcagcggtcccagaaggcagcagtgagcctgacaacaggactgagccggcccccaagtgtgtgcctgaggaactctggtgtccaggagccaatgtctgcataccttttgatgcttcgtgtaactctcatgtctgcatcaatgggtctgtgtcaaggctgggactctctagggccagctacacactatggaaggagttctttttttctgtgcctgcagggcctcccacacagtacttggtcaccctccacagccaggatgttcctatgctccctggtgacctcattggcttacagcatgatgctggcccaggtactctcctacagtgtcccctggcttccagctgtcctggccaagccctgtacctttcaaccaatgcctcagattggatgaccaacctgcctgtccatctggaagaagcttgggctggccctgtctgctccctacagctgcttttggtcacagagcggcttacaccactgctgggccttgggcctaaccctggactacagcatcctgggcactatgaggtccgggctacagtgggcaacagcgtatccaggcaaaacctgtcctgcagcttcagtgtggtctctccaatagctgggctgagagtcattcaccccatcccccttgatggccacatctatgtgccaaccaacggctcagtcttggtgcttcaggtagattctggtgctaatgccactgccacagctcagtggtttgggggcaatattagtgctcccttcgaggatgcctgccctcctgaggtggattttctcaagcaagattgcacagaagaggccaatggcaccctgttctcagtgttgatgttgcccaggctcaaggagggggatcacacagtggagattgtggcgcaaaatggagccagccaggccaacctcagcctgagagtgacagctgaggagcccatctgtggcctccgtgctgtgccaagccctgaggcccgagtgctacagggcatcctggtgcggtacagccccatggtagaggctggctcagacgtagccttccggtggaccattgatgataagcagtctctgactttccacaacactgtcttcaatgtaatataccagagtgctgccatcttcaagctctcgctgacagcctctaaccatgtgagcaacatcaccgtgaactacaatgtcactgtggagcggatgaacaagatgcatgggctgtgggtatctgcagtaccgactgtgttaccccccaatgccacactggcactgacaggtggtgtgctggtggattcagctgtggaggtggccttcctgtggaactttggggatggggagcaggtactccgccagttcaagcctccatacgacgagtccttccaggttccagaccccacagtggcgcaggtgctggtggaacacaataccacgcatatttacactaccccaggtgagtacaacttgaccgtccttgtgtccaatacctatgagaatctgacgcagcaggtgacagtaagtgtgcgcactgttcttcccaacgtggctattggcatgagcagcaacgtcctggtggccggccaacccatcaccttttccccatatccgctgccctcaactgatggtgtcctatatacgtgggactttggggatggctcacctgtcctaatacagagccagccagtactcaaccacacctacagcatgacgggcgcttacagaatcaccttggaggttaacaacacagtgagcagcgtgacagcacatgcagacatccgtgtcttccaggagttgcatggcctgactgtgtatttgagcccatctgtggagcagggagcccccatggtagtcagtgcttctgtggagtcaggtgacaacattacatggacatttgacatgggagatggcacagtattcacaggccctgaggctacagtacaacatgtatacctgcgggcccagaacttcacagtgacagtagaagcagccaaccctgctggccacttgtcccagagcctgcatgtgcaagtgtttgtcctggaggtattacacatagagccctctacctgcatccccacacagcccagtgcccagcttatggcccatgtcactggggaccctgtccattacctctttgattggacttttggagatggctcctccaatgtgactgtccatgggcatccatcagtgacacacaacttcactaggagtggcatattcccactggcattggttttgtcaagccatgtgaacaaggcacattattttaccagcatctgtgtggaacctgagatacgtaacatcaccctgcagcctgagaggcagtttgtgaagctgggggatgaagcccggctggttgcctattcttggcccccattcccgtaccgatatacctgggactttggcactgaagacactacccacacccaaactgggggctctgaagtgaaatttatctaccgagaaccaggctcctacttggtgatagtcactgtgtccaacaacatctcttctaccaatgactcagcttttgtggaggtccaggagcctgtgttagtcactggcatcaggatcaatggctcccatgtgttagagctgcagcagccttacctgttgtcagccatgggcagtgggagccctgccacctacctatgggaactgggagacggcagccagagtgagggccctgaggtcacccatatctacagcagcaccggtgatttcactgtcagggtgtctggctggaatgaggtgagccgtagtgaggcccaactcaacatcaccgtaaagcagcgtgtgcggggcctcaccatcaatgccagccgcacagtggtgcccctgaatggcagtgtgagctttagtaccttgttggaggtagggagtgatgtgcattactcctgggtgctctgtgacagatgcacacccattccaggcggacccactatttcctacaccttccgctctgtgggcaccttcaatattattgtcacagctgagaatgaggtgggttctgcccaggacagcatcttcatctatgttctccagttcattgaggggctgcaggtggcagggggtgataacggctgctgcttccctaccaactacacactgcagctgcaagctgcagttagggatggcaccaacatctcttatagctggacagcgcagcaggagggcagcctcatcacactcttcggcagtggcaagtgcttttctctcacttcactcaaagctagcacctactatgtccatcttagggccactaacatgttgggtagtgccgcagccaaccgtactatagactttgtggaacctgtggagagtctaatcctatctgcatcccctaatccagctgctgtcaacatgagtctcaccctttgtgctgaattggctggtggcagtggtgttgtttacacttggtacctggaggaaggactgagttggaagacctccatgccatctaccacccacacctttgctgcacctggcctgcacttggtcagagtcacagctgagaaccagttgggctcagtcaatgccacagttgaagtggccatacaggtgcctgtgggtggtctgagtatcagaaccagtgagccagatagcatctttgtggcagctggctccactctacccttctggggtcagctggctgaaggtaccaatgtgacttggtgctggaccttgccaggtgggagtaaggacagccagtacattgctgtgcgcttctccacggctggcagtttctctctgcagctcaatgcttccaatgcagtcagttgggtctcagccatgtacaacctcacagtagaggaacccattgtgaacctgatgctgtgggccagcagcaaggtggtggcgcctgggcagccagtccactttgaaatcctgctggcagctggctctgctcttactttccgacttcaggttggtgggtctgtccctgaagtgctccctagcccccacttctcccacagcttctttcgggttggagaccatctggtgaatgtgcaggctgagaaccacgtgagtcatgcccaggcacaggttcgcatcctagtgctggaggctgttgttgggctgcaggtacccaactgctgtgagccaggcatggccacaggcactgagaagaacttcacagcccgggtgcagcgaggttcccgagttgcctatgcctggtatttctccttgcaaaaggttcagggtgactcattggttattttatctggccgtgatgtcacctacacacctgtggctgcagggttactggagatccatgtgcgtgcctttaatgaactgggtggtgtgaacctcacactcatggtggaagtccaggatatcatccagtatgtaacacttcagagtggccgctgctttaccaaccgctcagccaggtttgaggctgctacaagccccagtccacggcgtgtaacctaccactgggactttggggatgggactccggtacagaaaacagaggaattctgggctgatcactactacctgcgtcctggggactaccatgtagaggtgaatgctacaaacctagtgagtttttttgtggcacaggccacagtgactgtccaggtgctggcctgtagggagcctgaagtggaggtagcactgcctctgcaggtgctgatgcggcgttcccagcgcaactacctggaggcccatgttgatctacgcaactgtgtctcctaccagactgaatatcggtgggagatatatcgtactgccagctgccaacggccaggacgtatggctcaaatggtactgcctggtgtggatgtgagccgaccccagctggtggtgccacggctggcactacctgtgggccactactgctttgtgtttgtggtttcatttggggacacaccattggcacggagcatccaggccaatgtgacagtggctgctgagcgtctggtacccatcattgaaggtggctcataccgggtatggtcagacacgcaggacctggtgctggatgggagcaagtcctatgaccctaatttagaggatggtgatcagacaccgctcaacttccattgggcctgcgtggcttcaacacagagtgagacaggtggctgcgtgctaaactttgggccccgtgggagcagtgtggtcaccatccccctggaacgcctagaagctggtgtagaatataccttcaacctcatagtgtggaaagcaggtcggaaggaagaagccaccaaccagacagtactgatccgaagtgggcgagtccccattgtgtcccttgagtgtgtgtcctgcaaggcacaagcagtgtatgaagtgagccgaagttcctatgtgtacttggagggtcactgccacaactgcagcagaggctacaagcaagggtgttgggccgctcgcactttcagcaataagactctggtgctgaacgagacgacaacatccacgggtagcaccggcatgaatctggtagtgcggccaggtgcactgcgtgatggggagggctacatcttcacactcactgtgctgggccactcaggggaagaagagggctgtgcctcaatccgcctctcacccaaccgccccccacttggaggctcctgccgcctattcccattggattctgtgcgtggcctcaccactaaggttcacttcgagtgcacaggttggcgtgatgcagaagatggtggagccccactggtatatgctctgttgctgaagcgctgtcgtcagagctactgtgagaatttctgcatctataagggtagtctttccacctatggggctgtcctgcctcctggattccagcccctttttgtggtgagcctagctgtggttgtacaagaccagctgggtgctgctgttgttgcactcaacaggtctctgaccattgtcctaccagaacccagtggaaatcctgcagacctcgtaccctggctgcacagcctgactgccagtgtgctgcctgggctcctgaagcaggctgaccctcagcatgtcatcgagtactctttagccctcattactgtgctcaatgagtatgagcaggccccagatgtgtcagagcccaacgttgagcagcagctgagagcccaaatgcgtaagaacatcacggagaccctgatctccctgcgggtcaacactgtggatgacatccagcagattactgctgcgttagcccagtgcatggtgtccagcagggagctcatgtgccgctcctgcctgaagaagatgctgcagaagctggagggcatgatgcgcatcttgcaagctgaaaccactgagggcaccttgacacccaccaccattgctgacagcattctcaatatcacaggtgacctcatccacctggccagcttggacatgcagggcccacagcccttggagctgggggtggagccgccctccctgatggtggcttctaaggcctacaacctctcgtcggccctcatgcgtatcctcatgcgttcccgagtgctcaatgaggagcctctgactctggctggcgaggagattgtggccctaggcaagcgctcagacccactcagtcttctgtgttatggcaaagccttggggcccagctgccacttctccattcctgaggccttcagtggggccctgtcgaacctcagtgatgtggtgcagctcatcttccttgtggattccaaccccttcccctttggctacattagcaactacactgtctccaccaaagtggcatctatggcattccagacacagactggtacccagatccctattgagcagttggctgcagagcgtgccatcacagtgaaggtgccaaacaactcagaccaggctgcccagagctctcacaatcctgtgggttctaccattgtacagccccagacctctgtcagcgctgtggtcactgcagacaacagtaaccctcaagctggcctgcatctgaggatcacttacacagtgcttaatgagcgctatctgtccgcggagcctgagccctacctggctgtgtacctgcactcagtgtcccagcccaatgagtacaactgctcggccagcaggaggatcagcctggaggtgctggagggtgctgaccacaggctctacaccttcttcattgccccaggaactgggaccttggacaggagttactacctgaacctgacgagccatttccactggtctgccctggaggtgtctgtgggcctctatacatccctgtgccagtacttcagtgaggagatgatgatgtggagaactgagggcattgtacccctggaggagacctcacccagccaggctgtctgcctcacccgccacctcactgccttcggtgccagcctttttgtgcctcccagccatgtccagttcatctttcctgaaccatctgcaagcatcaactacattgtcctcctgacgtgtgtcatatgcctggtgacctatgtggtcatggctatgatcctgcgcaagctggaccagctggatgttagccgtgtccgtgtcatcccgttctgtgggaaggggggccgcttcaaatatgaaatacttgttaagaccggctggagccgaggttcaggtaccactgctcacgtcggcatcatgctgtatggagaggacaaccgtagtggtcaccggcacttagatggggacagagcctttcatcgaaacagcctggacatcttccaaattgccaccccgcacagcctgggcagcgtgtggaagatccgagtgtggcatgataacaaagggctcagccctgcctggttcctgcaacacattatcgtccgggacctacagagtgcccgcagcaccttctttttggtcaatgactggctgtctgtggagactgaggccaacgggggcctagtggagaaggaggtgcttgcagcaaatgaagctgcgctgtggcagttccagcggctcctcgtggctgagttgcagagaggcttctttgacaagcacatctggctctccatatgggaccggccgcctcgtagccgctttactagagtccagagggttacctgctgtgttctcctcctctgcctcttcctggctgccaatgctgtgtggtacggagttgtgagagacaccacctacagcatgggacctgtgtccagtctgatctctcctggtgttgacacagttgctattggccttgtgtccagtgtggtagtctaccctgtctatttggctgtcctgttcctcttccggatgtctcggagtaaggtgtctggggaccaaaaccccacacccacagggcagcaggccctagatgttgacagctacctggacccatctgtgctagacagctccctcctcacactctctggcctcacagaggccttcgctgggcaagtgaagaatgacttgtttctggaagatgctaaaagcctggtgtgctggccgtccagtgagggtactcttagttggccagacctgctcagtgacccatctgttgtgagcagcacactgcagcgattgacccagggcaggcctggctgcatgttgggctcagaggaagatggtgcctctctggttagcccttccttgcctgccaaatacttgtcggcctcagatgaagacctgatccatcaggtactggctgatggggccaacaacctggttcctacccaggacaccctcctagaaacagacctgcttactagcctgtccagtgttcctggagagaaaacagaaactctgatactgcagacagtgggggaggagagaccagccagcatgggcctcagttgggaacagtccccggtgacaaggctttccaggacaggactggtggaaggttttcagaagcgactgctgcctgcctggtgtgccccactggcccatgggctcagcctgctcttggtagctgtagctgtagcagtttcagggtggattggtgccagcttccctcccagcgtgagtgtcatgtggcttctctcaagcagctccagcttcctggcctcctttcttggctgggagccccttaaggtcctgctggaagccctctacttctcccttgtggccaagcggcttcaccctgatgaagatgacactctggtagagagcccagctgtaacccctgtaagtgagcgtgtgccccgtgtgcggcccccacatggttttgcactcttcttggcaaaggaggaagctcgaaaggtcaagaggctccatgacatgttgaagagactcttggtgtacatgcttttcttactggtgacgctgctggccaactatggggatgcttcttgccatggtcacgcctatcgcctgcagagcgccatcaagcaggagctggatagccaagcctttctggccatcacccggtctgatgagttctggccatggatgtcacatgtgttcttgccctatgtccatgggaaccagtctagccctgagctgggccctccacggctacggcaggtgcgactgcaggaagcattctgcccagacccttctagctcggagcacatgtgttcagcggcaggaagcttgagcaccagtgactatggcattggctggcagagtgtggtgcaaaatggctctgagacatgggcctactctgcaccggacctgctgggcgcctggtactggggctactgtgcagtgtatgacagtgggggctacatacaggagttaggactgagtctggaggagagccgtgcacgactgggcttcctgcagctgcacaactggcttgacagcaggagccgtgcagtgtttgtagaactcacccgctacagccctgctgtggggctccatgctgctgttacactgcgcctcgagttccctgtagccggccatgcactggctgccttcagtgtccgcccatttgcgttacggcgactcagcactggcctgtctctaccactgctcacctcggtgtgcttgctgctgtttgccctatacttttccatggccgaggtgcagacctggcgcaaagatgggtgtgcgtgcacagcaaggcctgacacctgggcacggtgcctactggtgatactgacagcagctacagggcttgtacgcctggcccagctgggcattgctgatcgccagtggacccattttgtgcaggaccatccacgccacttcactagcttcgaccaagtggctcagctgggctctgtggcccgtggcctggcagcttcactactcttcctgcttttggtcaaggctgcccagcagctgcggtttgtgcgccagtggtctgtttttggcaagacattgtgtcgggccttgcccgagctcatgggagccaccttgggcctggtgctgcttggtgtggcctatgcacagatggccatcctgcttatttcctctggtgctgacactctctacaacatggcccgtgccttcctggtgctgtgccctggggcgagggtccccactctgtgcccttcagagtcctggtacctgtcccctctgctgtgtgtggggctctgggctctgcgagtgtggggtgccctgaggttaggggctatcctcctgcgttggcggtaccacgccttgcgtggcgagctctaccgcccagcatgggaaccccaggactatgagatggtggagcttttcctgcgtaggcttcggctctggatgggcttcagcaaggtcaaggagttccgccacaaagtccgctttgaaggaatggatccactgccttcccgctcatccaggggctccaagtcatccccagttgtgctcccacctagctcaggctcagaagcttcacacccatccacctcgtccagccaaccagacgggccgagcgccagcttaagccgctcgacgctgaagctggaaccagagccctctcgcctccatgctgtgtttgaaagtctgcttgtccagtttgaccgactcaaccaggccacagaggacgtctaccagctggagcaacaactccagagccttcaaggccatgggcacaatggacctccttcctctccctcccctggctgcttcccaggctctcagccagctttgcccagccgcctttctcgggccagtcaggggctggatcagactgtaggccccaacagggtgtccctgtggcctaataacaaggtccaccccagcagcacttaggccctaggggtcttggccattcccttccctgggaatgcctgagcttcacactggcctctcagagccagggtggacaccactcagtattaccttctgctgccctctaggttgggccaggcagaacggctgcatgccagttcttttgggtacaggtattgctgccttccttacctgtccacatatggggcttctgcactttaaaaaggctgtgtggccagccaggacccagggtcccctccccacaggaggacacagcagtattggaccaagtgagtacccagcctccaagatgctaatttattcgccccccctccccccaagtcctcaggttcagcgggctgcgcccagctctacccctaggtggccatctcctcttgctaaaaatctaagctggagggagggctaagctacacttctcctttatccgtcccctaagttattacctctcttgtttctgcagcatacttgccctctagctaccatctgctttctatgtccaccatcaataatttatatggggttaaaatgtatatatttttgtatgtcagtattttctacttgggctgaaaatgggtccattgttacccagggcaggggtacaggggaagctgcagctattactggatccgagtctggcctcgggcaggtgctggacagcaattggggccaccgtggccttgttttcctcccttgtctggggccaggcagcacgcagatctgctggtttcagatctgagcaagggcaggactactgtgggatgaaagatgccaagaagttactggacaagcccaccaatggggcttgcccccatggcgggtgggggaaagagaatgtatgtgatggcacctgcctgcttctgtgggctcatctgcagctctagcctggattctgccccaaccccaaacagacaagacaaagtcaaataaagaagctgtctgactgc

SEQ ID NO: 4-13 - skipped (10 entries)

SEQ ID NO: 14 - RNA, 10 nt, synthetic construct
  source 1..10: mol_type = other RNA
ggactggact

SEQ ID NO: 15 - RNA, 15 nt, synthetic construct
  source 1..15: mol_type = other RNA
ggactggactggact

SEQ ID NO: 16 - RNA, 13 nt, synthetic construct
  source 1..13: mol_type = other RNA
acggacttggact

SEQ ID NO: 17 - RNA, 10 nt, synthetic construct
  source 1..10: mol_type = other RNA
aaactaaact

SEQ ID NO: 18 - RNA, 12 nt, synthetic construct
  source 1..12: mol_type = other RNA
aaaaaaaaaaaa

SEQ ID NO: 19 - RNA, 10 nt, synthetic construct
  source 1..10: mol_type = other RNA
aaaaaaaaaa

SEQ ID NO: 20 - RNA, 10 nt, synthetic construct
  source 1..10: mol_type = other RNA
cccccccccc

SEQ ID NO: 21 - RNA, 10 nt, synthetic construct
  source 1..10: mol_type = other RNA
gggggggggg

SEQ ID NO: 22 - RNA, 10 nt, synthetic construct
  source 1..10: mol_type = other RNA
  modified_base 1: mod_base = OTHER; note = 2'-deoxy thymidine
  modified_base 2: mod_base = OTHER; note = 2'-deoxy thymidine
  modified_base 3: mod_base = OTHER; note = 2'-deoxy thymidine
  modified_base 4: mod_base = OTHER; note = 2'-deoxy thymidine
  modified_base 5: mod_base = OTHER; note = 2'-deoxy thymidine
  modified_base 6: mod_base = OTHER; note = 2'-deoxy thymidine
  modified_base 7: mod_base = OTHER; note = 2'-deoxy thymidine
  modified_base 8: mod_base = OTHER; note = 2'-deoxy thymidine
  modified_base 9: mod_base = OTHER; note = 2'-deoxy thymidine
  modified_base 10: mod_base = OTHER; note = 2'-deoxy thymidine
tttttttttt

SEQ ID NO: 23 - RNA, 10 nt, synthetic construct
  source 1..10: mol_type = other RNA
tttttttttt

SEQ ID NO: 24 - RNA, 16 nt, synthetic construct
  source 1..16: mol_type = other RNA
  modified_base 1: mod_base = OTHER; note = 2'-O-methyl guanosine
  modified_base 2: mod_base = OTHER; note = 2'-O-methyl cytidine
  modified_base 3: mod_base = OTHER; note = 2'-O-methyl adenosine
  modified_base 4: mod_base = OTHER; note = 2'-O-methyl uridine
  modified_base 5: mod_base = OTHER; note = 2'-O-methyl guanosine
  modified_base 6: mod_base = OTHER; note = 2'-O-methyl guanosine
  modified_base 7: mod_base = OTHER; note = 2'-O-methyl cytidine
  modified_base 8: mod_base = OTHER; note = 2'-O-methyl guanosine
  modified_base 9: mod_base = OTHER; note = 2'-O-methyl guanosine
  modified_base 10: mod_base = OTHER; note = 2'-O-methyl guanosine
  modified_base 11: mod_base = OTHER; note = 2'-O-methyl cytidine
  modified_base 12: mod_base = OTHER; note = 2'-O-methyl guanosine
  modified_base 13: mod_base = OTHER; note = 2'-O-methyl cytidine
  modified_base 14: mod_base = OTHER; note = 2'-O-methyl guanosine
  modified_base 15: mod_base = OTHER; note = 2'-O-methyl guanosine
  modified_base 16: mod_base = OTHER; note = 2'-O-methyl guanosine
gcatggcgggcgcggg

SEQ ID NO: 25 - RNA, 16 nt, synthetic construct
  source 1..16: mol_type = other RNA
  modified_base 1: mod_base = OTHER; note = 2'-O-methyl guanosine
  modified_base 2: mod_base = OTHER; note = 2'-O-methyl adenosine
  modified_base 3: mod_base = OTHER; note = 2'-O-methyl cytidine
  modified_base 4: mod_base = OTHER; note = 2'-O-methyl guanosine
  modified_base 5: mod_base = OTHER; note = 2'-O-methyl guanosine
  modified_base 6: mod_base = OTHER; note = 2'-O-methyl cytidine
  modified_base 7: mod_base = OTHER; note = 2'-O-methyl adenosine
  modified_base 8: mod_base = OTHER; note = 2'-O-methyl uridine
  modified_base 9: mod_base = OTHER; note = 2'-O-methyl guanosine
  modified_base 10: mod_base = OTHER; note = 2'-O-methyl guanosine
  modified_base 11: mod_base = OTHER; note = 2'-O-methyl cytidine
  modified_base 12: mod_base = OTHER; note = 2'-O-methyl guanosine
  modified_base 13: mod_base = OTHER; note = 2'-O-methyl guanosine
  modified_base 14: mod_base = OTHER; note = 2'-O-methyl guanosine
  modified_base 15: mod_base = OTHER; note = 2'-O-methyl cytidine
  modified_base 16: mod_base = OTHER; note = 2'-O-methyl guanosine
gacggcatggcgggcg

SEQ ID NO: 26 - RNA, 16 nt, synthetic construct
  source 1..16: mol_type = other RNA
  modified_base 1: mod_base = OTHER; note = 2'-O-methyl cytidine
  modified_base 2: mod_base = OTHER; note = 2'-O-methyl adenosine
  modified_base 3: mod_base = OTHER; note = 2'-O-methyl uridine
  modified_base 4: mod_base = OTHER; note = 2'-O-methyl guanosine
  modified_base 5: mod_base = OTHER; note = 2'-O-methyl guanosine
  modified_base 6: mod_base = OTHER; note = 2'-O-methyl cytidine
  modified_base 7: mod_base = OTHER; note = 2'-O-methyl cytidine
  modified_base 8: mod_base = OTHER; note = 2'-O-methyl cytidine
  modified_base 9: mod_base = OTHER; note = 2'-O-methyl cytidine
  modified_base 10: mod_base = OTHER; note = 2'-O-methyl guanosine
  modified_base 11: mod_base = OTHER; note = 2'-O-methyl cytidine
  modified_base 12: mod_base = OTHER; note = 2'-O-methyl cytidine
  modified_base 13: mod_base = OTHER; note = 2'-O-methyl guanosine
  modified_base 14: mod_base = OTHER; note = 2'-O-methyl uridine
  modified_base 15: mod_base = OTHER; note = 2'-O-methyl cytidine
  modified_base 16: mod_base = OTHER; note = 2'-O-methyl cytidine
catggccccgccgtcc

SEQ ID NO: 27 - RNA, 19 nt, synthetic construct
  source 1..19: mol_type = other RNA
  modified_base 1: mod_base = OTHER; note = 2'-O-methyl cytidine
  modified_base 2: mod_base = OTHER; note = 2'-O-methyl adenosine
  modified_base 3: mod_base = OTHER; note = 2'-O-methyl cytidine
  modified_base 4: mod_base = OTHER; note = 2'-O-methyl adenosine
  modified_base 5: mod_base = OTHER; note = 2'-O-methyl guanosine
  modified_base 6: mod_base = OTHER; note = 2'-O-methyl cytidine
  modified_base 7: mod_base = OTHER; note = 2'-O-methyl cytidine
  modified_base 8: mod_base = OTHER; note = 2'-O-methyl uridine
  modified_base 9: mod_base = OTHER; note = 2'-O-methyl cytidine
  modified_base 10: mod_base = OTHER; note = 2'-O-methyl uridine
  modified_base 11: mod_base = OTHER; note = 2'-O-methyl uridine
  modified_base 12: mod_base = OTHER; note = 2'-O-methyl uridine
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl uridine
  modified_base 18: mod_base = OTHER; note = 2'-O-methyl guanosine
  modified_base 19: mod_base = OTHER; note = 2'-O-methyl cytidine
cacagcctctttaaagtgc

SEQ ID NO: 28 - RNA, 16 nt, synthetic construct
  source 1..16: mol_type = other RNA
  modified_base 1: mod_base = OTHER; note = 2'-O-methyl uridine
  modified_base 2: mod_base = OTHER; note = 2'-O-methyl guanosine
  modified_base 3: mod_base = OTHER; note = 2'-O-methyl guanosine
  modified_base 4: mod_base = OTHER; note = 2'-O-methyl cytidine
  modified_base 5: mod_base = OTHER; note = 2'-O-methyl cytidine
  modified_base 6: mod_base = OTHER; note = 2'-O-methyl cytidine
  modified_base 7: mod_base = OTHER; note = 2'-O-methyl cytidine
  modified_base 8: mod_base = OTHER; note = 2'-O-methyl guanosine
  modified_base 9: mod_base = OTHER; note = 2'-O-methyl cytidine
  modified_base 10: mod_base = OTHER; note = 2'-O-methyl cytidine
  modified_base 11: mod_base = OTHER; note = 2'-O-methyl guanosine
  modified_base 12: mod_base = OTHER; note = 2'-O-methyl uridine
  modified_base 13: mod_base = OTHER; note = 2'-O-methyl cytidine
  modified_base 14: mod_base = OTHER; note = 2'-O-methyl cytidine
  modified_base 15: mod_base = OTHER; note = 2'-O-methyl cytidine
  modified_base 16: mod_base = OTHER; note = 2'-O-methyl cytidine
tggccccgccgtcccc

SEQ ID NO: 29 - RNA, 16 nt, synthetic construct
  source 1..16: mol_type = other RNA
  modified_base 1: mod_base = OTHER; note = 2'-O-methyl adenosine
  modified_base 2: mod_base = OTHER; note = 2'-O-methyl cytidine
  modified_base 3: mod_base = OTHER; note = 2'-O-methyl uridine
  modified_base 4: mod_base = OTHER; note = 2'-O-methyl guanosine
  modified_base 5: mod_base = OTHER; note = 2'-O-methyl guanosine
  modified_base 6: mod_base = OTHER; note = 2'-O-methyl adenosine
  modified_base 7: mod_base = OTHER; note = 2'-O-methyl adenosine
  modified_base 8: mod_base = OTHER; note = 2'-O-methyl adenosine
  modified_base 9: mod_base = OTHER; note = 2'-O-methyl adenosine
  modified_base 10: mod_base = OTHER; note = 2'-O-methyl cytidine
  modified_base 11: mod_base = OTHER; note = 2'-O-methyl adenosine
  modified_base 12: mod_base = OTHER; note = 2'-O-methyl adenosine
  modified_base 13: mod_base = OTHER; note = 2'-O-methyl cytidine
  modified_base 14: mod_base = OTHER; note = 2'-O-methyl adenosine
  modified_base 15: mod_base = OTHER; note = 2'-O-methyl cytidine
  modified_base 16: mod_base = OTHER; note = 2'-O-methyl cytidine
actggaaaacaacacc

SEQ ID NO: 30 - RNA, 26 nt, synthetic construct
  source 1..26: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-methyl uridine
  modified_base 11: mod_base = OTHER; note = 2'-O-methyl cytidine
  modified_base 12: mod_base = OTHER; note = 2'-O-methyl cytidine
  modified_base 13: mod_base = OTHER; note = 2'-O-methyl adenosine
  modified_base 14: mod_base = OTHER; note = 2'-O-methyl uridine
  modified_base 15: mod_base = OTHER; note = 2'-O-methyl uridine
  modified_base 16: mod_base = OTHER; note = 2'-O-methyl cytidine
  modified_base 17: mod_base = OTHER; note = 2'-O-methyl uridine
  modified_base 18: mod_base = OTHER; note = 2'-O-methyl guanosine
  modified_base 19: mod_base = OTHER; note = 2'-O-methyl cytidine
  modified_base 20: mod_base = OTHER; note = 2'-O-methyl cytidine
  modified_base 21: mod_base = OTHER; note = 2'-O-methyl uridine
  modified_base 22: mod_base = OTHER; note = 2'-O-methyl guanosine
  modified_base 23: mod_base = OTHER; note = 2'-O-methyl guanosine
  modified_base 24: mod_base = OTHER; note = 2'-O-methyl cytidine
  modified_base 25: mod_base = OTHER; note = 2'-O-methyl cytidine
  modified_base 26: mod_base = OTHER; note = 2'-O-methyl cytidine
ggactggactccattctgcctggccc

SEQ ID NO: 31 - RNA, 16 nt, synthetic construct
  source 1..16: mol_type = other RNA
  modified_base 1: mod_base = OTHER; note = 2'-O-methyl uridine
  modified_base 2: mod_base = OTHER; note = 2'-O-methyl uridine 5'-thiophosphate
  modified_base 3: mod_base = OTHER; note = 2'-O-methyl adenosine 5'-thiophosphate
  modified_base 4: mod_base = OTHER; note = 2'-O-methyl adenosine 5'-thiophosphate
  modified_base 5: mod_base = OTHER; note = 2'-O-methyl adenosine 5'-thiophosphate
  modified_base 6: mod_base = OTHER; note = 2'-O-methyl guanosine 5'-thiophosphate
  modified_base 7: mod_base = OTHER; note = 2'-O-methyl uridine 5'-thiophosphate
  modified_base 8: mod_base = OTHER; note = 2'-O-methyl guanosine 5'-thiophosphate
  modified_base 9: mod_base = OTHER; note = 2'-O-methyl cytidine 5'-thiophosphate
  modified_base 10: mod_base = OTHER; note = 2'-O-methyl uridine 5'-thiophosphate
  modified_base 11: mod_base = OTHER; note = 2'-O-methyl guanosine 5'-thiophosphate
  modified_base 12: mod_base = OTHER; note = 2'-O-methylene-4'-bridged (LNA) adenosine 5'-thiophosphate
  modified_base 13: mod_base = OTHER; note = 2'-O-methyl adenosine 5'-thiophosphate
  modified_base 14: mod_base = OTHER; note = 2'-O-methylene-4'-bridged (LNA) guanosine 5'-thiophosphate
  modified_base 15: mod_base = OTHER; note = 2'-O-methyl cytidine 5'-thiophosphate
  modified_base 16: mod_base = OTHER; note = 2'-O-methylene-4'-bridged (LNA) 5-methyl cytidine 5'-thiophosphate
ttaaagtgctgaagcc

SEQ ID NO: 32 - RNA, 25 nt, synthetic construct
  source 1..25: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-methyl uridine
  modified_base 11: mod_base = OTHER; note = 2'-O-methyl uridine 5'-thiophosphate
  modified_base 12: mod_base = OTHER; note = 2'-O-methyl adenosine 5'-thiophosphate
  modified_base 13: mod_base = OTHER; note = 2'-O-methyl adenosine 5'-thiophosphate
  modified_base 14: mod_base = OTHER; note = 2'-O-methyl adenosine 5'-thiophosphate
  modified_base 15: mod_base = OTHER; note = 2'-O-methyl guanosine 5'-thiophosphate
  modified_base 16: mod_base = OTHER; note = 2'-O-methyl uridine 5'-thiophosphate
  modified_base 17: mod_base = OTHER; note = 2'-O-methyl guanosine 5'-thiophosphate
  modified_base 18: mod_base = OTHER; note = 2'-O-methyl cytidine 5'-thiophosphate
  modified_base 19: mod_base = OTHER; note = 2'-O-methyl uridine 5'-thiophosphate
  modified_base 20: mod_base = OTHER; note = 2'-O-methyl guanosine 5'-thiophosphate
  modified_base 21: mod_base = OTHER; note = 2'-O-methylene-4'-bridged (LNA) adenosine 5'-thiophosphate
  modified_base 22: mod_base = OTHER; note = 2'-O-methyl adenosine 5'-thiophosphate
  modified_base 23: mod_base = OTHER; note = 2'-O-methylene-4'-bridged (LNA) guanosine 5'-thiophosphate
  modified_base 24: mod_base = OTHER; note = 2'-O-methyl cytidine 5'-thiophosphate
  modified_base 25: mod_base = OTHER; note = 2'-O-methylene-4'-bridged (LNA) 5-methyl cytidine 5'-thiophosphate
ggactggacttaaagtgctgaagcc

SEQ ID NO: 33 - skipped

SEQ ID NO: 34 - RNA, 16 nt, synthetic construct
  source 1..16: mol_type = other RNA
  modified_base 1: mod_base = OTHER; note = 2'-O-(2-methoxyethyl) thymidine
  modified_base 2: mod_base = OTHER; note = 2'-O-(2-methoxyethyl) thymidine 5'-thiophosphate
  modified_base 3: mod_base = OTHER; note = 2'-O-(2-methoxyethyl) adenosine 5'-thiophosphate
  modified_base 4: mod_base = OTHER; note = 2'-O-(2-methoxyethyl) adenosine 5'-thiophosphate
  modified_base 5: mod_base = OTHER; note = 2'-O-(2-methoxyethyl) adenosine 5'-thiophosphate
  modified_base 6: mod_base = OTHER; note = 2'-O-(2-methoxyethyl) guanosine 5'-thiophosphate
  modified_base 7: mod_base = OTHER; note = 2'-O-(2-methoxyethyl) thymidine 5'-thiophosphate
  modified_base 8: mod_base = OTHER; note = 2'-O-(2-methoxyethyl) guanosine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) thymidine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-methylene-4'-bridged (LNA) adenosine 5'-thiophosphate
  modified_base 13: mod_base = OTHER; note = 2'-O-(2-methoxyethyl) adenosine 5'-thiophosphate
  modified_base 14: mod_base = OTHER; note = 2'-O-methylene-4'-bridged (LNA) guanosine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-methylene-4'-bridged (LNA) 5-methyl cytidine 5'-thiophosphate
ttaaagtgctgaagcc

SEQ ID NO: 35 - RNA, 16 nt, synthetic construct
  source 1..16: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl cytidine 5'-thiophosphate
  modified_base 4: mod_base = OTHER; note = 2'-O-methyl cytidine 5'-thiophosphate
  modified_base 5: mod_base = OTHER; note = 2'-O-methyl cytidine 5'-thiophosphate
  modified_base 6: mod_base = OTHER; note = 2'-O-methyl cytidine 5'-thiophosphate
  modified_base 7: mod_base = OTHER; note = 2'-O-methyl guanosine 5'-thiophosphate
  modified_base 8: mod_base = OTHER; note = 2'-O-methyl cytidine 5'-thiophosphate
  modified_base 9: mod_base = OTHER; note = 2'-O-methyl cytidine 5'-thiophosphate
  modified_base 10: mod_base = OTHER; note = 2'-O-methyl guanosine 5'-thiophosphate
  modified_base 11: mod_base = OTHER; note = 2'-O-methyl uridine 5'-thiophosphate
  modified_base 12: mod_base = OTHER; note = 2'-O-methylene-4'-bridged (LNA) 5-methyl cytidine 5'-thiophosphate
  modified_base 13: mod_base = OTHER; note = 2'-O-methyl cytidine 5'-thiophosphate
  modified_base 14: mod_base = OTHER; note = 2'-O-methylene-4'-bridged (LNA) 5-methyl cytidine 5'-thiophosphate
  modified_base 15: mod_base = OTHER; note = 2'-O-methyl cytidine 5'-thiophosphate
  modified_base 16: mod_base = OTHER; note = 2'-O-methylene-4'-bridged (LNA) adenosine 5'-thiophosphate
ggccccgccgtcccca

SEQ ID NO: 36 - RNA, 14 nt, synthetic construct
  source 1..14: mol_type = other RNA
  modified_base 1: mod_base = OTHER; note = 2'-O-(2-methoxyethyl) thymidine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) guanosine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) 5-methyl cytidine 5'-thiophosphate
  modified_base 8: mod_base = OTHER; note = 2'-O-(2-methoxyethyl) guanosine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) 5-methyl cytidine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) thymidine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
tggccccgccgtcc

SEQ ID NO: 37 - RNA, 14 nt, synthetic construct
  source 1..14: mol_type = other RNA
  modified_base 1: mod_base = OTHER; note = 2'-O-(2-methoxyethyl) thymidine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) guanosine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) 5-methyl cytidine 5'-thiophosphate
  modified_base 8: mod_base = OTHER; note = 2'-O-(2-methoxyethyl) guanosine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-methylene-4'-bridged (LNA) 5-methyl cytidine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-methylene-4'-bridged (LNA) thymidine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-methylene-4'-bridged (LNA) 5-methyl cytidine 5'-thiophosphate
tggccccgccgtcc

SEQ ID NO: 38 - RNA, 16 nt, synthetic construct
  source 1..16: mol_type = other RNA
  modified_base 1: mod_base = OTHER; note = 2'-O-methylene-4'-bridged (LNA) guanosine
  modified_base 2: mod_base = OTHER; note = 2'-O-methylene-4'-bridged (LNA) adenosine 5'-thiophosphate
  modified_base 3: mod_base = OTHER; note = 2'-O-methylene-4'-bridged (LNA) adenosine 5'-thiophosphate
  modified_base 4: mod_base = OTHER; note = 2'-deoxy guanosine 5'-thiophosphate
  modified_base 5: mod_base = OTHER; note = 2'-deoxy adenosine 5'-thiophosphate
  modified_base 6: mod_base = OTHER; note = 2'-deoxy guanosine 5'-thiophosphate
  modified_base 7: mod_base = OTHER; note = 2'-deoxy adenosine 5'-thiophosphate
  modified_base 8: mod_base = OTHER; note = 2'-deoxy thymidine 5'-thiophosphate
  modified_base 9: mod_base = OTHER; note = 2'-deoxy guanosine 5'-thiophosphate
  modified_base 10: mod_base = OTHER; note = 2'-deoxy thymidine 5'-thiophosphate
  modified_base 11: mod_base = OTHER; note = 2'-deoxy thymidine 5'-thiophosphate
  modified_base 12: mod_base = OTHER; note = 2'-deoxy guanosine 5'-thiophosphate
  modified_base 13: mod_base = OTHER; note = 2'-deoxy thymidine 5'-thiophosphate
  modified_base 14: mod_base = OTHER; note = 2'-O-methylene-4'-bridged (LNA) thymidine 5'-thiophosphate
  modified_base 15: mod_base = OTHER; note = 2'-O-methylene-4'-bridged (LNA) guanosine 5'-thiophosphate
  modified_base 16: mod_base = OTHER; note = 2'-O-methylene-4'-bridged (LNA) guanosine 5'-thiophosphate
gaagagatgttgttgg

SEQ ID NO: 39 - RNA, 25 nt, synthetic construct
  source 1..25: mol_type = other RNA
  modified_base 1: mod_base = OTHER; note = 2'-O-(2-methoxyethyl) guanosine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) adenosine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) thymidine 5'-thiophosphate
  modified_base 6: mod_base = OTHER; note = 2'-O-(2-methoxyethyl) guanosine 5'-thiophosphate
  modified_base 7: mod_base = OTHER; note = 2'-O-(2-methoxyethyl) guanosine 5'-thiophosphate
  modified_base 8: mod_base = OTHER; note = 2'-O-(2-methoxyethyl) adenosine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-(2-methoxyethyl) thymidine 5'-thiophosphate
  modified_base 21: mod_base = OTHER; note = 2'-O-(2-methoxyethyl) 5-methyl cytidine 5'-thiophosphate
  modified_base 22: mod_base = OTHER; note = 2'-O-(2-methoxyethyl) 5-methyl cytidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-(2-methoxyethyl) guanosine 5'-thiophosphate
  modified_base 25: mod_base = OTHER; note = 2'-O-(2-methoxyethyl) guanosine 5'-thiophosphate
ggactggacggaccctgggtcctgg

SEQ ID NO: 40 - RNA, 16 nt, synthetic construct
  source 1..16: mol_type = other RNA
  modified_base 1: mod_base = OTHER; note = 2'-O-(2-methoxyethyl) thymidine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) guanosine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) 5-methyl cytidine 5'-thiophosphate
  modified_base 8: mod_base = OTHER; note = 2'-O-(2-methoxyethyl) guanosine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) 5-methyl cytidine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) thymidine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) 5-methyl cytidine 5'-thiophosphate
tggccccgccgtcccc

SEQ ID NO: 41 - RNA, 16 nt, synthetic construct
  source 1..16: mol_type = other RNA
  modified_base 1: mod_base = OTHER; note = 2'-O-methyl uridine
  modified_base 2: mod_base = OTHER; note = 2'-O-methyl uridine 5'-thiophosphate
  modified_base 3: mod_base = OTHER; note = 2'-O-methyl adenosine 5'-thiophosphate
  modified_base 4: mod_base = OTHER; note = 2'-O-methyl adenosine 5'-thiophosphate
  modified_base 5: mod_base = OTHER; note = 2'-O-methyl adenosine 5'-thiophosphate
  modified_base 6: mod_base = OTHER; note = 2'-O-methyl guanosine 5'-thiophosphate
  modified_base 7: mod_base = OTHER; note = 2'-O-methyl uridine 5'-thiophosphate
  modified_base 8: mod_base = OTHER; note = 2'-O-methyl guanosine 5'-thiophosphate
  modified_base 9: mod_base = OTHER; note = 2'-O-methyl cytidine 5'-thiophosphate
  modified_base 10: mod_base = OTHER; note = 2'-O-methyl uridine 5'-thiophosphate
  modified_base 11: mod_base = OTHER; note = 2'-O-methyl guanosine 5'-thiophosphate
  modified_base 12: mod_base = OTHER; note = 2'-O-methylene-4'-bridged (LNA) guanosine 5'-thiophosphate
  modified_base 13: mod_base = OTHER; note = 2'-O-methyl adenosine 5'-thiophosphate
  modified_base 14: mod_base = OTHER; note = 2'-O-methylene-4'-bridged (LNA) guanosine 5'-thiophosphate
  modified_base 15: mod_base = OTHER; note = 2'-O-methyl cytidine 5'-thiophosphate
  modified_base 16: mod_base = OTHER; note = 2'-O-methylene-4'-bridged (LNA) 5-methyl cytidine 5'-thiophosphate
ttaaagtgctggagcc

SEQ ID NO: 42 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) 5-methyl cytidine 5'-thiophosphate
  modified_base 11: mod_base = OTHER; note = 2'-O-(2-methoxyethyl) 5-methyl cytidine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) thymidine 5'-thiophosphate
  modified_base 14: mod_base = OTHER; note = 2'-O-(2-methoxyethyl) thymidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) 5-methyl cytidine 5'-thiophosphate
  modified_base 19: mod_base = OTHER; note = 2'-O-(2-methoxyethyl) 5-methyl cytidine 5'-thiophosphate
  modified_base 20: mod_base = OTHER; note = 2'-O-methylene-4'-bridged (LNA) thymidine 5'-thiophosphate
  modified_base 21: mod_base = OTHER; note = 2'-O-(2-methoxyethyl) guanosine 5'-thiophosphate
  modified_base 22: mod_base = OTHER; note = 2'-O-methylene-4'-bridged (LNA) guanosine 5'-thiophosphate
  modified_base 23: mod_base = OTHER; note = 2'-O-(2-methoxyethyl) 5-methyl cytidine 5'-thiophosphate
  modified_base 24: mod_base = OTHER; note = 2'-O-methylene-4'-bridged (LNA) 5-methyl cytidine 5'-thiophosphate
ggactggacccattctgcctggcc

SEQ ID NO: 43 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-methylene-4'-bridged (LNA) thymidine 5'-thiophosphate
  modified_base 21: mod_base = OTHER; note = 2'-O-(2-methoxyethyl) 5-methyl cytidine 5'-thiophosphate
  modified_base 22: mod_base = OTHER; note = 2'-O-methylene-4'-bridged (LNA) 5-methyl cytidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-methylene-4'-bridged (LNA) guanosine 5'-thiophosphate
ggactggacggaccctgggtcctg

SEQ ID NO: 44 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) 5-methyl cytidine 5'-thiophosphate
  modified_base 11: mod_base = OTHER; note = 2'-O-(2-methoxyethyl) 5-methyl cytidine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) adenosine 5'-thiophosphate
  modified_base 14: mod_base = OTHER; note = 2'-O-(2-methoxyethyl) thymidine 5'-thiophosphate
  modified_base 15: mod_base = OTHER; note = 2'-O-(2-methoxyethyl) adenosine 5'-thiophosphate
  modified_base 16: mod_base = OTHER; note = 2'-O-(2-methoxyethyl) 5-methyl cytidine 5'-thiophosphate
  modified_base 17: mod_base = OTHER; note = 2'-O-(2-methoxyethyl) thymidine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) 5-methyl cytidine 5'-thiophosphate
  modified_base 20: mod_base = OTHER; note = 2'-O-methylene-4'-bridged (LNA) thymidine 5'-thiophosphate
  modified_base 21: mod_base = OTHER; note = 2'-O-(2-methoxyethyl) guanosine 5'-thiophosphate
  modified_base 22: mod_base = OTHER; note = 2'-O-methylene-4'-bridged (LNA) thymidine 5'-thiophosphate
  modified_base 23: mod_base = OTHER; note = 2'-O-(2-methoxyethyl) guanosine 5'-thiophosphate
  modified_base 24: mod_base = OTHER; note = 2'-O-methylene-4'-bridged (LNA) thymidine 5'-thiophosphate
ggactggacccaatactgctgtgt

SEQ ID NO: 45 - RNA, 16 nt, synthetic construct
  source 1..16: mol_type = other RNA
  modified_base 1: mod_base = OTHER; note = 2'-O-(2-methoxyethyl) guanosine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) 5-methyl cytidine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) guanosine 5'-thiophosphate
  modified_base 8: mod_base = OTHER; note = 2'-O-(2-methoxyethyl) 5-methyl cytidine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) thymidine 5'-thiophosphate
  modified_base 12: mod_base = OTHER; note = 2'-O-(2-methoxyethyl) 5-methyl cytidine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) adenosine 5'-thiophosphate
ggccccgccgtcccca

SEQ ID NO: 46 - RNA, 16 nt, synthetic construct
  source 1..16: mol_type = other RNA
  modified_base 1: mod_base = OTHER; note = 2'-O-(2-methoxyethyl) guanosine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) 5-methyl cytidine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) guanosine 5'-thiophosphate
  modified_base 8: mod_base = OTHER; note = 2'-O-(2-methoxyethyl) 5-methyl cytidine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) thymidine 5'-thiophosphate
  modified_base 12: mod_base = OTHER; note = 2'-O-(2-methoxyethyl) 5-methyl cytidine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-methylene-4'-bridged (LNA) 5-methyl cytidine 5'-thiophosphate
  modified_base 16: mod_base = OTHER; note = 2'-O-methylene-4'-bridged (LNA) adenosine 5'-thiophosphate
ggccccgccgtcccca

SEQ ID NO: 47 - RNA, 15 nt, synthetic construct
  source 1..15: mol_type = other RNA
  modified_base 1: mod_base = OTHER; note = 2'-O-(2-methoxyethyl) guanosine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) 5-methyl cytidine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) guanosine 5'-thiophosphate
  modified_base 8: mod_base = OTHER; note = 2'-O-(2-methoxyethyl) 5-methyl cytidine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) thymidine 5'-thiophosphate
  modified_base 12: mod_base = OTHER; note = 2'-O-(2-methoxyethyl) 5-methyl cytidine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
ggccccgccgtcccc

SEQ ID NO: 48 - RNA, 14 nt, synthetic construct
  source 1..14: mol_type = other RNA
  modified_base 1: mod_base = OTHER; note = 2'-O-(2-methoxyethyl) guanosine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) 5-methyl cytidine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) guanosine 5'-thiophosphate
  modified_base 8: mod_base = OTHER; note = 2'-O-(2-methoxyethyl) 5-methyl cytidine 5'-thiophosphate
  modified_base 9: mod_base = OTHER; note = 2'-O-(2-methoxyethyl) 5-methyl cytidine 5'-thiophosphate
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) thymidine 5'-thiophosphate
  modified_base 12: mod_base = OTHER; note = 2'-O-(2-methoxyethyl) 5-methyl cytidine 5'-thiophosphate
  modified_base 13: mod_base = OTHER; note = 2'-O-methylene-4'-bridged (LNA) 5-methyl cytidine 5'-thiophosphate
  modified_base 14: mod_base = OTHER; note = 2'-O-methylene-4'-bridged (LNA) 5-methyl cytidine 5'-thiophosphate
ggccccgccgtccc

SEQ ID NO: 49 - RNA, 14 nt, synthetic construct
  source 1..14: mol_type = other RNA
  modified_base 1: mod_base = OTHER; note = 2'-O-(2-methoxyethyl) guanosine
  modified_base 2: mod_base = OTHER; note = 2'-O-(2-methoxyethyl) 5-methyl cytidine 5'-thiophosphate
  modified_base 3: mod_base = OTHER; note = 2'-O-(2-methoxyethyl) 5-methyl cytidine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) guanosine 5'-thiophosphate
  modified_base 7: mod_base = OTHER; note = 2'-O-(2-methoxyethyl) 5-methyl cytidine 5'-thiophosphate
  modified_base 8: mod_base = OTHER; note = 2'-O-(2-methoxyethyl) 5-methyl cytidine 5'-thiophosphate
  modified_base 9: mod_base = OTHER; note = 2'-O-(2-methoxyethyl) guanosine 5'-thiophosphate
  modified_base 10: mod_base = OTHER; note = 2'-O-(2-methoxyethyl) thymidine 5'-thiophosphate
  modified_base 11: mod_base = OTHER; note = 2'-O-(2-methoxyethyl) 5-methyl cytidine 5'-thiophosphate
  modified_base 12: mod_base = OTHER; note = 2'-O-(2-methoxyethyl) 5-methyl cytidine 5'-thiophosphate
  modified_base 13: mod_base = OTHER; note = 2'-O-methylene-4'-bridged (LNA) 5-methyl cytidine 5'-thiophosphate
  modified_base 14: mod_base = OTHER; note = 2'-O-methylene-4'-bridged (LNA) 5-methyl cytidine 5'-thiophosphate
gccccgccgtcccc

SEQ ID NO: 50 - RNA, 15 nt, synthetic construct
  source 1..15: mol_type = other RNA
  modified_base 1: mod_base = OTHER; note = 2'-O-(2-methoxyethyl) guanosine
  modified_base 2: mod_base = OTHER; note = 2'-O-(2-methoxyethyl) guanosine 5'-thiophosphate
  modified_base 3: mod_base = OTHER; note = 2'-O-(2-methoxyethyl) 5-methyl cytidine 5'-thiophosphate
  modified_base 4: mod_base = OTHER; note = 2'-O-(2-methoxyethyl) 5-methyl cytidine 5'-thiophosphate
  modified_base 5: mod_base = OTHER; note = 2'-O-(2-methoxyethyl) 5-methyl cytidine 5'-thiophosphate
  modified_base 6: mod_base = OTHER; note = 2'-O-(2-methoxyethyl) 5-methyl cytidine 5'-thiophosphate
  modified_base 7: mod_base = OTHER; note = 2'-O-(2-methoxyethyl) guanosine 5'-thiophosphate
  modified_base 8: mod_base = OTHER; note = 2'-O-(2-methoxyethyl) guanosine 5'-thiophosphate
  modified_base 9: mod_base = OTHER; note = 2'-O-(2-methoxyethyl) thymidine 5'-thiophosphate
  modified_base 10: mod_base = OTHER; note = 2'-O-(2-methoxyethyl) thymidine 5'-thiophosphate
  modified_base 11: mod_base = OTHER; note = 2'-O-(2-methoxyethyl) 5-methyl cytidine 5'-thiophosphate
  modified_base 12: mod_base = OTHER; note = 2'-O-(2-methoxyethyl) 5-methyl cytidine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-methylene-4'-bridged (LNA) 5-methyl cytidine 5'-thiophosphate
  modified_base 15: mod_base = OTHER; note = 2'-O-methylene-4'-bridged (LNA) guanosine 5'-thiophosphate
ggccccggttccccg

SEQ ID NO: 51 - skipped

SEQ ID NO: 52 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-(2-methoxyethyl) thymidine 5'-thiophosphate
  modified_base 21: mod_base = OTHER; note = 2'-O-(2-methoxyethyl) 5-methyl cytidine 5'-thiophosphate
  modified_base 22: mod_base = OTHER; note = 2'-O-(2-methoxyethyl) 5-methyl cytidine 5'-thiophosphate
  modified_base 23: mod_base = OTHER; note = 2'-O-methylene-4'-bridged (LNA) thymidine 5'-thiophosphate
  modified_base 24: mod_base = OTHER; note = 2'-O-methylene-4'-bridged (LNA) guanosine 5'-thiophosphate
ggactggacggaccctgggtcctg

SEQ ID NO: 53 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-methylene-4'-bridged (LNA) thymidine 5'-thiophosphate
  modified_base 21: mod_base = OTHER; note = 2'-O-(2-methoxyethyl) 5-methyl cytidine 5'-thiophosphate
  modified_base 22: mod_base = OTHER; note = 2'-O-methylene-4'-bridged (LNA) thymidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-methylene-4'-bridged (LNA) guanosine 5'-thiophosphate
ggactggacggaccctgggtcttg

SEQ ID NO: 54 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-methylene-4'-bridged (LNA) thymidine 5'-thiophosphate
  modified_base 21: mod_base = OTHER; note = 2'-O-(2-methoxyethyl) thymidine 5'-thiophosphate
  modified_base 22: mod_base = OTHER; note = 2'-O-methylene-4'-bridged (LNA) 5-methyl cytidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-methylene-4'-bridged (LNA) guanosine 5'-thiophosphate
ggactggacggaccctgggttctg

SEQ ID NO: 55 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-methylene-4'-bridged (LNA) thymidine 5'-thiophosphate
  modified_base 21: mod_base = OTHER; note = 2'-O-(2-methoxyethyl) 5-methyl cytidine 5'-thiophosphate
  modified_base 22: mod_base = OTHER; note = 2'-O-methylene-4'-bridged (LNA) 5-methyl cytidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-methylene-4'-bridged (LNA) guanosine 5'-thiophosphate
ggactggacggaccctgggtcctg

SEQ ID NO: 56 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-methylene-4'-bridged (LNA) thymidine
  modified_base 21: mod_base = OTHER; note = 2'-O-(2-methoxyethyl) 5-methyl cytidine 5'-thiophosphate
  modified_base 22: mod_base = OTHER; note = 2'-O-methylene-4'-bridged (LNA) 5-methyl cytidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-methylene-4'-bridged (LNA) guanosine 5'-thiophosphate
ggactggacggaccctgggtcctg

SEQ ID NO: 57 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-(2-methoxyethyl) thymidine 5'-thiophosphate
  modified_base 21: mod_base = OTHER; note = 2'-O-(2-methoxyethyl) 5-methyl cytidine 5'-thiophosphate
  modified_base 22: mod_base = OTHER; note = 2'-O-(2-methoxyethyl) 5-methyl cytidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-(2-methoxyethyl) guanosine 5'-thiophosphate
ggactggacggaccctgggtcctg

SEQ ID NO: 58 - RNA, 24 nt, synthetic construct
  source 1..24: mol_type = other RNA
  modified_base 1: mod_base = OTHER; note = 2'-O-methyl guanosine
  modified_base 2: mod_base = OTHER; note = 2'-O-methyl guanosine 5'-thiophosphate
  modified_base 3: mod_base = OTHER; note = 2'-O-methyl adenosine 5'-thiophosphate
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-O-methyl adenosine
  modified_base 9: mod_base = OTHER; note = 2'-O-methyl cytidine
  modified_base 10: mod_base = OTHER; note = 2'-O-(2-methoxyethyl) guanosine 5'-thiophosphate
  modified_base 11: mod_base = OTHER; note = 2'-O-(2-methoxyethyl) guanosine 5'-thiophosphate
  modified_base 12: mod_base = OTHER; note = 2'-O-(2-methoxyethyl) adenosine 5'-thiophosphate
  modified_base 13: mod_base = OTHER; note = 2'-O-(2-methoxyethyl) 5-methyl cytidine 5'-thiophosphate
  modified_base 14: mod_base = OTHER; note = 2'-O-(2-methoxyethyl) 5-methyl cytidine 5'-thiophosphate
  modified_base 15: mod_base = OTHER; note = 2'-O-(2-methoxyethyl) 5-methyl cytidine 5'-thiophosphate
  modified_base 16: mod_base = OTHER; note = 2'-O-(2-methoxyethyl) thymidine 5'-thiophosphate
  modified_base 17: mod_base = OTHER; note = 2'-O-(2-methoxyethyl) guanosine 5'-thiophosphate
  modified_base 18: mod_base = OTHER; note = 2'-O-(2-methoxyethyl) guanosine 5'-thiophosphate
  modified_base 19: mod_base = OTHER; note = 2'-O-(2-methoxyethyl) guanosine 5'-thiophosphate
  modified_base 20: mod_base = OTHER; note = 2'-O-(2-methoxyethyl) thymidine 5'-thiophosphate
  modified_base 21: mod_base = OTHER; note = 2'-O-(2-methoxyethyl) 5-methyl cytidine 5'-thiophosphate
  modified_base 22: mod_base = OTHER; note = 2'-O-(2-methoxyethyl) thymidine 5'-thiophosphate
  modified_base 23: mod_base = OTHER; note = 2'-O-(2-methoxyethyl) thymidine 5'-thiophosphate
  modified_base 24: mod_base = OTHER; note = 2'-O-(2-methoxyethyl) guanosine 5'-thiophosphate
ggactggacggaccctgggtcttg

SEQ ID NO: 59 - RNA, 16 nt, synthetic construct
  source 1..16: mol_type = other RNA
gcatggcgggcgcggg

SEQ ID NO: 60 - RNA, 16 nt, synthetic construct
  source 1..16: mol_type = other RNA
gacggcatggcgggcg

SEQ ID NO: 61 - RNA, 16 nt, synthetic construct
  source 1..16: mol_type = other RNA
catggccccgccgtcc

SEQ ID NO: 62 - RNA, 19 nt, synthetic construct
  source 1..19: mol_type = other RNA
cacagcctctttaaagtgc

SEQ ID NO: 63 - RNA, 16 nt, synthetic construct
  source 1..16: mol_type = other RNA
tggccccgccgtcccc

SEQ ID NO: 64 - RNA, 16 nt, synthetic construct
  source 1..16: mol_type = other RNA
actggaaaacaacacc

SEQ ID NO: 65 - RNA, 26 nt, synthetic construct
  source 1..26: mol_type = other RNA
ggactggactccattctgcctggccc

SEQ ID NO: 66 - RNA, 16 nt, synthetic construct
  source 1..16: mol_type = other RNA
ttaaagtgctgaagcc

SEQ ID NO: 67 - RNA, 25 nt, synthetic construct
  source 1..25: mol_type = other RNA
ggactggacttaaagtgctgaagcc

SEQ ID NO: 68 - skipped

SEQ ID NO: 69 - RNA, 16 nt, synthetic construct
  source 1..16: mol_type = other RNA
  modified_base 1: mod_base = OTHER; note = 2'-deoxy thymidine
  modified_base 2: mod_base = OTHER; note = 2'-deoxy thymidine
  modified_base 7: mod_base = OTHER; note = 2'-deoxy thymidine
  modified_base 10: mod_base = OTHER; note = 2'-deoxy thymidine
ttaaagtgctgaagcc

SEQ ID NO: 70 - RNA, 16 nt, synthetic construct
  source 1..16: mol_type = other RNA
ggccccgccgtcccca

SEQ ID NO: 71 - RNA, 14 nt, synthetic construct
  source 1..14: mol_type = other RNA
  modified_base 1: mod_base = OTHER; note = 2'-deoxy thymidine
  modified_base 12: mod_base = OTHER; note = 2'-deoxy thymidine
tggccccgccgtcc

SEQ ID NO: 72 - RNA, 16 nt, synthetic construct
  source 1..16: mol_type = other RNA
  modified_base 8: mod_base = OTHER; note = 2'-deoxy thymidine
  modified_base 10: mod_base = OTHER; note = 2'-deoxy thymidine
  modified_base 11: mod_base = OTHER; note = 2'-deoxy thymidine
  modified_base 13: mod_base = OTHER; note = 2'-deoxy thymidine
  modified_base 14: mod_base = OTHER; note = 2'-deoxy thymidine
gaagagatgttgttgg

SEQ ID NO: 73 - RNA, 25 nt, synthetic construct
  source 1..25: mol_type = other RNA
  modified_base 5: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggaccctgggtcctgg

SEQ ID NO: 74 - RNA, 16 nt, synthetic construct
  source 1..16: mol_type = other RNA
  modified_base 1: mod_base = OTHER; note = 2'-deoxy thymidine
  modified_base 12: mod_base = OTHER; note = 2'-deoxy thymidine
tggccccgccgtcccc

SEQ ID NO: 75 - RNA, 16 nt, synthetic construct
  source 1..16: mol_type = other RNA
ttaaagtgctggagcc

SEQ ID NO: 76 - RNA, 24 nt, synthetic construct
  source 1..24: mol_type = other RNA
  modified_base 13: mod_base = OTHER; note = 2'-deoxy thymidine
  modified_base 14: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
ggactggacccattctgcctggcc

SEQ ID NO: 77 - RNA, 24 nt, synthetic construct
  source 1..24: mol_type = other RNA
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggaccctgggtcctg

SEQ ID NO: 78 - RNA, 24 nt, synthetic construct
  source 1..24: mol_type = other RNA
  modified_base 14: mod_base = OTHER; note = 2'-deoxy thymidine
  modified_base 17: mod_base = OTHER; note = 2'-deoxy thymidine
  modified_base 20: mod_base = OTHER; note = 2'-deoxy thymidine
  modified_base 22: mod_base = OTHER; note = 2'-deoxy thymidine
  modified_base 24: mod_base = OTHER; note = 2'-deoxy thymidine
ggactggacccaatactgctgtgt

SEQ ID NO: 79 - RNA, 16 nt, synthetic construct
  source 1..16: mol_type = other RNA
  modified_base 11: mod_base = OTHER; note = 2'-deoxy thymidine
ggccccgccgtcccca

SEQ ID NO: 80 - RNA, 15 nt, synthetic construct
  source 1..15: mol_type = other RNA
  modified_base 11: mod_base = OTHER; note = 2'-deoxy thymidine
ggccccgccgtcccc

SEQ ID NO: 81 - RNA, 14 nt, synthetic construct
  source 1..14: mol_type = other RNA
  modified_base 11: mod_base = OTHER; note = 2'-deoxy thymidine
ggccccgccgtccc

SEQ ID NO: 82 - RNA, 14 nt, synthetic construct
  source 1..14: mol_type = other RNA
  modified_base 10: mod_base = OTHER; note = 2'-deoxy thymidine
gccccgccgtcccc

SEQ ID NO: 83 - RNA, 15 nt, synthetic construct
  source 1..15: mol_type = other RNA
  modified_base 9: mod_base = OTHER; note = 2'-deoxy thymidine
  modified_base 10: mod_base = OTHER; note = 2'-deoxy thymidine
ggccccggttccccg

SEQ ID NO: 84 - skipped

SEQ ID NO: 85 - RNA, 24 nt, synthetic construct
  source 1..24: mol_type = other RNA
  modified_base 16: mod_base = OTHER; note = 2'-deoxy thymidine
  modified_base 20: mod_base = OTHER; note = 2'-deoxy thymidine
  modified_base 22: mod_base = OTHER; note = 2'-deoxy thymidine
  modified_base 23: mod_base = OTHER; note = 2'-deoxy thymidine
ggactggacggaccctgggtcttg

SEQ ID NO: 86 - RNA, 24 nt, synthetic construct
  source 1..24: mol_type = other RNA
  modified_base 16: mod_base = OTHER; note = 2'-deoxy thymidine
  modified_base 20: mod_base = OTHER; note = 2'-deoxy thymidine
  modified_base 21: mod_base = OTHER; note = 2'-deoxy thymidine
  modified_base 23: mod_base = OTHER; note = 2'-deoxy thymidine
ggactggacggaccctgggttctg

SEQ ID NO: 87 - RNA, 15 nt, synthetic construct
  source 1..15: mol_type = other RNA
  modified_base 7: mod_base = OTHER; note = 2'-deoxy thymidine
  modified_base 11: mod_base = OTHER; note = 2'-deoxy thymidine
  modified_base 14: mod_base = OTHER; note = 2'-deoxy thymidine
ggaccctgggtcctg

SEQ ID NO: 88 - RNA, 15 nt, synthetic construct
  source 1..15: mol_type = other RNA
  modified_base 7: mod_base = OTHER; note = 2'-deoxy thymidine
  modified_base 11: mod_base = OTHER; note = 2'-deoxy thymidine
  modified_base 13: mod_base = OTHER; note = 2'-deoxy thymidine
  modified_base 14: mod_base = OTHER; note = 2'-deoxy thymidine
ggaccctgggtcttg

SEQ ID NO: 89 - RNA, 24 nt, synthetic construct
  source 1..24: mol_type = other RNA
  modified_base 14: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggactctgggtcctg

SEQ ID NO: 90 - RNA, 24 nt, synthetic construct
  source 1..24: mol_type = other RNA
  modified_base 13: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggatcctgggtcctg

SEQ ID NO: 91 - RNA, 24 nt, synthetic construct
  source 1..24: mol_type = other RNA
  modified_base 15: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggaccttgggtcctg

SEQ ID NO: 92 - RNA, 24 nt, synthetic construct
  source 1..24: mol_type = other RNA
  modified_base 14: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 21: mod_base = OTHER; note = 2'-deoxy thymidine
  modified_base 23: mod_base = OTHER; note = 2'-deoxy thymidine
ggactggacggactctgggttctg

SEQ ID NO: 93 - RNA, 24 nt, synthetic construct
  source 1..24: mol_type = other RNA
  modified_base 5: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggaccctgggtcctg

SEQ ID NO: 94 - RNA, 25 nt, synthetic construct
  source 1..25: mol_type = other RNA
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggaccctgggtcctgg

SEQ ID NO: 95 - RNA, 24 nt, synthetic construct
  source 1..24: mol_type = other RNA
  modified_base 5: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 22: mod_base = OTHER; note = 2'-deoxy thymidine
  modified_base 23: mod_base = OTHER; note = 2'-deoxy thymidine
ggactggacggaccctgggtcttg

SEQ ID NO: 96 - RNA, 24 nt, synthetic construct
  source 1..24: mol_type = other RNA
  modified_base 5: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 21: mod_base = OTHER; note = 2'-deoxy thymidine
  modified_base 23: mod_base = OTHER; note = 2'-deoxy thymidine
ggactggacggaccctgggttctg

SEQ ID NO: 97 - RNA, 24 nt, synthetic construct
  source 1..24: mol_type = other RNA
  modified_base 5: mod_base = OTHER; note = 2'-deoxy thymidine
  modified_base 14: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggactctgggtcctg

SEQ ID NO: 98 - RNA, 24 nt, synthetic construct
  source 1..24: mol_type = other RNA
  modified_base 5: mod_base = OTHER; note = 2'-deoxy thymidine
  modified_base 13: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggatcctgggtcctg

SEQ ID NO: 99 - RNA, 24 nt, synthetic construct
  source 1..24: mol_type = other RNA
  modified_base 5: mod_base = OTHER; note = 2'-deoxy thymidine
  modified_base 15: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 23: mod_base = OTHER; note = 2'-deoxy thymidine
ggactggacggaccttgggtcctg

SEQ ID NO: 100 - RNA, 25 nt, synthetic construct
  source 1..25: mol_type = other RNA
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccctgggtcctg

SEQ ID NO: 101 - RNA, 25 nt, synthetic construct
  source 1..25: mol_type = other RNA
  modified_base 10: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccctgggtcctg

SEQ ID NO: 102 - RNA, 25 nt, synthetic construct
  source 1..25: mol_type = other RNA
  modified_base 5: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccctgggtcctg

SEQ ID NO: 103 - RNA, 25 nt, synthetic construct
  source 1..25: mol_type = other RNA
  modified_base 5: mod_base = OTHER; note = 2'-deoxy thymidine
  modified_base 10: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccctgggtcctg

SEQ ID NO: 104 - RNA, 26 nt, synthetic construct
  source 1..26: mol_type = other RNA
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccctgggtcctgg

SEQ ID NO: 105 - RNA, 26 nt, synthetic construct
  source 1..26: mol_type = other RNA
  modified_base 10: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccctgggtcctgg

SEQ ID NO: 106 - RNA, 26 nt, synthetic construct
  source 1..26: mol_type = other RNA
  modified_base 5: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccctgggtcctgg

SEQ ID NO: 107 - RNA, 25 nt, synthetic construct
  source 1..25: mol_type = other RNA
  modified_base 17: mod_base = OTHER; note = 2'-deoxy thymidine
  modified_base 21: mod_base = OTHER; note = 2'-deoxy thymidine
  modified_base 23: mod_base = OTHER; note = 2'-deoxy thymidine
  modified_base 24: mod_base = OTHER; note = 2'-deoxy thymidine
ggactggactggaccctgggtcttg

SEQ ID NO: 108 - RNA, 25 nt, synthetic construct
  source 1..25: mol_type = other RNA
  modified_base 10: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 23: mod_base = OTHER; note = 2'-deoxy thymidine
  modified_base 24: mod_base = OTHER; note = 2'-deoxy thymidine
ggactggactggaccctgggtcttg

SEQ ID NO: 109 - RNA, 25 nt, synthetic construct
  source 1..25: mol_type = other RNA
  modified_base 5: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 23: mod_base = OTHER; note = 2'-deoxy thymidine
  modified_base 24: mod_base = OTHER; note = 2'-deoxy thymidine
ggactggactggaccctgggtcttg

SEQ ID NO: 110 - RNA, 25 nt, synthetic construct
  source 1..25: mol_type = other RNA
  modified_base 17: mod_base = OTHER; note = 2'-deoxy thymidine
  modified_base 21: mod_base = OTHER; note = 2'-deoxy thymidine
  modified_base 22: mod_base = OTHER; note = 2'-deoxy thymidine
  modified_base 24: mod_base = OTHER; note = 2'-deoxy thymidine
ggactggactggaccctgggttctg

SEQ ID NO: 111 - RNA, 25 nt, synthetic construct
  source 1..25: mol_type = other RNA
  modified_base 10: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 22: mod_base = OTHER; note = 2'-deoxy thymidine
  modified_base 24: mod_base = OTHER; note = 2'-deoxy thymidine
ggactggactggaccctgggttctg

SEQ ID NO: 112 - RNA, 25 nt, synthetic construct
  source 1..25: mol_type = other RNA
  modified_base 5: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 22: mod_base = OTHER; note = 2'-deoxy thymidine
  modified_base 24: mod_base = OTHER; note = 2'-deoxy thymidine
ggactggactggaccctgggttctg

SEQ ID NO: 113 - RNA, 25 nt, synthetic construct
  source 1..25: mol_type = other RNA
  modified_base 15: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggactctgggtcctg

SEQ ID NO: 114 - RNA, 25 nt, synthetic construct
  source 1..25: mol_type = other RNA
  modified_base 10: mod_base = OTHER; note = 2'-deoxy thymidine
  modified_base 15: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggactctgggtcctg

SEQ ID NO: 115 - RNA, 25 nt, synthetic construct
  source 1..25: mol_type = other RNA
  modified_base 5: mod_base = OTHER; note = 2'-deoxy thymidine
  modified_base 15: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggactctgggtcctg

SEQ ID NO: 116 - RNA, 25 nt, synthetic construct
  source 1..25: mol_type = other RNA
  modified_base 14: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggatcctgggtcctg

SEQ ID NO: 117 - RNA, 25 nt, synthetic construct
  source 1..25: mol_type = other RNA
  modified_base 10: mod_base = OTHER; note = 2'-deoxy thymidine
  modified_base 14: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggatcctgggtcctg

SEQ ID NO: 118 - RNA, 25 nt, synthetic construct
  source 1..25: mol_type = other RNA
  modified_base 5: mod_base = OTHER; note = 2'-deoxy thymidine
  modified_base 14: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggatcctgggtcctg

SEQ ID NO: 119 - RNA, 25 nt, synthetic construct
  source 1..25: mol_type = other RNA
  modified_base 16: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccttgggtcctg

SEQ ID NO: 120 - RNA, 25 nt, synthetic construct
  source 1..25: mol_type = other RNA
  modified_base 10: mod_base = OTHER; note = 2'-deoxy thymidine
  modified_base 16: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccttgggtcctg

SEQ ID NO: 121 - RNA, 25 nt, synthetic construct
  source 1..25: mol_type = other RNA
  modified_base 5: mod_base = OTHER; note = 2'-deoxy thymidine
  modified_base 16: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 24: mod_base = OTHER; note = 2'-deoxy thymidine
ggactggactggaccttgggtcctg

SEQ ID NO: 122 - RNA, 25 nt, synthetic construct
  source 1..25: mol_type = other RNA
  modified_base 15: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 22: mod_base = OTHER; note = 2'-deoxy thymidine
  modified_base 24: mod_base = OTHER; note = 2'-deoxy thymidine
ggactggactggactctgggttctg

SEQ ID NO: 123 - RNA, 25 nt, synthetic construct
  source 1..25: mol_type = other RNA
  modified_base 10: mod_base = OTHER; note = 2'-deoxy thymidine
  modified_base 15: mod_base = OTHER; note = 2'-deoxy thymidine
  modified_base 17: mod_base = OTHER; note = 2'-deoxy thymidine
  modified_base 21: mod_base = OTHER; note = 2'-deoxy thymidine
  modified_base 22: mod_base = OTHER; note = 2'-deoxy thymidine
  modified_base 24: mod_base = OTHER; note = 2'-deoxy thymidine
ggactggactggactctgggttctg

SEQ ID NO: 124 - RNA, 24 nt, synthetic construct
  source 1..24: mol_type = other RNA
  modified_base 5: mod_base = OTHER; note = 2'-deoxy thymidine
  modified_base 14: mod_base = OTHER; note = 2'-deoxy thymidine
  modified_base 16: mod_base = OTHER; note = 2'-deoxy thymidine
  modified_base 20: mod_base = OTHER; note = 2'-deoxy thymidine
  modified_base 21: mod_base = OTHER; note = 2'-deoxy thymidine
  modified_base 23: mod_base = OTHER; note = 2'-deoxy thymidine
ggactggacggactctgggttctg